(19) United States
(12) Patent Application Publication
(10) Pub. No.: US 2023/0212191 A1
(43) Pub. Date: Jul. 6, 2023

(54) SPIRO-SULFONIMIDAMIDE DERIVATIVES AS INHIBITORS OF MYELOID CELL LEUKEMIA-1 (MCL-1) PROTEIN

(71) Applicant: PRELUDE THERAPEUTICS, INCORPORATED, Wilmingon, DE (US)

(72) Inventors: Jincong ZHUO, Garnet Valley, PA (US); Andrew Paul COMBS, Kennett Square, PA (US)

(73) Assignee: PRELUDE THERAPEUTICS, INCORPORATED, Wilmingon, DE (US)

(21) Appl. No.: 17/996,305

(22) PCT Filed: Apr. 16, 2021
(86) PCT No.: PCT/US2021/027602

§ 371 Date: Oct. 14, 2022

Related U.S. Application Data
(60) Provisional application No. 63/010,809, filed on Apr. 16, 2020.

(51) Int. Cl. C07D 515/10; A61P 35/00

(52) U.S. Cl. CPC C07D 515/10; A61P 35/00

(57) ABSTRACT

The disclosure is directed to compounds of Formula (I). Pharmaceutical compositions comprising compounds of Formula (I) as well as methods of their use and preparation, are also described.

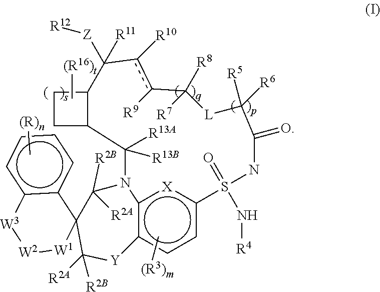

### CROSS-REFERENCE TO RELATED APPLICATION

This application claims the benefit of priority to U.S. Provisional Application No. 63/010,809, filed on Apr. 16, 2020, the entirety of which is incorporated by reference herein.

### TECHNICAL FIELD

The disclosure is directed to MCL-1 inhibitors and methods of their use.

### BACKGROUND

Apoptosis (programmed cell death) is a highly conserved cellular process that is required for embryonic development and normal tissue homeostasis (Ashkenazi A. et al., Nat. Rev. Drug Discov. 2017, 16, 273-284). Apoptotic-type cell death involves morphological changes such as condensation of the nucleus, DNA fragmentation as well as biochemical phenomena such as the activation of caspases which cause damage to key structural components of the cell, resulting in its disassembly and death. Regulation of the process of apoptosis is complex and involves the activation or repression of several intracellular signaling pathways (Cory S. et al., Nature Review Cancer 2002, 2, 647-656; Thomas L. W. et al., FEBS Lett. 2010, 584, 2981-2989; Adams J. M. et al., Oncogene 2007, 26, 1324-1337)

The Bcl-2 protein family, which includes both pro-apoptotic and anti-apoptotic members, plays a pivotal role in the regulation of the apoptosis process (Youle R. J. et al., Nat. Rev. Mol. Cell Biol. 2008, 9, 47-59; Kelly G. L. et al., Adv. Cancer Res. 2011, 111, 39-96). Bcl-2, Bcl-XL, Bcl-W, Mcl-1 and A1 are anti-apoptotic proteins and they share a common BH regions. In contrast, the pro-apoptotic family members are divided into two groups. The multi-region pro-apoptotic proteins, such as Bax, Bak and Bok, are conventionally thought to have BH1-3 regions, whereas the BH3-only proteins are proposed to share homology in the BH3 region only. Members of BH3-only proteins include Bad, Bim, Bid, Noxa, Puma, Bik/Blk, Bmf, Hrk/DP5, Beclin-1 and Mule (Xu G. et al., Bioorg. Med. Chem. 2017, 25, 5548-5556; Hardwick J. M. et al., Cell. 2009, 138, 404; Reed J. C., Cell Death Differ. 2018, 25, 3-6; Kang M. H. et al., Clin Cancer Res 2009, 15, 1126-1132). The pro-apoptotic members (such as BAX and BAK), upon activation, form a homo-oligomer in the outer mitochondrial membrane that leads to pore formation and the escape of mitochondrial contents, a step into triggering apoptosis. Antiapoptotic members of the Bcl-2 family (such as Bel-2, Bel-XL, and Mcl-1) block the activity of BAX and BAK. In normal cells, this process is tightly regulated. Abnormal cells can dysregulate this process to avoid cell death. One of the ways that cancer cells can accomplish this is by upregulating the antiapoptotic members of the Bcl-2 family of proteins. Overexpression or up-regulation of the anti-apoptotic Bcl-2 family proteins enhance cancer cell survival and cause resistance to a variety of anticancer therapies.

Aberrant expression or function of the proteins responsible for apoptotic signaling contributes to numerous human pathologies including auto-immune diseases, neurodegeneration (such as Parkinson's disease, Alzheimer's disease and ischaemia), inflammatory diseases, viral infections and cancer (such as colon cancer, breast cancer, small-cell lung cancer, non-small-cell lung cancer, bladder cancer, ovarian cancer, prostate cancer, chronic lymphoid leukemia, lymphoma, myeloma, acute myeloid leukemia, pancreatic cancer, etc.) (Hanahan D. et al., Cell 2000, 100. 57-70). Herein, it is prospective to target key apoptosis regulators for cancer treatment (Kale J. et al., Cell Death Differ. 2018, 25, 65-80; Vogler M. et al., Cell Death Differ. 2009, 16, 360-367).

By overexpressing one or more of these pro-survival proteins, cancer cells can evade elimination by normal physiological processes and thus gain a survival advantage. Myeloid Cell Leukemia-1 (Mcl-1) is a member of the pro-survival Bcl-2 family of proteins. Mcl-1 has the distinct trait of being essential for embryonic development as well as the survival of all hematopoietic lineages and progenitor populations. Mcl-1 is one of the most common genetic aberrations in human cancer and is highly expressed in many tumor types. Mcl-1 overexpression in human cancers is associated with high tumor grade and poor survival (Beroukhim R. et al., Nature 2010, 463, 899-905). Mcl-1 overexpression prevents cancer cells from undergoing programmed cell death (apoptosis), allowing the cells to survive despite widespread genetic damage. Further, its amplification is associated with both intrinsic and acquired resistance to a wide variety of antitumorigenic agents including chemotherapeutic agents such as microtubule binding agents, paclitaxel and gemcitabine, as well as apoptosis-inducing agents such as TRAIL, the Bcl-2 inhibitor, venetoclax, and the Bcl-2/Bcl-XL dual inhibitor navitoclax. Not only do gene silencing approaches that specifically target Mcl-1 circumvent this resistance phenotype, but certain cancer cell types frequently undergo cell death in response to Mcl-1 silencing, indicating a dependence on Mcl-1 for survival. Consequently, approaches that inhibit Mcl-1 function are of considerable interest for cancer therapy (Wertz I. E et al., Nature 2011, 471, 110-114; Zhang B. et al., Blood 2002, 99, 1885-1893).

### SUMMARY

The disclosure is directed to compounds of Formula I:

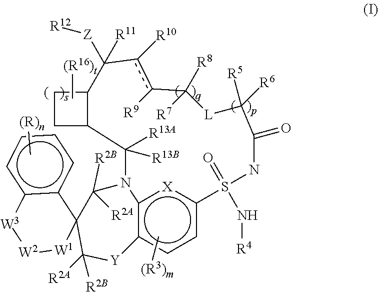

or a pharmaceutically acceptable salt or solvate thereof;

wherein
L is absent, —NR¹⁴—, —O—, —S—, —S(O)—, or —S(O)₂—, arylene, —O-arylene, cycloalkylene, —O— cycloalkylene, cycloalkenylene, spirocycloalkylene, heteroarylene, heterocycloalkylene, —O-heterocycloalkylene, heterocycloalkenylene, or spiroheterocycloalkylene wherein said arylene, cycloalkylene, cycloalkenylene, spirocycloalkylene, heteroarylene, heterocycloalkylene, heterocycloalkenylene, or spiroheterocycloalkylene is optionally substituted;
[symbol] is single bond or double bond;
X is CH or N;
Y is —O—, —S—, —S(O)—, or —S(O)₂—;
Z is —NR¹⁵—, —O—, or —S—;
the moiety —W¹—W²—W³ is —CR^{1A}R^{1B}—CR^{1C}R^{1D}—CR^{1A}R^{1B}—, —O—CR^{1C}R^{1D}—CR^{1A}R^{1B}—, —CR^{1A}R^{1B}—CR^{1C}R^{1D}—O—, —NR^{1C}—CR^{1C}R^{1D}—CR^{1A}R^{1B}—, —CR^{1A}R^{1B}—CR^{1C}R^{1D}—NR^{1C}—, —S—CR^{1C}R^{1D}—CR^{1A}R^{1B}, or —CR^{1A}R^{1B}—CR^{1C}R^{1D}—S—;
each n is independently 0-3;
each m is independently 0-2;
each p is independently 0-4;
each q is independently 0-4;
each s is independently 0-3;
each t is independently 0-6;
each R is independently -D, -halo, —CN, —NO₂, —C₁-C₆alkyl, —C₂-C₆alkenyl, —C₁-C₆alkoxy, -cycloalkyl, —OR^{a}, —SR^{a}, —C(O)R^{b}, —C(O)OR^{b}, —NR^{c}R^{d}, —NR^{a}R^{c}, —C(O)NR^{c}R^{d}, —S(O)R^{b}, —S(O)₂R^{b}, wherein said —C₁-C₆alkyl, —C₂-C₆alkenyl, —C₁-C₆alkoxy, or -cycloalkyl is optionally substituted;
each R^{1A}, or R^{1B} is independently H, D, halo, optionally substituted C₁-C₆alkyl, or R^{1A} and R^{1B} that are attached to the same carbon atom may, together with the carbon atom to which they are both attached, form an optionally substituted cycloalkyl ring;
each R^{1C} and R^{1D} is independently H, D, fluoro, optionally substituted C₁-C₆alkyl, or R^{1C} and R^{1D} may, together with the carbon atom to which they are both attached, form an optionally substituted cycloalkyl ring;
or R^{1B} and R^{1C} that are attached to adjacent carbon atoms may, together with the carbon atoms to which they are attached, form an optionally substituted cycloalkyl ring;
each R^{2A} and R^{2B} is independently H, D, fluoro, optionally substituted C₁-C₆alkyl, or R^{2A} and R^{2B} may, together with the carbon atom to which they are both attached, form an optionally substituted cycloalkyl ring;
each R³ is independently -D, -halo, —CN, —NO₂, —C₁-C₆alkyl, —C₂-C₆alkenyl, —C₂-C₆alkynyl, —OR^{a}, —SR^{a}, —NR^{c}R^{d}, —NR^{a}R^{c}, —C(O)R^{b}, —OC(O)R^{b}, —C(O)OR^{b}, —C(O)NR^{c}R^{d}, —S(O)₂R^{b};
aryl, -heteroaryl, -cycloalkyl, or -heterocycloalkyl, wherein said —C₁-C₆alkyl, —C₂-C₆alkenyl, —C₂-C₆alkynyl, -cycloalkyl, -heterocycloalkyl, -aryl, or -heteroaryl is optionally substituted;
R⁴ is H, —C(O)OR^{4A}, —C(O)R^{4B}, —C(O)NR^{4C}R^{4D}, —S(O)R^{4B}, —S(O)₂R^{4B}, —S(O)NR^{4C}R^{4D}, or —S(O)₂NR^{4C}R^{4D};
each R^{4A} is independently —C₁-C₁₀alkyl, —C₃-C₁₀ alkenyl, —C₃-C₁₀ alkynyl, aryl, cycloalkyl, C₁-C₆heteroalkyl, heteroaryl, heterocycloalkyl, or heterocycloalkenyl wherein said C₁-C₁₀ alkyl, C₃-C₁₀ alkenyl, C₃-C₁₀ alkynyl, aryl, cycloalkyl, heteroaryl, heterocycloalkyl, or heterocycloalkenyl is optionally substituted;
each R^{4B} is independently H, D, —C₁-C₆ alkyl, —C₂-C₆ alkenyl, —C₂-C₆ alkynyl, aryl, cycloalkyl, C₁-C₆heteroalkyl, heteroaryl, heterocycloalkyl, or heterocycloalkenyl wherein said —C₁-C₆ alkyl, —C₂-C₆ alkenyl, —C₂-C₆ alkynyl, aryl, cycloalkyl, heteroaryl, heterocycloalkyl, or heterocycloalkenyl is optionally substituted;
each R^{4C} or R^{4D} is independently H, D, —C₁-C₁₀ alkyl, —C₂-C₆ alkenyl, —C₂-C₆ alkynyl, —OC₁-C₆alkyl, —O-cycloalkyl, aryl, heteroaryl, cycloalkyl, C₁-C₆heteroalkyl, heterocycloalkyl, or heterocycloalkenyl, wherein said C₁-C₁₀ alkyl, C₂-C₆ alkenyl, C₂-C₆ alkynyl, —OC₁-C₆alkyl, —O-cycloalkyl, aryl, heteroaryl, cycloalkyl, heterocycloalkyl, or heterocycloalkenyl are each optionally substituted;
or R^{4C} and R^{4D}, together with the N atom to which they are both attached, form an optionally substituted monocyclic or multicyclic heterocycloalkyl, or optionally substituted monocyclic or multicyclic heterocycloalkenyl group;
each R⁵, R⁷ or R¹¹ is independently H, D, halo, —OH, —CN, —NO₂, —C₁-C₆alkyl, —C₂-C₆alkenyl, —C₂-C₆alkynyl, aryl, heteroaryl, cycloalkyl, heterocycloalkyl, heterocycloalkenyl, —OR^{a}, —SR^{a}, —NR^{c}R^{d}, —NR^{a}R^{c}, —C(O)R^{b}, —OC(O)R^{b}, —C(O)OR^{b}, —C(O)NR^{c}R^{d}, —S(O)R^{b}, or —S(O)₂R^{b}, wherein said C₁-C₆alkyl, —C₂-C₆alkenyl, —C₂-C₆alkynyl, aryl, heteroaryl, cycloalkyl, heterocycloalkenyl, or heterocycloalkyl is optionally substituted;
each R⁶ or R⁸ is independently H, D, halo, —C₁-C₆alkyl, —C₂-C₆alkenyl, —C₂-C₆alkynyl, —C(O)R^{b}, —C(O)OR^{a}, —C(O)NR^{c}R^{d}, aryl, heteroaryl, cycloalkyl, cycloalkenyl, heterocycloalkyl, or heterocycloalkenyl, wherein said C₁-C₆alkyl, —C₂-C₆alkenyl, —C₂-C₆alkynyl, aryl, heteroaryl, cycloalkyl, heterocycloalkyl, or heterocycloalkenyl is optionally substituted;
or R⁵ and R⁶ together with the C atom to which they are attached form a cycloalkyl, heterocycloalkyl, or heterocycloalkenyl ring, each optionally substituted;
or an R⁵ and an R⁶ attached to adjacent carbon atoms, together with the C atoms to which they are attached, form a cycloalkyl, heterocycloalkyl, or heterocycloalkenyl ring, each optionally substituted;
or R⁷ and R⁸ together with the C atom to which they are both attached form a cycloalkyl, heterocycloalkyl, or heterocycloalkenyl ring, each optionally substituted, each optionally substituted;
or an R⁷ and an R⁸ attached to adjacent carbon atoms, together with the C atoms to which they are attached, form a cycloalkyl, heterocycloalkyl, or heterocycloalkenyl ring, each optionally substituted;
each R⁹ or R¹⁰ is independently H, D, -Me, CN, —CH₂CN, —CH₂F, —CHF₂, —CF₃ or —F;
each R¹² is H, D, —C(O)R^{b}, —C(O)OR^{c}, —C(O)NR^{c}R^{d}, P(OR^{c})₂, P(O)R^{c}R^{b}, P(O)OR^{c}OR^{b}, S(O)R^{b}, S(O)NR^{c}R^{d}, S(O)₂R^{b}, S(O)₂NR^{c}R^{d}, B(OR)(OR^{b}), SiR^{b}₃, C₁-C₁₀alkyl, C₂-C₁₀ alkenyl, C₂-C₁₀ alkynyl, —(CH₂CH₂O)ₒR^{a} wherein o=1 to 10, aryl, cycloalkyl, heteroaryl, or heterocycloalkyl, wherein said C₁-C₁₀ alkyl, C₂-C₁₀alkenyl, C₂-C₁₀ alkynyl, aryl, cycloalkyl, heteroaryl, or heterocycloalkyl is optionally substituted;
or R¹² and R¹¹ form an optionally substituted monocyclic or multicyclic heterocycloalkyl, or optionally substituted monocyclic or multicyclic heterocycloalkenyl group;
each R^{13A} or R^{13B} is independently H, D, optionally substituted C₁-C₆alkyl;
or R^{13A} and R^{13B} that are attached to the same carbon atom may, together with the carbon atom to which they are both attached, form an optionally substituted cycloalkyl ring;
each R¹⁴ or R¹⁵ is independently H, D, —C₁-C₆alkyl, —C₂-C₆alkenyl, —C₂-C₆alkynyl, —OC₁-C₆alkyl, —C(O)R^{b}, —C(O)OR^{b}, —C(O)NR^{c}R^{d}, —S(O)R^{b} or —S(O)₂R^{b}, aryl, heteroaryl, cycloalkyl, heterocycloalkyl, or heterocycloalkenyl group, wherein said C₁-C₆alkyl, —C₂-C₆alkenyl, —C₂-C₆alkynyl, —OC₁-C₆alkyl, aryl, heteroaryl, cycloalkyl, heterocycloalkyl, or heterocycloalkenyl ring, is optionally substituted;
or R¹⁴ together with an R⁴, R⁵, R⁷ or an R⁸ form an optionally substituted monocyclic or multicyclic heterocycloalkyl, or optionally substituted monocyclic or multicyclic heterocycloalkenyl group;
each R¹⁶ is independently H, D, —OH, -Me, —CH₂F, —CHF₂, —CF₃ or —F;
each R^{a} is independently H, D, —C(O)R^{b}, —C(O)OR^{c}, —C(O)NR^{c}R^{d}, —P(OR^{c})₂, —P(O)R^{c}R^{b}, —P(O)OR^{c}OR^{b}, —S(O)R^{b}, —S(O)NR^{c}R^{d}, —S(O)₂R^{b}, —S(O)₂NR^{c}R^{d}, —B(OR^{c})(OR^{b}), SiR^{b}₃, —C₁-C₁₀alkyl, —C₂-C₁₀ alkenyl, —C₂-C₁₀ alkynyl, aryl, cycloalkyl, heteroaryl, heterocycloalkyl, or heterocycloalkenyl wherein said C₁-C₁₀ alkyl, C₂-C₁₀ alkenyl, C₂-C₁₀ alkynyl, aryl, cycloalkyl, heteroaryl, heterocycloalkyl, or heterocycloalkenyl is optionally substituted;
each R^{b}, is independently H, D, —C₁-C₆ alkyl, —C₂-C₆ alkenyl, —C₂-C₆ alkynyl, aryl, cycloalkyl, heteroaryl, heterocycloalkyl, or heterocycloalkenyl wherein said —C₁-C₆ alkyl, —C₂-C₆ alkenyl, —C₂-C₆ alkynyl, aryl, cycloalkyl, heteroaryl, heterocycloalkyl, or heterocycloalkenyl is optionally substituted;
each R^{c} or R^{d} is independently H, D, —C₁-C₁₀ alkyl, —C₂-C₆ alkenyl, —C₂-C₆ alkynyl, —OC₁-C₆alkyl, —O-cycloalkyl, aryl, heteroaryl, cycloalkyl, heterocycloalkyl, or heterocycloalkenyl, wherein said C₁-C₁₀ alkyl, C₂-C₆ alkenyl, C₂-C₆ alkynyl, —OC₁-C₆alkyl, —O-cycloalkyl, aryl, heteroaryl, cycloalkyl, heterocycloalkyl, or heterocycloalkenyl are each optionally substituted;
or R^{c} and R^{d}, together with the N atom to which they are both attached, form an optionally substituted monocyclic or multicyclic heterocycloalkyl, or optionally substituted monocyclic or multicyclic heterocycloalkenyl group.

Stereoisomers of the compounds of Formula I and the pharmaceutical salts and solvates thereof, are also contemplated, described, and encompassed herein. Methods of using compounds of Formula I are described, as well as pharmaceutical compositions including the compounds of Formula I.

### DETAILED DESCRIPTION OF ILLUSTRATIVE EMBODIMENTS

The disclosure may be more fully appreciated by reference to the following description, including the following definitions and examples. Certain features of the disclosed compositions and methods which are described herein in the context of separate aspects, may also be provided in combination in a single aspect. Alternatively, various features of the disclosed compositions and methods that are, for brevity, described in the context of a single aspect, may also be provided separately or in any subcombination.

The term “alkyl,” when used alone or as part of a substituent group, refers to a straight- or branched-chain hydrocarbon group having from 1 to 12 carbon atoms (“C₁-C₁₂”), preferably 1 to 6 carbons atoms (“C₁-C₆”), in the group. Examples of alkyl groups include methyl (Me, C₁alkyl), ethyl (Et, C₂alkyl), n-propyl (C₃alkyl), isopropyl (C₃alkyl), butyl (C₄alkyl), isobutyl (C₄alkyl), sec-butyl (C₄alkyl), tert-butyl (C₄alkyl), pentyl (C₅alkyl), isopentyl (C₅alkyl), tert-pentyl (C₅alkyl), hexyl (C₆alkyl), isohexyl (C₆alkyl), and the like.

The term “haloalkyl,” when used alone or as part of a substituent group, refers to a straight- or branched-chain hydrocarbon group having from 1 to 12 carbon atoms (“C₁-C₁₂”), preferably 1 to 6 carbons atoms (“C₁-C₆”), in the group, wherein one or more of the hydrogen atoms in the group have been replaced by a halogen atom. Examples of haloalkyl groups include trifluoromethyl (—CF₃, C₁haloalkyl), trifluoroethyl (—CH₂CF₃, C₂haloalkyl), and the like.

The term “heteroalkyl” refers to an alkyl group in which one or more of the carbon atoms (and any associated hydrogen atoms) are each independently replaced with the same or different heteroatomic group. The term “heteroalkyl” includes unbranched or branched saturated chain having carbon and heteroatoms selected from nitrogen, sulfur, phosphorus, and oxygen. The heteroatoms within the “heteroalkyl” may be oxidized, e.g. —N(O)—, —S(O)—, —S(O)₂—. Examples of heteroalkyl groups include —OCH₃, —CH₂OCH₃, —SCH₃, —CH₂SCH₃, —NRCH₃, and —CH₂NRCH₃, where R is hydrogen or alkyl.

As used herein, “cycloalkyl” refers to non-aromatic cyclic hydrocarbons including cyclized alkyl and/or alkenyl groups. Cycloalkyl groups thus also encompass cycloalkenyl groups. Cycloalkyl groups can include mono- or polycyclic (e.g., having 2 fused rings) groups, spirocycles, and bridged rings (e.g., a bridged bicycloalkyl group). Ring-forming carbon atoms of a cycloalkyl group can be optionally substituted by oxo or sulfido (e.g., C(O) or C(S)). Also included in the definition of cycloalkyl are moieties that have one or more aromatic rings fused (i.e., having a bond in common with) to the cycloalkyl ring, for example, benzo or thienyl derivatives of cyclopentane, cyclohexane, and the like. A cycloalkyl group containing a fused aromatic ring can be attached through any ring-forming atom including a ring-forming atom of the fused aromatic ring. Cycloalkyl groups can have 3, 4, 5, 6, 7, 8, 9, or 10 ring-forming carbons (C₃-10). In some embodiments, the cycloalkyl is a C₃-10 monocyclic or bicyclic cycloalkyl. In some embodiments, the cycloalkyl is a C₃-10 monocyclic or bicyclic cycloalkyl which is optionally substituted by CH₂F, CHF₂, CF₃, and CF₂CF₃. In some embodiments, the cycloalkyl is a C₃₋₇ monocyclic cycloalkyl. In some embodiments, the cycloalkyl is a C₄₋₁₀ spirocycle or bridged cycloalkyl. Example cycloalkyl groups include cyclopropyl, cyclobutyl, cyclopentyl, cyclohexyl, cycloheptyl, cyclopentenyl, cyclohexenyl, cyclohexadienyl, cycloheptatrienyl, norbornyl, norpinyl, norcarnyl, cubane, adamantane, bicyclo[1. 1. 1]pentyl, bicyclo[2.1.1]hexyl, bicyclo[2.2.1]heptanyl, bicyclo[3.1.1]heptanyl, bicyclo[2.2.2]octanyl, spiro[3.3]heptanyl, and the like. In some embodiments, cycloalkyl is cyclopropyl, cyclobutyl, cyclopentyl, or cyclohexyl. In some embodiments, cycloalkyl are cyclic-containing, non-aromatic hydrocarbon groups having from 3 to 12 carbon atoms (“C₃-C₁₂”), preferably from 3 to 6 carbon atoms (“C₃-C₆”). Examples of cycloalkyl groups include, for example, cyclopropyl (C₃; 3-membered), cyclobutyl (C₄; 4-membered), cyclopropylmethyl (C₄), cyclopentyl (C₅), cyclohexyl (C₆), 1-methylcyclopropyl (C₄), 2-methylcyclopentyl (C₄), adamantanyl (C₁₀), and the like.

The term “cycloalkylene” when used alone or as part of a substituent group refers to a cycloalkyl diradical, i.e., a cyclic-containing, non-aromatic hydrocarbon group having from 3 to 14 carbon atoms (“C₃-C₁₄”; or 3-14 membered), for example 3 to 12 carbon atoms (“C₃-C₁₂”), preferably from preferably from 3 to 7 carbon atoms (“C₃-C₇”, or 3-7 membered) or 3 to 6 carbon atoms (“C₃-C₆”), wherein the group is directly attached to two other variable groups. Cycloalkylene groups include spirocycloalkylene groups.

The term “cycloalkenylene” refers to a cycloalkenylene diradical.

The term “spirocycloalkyl” when used alone or as part of a substituent group refers to a non-aromatic hydrocarbon group containing two cycloalkyl rings, and wherein the two cycloalyl rings share a single carbon atom in common.

The term “spirocycloalkylene” when used alone or as part of a substituent group refers to a spirocycloalkyl diradical, i.e., a non-aromatic hydrocarbon group containing two cycloalkyl rings, and wherein the two cycloalyl rings share a single carbon atom in common, and wherein the group is directly attached to two other variable groups.

As used herein, “heterocycloalkyl” refers to monocyclic or polycyclic heterocycles having at least one non-aromatic ring (saturated or partially unsaturated ring), wherein one or more of the ring-forming carbon atoms of the heterocycloalkyl is replaced by a heteroatom selected from N, O, S and B, and wherein the ring-forming carbon atoms and heteroatoms of the heterocycloalkyl group can be optionally substituted by one or more oxo or sulfido (e.g., C(O), S(O), C(S), or S(O)₂, etc.). Heterocycloalkyl groups include monocyclic and polycyclic (e.g., having 2 fused rings) systems. Included in heterocycloalkyl are monocyclic and polycyclic 3-10, 4-10, 3-7, 4-7, and 5-6 membered heterocycloalkyl groups. Heterocycloalkyl groups can also include spirocycles and bridged rings (e.g., a 5-10 membered bridged biheterocycloalkyl ring having one or more of the ring-forming carbon atoms replaced by a heteroatom independently selected from N, O, S and B). The heterocycloalkyl group can be attached through a ring-forming carbon atom or a ring-forming heteroatom. In some embodiments, the heterocycloalkyl group contains 0 to 3 double bonds. In some embodiments, the heterocycloalkyl group contains 0 to 2 double bonds.

Also included in the definition of heterocycloalkyl are moieties that have one or more aromatic rings fused (i.e., having a bond in common with) to the non-aromatic heterocyclic ring, for example, benzo or thienyl derivatives of piperidine, morpholine, azepine, etc. A heterocycloalkyl group containing a fused aromatic ring can be attached through any ring-forming atom including a ring-forming atom of the fused aromatic ring. In some embodiments, the heterocycloalkyl group contains 3 to 10 ring-forming atoms, 4 to 10 ring-forming atoms, 3 to 7 ring-forming atoms, or 5 to 6 ring-forming atoms. In some embodiments, the heterocycloalkyl group has 1 to 4 heteroatoms, 1 to 3 heteroatoms, 1 to 2 heteroatoms or 1 heteroatom. In some embodiments, the heterocycloalkyl is a monocyclic 4-6 membered heterocycloalkyl having 1 or 2 heteroatoms independently selected from N, O, S and B and having one or more oxidized ring members.

Example heterocycloalkyl groups include pyrrolidin-2-one, 1,3-isoxazolidin-2-one, pyranyl, tetrahydropyran, oxetanyl, azetidinyl, morpholino, thiomorpholino, piperazinyl, tetrahydrofuranyl, tetrahydrothienyl, piperidinyl, pyrrolidinyl, isoxazolidinyl, isothiazolidinyl, pyrazolidinyl, oxazolidinyl, thiazolidinyl, imidazolidinyl, azepanyl, benzazapene, 1,2,3,4-tetrahydroisoquinoline, azabicyclo[3.1.0]hexanyl, diazabicyclo[3. 1.0]hexanyl, oxabicyclo[2.1.1]hexanyl, azabicyclo[2.2.1]heptanyl, diazabicyclo[2.2.1]heptanyl, azabicyclo[3.1. 1]heptanyl, diazabicyclo[3.1.1]heptanyl, azabicyclo[3.2. 1]octanyl, diazabicyclo[3.2.1]octanyl, oxabicyclo[2.2.2]octanyl, azabicyclo[2.2.2]octanyl, azaadamantanyl, diazaadamantanyl, oxa-adamantanyl, azaspiro[3.3]heptanyl, diazaspiro[3.3]heptanyl, oxa-azaspiro[3.3]heptanyl, azaspiro[3.4]octanyl, diazaspiro[3.4]octanyl, oxa-azaspiro[3.4]octanyl, azaspiro[2.5]octanyl, diazaspiro[2.5]octanyl, azaspiro[4.4]nonanyl, diazaspiro[4.4]nonanyl, oxa-azaspiro[4.4]nonanyl, azaspiro[4.5]decanyl, diazaspiro[4.5]decanyl, diazaspiro[4.4]nonanyl, oxa-diazaspiro[4.4]nonanyl and the like.

In some embodiments, heterocycloalkyl refers to any three to ten membered monocyclic or bicyclic, saturated ring structure containing at least one heteroatom selected from the group consisting of O, N and S. The heterocycloalkyl group may be attached at any heteroatom or carbon atom of the ring such that the result is a stable structure. Examples of suitable heterocycloalkyl groups include, but are not limited to, azepanyl, aziridinyl, azetidinyl, pyrrolidinyl, dioxolanyl, imidazolidinyl, pyrazolidinyl, piperazinyl, piperidinyl, dioxanyl, morpholinyl, dithianyl, thiomorpholinyl, oxazepanyl, oxiranyl, oxetanyl, quinuclidinyl, tetrahydrofuranyl, tetrahydropyranyl, piperazinyl, and the like.

The term “heterocycloalkylene” when used alone or as part of a substituent group refers to a heterocycloalkyl diradical. In some embodiments heterocycloalkylene any three to ten membered monocyclic or bicyclic, saturated ring structure containing at least one heteroatom selected from the group consisting of O, N and S, wherein the ring structure is directly attached to two other variable groups.

In some embodiments, the term “spiroheterocycloalkyl” when used alone or as part of a substituent group refers to a non-aromatic group containing two rings, at least one of which is a heterocycloalkyl ring, and wherein the two rings share a single carbon atom in common.

In some embodiments, the term “spiroheterocycloalkylene” when used alone or as part of a substituent group refers to a spiroheterocycloalkyl diradical. In some embodiments, spiroheterocycloalkylene is a non-aromatic group containing two rings, at least one of which is a heterocycloalkyl ring, and wherein the two rings share a single carbon atom in common, and wherein the group is directly attached to two other variable groups.

The term “alkenyl” when used alone or as part of a substituent group refers to a straight- or branched-chain group having from 2 to 12 carbon atoms (“C₂-C₁₂”), preferably 2 to 4 carbons atoms (“C₂-C₄”), in the group, wherein the group includes at least one carbon-carbon double bond. Examples of alkenyl groups include vinyl (—CH═CH₂; C₂alkenyl), allyl (—CH₂—CH═CH₂; C₃alkenyl), propenyl (—CH═CHCH₃; C₃alkenyl); isopropenyl (—C(CH₃)═CH₂; C₃alkenyl), butenyl (—CH═CHCH₂CH₃; C₄alkenyl), sec-butenyl (—C(CH₃)═CHCH₃; C₄alkenyl), iso-butenyl (—CH═C(CH₃)₂; C₄alkenyl), 2-butenyl (—CH₂CH═CHCH₃; C₄alkyl), pentenyl (CH═CHCH₂CH₂CH₃ or CH₂═CHCH₂CH₂CH₂—; C₅alkenyl), and the like.

The term “alkenylene” when used alone or as part of a substituent group refers to a alkenyl diradical, i.e., a straight- or branched-chain group having from 2 to 12 carbon atoms (“C₂-C₁₂”), preferably 2 to 4 carbons atoms (“C₂-C₄”), in the group, wherein the group includes at least one carbon-carbon double bond, and wherein the group is directly attached to two other variable groups.

The term “alkynyl” when used alone or as part of a substituent group refers to a straight- or branched-chain group having from 2 to 12 carbon atoms (“C₂-C₁₂”), preferably 2 to 4 carbons atoms (“C₂-C₄”), in the group, wherein the group includes at least one carbon-carbon triple bond. Examples of alkynyl groups include ethynyl (—C≡CH; C₂alkynyl), propragyl (—CH₂—CH≡CH; C₃alkynyl), and the like.

The term “alkynylene” when used alone or as part of a substituent group refers to an alkynyl diradical, i.e., a straight- or branched-chain group having from 2 to 12 carbon atoms (“C₂-C₁₂”), preferably 2 to 4 carbons atoms (“C₂-C₄”), in the group, wherein the group includes at least one carbon-carbon triple bond, and wherein the group is directly attached to two other variable groups.

The term “aryl” when used alone or as part of a substituent group refers to a monocyclic all carbon aromatic ring or a multicyclic all carbon ring system wherein the rings are aromatic. Thus, aryl also includes multiple condensed ring systems (e.g., ring systems comprising 2, 3 or 4 aryl rings) having about 9 to 14 carbon atoms. In some embodiments, “aryl” refers to a mono- or bicyclic-aromatic hydrocarbon ring structure having 6 or 10 carbon atoms in the ring, wherein one or more of the carbon atoms in the ring is optionally substituted. Exemplary substituents include halogen atoms, —C₁-C₃ alkyl groups, and C₁-C₃haloalkyl groups. Halogen atoms include chlorine, fluorine, bromine, and iodine. C₁-C₃haloalkyl groups include, for example, —CF₃, —CH₂CF₃, and the like.

The term “arylene” when used alone or as part of a substituent group refers to an aryl diradical. In some embodiments, “arylene” refers to a mono- or bicyclic-aromatic hydrocarbon ring structure having 6 or 10 carbon atoms in the ring, wherein one or more of the carbon atoms in the ring is optionally substituted, and wherein the ring structure is directly attached to two other variable groups.

The term “heteroaryl” when used alone or as part of a substituent group, the term “heteroaryl” as used herein refers to a monocyclic aromatic ring that has at least one atom other than carbon in the ring, wherein the atom is selected from the group consisting of oxygen, nitrogen and sulfur; “heteroaryl” also includes multicyclic ring systems that have at least one such aromatic ring. Thus, “heteroaryl” includes single aromatic rings of from about 1 to 6 carbon atoms and about 1-4 heteroatoms selected from the group consisting of oxygen, nitrogen and sulfur. The sulfur and nitrogen atoms may also be present in an oxidized form provided the ring is aromatic. “Heteroaryl” also includes multiple condensed ring systems (e.g., ring systems comprising 2, 3 or 4 rings) wherein a heteroaryl group is condensed with one or more rings selected from heteroaryls or aryls. Thus, a heteroaryl (a single aromatic ring or multiple condensed ring system) has about 1-20 carbon atoms and about 1-6 heteroatoms within the heteroaryl ring system. A heteroaryl (a monocyclic aromatic ring or multicyclic condensed ring system) can also have about 5 to 12 or about 5 to 10 members within the heteroaryl ring. The rings of a multicyclic ring system can be connected to each other via fused, spiro and bridged bonds when allowed by valency requirements. In some embodiments, “heteroaryl” refers to a mono- or bicyclic-aromatic ring structure including carbon atoms as well as up to four heteroatoms selected from nitrogen, oxygen, and sulfur. In such embodiments, heteroaryl rings can include a total of 5, 6, 9, or 10 ring atoms. The heteroaryl moiety can be optionally substituted. Exemplary substituents include halogen atoms; —C₁-C₃ alkyl groups, and C₁-C₃haloalkyl groups. Halogen atoms include chlorine, fluorine, bromine, and iodine.

The term “heteroarylene” when used alone or as part of a substituent group refers to a heteroaryl diradical. In some embodiments, heteroarylene is a mono- or bicyclic-aromatic ring structure including carbon atoms as well as up to four heteroatoms selected from nitrogen, oxygen, and sulfur, wherein the ring structure is directly attached to two other variable groups.

The term “halo” refers to a halogen substituent (i.e., —F, —Cl, —Br, or —I).

The term “oxo” refers to an oxygen substituent that is connected by a double bond (i.e., ═O).

The term “alkoxy” when used alone or as part of a substituent group refers to an oxygen radical attached to an alkyl group by a single bond. Examples of alkoxy groups include methoxy (—OCH₃), ethoxy (—OCH₂CH₃), isopropoxy (—OCH(CH₃)₂) and the like.

The term “haloalkoxy” when used alone or as part of a substituent group refers to an oxygen radical attached to a haloalkyl group by a single bond. Examples of haloalkoxy groups include —OCF₃, —OCH₂CF₃, —OCH(CF₃)₂, and the like.

When a range of carbon atoms is used herein, for example, C₁-C₆, all ranges, as well as individual numbers of carbon atoms are encompassed. For example, “C₁-C₃” includes C₁-C₃, C₁-C₂, C₂-C₃, C₁, C₂, and C₃.

The term “C₁-C₆alk” when used alone or as part of a substituent group refers to an aliphatic linker having 1, 2, 3, 4, 5, or 6 carbon atoms and includes, for example, —CH₂—, —CH(CH₃)—, —CH(CH₃)—CH₂—, and —C(CH₃)₂—. The term “—C₀alk-” refers to a bond. In some aspects, the C₁-C₆alk can be substituted with one or more substituents.

In some embodiments wherein a group is described as “optionally substituted” (e.g., when a C₁-C₆alkyl, —C₁-C₆alkylene-, C₁-C₁₀ alkyl, —C₂-C₁₀alkenyl, —C₂-C₁₀alkynyl, —C₂-C₆ alkenylene-, —C₂-C₆ alkynylene-, cycloalkyl, cycloalkylene, heterocycloalkyl, heterocycloalkylene, aryl, arylene, heteroaryl, or heteroarylene group is optionally substituted), the optional substituent may be one or more of D, halo, oxo, C₁-C₆ alkyl, —C₁-C₆alkylene-, C₁-C₆ heteroalkyl, C₂-C₆ alkenyl, —C₂-C₆ alkenylene, C₂-C₆ alkynyl, —C₂-C₆ alkynylene, C₁-C₆ haloalkyl, C₁-C₆ alkyl-NR^{c1}R^{d1}, —(CH₂CH₂O)ₒC₁-C₆alkyl wherein o is 1-10; C₂₋₆ alkenyl-NR^{c1}R^{d1}, C₂₋₆ alkynyl-NR^{c1}R^{d1}, OC₂₋₆ alkyl-NR^{c1}R^{d1}, CN, NO₂, N₃, OR^{a1}, SR^{a1}, C(O)R^{b1}, C(O)NR^{c1}R^{d1}, —CH₂C(O)NR^{c1}R^{d1}, C(O)OR^{d1}, OC(O)R^{b1}, OC(O)NR^{c1}R^{d1}, —NR^{c1}R^{d1}, —NR^{c1}C(O)R^{b1}, NR^{c1}C(O)NR^{c1}NR^{c1}C(O)OR^{a1}, C(═NR^{g1})NR^{c1}R^{d1}, NR^{c1}C(═NR^{g1})NR^{c1}R^{d1}, P(R)₂, P(OR^{e1})₂, P(O)R^{e1}R^{f1}, P(O)OR^{e1}R^{f1}, S(O)R^{b1}, >SO(═NR^{b1}); S(O)NR^{c1}R^{d1}, S(O)₂R^{b1}, NR^{c1}S(O)₂R^{b1}, S(O)₂NR^{c1}R^{d1}; aryl, heteroaryl, spirocycloalkyl, spiroheterocycloalkyl, cycloalkyl, or heterocycloalkyl, wherein the C₁-C₆alkyl, —C₁-C₆alkylene-, —C₂-C₆alkenyl, —C₂-C₆alkynyl, —C₂-C₆ alkenylene-, —C₂-C₆ alkynylene-, aryl, heteroaryl, spirocycloalkyl, spiroheterocycloalkyl, cycloalkyl, heterocycloalkyl, are optionally substituted with D, halo, oxo, C₁-C₆ alkyl, C₁-C₆ heteroalkyl, C₂-C₆ alkenyl, C₂-C₆ alkynyl, C₁-C₆ haloalkyl, cycloalkyl, heterocycloalkyl, aryl, heteroaryl, C₁-C₆ alkyl-NR^{c1}R^{d1}, C₂₋₆ alkenyl-NR^{c1}R^{d1}, C₂₋₆ alkynyl-NR^{c1}R^{d1}, OC₂₋₆ alkyl-NR^{c1}R^{d1}, CN, NO₂, N₃, OR^{a1}, SR^{a1}, C(O)R^{b1}, C(O)NR^{c1}R^{d1}, —CH₂C(O)NR^{c1}R^{d1}, C(O)OR^{a1}, OC(O)R^{b1}, OC(O)NR^{c1}R^{d1}, —NR^{c1}R^{d1}, NR^{c1}C(O)R^{b1}, NR^{c1}C(O)NR^{c1}R^{d1}, NR^{c1}C(O)OR^{d1}, C(═NR^{g1})NR^{c1}R^{d1}, NR^{c1}C(═NR^{g1})NR^{c1}R^{d1}, P(R^{f1})₂, P(OR^{e1})₂, P(O)R^{e1}R^{f1}, P(O)OR^{e1}OR^{f1}, S(O)R^{b1}, S(O)NR^{c1}R^{d1}, S(O)₂R^{b1}, NR^{c1}S(O)₂R^{b1}, S(O)₂NR^{c1}R^{d1}.

In these optional substituents, each R^{a1} is independently H, D, C₁-C₆ alkyl, C₁-C₆ haloalkyl, C₂-C₆ alkenyl, C₂-C₆ alkynyl, aryl, cycloalkyl, heteroaryl, heterocycloalkyl, arylalkyl, heteroarylalkyl, cycloalkylalkyl, or heterocycloalkylalkyl, wherein said C₁-C₆ alkyl, C₁-C₆ haloalkyl, C₂-C₆ alkenyl, C₂-C₆ alkynyl, aryl, cycloalkyl, heteroaryl, heterocycloalkyl, arylalkyl, heteroarylalkyl, cycloalkylalkyl, or heterocycloalkylalkyl is optionally substituted with 1, 2, or 3 substituents independently selected from OH, CN, amino, halo, C₁-C₆ alkyl, C₁-C₆ alkoxy, C₁-C₆ haloalkyl, and C₁-C₆ haloalkoxy, C₃-C₁a cycloalkyl, C₁-C₆ heteroalkyl, 3-12 membered heterocyclyl, C₆-C₁₀ aryl, 5-10 membered heteroaryl, —C(O)H, —C(O)NH₂, —C(O)NH(C₁-C₆ alkyl), —C(O)N(C₁-C₆ alkyl)₂, —COOH, —C(O)C₁-C₆ alkyl, —C(O)OC₁-C₆ alkyl.

In these optional substituents each R^{b1} is independently H, D, C₁-C₆ alkyl, C₁-C₆ haloalkyl, C₂-C₆ alkenyl, C₂-C₆ alkynyl, aryl, cycloalkyl, heteroaryl, heterocycloalkyl, arylalkyl, heteroarylalkyl, cycloalkylalkyl, or heterocycloalkylalkyl, wherein said C₁-C₆ alkyl, C₁-C₆ haloalkyl, C₂-C₆ alkenyl, C₂-C₆ alkynyl, aryl, cycloalkyl, heteroaryl, heterocycloalkyl, arylalkyl, heteroarylalkyl, cycloalkylalkyl, or heterocycloalkylalkyl is optionally substituted with 1, 2, or 3 substituents independently selected from OH, CN, amino, halo, C₁-C₆ alkyl, C₁-C₆ alkoxy, C₁-C₆ haloalkyl, and C₁-C₆ haloalkoxy, C₃-C₁₀ cycloalkyl, C₁-C₆ heteroalkyl, 3-12 membered heterocyclyl, C₆-C₁₀ aryl, 5-10 membered heteroaryl, —C(O)H, —C(O)NH₂, —C(O)NH(C₁-C₆ alkyl), —C(O)N(C₁-C₆ alkyl)₂, —COOH, —C(O)C₁-C₆ alkyl, —C(O)OC₁-C₆ alkyl.

In these optional substituents R^{c1} or R^{d1} is independently H, C₁-C₁₀ alkyl, C₁-C₆ haloalkyl, C₂-C₆ alkenyl, C₂-C₆ alkynyl, aryl, heteroaryl, cycloalkyl, heterocycloalkyl, arylalkyl, heteroarylalkyl, cycloalkylalkyl, heterocycloalkylalkyl, arylcycloalkyl, arylheterocycloalkyl, arylheteroaryl, biaryl, heteroarylcycloalkyl, heteroarylheterocycloalkyl, heteroarylaryl, and biheteroaryl, wherein said C₁-C₁₀ alkyl, C₁-C₆ haloalkyl, C₂-C₆ alkenyl, C₂-C₆ alkynyl, aryl, heteroaryl, cycloalkyl, heterocycloalkyl, arylalkyl, heteroarylalkyl, cycloalkylalkyl, heterocycloalkylalkyl, arylcycloalkyl, arylheterocycloalkyl, arylheteroaryl, biaryl, heteroarylcycloalkyl, heteroarylheterocycloalkyl, heteroarylaryl, and biheteroaryl are each optionally substituted with 1, 2, or 3 substituents independently selected from OH, CN, amino, halo, C₁-C₆ alkyl, C₁-C₆ alkoxy, C₁-C₆ haloalkyl, C₁-C₆ haloalkoxy, hydroxyalkyl, cyanoalkyl, aryl, heteroaryl, C(O)OR^{a1}, C(O)R^{b1}, S(O)₂R^{b1}, alkoxyalkyl, and alkoxyalkoxy; C₃-C₁₀ cycloalkyl, C₁-C₆ heteroalkyl, 3-12 membered heterocyclyl, C₆-C₁₀ aryl, 5-10 membered heteroaryl, —C(O)NH₂, —C(O)NH(C₁-C₆ alkyl), or —C(O)N(C₁-C₆ alkyl)₂.

Alternatively, in some embodiments, these optional substituents R^{c2} and R^{d2} together with the N atom to which they are attached form a 4-, 5-, 6- or 7-membered heterocycloalkyl group or heteroaryl group, each optionally substituted with 1, 2, or 3 substituents independently selected from OH, CN, amino, halo, C₁-C₆ alkyl, C₁-C₆ alkoxy, C₁-C₆ haloalkyl, C₁-C₆ haloalkoxy, hydroxyalkyl, cyanoalkyl, aryl, heteroaryl, C(O)OR^{d1}, C(O)R^{b1}, S(O)₂R^{b1}, alkoxyalkyl, and alkoxyalkoxy;

In these optional substituents R^{e1} is independently H, C₁-C₆ alkyl, C₁-C₆ haloalkyl, C₂-C₆ alkenyl, (C₁-C₆ alkoxy)-C₁-C₆ alkyl, C₂-C₆ alkynyl, aryl, cycloalkyl, heteroaryl, heterocycloalkyl, arylalkyl, cycloalkylalkyl, heteroarylalkyl, or heterocycloalkylalkyl.

In these optional substituents R^{f1} is independently H, C₁-C₆ alkyl, C₁-C₆ haloalkyl, C₂-C₆ alkenyl, C₂-C₆ alkynyl, aryl, cycloalkyl, heteroaryl, or heterocycloalkyl.

In these optional substituents R^{g1} is independently H, CN, or NO₂.

“Pharmaceutically acceptable” means approved or approvable by a regulatory agency of the Federal or a state government or the corresponding agency in countries other than the United States, or that is listed in the U.S. Pharmacopoeia or other generally recognized pharmacopoeia for use in animals, e.g., in humans.

“Pharmaceutically acceptable salt” refers to a salt of a compound of the disclosure that is pharmaceutically acceptable and that possesses the desired pharmacological activity of the parent compound. In particular, such salts are non-toxic may be inorganic or organic acid addition salts and base addition salts. Specifically, such salts include: (1) acid addition salts, formed with inorganic acids such as hydrochloric acid, hydrobromic acid, sulfuric acid, nitric acid, phosphoric acid, and the like; or formed with organic acids such as acetic acid, propionic acid, hexanoic acid, cyclopentanepropionic acid, glycolic acid, pyruvic acid, lactic acid, malonic acid, succinic acid, malic acid, maleic acid, fumaric acid, tartaric acid, citric acid, benzoic acid, 3-(4-hydroxybenzoyl)benzoic acid, cinnamic acid, mandelic acid, methanesulfonic acid, ethanesulfonic acid, 1,2-ethane-disulfonic acid, 2-hydroxyethanesulfonic acid, benzenesulfonic acid, 4-chlorobenzenesulfonic acid, 2-naphthalenesulfonic acid, 4-toluenesulfonic acid, camphorsulfonic acid, 4-methylbicyclo[2.2.2]-oct-2-ene-1-carboxylic acid, glucoheptonic acid, 3-phenylpropionic acid, trimethylacetic acid, tertiary butylacetic acid, lauryl sulfuric acid, gluconic acid, glutamic acid, hydroxynaphthoic acid, salicylic acid, stearic acid, muconic acid, and the like; or (2) salts formed when an acidic proton present in the parent compound either is replaced by a metal ion, e.g., an alkali metal ion, an alkaline earth ion, or an aluminum ion; or coordinates with an organic base such as ethanolamine, diethanolamine, triethanolamine, N-methylglucamine and the like. Salts further include, by way of example only, sodium, potassium, calcium, magnesium, ammonium, tetraalkylammonium, and the like; and when the compound contains a basic functionality, salts of non-toxic organic or inorganic acids, such as hydrochloride, hydrobromide, tartrate, mesylate, acetate, maleate, oxalate and the like.

A “pharmaceutically acceptable excipient” refers to a substance that is non-toxic, biologically tolerable, and otherwise biologically suitable for administration to a subject, such as an inert substance, added to a pharmacological composition or otherwise used as a vehicle, carrier, or diluent to facilitate administration of an agent and that is compatible therewith. Examples of excipients include calcium carbonate, calcium phosphate, various sugars and types of starch, cellulose derivatives, gelatin, vegetable oils, and polyethylene glycols.

A “solvate” refers to a physical association of a compound of Formula I with one or more solvent molecules.

“Subject” includes humans. The terms “human,” “patient,” and “subject” are used interchangeably herein.

“Treating” or “treatment” of any disease or disorder refers, in one embodiment, to ameliorating the disease or disorder (i.e., arresting or reducing the development of the disease or at least one of the clinical symptoms thereof). In another embodiment “treating” or “treatment” refers to ameliorating at least one physical parameter, which may not be discernible by the subject. In yet another embodiment, “treating” or “treatment” refers to modulating the disease or disorder, either physically, (e.g., stabilization of a discernible symptom), physiologically, (e.g., stabilization of a physical parameter), or both. In yet another embodiment, “treating” or “treatment” refers to delaying the onset of the disease or disorder.

“Compounds of the present disclosure,” and equivalent expressions, are meant to embrace compounds of Formula I as described herein, as well as the respective subgenera, which expression includes the stereoisomers (e.g., enantiomers, diastereomers) and constitutional isomers (e.g., tautomers) of compounds of Formula I as well as the pharmaceutically acceptable salts, where the context so permits.

As used herein, the term “isotopic variant” refers to a compound that contains proportions of isotopes at one or more of the atoms that constitute such compound that is greater than natural abundance. For example, an “isotopic variant” of a compound can be radiolabeled, that is, contain one or more radioactive isotopes, or can be labeled with non-radioactive isotopes such as for example, deuterium (H or D), carbon-13 (¹³C), nitrogen-15 (¹⁵N), or the like. It will be understood that, in a compound where such isotopic substitution is made, the following atoms, where present, may vary, so that for example, any hydrogen may be ²H/D, any carbon may be ¹³C, or any nitrogen may be ¹⁵N, and that the presence and placement of such atoms may be determined within the skill of the art.

It is also to be understood that compounds that have the same molecular formula but differ in the nature or sequence of bonding of their atoms or the arrangement of their atoms in space are termed “isomers.” Isomers that differ in the arrangement of their atoms in space are termed “stereoisomers,” for example, diastereomers, enantiomers, and atropisomers. The compounds of this disclosure may possess one or more asymmetric centers; such compounds can therefore be produced as individual (R)- or (S)-stereoisomers at each asymmetric center, or as mixtures thereof.

Unless indicated otherwise, the description or naming of a particular compound in the specification and claims is intended to include all stereoisomers and mixtures, racemic or otherwise, thereof. Where one chiral center exists in a structure, but no specific stereochemistry is shown for that center, both enantiomers, individually or as a mixture of enantiomers, are encompassed by that structure. Where more than one chiral center exists in a structure, but no specific stereochemistry is shown for the centers, all enantiomers and diastereomers, individually or as a mixture, are encompassed by that structure. The methods for the determination of stereochemistry and the separation of stereoisomers are well-known in the art.

In some aspects, the disclosure is directed to a compound of Formula (I):

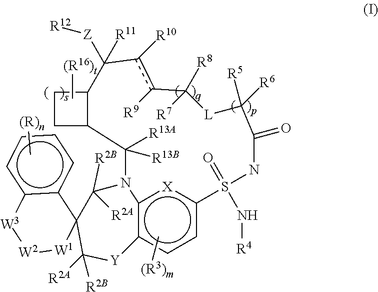

or a pharmaceutically acceptable salt or solvate thereof.

In some aspects, L in Formula (I) is absent, —NR¹⁴—, —O—, —S—, —S(O)—, —S(O)₂—, arylene, —O-arylene, cycloalkylene, —O-cycloalkylene, cycloalkenylene, spirocycloalkylene, heteroarylene, heterocycloalkylene, —O-heterocycloalkylene, heterocycloalkenylene, or spiroheterocycloalkylene wherein the arylene, cycloalkylene, cycloalkenylene, spirocycloalkylene, heteroarylene, heterocycloalkylene, heterocycloalkenylene, or spiroheterocycloalkylene is optionally substituted.

In some embodiments, L in Formula (I) is absent.

In other embodiments, L in Formula (I) is —NR¹⁴. In some embodiments wherein R¹⁴ is —C₁-C₆alkyl, L in Formula (I) is —N(C₁-C₆alkyl), such as, for example, —N(CH₃)—.

In other embodiments, L in Formula (I) is —O—.

In some aspects, [symbol] in Formula (I) is single bond or double bond. In some embodiments, [symbol] is a single bond. In other embodiments, [symbol] is a double bond.

In some aspects, X in Formula (I) is CH or N. In some embodiments, X is CH. In other embodiments, X is N.

In some aspects, Y in Formula (I) is —O—, —S—, —S(O)—, or —S(O)₂—. In some embodiments, Y is —O—. In some embodiments, Y is —S—. In some embodiments, Y is —S(O)—. In some embodiments, Y is —S(O)₂—.

In some aspects, Z in Formula (I) is —NR¹⁵—, —O—, or —S—. In some embodiments, Z is —NR¹⁵—. In other embodiments, Z is —O—. In yet other embodiments, Z is —S—

In some aspects, the moiety —W¹—W²—W³ in Formula (I) is —CR^{1A}R^{1B}—CR^{1A}R^{1B}—CR^{1A}R^{1B}—, —O—CR^{1C}R^{1D}—CR^{1A}R^{1B}—, —CR^{1A}R^{1B}—CR^{1C}R^{1D}—O—, —NR^{1C}—CR^{1C}R^{1D}—CR^{1A}R^{1B}—, —CR^{1A}R^{1B}—CR^{1C}R^{1D}—NR^{1C}—, —S—CR^{1C}R^{1D}—CR^{1A}R^{1B}, or —CR^{1A}R^{1B}—CR^{1C}R^{1D}—S—.

In some embodiments, the moiety —W¹—W²—W³ in Formula (I) is —CR^{1A}R^{1B}—CR^{1A}R^{1B}—CR^{1A}R^{1B}—. In some embodiments, the moiety —W¹—W²—W³ in Formula (I) is —O—CR^{1C}R^{1D}—CR^{1A}R^{1B}—.

In some embodiments, the moiety —W¹—W²—W³ in Formula (I) —CR^{1A}R^{1B}—CR^{1C}R^{1D}—O—. In some embodiments, the moiety —W¹—W²—W³ in Formula (I) is —NR^{1C}—CR^{1C}R^{1D}—CR^{1A}R^{1B}—. In some embodiments, the moiety —W¹—W²—W³ in Formula (I) —CR^{1A}R^{1B}—CR^{1C}R^{1D}—NR^{1C}—. In some embodiments, the moiety —W¹—W²—W³ in Formula (I) is —S—CR^{1C}R^{1D}—CR^{1A}R^{1B} In some embodiments, the moiety —W¹—W²—W³ in Formula (I) is —CR^{1A}R^{1B}—CR^{1C}R^{1D}—S—.

In some aspects, in Formula (I), each n is independently 0-3. In some embodiments, n=0.

In other embodiments, n=1. In other embodiments, n=2. In other embodiments, n=3.

In some aspects, in Formula (I), each m is independently 0-2. In some embodiments, m=0.

In other embodiments, m=1. In other embodiments, m=2.

In some aspects, in Formula (I), each p is independently 0-4. In some embodiments, p=0.

In other embodiments, p=1. In other embodiments, p=2. In other embodiments, p=3. In other embodiments, p=4.

In some aspects, in Formula (I), each q is independently 0-4. In some embodiments, q=0.

In other embodiments, q=1. In other embodiments, q=2. In other embodiments, q=3. In yet other embodiments, q=4.

In some aspects, in Formula (I), each s is independently 0-3. In some embodiments, s=0.

In other embodiments, s=1. In other embodiments, s=2. In other embodiments, s=3.

In some aspects, in Formula (I), each t is independently 0-4. In some embodiments, t=0. In other embodiments, t=1. In other embodiments, t=2. In other embodiments, t=3. In other embodiments, t=4. In other embodiments, t=5. In other embodiments, t=6.

In some aspects, each R in Formula (I) is independently -D, -halo, —CN, —NO₂, —C₁-C₆alkyl, —C₂-C₆alkenyl, —C₁-C₆alkoxy, -cycloalkyl, —OR^{a}, —SR^{a}, —C(O)R^{b}, —C(O)OR^{b}, —NR^{c}R^{d}, —NR^{a}R^{c}, —C(O)NR^{c}R^{d}, or —S(O)₂R^{b}; wherein said —C₁-C₆alkyl, —C₂-C₆alkenyl, —C₁-C₆alkoxy, or -cycloalkyl is optionally substituted.

In some embodiments, R is halo, for example, Cl or F. In some embodiments, R is —SR^{a}, for example —SCH₃. In other embodiments, R is —C₁-C₆alkyl, for example —CH₃.

In some aspects, each R³ in Formula (I) is independently -D, -halo, —CN, —NO₂, —C₁-C₆alkyl, —C₂-C₆alkenyl, —C₂-C₆alkynyl, —OR^{a}, —SR^{a}, —NR^{c}R^{d}, —NR^{a}R^{c}, —C(O)R^{b}, —OC(O)R^{b}, —C(O)OR^{b}, —C(O)NR^{c}R^{d}, —S(O)₂R^{b}; -aryl, -heteroaryl, -cycloalkyl, or -heterocycloalkyl, wherein the —C₁-C₆alkyl, —C₂-C₆alkenyl, —C₂-C₆alkynyl, -cycloalkyl, -heterocycloalkyl, -aryl, or -heteroaryl is optionally substituted.

In some embodiments, R³ in Formula I is halo, for example, Cl or F. In other embodiments, R³ in Formula I is —C₁-C₆alkyl substituted with fluorine, for example —CF₃. In other embodiments, R³ in Formula I is —C₁-C₆alkyl, for example, —C₆alkyl, —C₅alkyl, —C₄alkyl, —C₃alkyl, —C₂alkyl, —C₁alkyl. In some embodiments, R³ in Formula I is —C₁alkyl substituted with —CN. In other embodiments, R³ in Formula I is cycloalkyl, for example, cyclopropane, cyclobutane, cyclopentane, and the like. In other embodiments, R³ in Formula I is cyclopropane substituted with —CN.

In some aspects, each R^{1A}, or R^{1B} is independently H, D, halo, optionally substituted C₁-C₆alkyl, or R^{1A} and R^{1B} that are attached to the same carbon atom may, together with the carbon atom to which they are both attached, form an optionally substituted cycloalkyl ring.

In some embodiments, R^{1A}, and R^{1B} in Formula (I) are each H.

In other embodiments, R^{1A} and R^{1B} that are attached to the same carbon atom, together with the carbon atom to which they are both attached, form an optionally substituted 3-6 membered cycloalkyl ring. In some embodiments, R^{1A} and R^{1B} that are attached to the same carbon atom, together with the carbon atom to which they are both attached, form a cyclopropane ring.

In some aspects, each R^{1C} and R^{1D} in Formula I is independently H, D, fluoro, optionally substituted C₁-C₆alkyl, or R^{1C} and R^{1D} may, together with the carbon atom to which they are both attached, form an optionally substituted cycloalkyl ring.

In some embodiments, R^{1C} and R^{1D} in Formula I are each H.

In other embodiments, R^{1C} and R^{1D} that are attached to the same carbon atom together with the carbon atom to which they are both attached, form a cyclopropane ring.

In some aspects, R^{1B} and R^{1C} that are attached to adjacent carbon atoms may, together with the carbon atoms to which they are attached, form an optionally substituted cycloalkyl ring.

In some aspects, each R^{2A} and R^{2B} is independently H, D, fluoro, optionally substituted C₁-C₆alkyl, or R^{2A} and R^{2B} may, together with the carbon atom to which they are both attached, form an optionally substituted cycloalkyl ring.

In some embodiments, each R^{2A} and R^{2B} is H.

In some aspects, R⁴ is H, —C(O)OR^{4A}, —C(O)R^{4B}, —C(O)NR^{4C}R^{4D}, —S(O)R^{4B}, —S(O)₂R^{4B}, —S(O)NR^{4C}R^{4D}, or —S(O)₂NR^{4C}R^{4D}.

In some embodiments, R⁴ is H.

In some embodiments, R⁴ is —C(O)R^{4B}, or —C(O)NR^{4C}R^{4D}. In some embodiments, R⁴ is —C(O)R^{4B}. In other embodiments, R⁴ is —C(O)NR^{4C}R^{4D}.

In some aspects, each R^{4A} is independently —C₁-C₁₀alkyl, —C₃-C₁₀ alkenyl, —C₃-C₁₀ alkynyl, aryl, cycloalkyl, C₁-C₆heteroalkyl, heteroaryl, heterocycloalkyl, or heterocycloalkenyl wherein the C₁-C₁₀ alkyl, C₃-C₁₀ alkenyl, C₃-C₁₀ alkynyl, aryl, cycloalkyl, heteroaryl, heterocycloalkyl, or heterocycloalkenyl is optionally substituted.

In some aspects, each R^{4B} is independently H, D, —C₁-C₆ alkyl, —C₂-C₆ alkenyl, —C₂-C₆ alkynyl, aryl, cycloalkyl, C₁-C₆heteroalkyl, heteroaryl, heterocycloalkyl, or heterocycloalkenyl wherein the —C₁-C₆ alkyl, —C₂-C₆ alkenyl, —C₂-C₆ alkynyl, aryl, cycloalkyl, heteroaryl, heterocycloalkyl, or heterocycloalkenyl is optionally substituted.

In some embodiments, R^{4B} is optionally substituted aryl.

In some embodiments, R^{4B} is phenyl.

In some embodiments, R^{4B} is substituted phenyl. In some embodiments, R^{4B} is phenyl substituted with halogen. In some embodiments, R^{4B} is phenyl substituted with bromine. In some embodiments, R^{4B} is 4-bromophenyl.

In other embodiments, R^{4B} is optionally substituted —C₁-C₆ alkyl, for example, C₆ alkyl, C₅ alkyl, C₄ alkyl, C₃ alkyl, C₂ alkyl, C₁ alkyl.

In some embodiments, R^{4B} is —CH(CH₃)₂.

In other embodiments, R^{4B} is methyl.

In other embodiments, R^{4B} is ethyl.

In yet other embodiments, R^{4B} is substituted —C₁-C₆ alkyl.

In some embodiments, R^{4B} is —C₁-C₆ alkyl substituted with cycloalkyl.

In some embodiments, R^{4B} is —CH₂— substituted with cycloalkyl. In some embodiments, R^{4B} is —CH₂-cyclopropyl.

In some embodiments, R^{4B} is optionally substituted cycloalkyl, for example, cyclopropyl, cyclobutyl, cyclopentyl, cyclohexyl and the like. In some embodiments, R^{4B} is cyclobutyl.

In other embodiments, R^{4B} is optionally substituted heterocycloalkyl. In some embodiments, R^{4B} is tetrahydro-2H-pyran-4-yl.

In some embodiments, R^{4B} is optionally substituted heteroaryl. In some embodiments, R^{4B} is 1-methyl-1H-pyrazol-4-yl, optionally substituted with methoxy or methyl. In some embodiments, R^{4B} is 3-methoxy-1-methyl-1H-pyrazol-4-yl. In other embodiments, R^{4B} is 3-methyl-1-methyl-1H-pyrazol-4-yl. In other embodiments, R^{4B} is 5-methyl-1-methyl-1H-pyrazol-4-yl. In other embodiments, R^{4B} is 5-methyl-1-methyl-1H-pyrazol-3-yl.

In some aspects, each R^{4C} or R^{4D} is independently H, D, —C₁-C₁₀ alkyl, —C₂-C₆ alkenyl, —C₂-C₆ alkynyl, —OC₁-C₆alkyl, —O-cycloalkyl, aryl, heteroaryl, cycloalkyl, C₁-C₆heteroalkyl, heterocycloalkyl, or heterocycloalkenyl, wherein said C₁-C₁₀ alkyl, C₂-C₆ alkenyl, C₂-C₆ alkynyl, —OC₁-C₆alkyl, —O-cycloalkyl, aryl, heteroaryl, cycloalkyl, heterocycloalkyl, or heterocycloalkenyl are each optionally substituted.

In other embodiments, R^{4C} is H, and R^{4D} is optionally substituted —C₁-C₁₀ alkyl, for example, C₆ alkyl, C₅ alkyl, C₄ alkyl, C₃ alkyl, C₂ alkyl, C₁ alkyl, and the like. In some embodiments, R^{4C} is H, and R^{4D} is —CH(CH₃)₂. In other embodiments, R^{4C} is H, and R^{4D} is —CH₂CH₃.

In some embodiments, R^{4C} is H, and R^{4D} is optionally substituted cycloalkyl, for example, cyclopropyl, cyclobutyl, cyclopentyl, cyclohexyl and the like. In some embodiments, R^{4C} is H, and R^{4D} is cyclobutyl. In some embodiments, R^{4C} is H, and R^{4D} is cyclopropyl.

In some embodiments, R^{4C} is H, and R^{4D} is optionally substituted cyclohexyl. In some embodiments, R^{4C} is H, and R^{4D} is cyclohexyl substituted with —OCH₃.

In some embodiments, R^{4C} is —C₁-C₁₀ alkyl, and R^{4D} is —C₁-C₁₀ alkyl. In some embodiments, R^{4C} is —CH₃, and R^{4D} is —CH₃.

In some embodiments, each R^{4C} is H, and R^{4D} is optionally substituted heterocycloalkyl.

In some embodiments, each R^{4C} is H, and R^{4D} is optionally substituted tetrahydro-2H-pyran.

In some embodiments, R^{4C} is H, and R^{4D} is optionally substituted heteroaryl. In some embodiments, R^{4C} is H, and R^{4D} is 1-methyl-1H-pyrazol-4-yl, optionally substituted with methoxy or methyl. In some embodiments, R^{4C} is H, and R^{4D} is 3-methoxy-1-methyl-1H-pyrazol-4-yl. In other embodiments, R^{4C} is H, and R^{4D} is 3-methyl-1-methyl-1H-pyrazol-4-yl. In other embodiments, R^{4C} is H, and R^{4D} is 5-methyl-1-methyl-1H-pyrazol-4-yl. In other embodiments, R^{4C} is H, and R^{4D} is 5-methyl-1-methyl-1H-pyrazol-3-yl.

In some aspects, R^{4C} and R^{4D}, together with the N atom to which they are both attached, form an optionally substituted monocyclic or multicyclic heterocycloalkyl, or optionally substituted monocyclic or multicyclic heterocycloalkenyl group.

In some embodiments, R^{4C} and R^{4D}, together with the N atom to which they are both attached, form an optionally substituted monocyclic heterocycloalkyl.

In some embodiments, R^{4C} and R^{4D}, together with the N atom to which they are both attached, form an optionally substituted azetidine-1-yl group.

In some embodiments, R^{4C} and R^{4D}, together with the N atom to which they are both attached, form an azetidine-1-yl group.

In some embodiments, R^{4C} and R^{4D}, together with the N atom to which they are both attached, form an azetidine-1-yl group substituted with a hydroxyl group.

In some embodiments, R^{4C} and R^{4D}, together with the N atom to which they are both attached, form an azetidine-1-yl group substituted with a methoxy group.

In some embodiments, R^{4C} and R^{4D}, together with the N atom to which they are both attached, form an azetidine-1-yl group substituted with a dialkylamino group, for example, a dimethylamino group.

In some aspects, each R⁵, R⁷ or R¹¹ is independently H, D, halo, —OH, —CN, —NO₂, —C₁-C₆alkyl, —C₂-C₆alkenyl, —C₂-C₆alkynyl, aryl, heteroaryl, cycloalkyl, heterocycloalkyl, heterocycloalkenyl, —OR^{a}, —SR^{a}, —NR^{c}R^{d}, —NR^{a}R^{c}, —C(O)R^{b}, —OC(O)R^{b}, —C(O)OR^{b}, —C(O)NR^{c}R^{d}, —S(O)R^{b}, or —S(O)₂R^{b}, wherein the C₁-C₆alkyl, —C₂-C₆alkenyl, —C₂-C₆alkynyl, aryl, heteroaryl, cycloalkyl, heterocycloalkenyl, or heterocycloalkyl is optionally substituted.

In some embodiments, R⁵ is Formula I is H. In other embodiments, R⁵ in Formula I is —C₁-C₆alkyl, for example, C₆alkyl, C₅alkyl, C₄alkyl, C₃alkyl, C₂alkyl, C₁alkyl, —CH₃, and the like. In some embodiments, R⁵ in Formula (I) is —CH₃.

In some embodiments, R⁷ in Formula I is H. In other embodiments, R⁷ in Formula I is —C₁-C₆alkyl, for example, C₆alkyl, C₅alkyl, C₄alkyl, C₃alkyl, C₂alkyl, C₁alkyl, —CH₃, and the like. In some embodiments, R⁷ in Formula I is —CH₃. In other embodiments, R⁷ in Formula I is —OR^{a}, for example, —OCH₃.

In some embodiments, R¹¹ in Formula (I) is H. In other embodiments, R¹¹ in Formula I is —C₁-C₆alkyl, for example, C₆alkyl, C₅alkyl, C₄alkyl, C₃alkyl, C₂alkyl, C₁alkyl, —CH₃, and the like.

In some aspects, each R⁶ or R⁸ in Formula (I) is independently H, D, halo, —C₁-C₆alkyl, —C₂-C₆alkenyl, —C₂-C₆alkynyl, —C(O)R^{b}, —C(O)OR^{a}, —C(O)NR^{c}R^{d}, aryl, heteroaryl, cycloalkyl, heterocycloalkyl, or heterocycloalkenyl, wherein said C₁-C₆alkyl, —C₂-C₆alkenyl, —C₂-C₆alkynyl, aryl, heteroaryl, cycloalkyl, heterocycloalkyl, or heterocycloalkenyl is optionally substituted.

In some embodiments, R⁶ in Formula (I) is H. In other embodiments, R⁶ in Formula (I) is —C₁-C₆alkyl, for example, C₆alkyl, C₅alkyl, C₄alkyl, C₃alkyl, C₂alkyl, C₁alkyl, —CH₃, and the like. In some embodiments, R⁶ in Formula (I) is —CH₃.

In some embodiments, R⁸ in Formula (I) is H. In other embodiments, R⁸ in Formula (I) is —C₁-C₆alkyl, for example, C₆alkyl, C₅alkyl, C₄alkyl, C₃alkyl, C₂alkyl, C₁alkyl, —CH₃, and the like. In some embodiments, R⁸ in Formula (I) is —CH₃.

In some embodiments, R⁵ and R⁶ together with the C atom to which they are attached form a cycloalkyl, heterocycloalkyl, or heterocycloalkenyl ring, each optionally substituted.

In other embodiments, an R⁵ and an R⁶ attached to adjacent carbon atoms, together with the C atoms to which they are attached, form a cycloalkyl, heterocycloalkyl, or heterocycloalkenyl ring, each optionally substituted

In some embodiments, R⁷ and R⁸ together with the C atom to which they are both attached form a cycloalkyl, heterocycloalkyl, or heterocycloalkenyl ring, each optionally substituted, each optionally substituted.

In other embodiments, an R⁷ and an R⁸ attached to adjacent carbon atoms, together with the C atoms to which they are attached, form a cycloalkyl, heterocycloalkyl, or heterocycloalkenyl ring, each optionally substituted.

In some aspects, R⁹ and R¹⁰ in Formula (I) are independently H, D, -Me, CN, —CH₂CN, —CH₂F, —CHF₂, —CF₃ or —F.

In some embodiments, R⁹ is H and R¹⁰ is H.

In some aspects, R¹² in Formula (I) is H, D, —C(O)R^{b}, —C(O)OR^{c}, —C(O)NR^{c}R^{d}, P(OR^{c})₂, P(O)R^{c}R^{b}, P(O)OR^{c}OR^{b}, S(O)R^{b}, S(O)NR^{c}R^{d}, S(O)₂R^{b}, S(O)₂NR^{c}R^{d}, B(OR^{c})(OR^{b}), SiR^{b}₃, C₁-C₁₀alkyl, C₂-C₁₀ alkenyl, C₂-C₁₀ alkynyl, —(CH₂CH₂O)ₒR^{a} wherein o=1 to 10, aryl, cycloalkyl, heteroaryl, or heterocycloalkyl, wherein said C₁-C₁₀ alkyl, C₂-C₁₀alkenyl, C₂-C₁₀ alkynyl, aryl, cycloalkyl, heteroaryl, or heterocycloalkyl is optionally substituted.

In some embodiments, R¹² is H.

In some embodiments, R¹² is optionally substituted C₁-C₁₀alkyl. In other embodiments, R¹² is —CH₃.

In other embodiments, R¹² is C₁-C₁₀alkyl substituted with —NR^{c1}R^{d1} wherein R^{c1} and R^{d1} are independently C₁-C₁₀ alkyl. In some embodiments, R¹² is —CH₂CH₂—N(CH₃)₂.

In other embodiments, R¹² is C₁-C₁₀alkyl substituted with heterocycloalkyl. In some embodiments, R¹² is

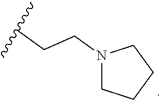 [SMILES: *CCN1CCCC1]

In some embodiments, R¹² is —C(O)NR^{c}R^{d} wherein R^{c} and R^{d} each —C₁-C₁₀ alkyl. In some embodiments, R¹² is —C(O)N(CH₃)₂.

In some embodiments, R¹² and R¹¹ form an optionally substituted monocyclic or multicyclic heterocycloalkyl, or optionally substituted monocyclic or multicyclic heterocycloalkenyl group.

In some aspects, R^{13A} and R^{13B} in Formula (I) are independently H, D, or optionally substituted C₁-C₆alkyl; or R^{13A} and R^{13B} may, together with the carbon atom to which they are both attached, form an optionally substituted cycloalkyl ring.

In some embodiments, R^{13A} and R^{13B} in Formula (I) are both H.

In some aspects, R¹⁴ and R¹⁵ in Formula (I) are independently H, D, —C₁-C₆alkyl, —C₂-C₆alkenyl, —C₂-C₆alkynyl, —OC₁-C₆alkyl, —C(O)R^{b}, —C(O)OR^{b}, —C(O)NR^{c}R^{d}, —S(O)R^{b} or —S(O)₂R^{b}, aryl, heteroaryl, cycloalkyl, heterocycloalkyl, or heterocycloalkenyl group, wherein said C₁-C₆alkyl, —C₂-C₆alkenyl, —C₂-C₆alkynyl, —OC₁-C₆alkyl, aryl, heteroaryl, cycloalkyl, heterocycloalkyl, or heterocycloalkenyl ring, is optionally substituted; or R¹⁴ together with an R⁵, R⁶, R⁷ or an R⁸ form an optionally substituted monocyclic or multicyclic heterocycloalkyl, or optionally substituted monocyclic or multicyclic heterocycloalkenyl group.

In some embodiments, R¹⁴ is —C₁-C₆alkyl. -In other embodiments, R¹⁴ is —CH₃.

In some aspects, each R¹⁶ in Formula (I) is independently H, D, —OH, -Me, —CH₂F, —CHF₂, —CF₃ or —F.

In some aspects, each R^{a} in Formula (I) is independently H, D, —C(O)R^{b}, —C(O)OR^{c}, —C(O)NR^{c}R^{d}, —P(OR^{c})₂, —P(O)R^{c}R^{b}, —P(O)OR^{c}OR^{b}, —S(O)R^{b}, —S(O)NR^{c}R^{d}, —S(O)₂R^{b}, —S(O)₂NR^{c}R^{d}, —B(OR^{c})(OR^{b}), SiR^{b}₃, —C₁-C₁₀alkyl, —C₂-C₁₀ alkenyl, —C₂-C₁₀ alkynyl, aryl, cycloalkyl, heteroaryl, heterocycloalkyl, or heterocycloalkenyl wherein the C₁-C₁₀ alkyl, C₂-C₁₀ alkenyl, C₂-C₁₀ alkynyl, aryl, cycloalkyl, heteroaryl, heterocycloalkyl, or heterocycloalkenyl is optionally substituted.

In some aspects, each R^{b} in Formula (I) is independently H, D, —C₁-C₆ alkyl, —C₂-C₆ alkenyl, —C₂-C₆ alkynyl, aryl, cycloalkyl, heteroaryl, heterocycloalkyl, or heterocycloalkenyl wherein the —C₁-C₆ alkyl, —C₂-C₆ alkenyl, —C₂-C₆ alkynyl, aryl, cycloalkyl, heteroaryl, heterocycloalkyl, or heterocycloalkenyl is optionally substituted.

In some aspects, each R^{c} or R^{d} in Formula (I) is independently H, D, —C₁-C₁₀ alkyl, —C₂-C₆ alkenyl, —C₂-C₆ alkynyl, —OC₁-C₆alkyl, —O-cycloalkyl, aryl, heteroaryl, cycloalkyl, heterocycloalkyl, or heterocycloalkenyl, wherein said C₁-C₁₀ alkyl, C₂-C₆ alkenyl, C₂-C₆ alkynyl, —OC₁-C₆alkyl, —O-cycloalkyl, aryl, heteroaryl, cycloalkyl, heterocycloalkyl, or heterocycloalkenyl are each optionally substituted; or R^{c} and R^{d}, together with the N atom to which they are both attached, form an optionally substituted monocyclic or multicyclic heterocycloalkyl, or optionally substituted monocyclic or multicyclic heterocycloalkenyl group.

In some embodiments of the compound of Formula (I), s=1; t=0; R^{2A} is H; R^{2B} is H; R^{13A} is H; and R^{13B} is H.

In some embodiments of the compound of Formula (I), m=0; s=1; t=0; R^{2A} is H; R^{2B} is H; R^{13A} is H; and R^{13B} is H.

In some embodiments of the compound of Formula (I), the compound is a compound of Formula (IA):

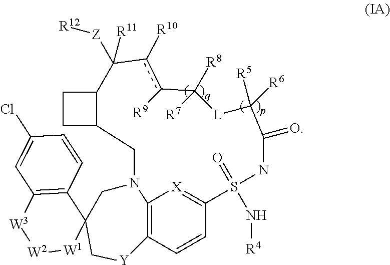

wherein the variables have the values of Formula (I).

In some embodiments of the compound of Formula (IA), Y is O.

In other embodiments of the compound of Formula (IA), X is CH.

In some embodiments of the compound of Formula (I), the compound is a compound of Formula (IB).

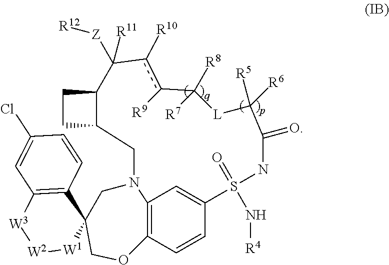

wherein the variables have the values of Formula (I).

In some embodiments of the compound of Formula (IB), the moiety —W¹—W²—W³— is —CR^{1A}R^{1B}—CR^{1C}R^{1D}—O—.

In some embodiments wherein the moiety —W¹—W²—W³— is —CR^{1A}R^{1B}—CR^{1C}R^{1D}—O—, R^{1A} is H; R^{1B} is H; R^{1C} is H; and R^{1D} is H.

In some embodiments of the compound of Formula (IB), the moiety —W¹—W²—W³— is —O—CR^{1C}R^{1D}—CR^{1A}R^{1B}—.

In some embodiments wherein the moiety —W¹—W²—W³— is —O—CR^{1C}R^{1D}—CR^{1A}R^{1B}—, R^{1A} is H; R^{1B} is H; R^{1C} is H; and R^{1D} is H.

In some embodiments of the compound of Formula (IB), the moiety —W¹—W²—W³ is —CR^{1A}R^{1B}—CR^{1C}R^{1D}—CR^{1A}R^{1B}.

In some embodiments wherein the moiety —W¹—W²—W³— is —CR^{1A}R^{1B}—CR^{1C}R^{1D}—CR^{1A}R^{1B}—, R^{1A} is H; R^{1B} is H; R^{1C} is H; and R^{1D} is H.

In some embodiments of the compound of Formula (IB), [symbol] is a single bond.

In some embodiments of the compound of Formula (IB), L is absent.

In some embodiments of the compound of Formula (IB), p=0.

In other embodiments of the compound of Formula (IB), q=0.

In some embodiments of the compound of Formula (IB), [symbol] is a double bond.

In some embodiments of the compound of Formula (IB), [symbol] is a double bond, and L is absent.

In some embodiments of the compound of Formula (IB), [symbol] is a double bond, and p=0.

In some embodiments of the compound of Formula (IB), [symbol] is a double bond, and p=1.

In some embodiments of the compound of Formula (IB), L is NR¹⁴.

In some embodiments of the compound of Formula (IB), R¹⁴ is —C₁-C₆alkyl.

In other embodiments of the compound of Formula (IB), R¹⁴ is —CH₃.

In some embodiments of the compound of Formula (IB), R⁹ is H and R¹⁰ is H.

Those of skill in the art will recognize that the sulfur atom in the sulfonimidamide moiety in the compounds of Formula (I) (and subgenera thereof) is a center of asymmetry. Thus, the compounds of Formula (I) (and subgenera thereof) may exist as a pair of diastereoisomers that differ in the absolute configuration at the sulfur atom of the sulfonimidamide moiety. For example, the compound of Formula (IB)(and subgenera thereof) may exist as (IB-1) and (IB-2):

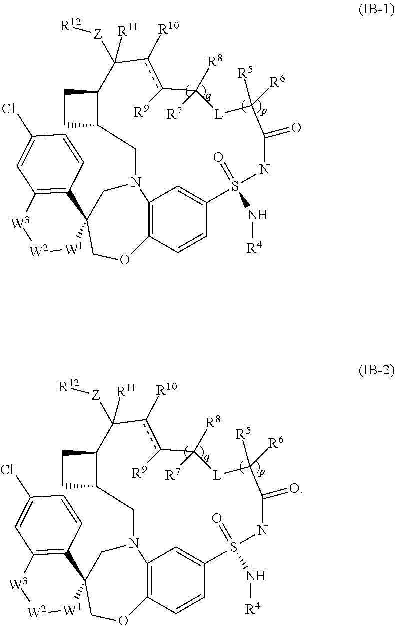

In some embodiments, the compound of Formula (IB) is a compound of Formula (IB-1).

In other embodiments, the compound of Formula IB is a compound of Formula (IB-2).

In some embodiments, the compound of Formula (IB) is a compound of Formula (IC).

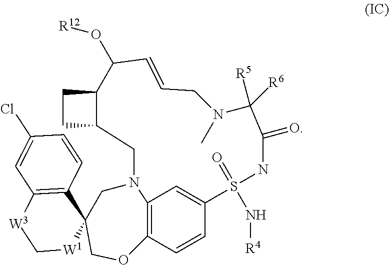

In other embodiments, the compound of Formula (IB) is a compound of Formula (ID):

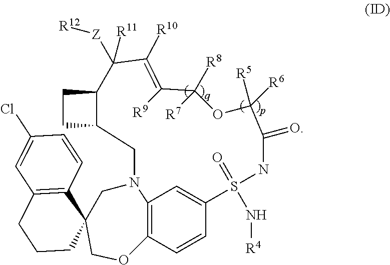

In some embodiments of the compound of Formula (ID), R⁹ is H; R¹⁰ is H; R¹¹ is H; p=1; and q=1.

In some embodiments, the compound of Formula (IB) is a compound of Formula (IE):

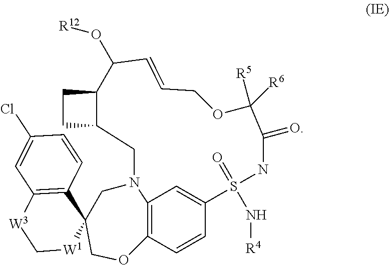

In some embodiments, the compound of Formula (IE) is a compound of Formula (IE-1):

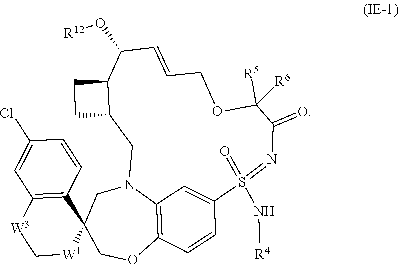

In some embodiments of the compound of Formula (IE-1), both W³ and W¹ are —CH₂—.

In some embodiments of the compound of Formula (IE-1), R¹² is H, optionally substituted C₁-C₁₀alkyl, or —C(O)NR^{c}R^{d}.

In some embodiments of the compound of Formula (IE-1), R¹² is H.

In some embodiments of the compound of Formula (IE-1), R¹² is —C₁-C₁₀alkyl.

In some embodiments of the compound of Formula (IE-1), R¹² is —CH₃.

In some embodiments of the compound of Formula (IE-1), R¹² is C₁-C₁₀alkyl substituted with —NR^{c1}R^{d1}.

In some embodiments of the compound of Formula (IE-1), R¹² is —CH₂CH₂NR^{c1}R^{d1}.

In some embodiments of the compound of Formula (IE-1), R¹² is —CH₂CH₂NR^{c1}R^{d1} wherein R^{c1} and R^{d1} are independently C₁-C₁₀ alkyl.

In some embodiments of the compound of Formula (IE-1), R¹² is —CH₂CH₂—N(CH₃)₂.

In some embodiments of the compound of Formula (IE-1), R¹² is C₁-C₁₀alkyl substituted with heterocycloalkyl.

In some embodiments of the compound of Formula (IE-1), R¹² is

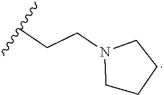 [SMILES: *CCN1CCCC1]

In some embodiments of the compound of Formula (IE-1), R¹² is C(O)NR^{c}R^{d}.

In some embodiments of the compound of Formula (IE-1), R¹² is —C(O)NR^{c}R^{d} wherein R^{c} and R^{d} are each independently —C₁-C₁₀ alkyl.

In some embodiments of the compound of Formula (IE-1), R¹² is —C(O)N(CH₃)₂.

In some embodiments of the compound of Formula (IE-1), both W³ and W¹ are —CH₂— and R¹² is H, optionally substituted C₁-C₁₀alkyl, or —C(O)NR^{c}R^{d}.

In some embodiments of the compound of Formula (IE-1), both W³ and W¹ are —CH₂—, and R¹² is —C₁-C₁₀alkyl.

In some embodiments of the compound of Formula (IE-1), both W³ and W¹ are —CH₂—, and R¹² is —CH₃.

In some embodiments, the compound of Formula (IE-1) is a compound of Formula (IE-1-1).

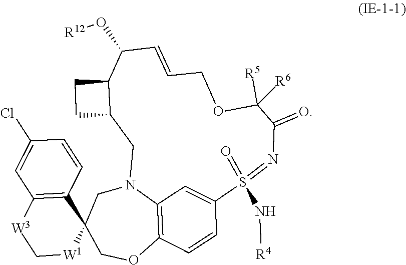

In other embodiments, the compound of Formula (IE-1) is a compound of Formula (IE-1-2):

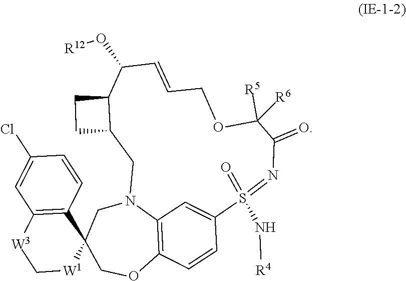

In some embodiments, the compound of Formula (IE) is a compound of Formula (IE-2):

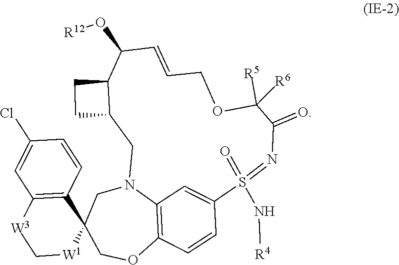

In some embodiments of the compound of Formula (IE-2), both W³ and W¹ are —CH₂—.

In some embodiments of the compound of Formula (IE-2), R¹² is H, optionally substituted C₁-C₁₀alkyl, or —C(O)NR^{c}R^{d}.

In some embodiments of the compound of Formula (IE-2), R¹² is H.

In some embodiments of the compound of Formula (IE-2), R¹² is —C₁-C₁₀alkyl.

In some embodiments of the compound of Formula (IE-2), R¹² is —CH₃.

In some embodiments of the compound of Formula (IE-2), R¹² is C₁-C₁₀alkyl substituted with —NR^{c1}R^{d1}.

In some embodiments of the compound of Formula (IE-2), R¹² is —CH₂CH₂NR^{c1}R^{d1}.

In some embodiments of the compound of Formula (IE-2), R¹² is —CH₂CH₂NR^{c1}R^{d1} wherein R^{c1} and R^{d1} are independently C₁-C₁₀ alkyl.

In some embodiments of the compound of Formula (IE-2), R¹² is —CH₂CH₂—N(CH₃)₂.

In some embodiments of the compound of Formula (IE-2), R¹² is C₁-C₁₀alkyl substituted with heterocycloalkyl.

In some embodiments of the compound of Formula (IE-2), R¹² is

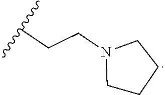 [SMILES: *CCN1CCCC1]

In some embodiments of the compound of Formula (IE-2), R¹² is C(O)NR^{c}R^{d}.

In some embodiments of the compound of Formula (IE-2), R¹² is —C(O)NR^{c}R^{d} wherein R^{c} and R^{d} are each independently —C₁-C₁₀ alkyl.

In some embodiments of the compound of Formula (IE-2), R¹² is —C(O)N(CH₃)₂.

In some embodiments of the compound of Formula (IE-2), both W³ and W¹ are —CH₂— and R¹² is H, optionally substituted C₁-C₁₀alkyl, or —C(O)NR^{c}R^{d}.

In some embodiments of the compound of Formula (IE-2), both W³ and W¹ are —CH₂— and R¹² is optionally substituted —C₁-C₁₀alkyl.

In some embodiments of the compound of Formula (IE-2), both W³ and W¹ are —CH₂— and R¹² is —CH₃.

In some embodiments of the compound of Formula (IE-2), both W³ and W¹ are —CH₂— and R¹² is —CH₂CH₂NR^{c1}R^{d1}.

In some embodiments of the compound of Formula (IE-2), both W³ and W¹ are —CH₂— and R¹² is C(O)NR^{c}R^{d}.

In some embodiments of the compound of Formula (IE-2), R⁵ is optionally substituted —C₁-C₆alkyl; and R⁶ is optionally substituted —C₁-C₆alkyl.

In some embodiments of the compound of Formula (IE-2), R⁵ is —CH₃; and R⁶ is CH₃.

In some embodiments of the compound of Formula (IE-2), The compound according to any one of the preceding claims, wherein R⁴ is —C(O)R^{4B}, or —C(O)NR^{4C}R^{4D}.

In some embodiments, the compound of Formula (IE-2) is a compound of Formula (IE-2-1):

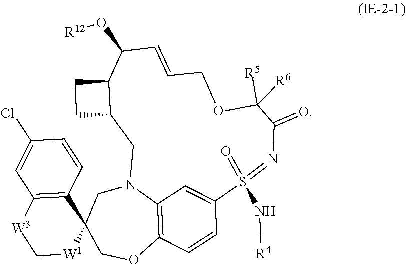

In other embodiments, the compound of Formula (IE-2) is a compound of Formula (IE-2-2):

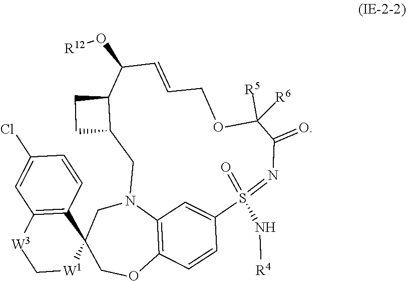

In some embodiments, the compounds of the disclosure include those in the table below.

Some entries in the table identify two Examples. In these entries, the two Example compounds differ by the absolute configuration at the sulfonimdamide moiety. However, the absolute configuration of each specific example has not been ascertained.

| Ex. | STRUCTURE | MW |
|---|---|---|
| 1, 2 | 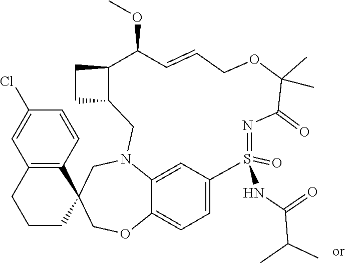 [SMILES: CO[C@H]1/C=C/COC(C)(C)C(=O)N=[S@@](=O)(NC(=O)C(C)C)c2ccc3c(c2)N(C[C@@H]2CC[C@H]21)C[C@@]1(CCCc2cc(Cl)ccc21)CO3] | 684.3 |
| | 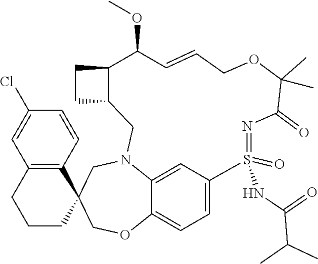 [SMILES: CO[C@H]1/C=C/COC(C)(C)C(=O)N=[S@](=O)(NC(=O)C(C)C)c2ccc3c(c2)N(C[C@@H]2CC[C@H]21)C[C@@]1(CCCc2cc(Cl)ccc21)CO3] | |
| 3, 4 | 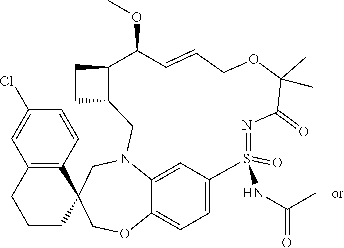 [SMILES: CO[C@H]1/C=C/COC(C)(C)C(=O)N=[S@@](=O)(NC(C)=O)c2ccc3c(c2)N(C[C@@H]2CC[C@H]21)C[C@@]1(CCCc2cc(Cl)ccc21)CO3] | 656.2 |
| | 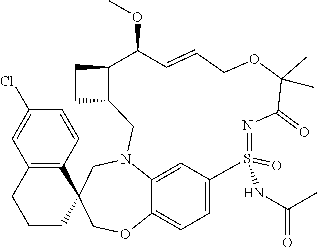 [SMILES: CO[C@H]1/C=C/COC(C)(C)C(=O)N=[S@](=O)(NC(C)=O)c2ccc3c(c2)N(C[C@@H]2CC[C@H]21)C[C@@]1(CCCc2cc(Cl)ccc21)CO3] | |
| 5, 6 | 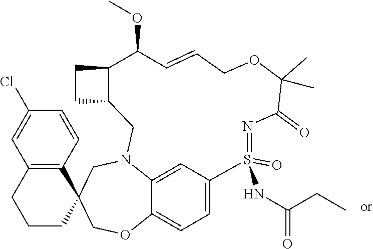 [SMILES: CCC(=O)N[S@]1(=O)=NC(=O)C(C)(C)OC/C=C/[C@H](OC)[C@@H]2CC[C@H]2CN2C[C@@]3(CCCc4cc(Cl)ccc43)COc3ccc1cc32] | 670.3 |
| | 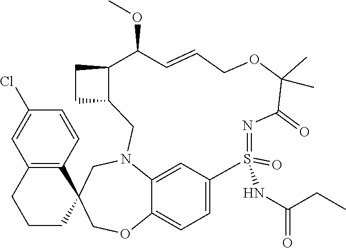 [SMILES: CCC(=O)N[S@@]1(=O)=NC(=O)C(C)(C)OC/C=C/[C@H](OC)[C@@H]2CC[C@H]2CN2C[C@@]3(CCCc4cc(Cl)ccc43)COc3ccc1cc32] | |
| 7, 8 | 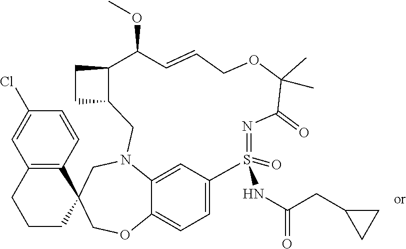 [SMILES: CO[C@H]1/C=C/COC(C)(C)C(=O)N=[S@@](=O)(NC(=O)CC2CC2)c2ccc3c(c2)N(C[C@@H]2CC[C@H]21)C[C@@]1(CCCc2cc(Cl)ccc21)CO3] | 696.3 |
| | 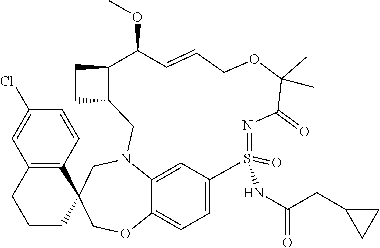 [SMILES: CO[C@H]1/C=C/COC(C)(C)C(=O)N=[S@](=O)(NC(=O)CC2CC2)c2ccc3c(c2)N(C[C@@H]2CC[C@H]21)C[C@@]1(CCCc2cc(Cl)ccc21)CO3] | |
| 9, 10 | 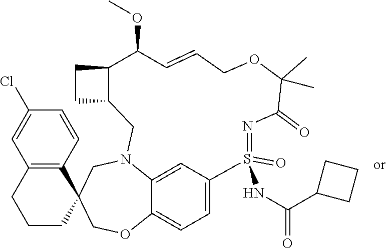 [SMILES: CO[C@H]1/C=C/COC(C)(C)C(=O)N=[S@@](=O)(NC(=O)C2CCC2)c2ccc3c(c2)N(C[C@@H]2CC[C@H]21)C[C@@]1(CCCc2cc(Cl)ccc21)CO3] | 696.3 |
| | 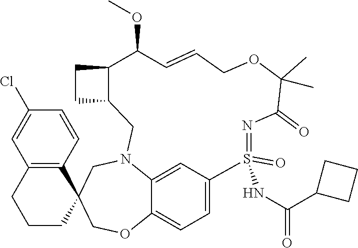 [SMILES: CO[C@H]1/C=C/COC(C)(C)C(=O)N=[S@](=O)(NC(=O)C2CCC2)c2ccc3c(c2)N(C[C@@H]2CC[C@H]21)C[C@@]1(CCCc2cc(Cl)ccc21)CO3] | |
| 11 | 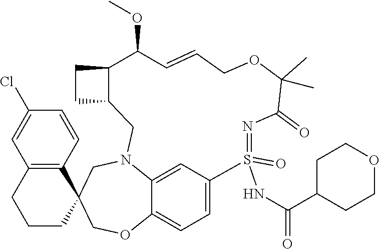 [SMILES: CO[C@H]1/C=C/COC(C)(C)C(=O)N=S(=O)(NC(=O)C2CCOCC2)c2ccc3c(c2)N(C[C@@H]2CC[C@H]21)C[C@@]1(CCCc2cc(Cl)ccc21)CO3] | 726.3 |
| 12 | 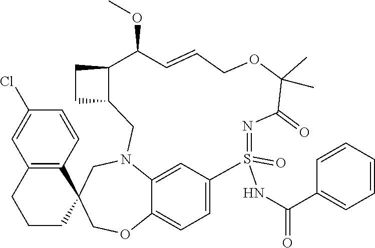 [SMILES: CO[C@H]1/C=C/COC(C)(C)C(=O)N=S(=O)(NC(=O)c2ccccc2)c2ccc3c(c2)N(C[C@@H]2CC[C@H]21)C[C@@]1(CCCc2cc(Cl)ccc21)CO3] | 717.3 |
| 13, 14 | 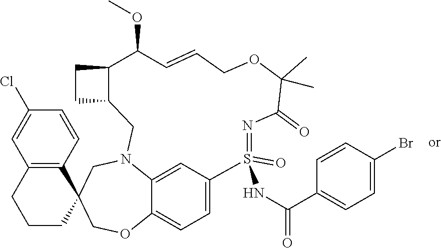 [SMILES: CO[C@H]1/C=C/COC(C)(C)C(=O)N=[S@@](=O)(NC(=O)c2ccc(Br)cc2)c2ccc3c(c2)N(C[C@@H]2CC[C@H]21)C[C@@]1(CCCc2cc(Cl)ccc21)CO3] | 797.2 |
| | 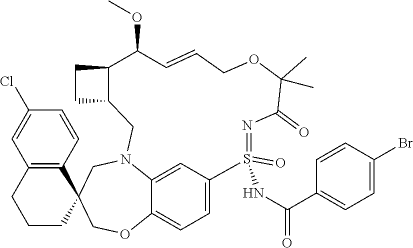 [SMILES: CO[C@H]1/C=C/COC(C)(C)C(=O)N=[S@](=O)(NC(=O)c2ccc(Br)cc2)c2ccc3c(c2)N(C[C@@H]2CC[C@H]21)C[C@@]1(CCCc2cc(Cl)ccc21)CO3] | |
| 15 | 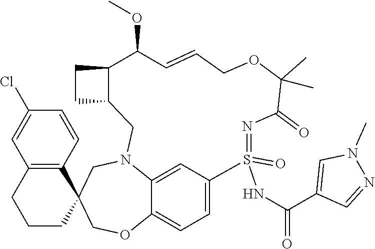 [SMILES: CO[C@H]1/C=C/COC(C)(C)C(=O)N=S(=O)(NC(=O)c2cnn(C)c2)c2ccc3c(c2)N(C[C@@H]2CC[C@H]21)C[C@@]1(CCCc2cc(Cl)ccc21)CO3] | 722.3 |
| 16 | 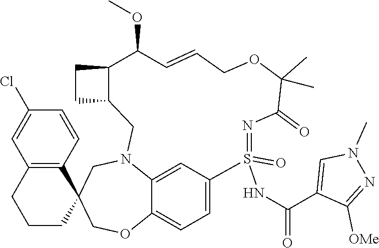 [SMILES: COc1nn(C)cc1C(=O)NS1(=O)=NC(=O)C(C)(C)OC/C=C/[C@H](OC)[C@@H]2CC[C@H]2CN2C[C@@]3(CCCc4cc(Cl)ccc43)COc3ccc1cc32] | 752.3 |
| 17 | 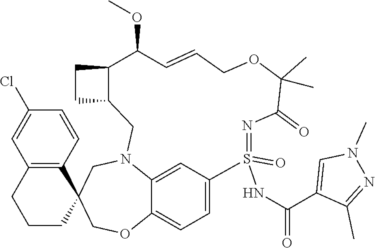 [SMILES: CO[C@H]1/C=C/COC(C)(C)C(=O)N=S(=O)(NC(=O)c2cn(C)nc2C)c2ccc3c(c2)N(C[C@@H]2CC[C@H]21)C[C@@]1(CCCc2cc(Cl)ccc21)CO3] | 736.3 |
| 18 | 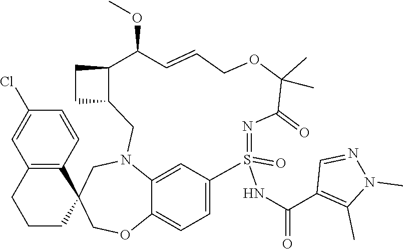 [SMILES: CO[C@H]1/C=C/COC(C)(C)C(=O)N=S(=O)(NC(=O)c2cnn(C)c2C)c2ccc3c(c2)N(C[C@@H]2CC[C@H]21)C[C@@]1(CCCc2cc(Cl)ccc21)CO3] | 736.3 |
| 19 | 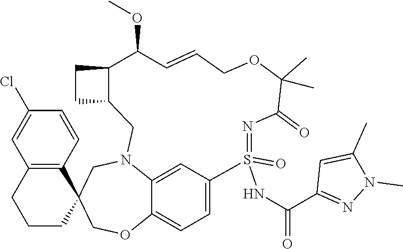 [SMILES: CO[C@H]1/C=C/COC(C)(C)C(=O)N=S(=O)(NC(=O)c2cc(C)n(C)n2)c2ccc3c(c2)N(C[C@@H]2CC[C@H]21)C[C@@]1(CCCc2cc(Cl)ccc21)CO3] | 736.3 |
| 20 | 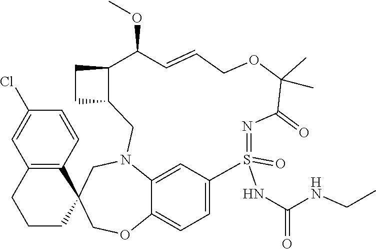 [SMILES: CCNC(=O)NS1(=O)=NC(=O)C(C)(C)OC/C=C/[C@H](OC)[C@@H]2CC[C@H]2CN2C[C@@]3(CCCc4cc(Cl)ccc43)COc3ccc1cc32] | 785.3 |
| 21, 22 | 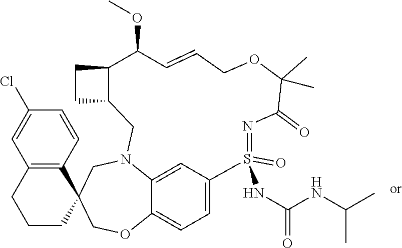 [SMILES: CO[C@H]1/C=C/COC(C)(C)C(=O)N=[S@@](=O)(NC(=O)NC(C)C)c2ccc3c(c2)N(C[C@@H]2CC[C@H]21)C[C@@]1(CCCc2cc(Cl)ccc21)CO3] | 699.3 |
| | 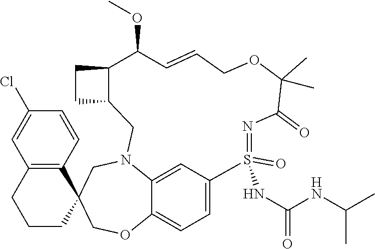 [SMILES: CO[C@H]1/C=C/COC(C)(C)C(=O)N=[S@](=O)(NC(=O)NC(C)C)c2ccc3c(c2)N(C[C@@H]2CC[C@H]21)C[C@@]1(CCCc2cc(Cl)ccc21)CO3] | |
| 23, 24 | 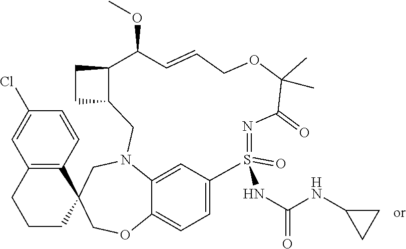 [SMILES: CO[C@H]1/C=C/COC(C)(C)C(=O)N=[S@@](=O)(NC(=O)NC2CC2)c2ccc3c(c2)N(C[C@@H]2CC[C@H]21)C[C@@]1(CCCc2cc(Cl)ccc21)CO3] | 697.3 |
| | 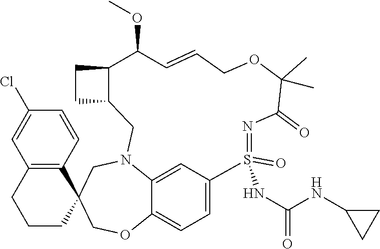 [SMILES: CO[C@H]1/C=C/COC(C)(C)C(=O)N=[S@](=O)(NC(=O)NC2CC2)c2ccc3c(c2)N(C[C@@H]2CC[C@H]21)C[C@@]1(CCCc2cc(Cl)ccc21)CO3] | |
| 25, 26 | 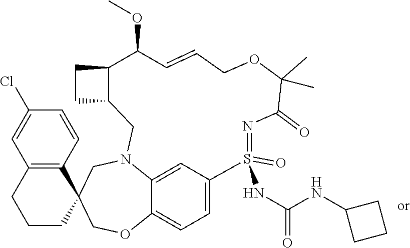 [SMILES: CO[C@H]1/C=C/COC(C)(C)C(=O)N=[S@@](=O)(NC(=O)NC2CCC2)c2ccc3c(c2)N(C[C@@H]2CC[C@H]21)C[C@@]1(CCCc2cc(Cl)ccc21)CO3] | 711.3 |
| | 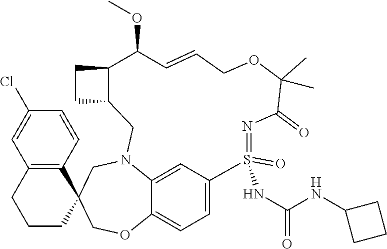 [SMILES: CO[C@H]1/C=C/COC(C)(C)C(=O)N=[S@](=O)(NC(=O)NC2CCC2)c2ccc3c(c2)N(C[C@@H]2CC[C@H]21)C[C@@]1(CCCc2cc(Cl)ccc21)CO3] | |
| 27, 28 | 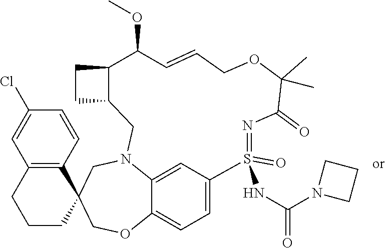 [SMILES: CO[C@H]1/C=C/COC(C)(C)C(=O)N=[S@@](=O)(NC(=O)N2CCC2)c2ccc3c(c2)N(C[C@@H]2CC[C@H]21)C[C@@]1(CCCc2cc(Cl)ccc21)CO3] | 697.3 |
| | 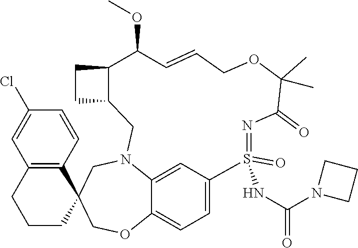 [SMILES: CO[C@H]1/C=C/COC(C)(C)C(=O)N=[S@](=O)(NC(=O)N2CCC2)c2ccc3c(c2)N(C[C@@H]2CC[C@H]21)C[C@@]1(CCCc2cc(Cl)ccc21)CO3] | |
| 29, 30 | 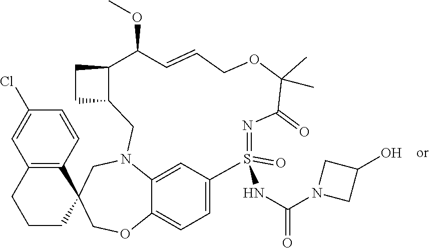 [SMILES: CO[C@H]1/C=C/COC(C)(C)C(=O)N=[S@@](=O)(NC(=O)N2CC(O)C2)c2ccc3c(c2)N(C[C@@H]2CC[C@H]21)C[C@@]1(CCCc2cc(Cl)ccc21)CO3] | 713.3 |
| | 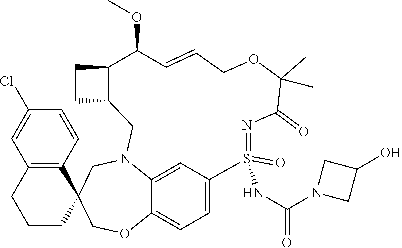 [SMILES: CO[C@H]1/C=C/COC(C)(C)C(=O)N=[S@](=O)(NC(=O)N2CC(O)C2)c2ccc3c(c2)N(C[C@@H]2CC[C@H]21)C[C@@]1(CCCc2cc(Cl)ccc21)CO3] | |
| 31, 32 | 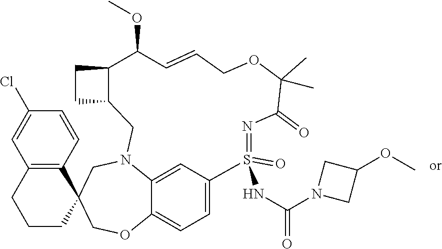 [SMILES: COC1CN(C(=O)N[S@]2(=O)=NC(=O)C(C)(C)OC/C=C/[C@H](OC)[C@@H]3CC[C@H]3CN3C[C@@]4(CCCc5cc(Cl)ccc54)COc4ccc2cc43)C1] | 727.3 |
| | 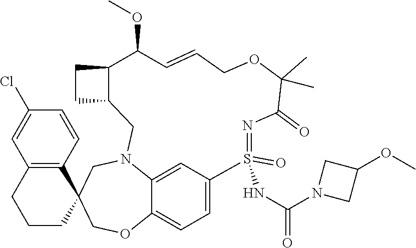 [SMILES: COC1CN(C(=O)N[S@@]2(=O)=NC(=O)C(C)(C)OC/C=C/[C@H](OC)[C@@H]3CC[C@H]3CN3C[C@@]4(CCCc5cc(Cl)ccc54)COc4ccc2cc43)C1] | |
| 33 | 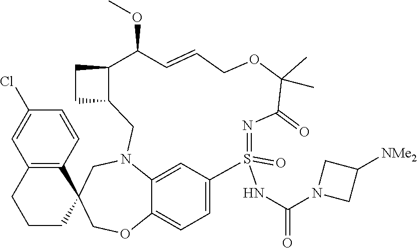 [SMILES: CO[C@H]1/C=C/COC(C)(C)C(=O)N=S(=O)(NC(=O)N2CC(N(C)C)C2)c2ccc3c(c2)N(C[C@@H]2CC[C@H]21)C[C@@]1(CCCc2cc(Cl)ccc21)CO3] | 740.4 |
| 34, 35 | 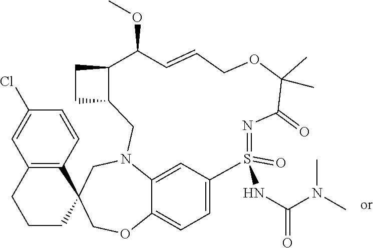 [SMILES: CO[C@H]1/C=C/COC(C)(C)C(=O)N=[S@@](=O)(NC(=O)N(C)C)c2ccc3c(c2)N(C[C@@H]2CC[C@H]21)C[C@@]1(CCCc2cc(Cl)ccc21)CO3] | 685.3 |
| | 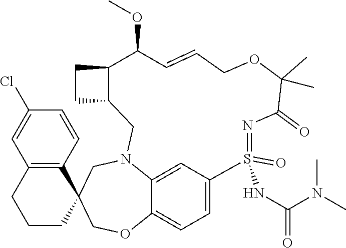 [SMILES: CO[C@H]1/C=C/COC(C)(C)C(=O)N=[S@](=O)(NC(=O)N(C)C)c2ccc3c(c2)N(C[C@@H]2CC[C@H]21)C[C@@]1(CCCc2cc(Cl)ccc21)CO3] | |
| 36, 37 | 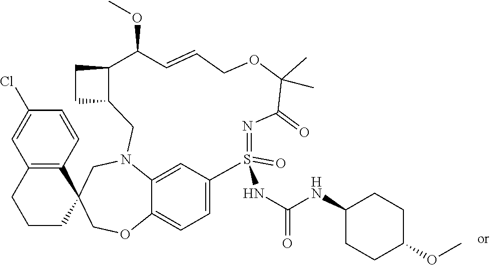 [SMILES: CO[C@H]1/C=C/COC(C)(C)C(=O)N=[S@@](=O)(NC(=O)N[C@H]2CC[C@H](OC)CC2)c2ccc3c(c2)N(C[C@@H]2CC[C@H]21)C[C@@]1(CCCc2cc(Cl)ccc21)CO3] | 769.4 |
| | 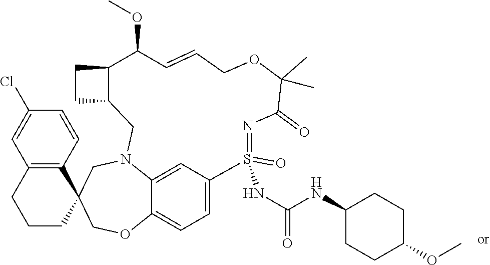 [SMILES: CO[C@H]1/C=C/COC(C)(C)C(=O)N=[S@](=O)(NC(=O)N[C@H]2CC[C@H](OC)CC2)c2ccc3c(c2)N(C[C@@H]2CC[C@H]21)C[C@@]1(CCCc2cc(Cl)ccc21)CO3] | |
| 38 | 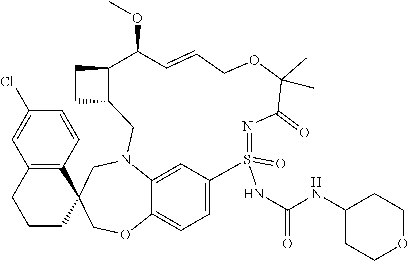 [SMILES: CO[C@H]1/C=C/COC(C)(C)C(=O)N=S(=O)(NC(=O)NC2CCOCC2)c2ccc3c(c2)N(C[C@@H]2CC[C@H]21)C[C@@]1(CCCc2cc(Cl)ccc21)CO3] | 741.3 |
| 39, 40 | 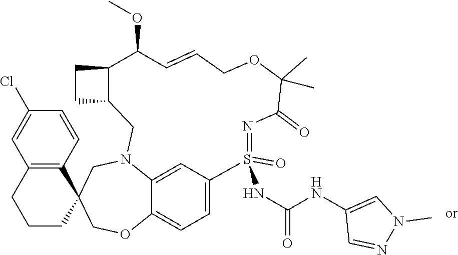 [SMILES: CO[C@H]1/C=C/COC(C)(C)C(=O)N=[S@@](=O)(NC(=O)Nc2cnn(C)c2)c2ccc3c(c2)N(C[C@@H]2CC[C@H]21)C[C@@]1(CCCc2cc(Cl)ccc21)CO3] | 737.3 |
| | 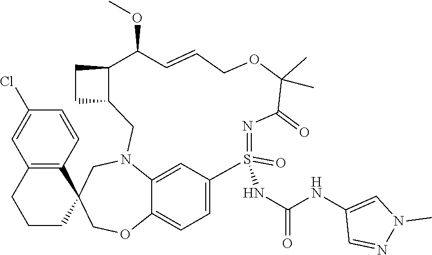 [SMILES: CO[C@H]1/C=C/COC(C)(C)C(=O)N=[S@](=O)(NC(=O)Nc2cnn(C)c2)c2ccc3c(c2)N(C[C@@H]2CC[C@H]21)C[C@@]1(CCCc2cc(Cl)ccc21)CO3] | |
| 41, 42 | 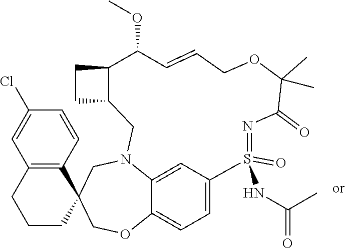 [SMILES: CO[C@@H]1/C=C/COC(C)(C)C(=O)N=[S@@](=O)(NC(C)=O)c2ccc3c(c2)N(C[C@@H]2CC[C@H]21)C[C@@]1(CCCc2cc(Cl)ccc21)CO3] | 656.2 |
| | 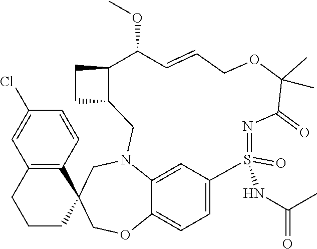 [SMILES: CO[C@@H]1/C=C/COC(C)(C)C(=O)N=[S@](=O)(NC(C)=O)c2ccc3c(c2)N(C[C@@H]2CC[C@H]21)C[C@@]1(CCCc2cc(Cl)ccc21)CO3] | |
| 43, 44 | 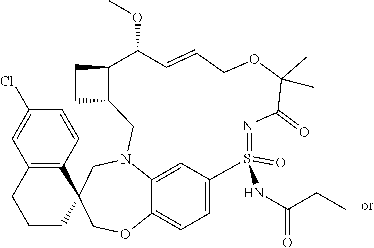 [SMILES: CCC(=O)N[S@]1(=O)=NC(=O)C(C)(C)OC/C=C/[C@@H](OC)[C@@H]2CC[C@H]2CN2C[C@@]3(CCCc4cc(Cl)ccc43)COc3ccc1cc32] | 670.3 |
| | 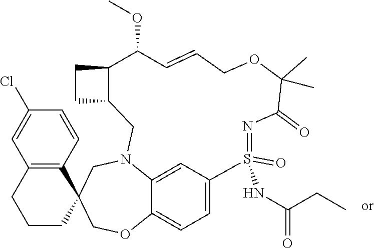 [SMILES: CCC(=O)N[S@@]1(=O)=NC(=O)C(C)(C)OC/C=C/[C@@H](OC)[C@@H]2CC[C@H]2CN2C[C@@]3(CCCc4cc(Cl)ccc43)COc3ccc1cc32] | |
| 45, 46 | 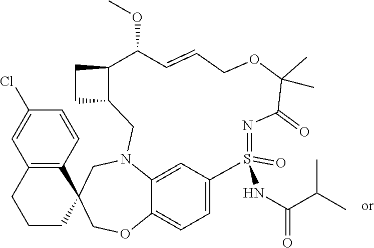 [SMILES: CO[C@@H]1/C=C/COC(C)(C)C(=O)N=[S@@](=O)(NC(=O)C(C)C)c2ccc3c(c2)N(C[C@@H]2CC[C@H]21)C[C@@]1(CCCc2cc(Cl)ccc21)CO3] | 684.3 |
| | 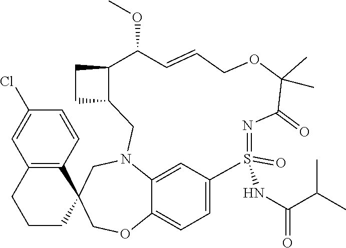 [SMILES: CO[C@@H]1/C=C/COC(C)(C)C(=O)N=[S@](=O)(NC(=O)C(C)C)c2ccc3c(c2)N(C[C@@H]2CC[C@H]21)C[C@@]1(CCCc2cc(Cl)ccc21)CO3] | |
| 47, 48 | 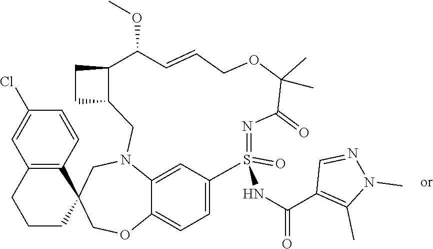 [SMILES: CO[C@@H]1/C=C/COC(C)(C)C(=O)N=[S@@](=O)(NC(=O)c2cnn(C)c2C)c2ccc3c(c2)N(C[C@@H]2CC[C@H]21)C[C@@]1(CCCc2cc(Cl)ccc21)CO3] | 736.3 |
| | 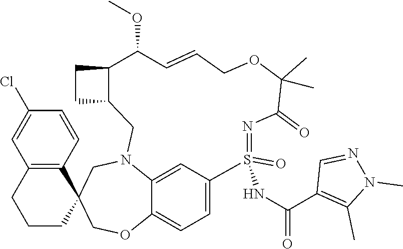 [SMILES: CO[C@@H]1/C=C/COC(C)(C)C(=O)N=[S@](=O)(NC(=O)c2cnn(C)c2C)c2ccc3c(c2)N(C[C@@H]2CC[C@H]21)C[C@@]1(CCCc2cc(Cl)ccc21)CO3] | |
| 49, 50 | 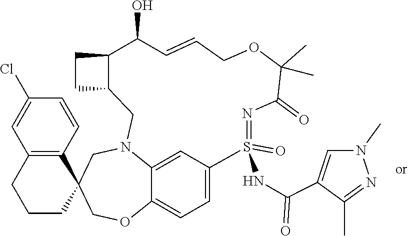 [SMILES: Cc1nn(C)cc1C(=O)N[S@]1(=O)=NC(=O)C(C)(C)OC/C=C/[C@H](O)[C@@H]2CC[C@H]2CN2C[C@@]3(CCCc4cc(Cl)ccc43)COc3ccc1cc32] | 722.3 |
| | 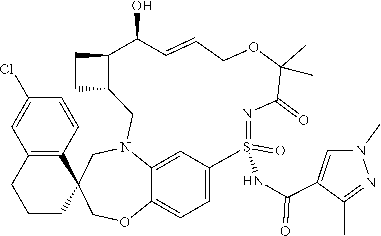 [SMILES: Cc1nn(C)cc1C(=O)N[S@@]1(=O)=NC(=O)C(C)(C)OC/C=C/[C@H](O)[C@@H]2CC[C@H]2CN2C[C@@]3(CCCc4cc(Cl)ccc43)COc3ccc1cc32] | |
| 51 | 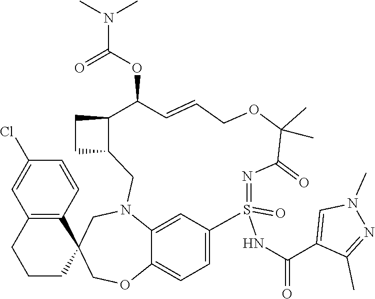 [SMILES: Cc1nn(C)cc1C(=O)NS1(=O)=NC(=O)C(C)(C)OC/C=C/[C@H](OC(=O)N(C)C)[C@@H]2CC[C@H]2CN2C[C@@]3(CCCc4cc(Cl)ccc43)COc3ccc1cc32] | 793.4 |
| 52 | 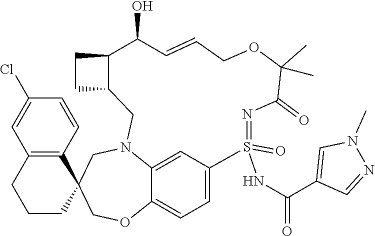 [SMILES: Cn1cc(C(=O)NS2(=O)=NC(=O)C(C)(C)OC/C=C/[C@H](O)[C@@H]3CC[C@H]3CN3C[C@@]4(CCCc5cc(Cl)ccc54)COc4ccc2cc43)cn1] | 708.3 |
| 53 | 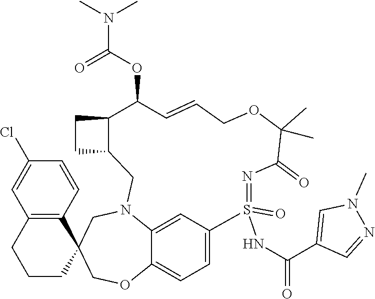 [SMILES: CN(C)C(=O)O[C@H]1/C=C/COC(C)(C)C(=O)N=S(=O)(NC(=O)c2cnn(C)c2)c2ccc3c(c2)N(C[C@@H]2CC[C@H]21)C[C@@]1(CCCc2cc(Cl)ccc21)CO3] | 779.4 |
| 54 | 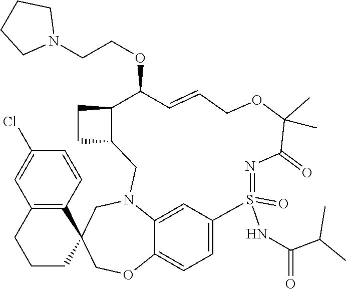 [SMILES: CC(C)C(=O)NS1(=O)=NC(=O)C(C)(C)OC/C=C/[C@H](OCCN2CCCC2)[C@@H]2CC[C@H]2CN2C[C@@]3(CCCc4cc(Cl)ccc43)COc3ccc1cc32] | 767.4 |
| 55 | 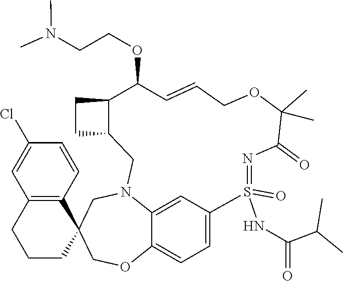 [SMILES: CC(C)C(=O)NS1(=O)=NC(=O)C(C)(C)OC/C=C/[C@H](OCCN(C)C)[C@@H]2CC[C@H]2CN2C[C@@]3(CCCc4cc(Cl)ccc43)COc3ccc1cc32] | 741.4 |

It will be apparent that the compounds of Formula I, including all subgenera described herein, have multiple stereogenic centers. As a result, there exist multiple stereoisomers (enantiomers and diastereomers) of the compounds of Formula I (subgenera described herein). The present disclosure contemplates and encompasses each stereoisomer of any compound of Formula I (and subgenera described herein), as well as mixtures of said stereoisomers.

Pharmaceutically acceptable salts and solvates of the compounds of Formula I (including all subgenera described herein) are also within the scope of the disclosure.

Isotopic variants of the compounds of Formula I (including all subgenera described herein) are also contemplated by the present disclosure.

### Pharmaceutical Compositions and Methods of Administration

The subject pharmaceutical compositions are typically formulated to provide a therapeutically effective amount of a compound of the present disclosure as the active ingredient, or a pharmaceutically acceptable salt, ester, prodrug, solvate, hydrate or derivative thereof. Where desired, the pharmaceutical compositions contain pharmaceutically acceptable salt and/or coordination complex thereof, and one or more pharmaceutically acceptable excipients, carriers, including inert solid diluents and fillers, diluents, including sterile aqueous solution and various organic solvents, permeation enhancers, solubilizers and adjuvants.

The subject pharmaceutical compositions can be administered alone or in combination with one or more other agents, which are also typically administered in the form of pharmaceutical compositions. Where desired, the one or more compounds of the invention and other agent(s) may be mixed into a preparation or both components may be formulated into separate preparations to use them in combination separately or at the same time.

In some embodiments, the concentration of one or more compounds provided in the pharmaceutical compositions of the present invention is less than 100%, 90%, 80%, 70%, 60%, 50%, 40%, 30%, 20%, 19%, 18%, 1%, %16%, 15%, 14%, 13%, 12%, 11%, 10%, 9%, 8%, 7%, 6%, 5%, 4%, 3%, 2%, 1%, 0.9%, 0.8%, 0.7%, 0.6% 0.5%, 0.4%, 0.3%, 0.2%, 0.1%, 0.09%, 0.08%, 0.07%, 0.06%, 0.05%, 0.04%, 0.03%, 0.02%, 0.01%, 0.009%, 0.008%, 0.007%, 0.006%, 0.005%, 0.004%, 0.003%, 0.002%, 0.001%, 0.0009%, 0.0008%, 0.0007%, 0.0006%, 0.0005%, 0.0004%, 0.0003%, 0.0002%, or 0.0001% (or a number in the range defined by and including any two numbers above) w/w, w/v or v/v.

In some embodiments, the concentration of one or more compounds of the invention is greater than 90%, 80%, 70%, 60%, 50%, 40%, 30%, 20%, 19.75%, 19.50%1, 9.25%, 19%, 18.75%, 18.50%, 18.25% 18%, 17.75%, 17.50%, 17.25% 17%, 16.75%, 16.50%, 16.25%, 16%, 15.75%, 15.50%, 15.25% 15%, 14.75%, 14.50%, 14.25% 14%, 13.75%, 13.50%, 13.25%, 13%, 12.75%, 12.50%, 12.25%, 12%, 11.75%, 11.50%, 11.25% 11%, 10.75%, 10.50%, 10.25% 10%, 9.75%, 9.50%, 9.25%, 9%, 8.75%, 8.50%, 8.25% 8%, 7.75%, 7.50%, 7.25%, 7%, 6.75%, 6.50%, 6.25%, 6%, 5.75%, 5.50%, 5.25%, 5%, 4.75%, 4.50%, 4.25%, 4%, 3.75%, 3.50%, 3.25%, 3%, 2.75%, 2.50%, 2.25%, 2%, 1.75%, 1.50%, 1.25%, 1%, 0.9%, 0.8% 0.7% 0.6% 0.5%, 0.4%, 0.3%, 0.2%, 0.1%, 0.09%, 0.08%, 0.07%, 0.06%, 0.05%, 0.04%, 0.03%, 0.02%, 0.01%, 0.009%, 0.008%, 0.007%, 0.006%, 0.005%, 0.004%, 0.003%, 0.002%, 0.001%, 0.0009%, 0.0008%, 0.0007%, 0.0006%, 0.0005%, 0.0004%, 0.0003%, 0.0002%, or 0.0001% (or a number in the range defined by and including any two numbers above) w/w, w/v, or v/v.

In some embodiments, the concentration of one or more compounds of the invention is in the range from approximately 0.0001% to approximately 50%, approximately 0.001% to approximately 40%, approximately 0.01% to approximately 30%, approximately 0.02% to approximately 29%, approximately 0.03% to approximately 28%, approximately 0.04% to approximately 27%, approximately 0.05% to approximately 26%, approximately 0.06% to approximately 25%, approximately 0.07% to approximately 24%, approximately 0.08% to approximately 23%, approximately 0.09% to approximately 22%, approximately 0.1% to approximately 21%, approximately 0.2% to approximately 20%, approximately 0.3% to approximately 19%, approximately 0.4% to approximately 18%, approximately 0.5% to approximately 17%, approximately 0.6% to approximately 16%, approximately 0.7% to approximately 15%, approximately 0.8% to approximately 14%, approximately 0.9% to approximately 12%, approximately 1% to approximately 10% w/w, w/v or v/v.

In some embodiments, the concentration of one or more compounds of the invention is in the range from approximately 0.001% to approximately 10%, approximately 0.01% to approximately 5%, approximately 0.02% to approximately 4.5%, approximately 0.03% to approximately 4%, approximately 0.04% to approximately 3.5%, approximately 0.05% to approximately 3%, approximately 0.06% to approximately 2.5%, approximately 0.07% to approximately 2%, approximately 0.08% to approximately 1.5%, approximately 0.09% to approximately 1%, approximately 0.1% to approximately 0.9% w/w, w/v or v/v.

In some embodiments, the amount of one or more compounds of the invention is equal to or less than 10 g, 9.5 g, 9.0 g, 8.5 g, 8.0 g, 7.5 g, 7.0 g, 6.5 g, 6.0 g, 5.5 g, 5.0 g, 4.5 g, 4.0 g, 3.5 g, 3.0 g, 2.5 g, 2.0 g, 1.5 g, 1.0 g, 0.95 g, 0.9 g, 0.85 g, 0.8 g, 0.75 g, 0.7 g, 0.65 g, 0.6 g, 0.55 g, 0.5 g, 0.45 g, 0.4 g, 0.35 g, 0.3 g, 0.25 g, 0.2 g, 0.15 g, 0.1 g, 0.09 g, 0.08 g, 0.07 g, 0.06 g, 0.05 g, 0.04 g, 0.03 g, 0.02 g, 0.01 g, 0.009 g, 0.008 g, 0.007 g, 0.006 g, 0.005 g, 0.004 g, 0.003 g, 0.002 g, 0.001 g, 0.0009 g, 0.0008 g, 0.0007 g, 0.0006 g, 0.0005 g, 0.0004 g, 0.0003 g, 0.0002 g, or 0.0001 g (or a number in the range defined by and including any two numbers above).

In some embodiments, the amount of one or more compounds of the invention is more than 0.0001 g, 0.0002 g, 0.0003 g, 0.0004 g, 0.0005 g, 0.0006 g, 0.0007 g, 0.0008 g, 0.0009 g, 0.001 g, 0.0015 g, 0.002 g, 0.0025 g, 0.003 g, 0.0035 g, 0.004 g, 0.0045 g, 0.005 g, 0.0055 g, 0.006 g, 0.0065 g, 0.007 g, 0.0075 g, 0.008 g, 0.0085 g, 0.009 g, 0.0095 g, 0.01 g, 0.015 g, 0.02 g, 0.025 g, 0.03 g, 0.035 g, 0.04 g, 0.045 g, 0.05 g, 0.055 g, 0.06 g, 0.065 g, 0.07 g, 0.075 g, 0.08 g, 0.085 g, 0.09 g, 0.095 g, 0.1 g, 0.15 g, 0.2 g, 0.25 g, 0.3 g, 0.35 g, 0.4 g, 0.45 g, 0.5 g, 0.55 g, 0.6 g, 0.65 g, 0.7 g, 0.75 g, 0.8 g, 0.85 g, 0.9 g, 0.95 g, 1 g, 1.5 g, 2 g, 2.5, 3 g, 3.5, 4 g, 4.5 g, 5 g, 5.5 g, 6 g, 6.5 g, 7 g, 7.5 g, 8 g, 8.5 g, 9 g, 9.5 g, or 10 g (or a number in the range defined by and including any two numbers above).

In some embodiments, the amount of one or more compounds of the invention is in the range of 0.0001-10 g, 0.0005-9 g, 0.001-8 g, 0.005-7 g, 0.01-6 g, 0.05-5 g, 0.1-4 g, 0.5-4 g, or 1-3 g.

The compounds according to the invention are effective over a wide dosage range. For example, in the treatment of adult humans, dosages from 0.01 to 1000 mg, from 0.5 to 100 mg, from 1 to 50 mg per day, and from 5 to 40 mg per day are examples of dosages that may be used. An exemplary dosage is 10 to 30 mg per day. The exact dosage will depend upon the route of administration, the form in which the compound is administered, the subject to be treated, the body weight of the subject to be treated, and the preference and experience of the attending physician.

A pharmaceutical composition of the invention typically contains an active ingredient (i.e., a compound of the disclosure) of the present invention or a pharmaceutically acceptable salt and/or coordination complex thereof, and one or more pharmaceutically acceptable excipients, carriers, including but not limited to inert solid diluents and fillers, diluents, sterile aqueous solution and various organic solvents, permeation enhancers, solubilizers and adjuvants.

Described below are non-limiting exemplary pharmaceutical compositions and methods for preparing the same.

### Pharmaceutical Compositions for Oral Administration.

In some embodiments, the invention provides a pharmaceutical composition for oral administration containing a compound of the invention, and a pharmaceutical excipient suitable for oral administration.

In some embodiments, the invention provides a solid pharmaceutical composition for oral administration containing: (i) an effective amount of a compound of the invention; optionally (ii) an effective amount of a second agent; and (iii) a pharmaceutical excipient suitable for oral administration. In some embodiments, the composition further contains: (iv) an effective amount of a third agent.

In some embodiments, the pharmaceutical composition may be a liquid pharmaceutical composition suitable for oral consumption. Pharmaceutical compositions of the invention suitable for oral administration can be presented as discrete dosage forms, such as capsules, cachets, or tablets, or liquids or aerosol sprays each containing a predetermined amount of an active ingredient as a powder or in granules, a solution, or a suspension in an aqueous or non-aqueous liquid, an oil-in-water emulsion, or a water-in-oil liquid emulsion. Such dosage forms can be prepared by any of the methods of pharmacy, but all methods include the step of bringing the active ingredient into association with the carrier, which constitutes one or more necessary ingredients. In general, the compositions are prepared by uniformly and intimately admixing the active ingredient with liquid carriers or finely divided solid carriers or both, and then, if necessary, shaping the product into the desired presentation. For example, a tablet can be prepared by compression or molding, optionally with one or more accessory ingredients. Compressed tablets can be prepared by compressing in a suitable machine the active ingredient in a free-flowing form such as powder or granules, optionally mixed with an excipient such as, but not limited to, a binder, a lubricant, an inert diluent, and/or a surface active or dispersing agent. Molded tablets can be made by molding in a suitable machine a mixture of the powdered compound moistened with an inert liquid diluent.

This invention further encompasses anhydrous pharmaceutical compositions and dosage forms comprising an active ingredient, since water can facilitate the degradation of some compounds. For example, water may be added (e.g., 5%) in the pharmaceutical arts as a means of simulating long-term storage in order to determine characteristics such as shelf-life or the stability of formulations over time. Anhydrous pharmaceutical compositions and dosage forms of the invention can be prepared using anhydrous or low moisture containing ingredients and low moisture or low humidity conditions. Pharmaceutical compositions and dosage forms of the invention which contain lactose can be made anhydrous if substantial contact with moisture and/or humidity during manufacturing, packaging, and/or storage is expected. An anhydrous pharmaceutical composition may be prepared and stored such that its anhydrous nature is maintained. Accordingly, anhydrous compositions may be packaged using materials known to prevent exposure to water such that they can be included in suitable formulary kits. Examples of suitable packaging include, but are not limited to, hermetically sealed foils, plastic or the like, unit dose containers, blister packs, and strip packs.

An active ingredient can be combined in an intimate admixture with a pharmaceutical carrier according to conventional pharmaceutical compounding techniques. The carrier can take a wide variety of forms depending on the form of preparation desired for administration. In preparing the compositions for an oral dosage form, any of the usual pharmaceutical media can be employed as carriers, such as, for example, water, glycols, oils, alcohols, flavoring agents, preservatives, coloring agents, and the like in the case of oral liquid preparations (such as suspensions, solutions, and elixirs) or aerosols; or carriers such as starches, sugars, micro-crystalline cellulose, diluents, granulating agents, lubricants, binders, and disintegrating agents can be used in the case of oral solid preparations, in some embodiments without employing the use of lactose. For example, suitable carriers include powders, capsules, and tablets, with the solid oral preparations. If desired, tablets can be coated by standard aqueous or nonaqueous techniques.

Binders suitable for use in pharmaceutical compositions and dosage forms include, but are not limited to, corn starch, potato starch, or other starches, gelatin, natural and synthetic gums such as acacia, sodium alginate, alginic acid, other alginates, powdered tragacanth, guar gum, cellulose and its derivatives (e.g., ethyl cellulose, cellulose acetate, carboxymethyl cellulose calcium, sodium carboxymethyl cellulose), polyvinyl pyrrolidone, methyl cellulose, pre-gelatinized starch, hydroxypropyl methyl cellulose, microcrystalline cellulose, and mixtures thereof.

Examples of suitable fillers for use in the pharmaceutical compositions and dosage forms disclosed herein include, but are not limited to, talc, calcium carbonate (e.g., granules or powder), microcrystalline cellulose, powdered cellulose, dextrates, kaolin, mannitol, silicic acid, sorbitol, starch, pre-gelatinized starch, and mixtures thereof.

Disintegrants may be used in the compositions of the invention to provide tablets that disintegrate when exposed to an aqueous environment. Too much of a disintegrant may produce tablets which may disintegrate in the bottle. Too little may be insufficient for disintegration to occur and may thus alter the rate and extent of release of the active ingredient(s) from the dosage form.

Thus, a sufficient amount of disintegrant that is neither too little nor too much to detrimentally alter the release of the active ingredient(s) may be used to form the dosage forms of the compounds disclosed herein. The amount of disintegrant used may vary based upon the type of formulation and mode of administration, and may be readily discernible to those of ordinary skill in the art. About 0.5 to about 15 weight percent of disintegrant, or about 1 to about 5 weight percent of disintegrant, may be used in the pharmaceutical composition. Disintegrants that can be used to form pharmaceutical compositions and dosage forms of the invention include, but are not limited to, agar-agar, alginic acid, calcium carbonate, microcrystalline cellulose, croscarmellose sodium, crospovidone, polacrilin potassium, sodium starch glycolate, potato or tapioca starch, other starches, pre-gelatinized starch, other starches, clays, other algins, other celluloses, gums or mixtures thereof.

Lubricants which can be used to form pharmaceutical compositions and dosage forms of the invention include, but are not limited to, calcium stearate, magnesium stearate, mineral oil, light mineral oil, glycerin, sorbitol, mannitol, polyethylene glycol, other glycols, stearic acid, sodium lauryl sulfate, talc, hydrogenated vegetable oil (e.g., peanut oil, cottonseed oil, sunflower oil, sesame oil, olive oil, corn oil, and soybean oil), zinc stearate, ethyl oleate, ethyl laureate, agar, or mixtures thereof. Additional lubricants include, for example, a syloid silica gel, a coagulated aerosol of synthetic silica, or mixtures thereof. A lubricant can optionally be added, in an amount of less than about 1 weight percent of the pharmaceutical composition.

When aqueous suspensions and/or elixirs are desired for oral administration, the active ingredient therein may be combined with various sweetening or flavoring agents, coloring matter or dyes and, if so desired, emulsifying and/or suspending agents, together with such diluents as water, ethanol, propylene glycol, glycerin and various combinations thereof.

The tablets can be uncoated or coated by known techniques to delay disintegration and absorption in the gastrointestinal tract and thereby provide a sustained action over a longer period.

For example, a time delay material such as glyceryl monostearate or glyceryl distearate can be employed. Formulations for oral use can also be presented as hard gelatin capsules wherein the active ingredient is mixed with an inert solid diluent, for example, calcium carbonate, calcium phosphate or kaolin, or as soft gelatin capsules wherein the active ingredient is mixed with water or an oil medium, for example, peanut oil, liquid paraffin or olive oil.

Surfactant which can be used to form pharmaceutical compositions and dosage forms of the invention include, but are not limited to, hydrophilic surfactants, lipophilic surfactants, and mixtures thereof. That is, a mixture of hydrophilic surfactants may be employed, a mixture of lipophilic surfactants may be employed, or a mixture of at least one hydrophilic surfactant and at least one lipophilic surfactant may be employed.

A suitable hydrophilic surfactant may generally have an HLB value of at least 10, while suitable lipophilic surfactants may generally have an HLB value of or less than about 10. An empirical parameter used to characterize the relative hydrophilicity and hydrophobicity of non-ionic amphiphilic compounds is the hydrophilic-lipophilic balance (“HLB” value). Surfactants with lower HLB values are more lipophilic or hydrophobic, and have greater solubility in oils, while surfactants with higher HLB values are more hydrophilic, and have greater solubility in aqueous solutions.

Hydrophilic surfactants are generally considered to be those compounds having an HLB value greater than about 10, as well as anionic, cationic, or zwitterionic compounds for which the HLB scale is not generally applicable. Similarly, lipophilic (i.e., hydrophobic) surfactants are compounds having an HLB value equal to or less than about 10. However, HLB value of a surfactant is merely a rough guide generally used to enable formulation of industrial, pharmaceutical and cosmetic emulsions.

Hydrophilic surfactants may be either ionic or non-ionic. Suitable ionic surfactants include, but are not limited to, alkylammonium salts; fusidic acid salts; fatty acid derivatives of amino acids, oligopeptides, and polypeptides; glyceride derivatives of amino acids, oligopeptides, and polypeptides; lecithins and hydrogenated lecithins; lysolecithins and hydrogenated lysolecithins; phospholipids and derivatives thereof; lysophospholipids and derivatives thereof; carnitine fatty acid ester salts; salts of alkylsulfates; fatty acid salts; sodium docusate; acyl lactylates; mono- and diacetylated tartaric acid esters of mono- and di-glycerides; succinylated mono- and di-glycerides; citric acid esters of mono- and di-glycerides; and mixtures thereof.

Within the aforementioned group, ionic surfactants include, by way of example: lecithins, lysolecithin, phospholipids, lysophospholipids and derivatives thereof; carnitine fatty acid ester salts; salts of alkylsulfates; fatty acid salts; sodium docusate; acylactylates; mono- and di-acetylated tartaric acid esters of mono- and di-glycerides; succinylated mono- and di-glycerides; citric acid esters of mono- and di-glycerides; and mixtures thereof.

Ionic surfactants may be the ionized forms of lecithin, lysolecithin, phosphatidylcholine, phosphatidylethanolamine, phosphatidylglycerol, phosphatidic acid, phosphatidylserine, lysophosphatidylcholine, lysophosphatidylethanolamine, lysophosphatidylglycerol, lysophosphatidic acid, lysophosphatidylserine, PEG-phosphatidylethanolamine, PVP-phosphatidylethanolamine, lactylic esters of fatty acids, stearoyl-2-lactylate, stearoyl lactylate, succinylated monoglycerides, mono/diacetylated tartaric acid esters of mono/diglycerides, citric acid esters of mono/diglycerides, cholylsarcosine, caproate, caprylate, caprate, laurate, myristate, palmitate, oleate, ricinoleate, linoleate, linolenate, stearate, lauryl sulfate, teracecyl sulfate, docusate, lauroyl carnitines, palmitoyl carnitines, myristoyl carnitines, and salts and mixtures thereof.

Hydrophilic non-ionic surfactants may include, but are not limited to, alkylglucosides; alkylmaltosides; alkylthioglucosides; lauryl macrogolglycerides; polyoxyalkylene alkyl ethers such as polyethylene glycol alkyl ethers; polyoxyalkylene alkylphenols such as polyethylene glycol alkyl phenols; polyoxyalkylene alkyl phenol fatty acid esters such as polyethylene glycol fatty acids monoesters and polyethylene glycol fatty acids diesters; polyethylene glycol glycerol fatty acid esters; polyglycerol fatty acid esters; polyoxyalkylene sorbitan fatty acid esters such as polyethylene glycol sorbitan fatty acid esters; hydrophilic transesterification products of a polyol with at least one member of the group consisting of glycerides, vegetable oils, hydrogenated vegetable oils, fatty acids, and sterols; polyoxyethylene sterols, derivatives, and analogues thereof, polyoxyethylated vitamins and derivatives thereof, polyoxyethylene-polyoxypropylene block copolymers; and mixtures thereof, polyethylene glycol sorbitan fatty acid esters and hydrophilic transesterification products of a polyol with at least one member of the group consisting of triglycerides, vegetable oils, and hydrogenated vegetable oils. The polyol may be glycerol, ethylene glycol, polyethylene glycol, sorbitol, propylene glycol, pentaerythritol, or a saccharide.

Other hydrophilic-non-ionic surfactants include, without limitation, PEG-10 laurate, PEG-12 laurate, PEG-20 laurate, PEG-32 laurate, PEG-32 dilaurate, PEG-12 oleate, PEG-15 oleate, PEG-20 oleate, PEG-20 dioleate, PEG-32 oleate, PEG-200 oleate, PEG-400 oleate, PEG-15 stearate, PEG-32 distearate, PEG-40 stearate, PEG-100 stearate, PEG-20 dilaurate, PEG-25 glyceryl trioleate, PEG-32 dioleate, PEG-20 glyceryl laurate, PEG-30 glyceryl laurate, PEG-20 glyceryl stearate, PEG-20 glyceryl oleate, PEG-30 glyceryl oleate, PEG-30 glyceryl laurate, PEG-40 glyceryl laurate, PEG-40 palm kernel oil, PEG-50 hydrogenated castor oil, PEG-40 castor oil, PEG-35 castor oil, PEG-60 castor oil, PEG-40 hydrogenated castor oil, PEG-60 hydrogenated castor oil, PEG-60 corn oil, PEG-6 caprate/caprylate glycerides, PEG-8 caprate/caprylate glycerides, polyglyceryl-10 laurate, PEG-30 cholesterol, PEG-25 phyto sterol, PEG-30 soya sterol, PEG-20 trioleate, PEG-40 sorbitan oleate, PEG-80 sorbitan laurate, polysorbate 20, polysorbate 80, POE-9 lauryl ether, POE-23 lauryl ether, POE-10 oleyl ether, POE-20 oleyl ether, POE-20 stearyl ether, tocopheryl PEG-100 succinate, PEG-24 cholesterol, polyglyceryl-lOoleate, Tween 40, Tween 60, sucrose monostearate, sucrose mono laurate, sucrose monopalmitate, PEG 10-100 nonyl phenol series, PEG 15-100 octyl phenol series, and poloxamers.

Suitable lipophilic surfactants include, by way of example only: fatty alcohols; glycerol fatty acid esters; acetylated glycerol fatty acid esters; lower alcohol fatty acids esters; propylene glycol fatty acid esters; sorbitan fatty acid esters; polyethylene glycol sorbitan fatty acid esters; sterols and sterol derivatives; polyoxyethylated sterols and sterol derivatives; polyethylene glycol alkyl ethers; sugar esters; sugar ethers; lactic acid derivatives of mono- and di-glycerides; hydrophobic transesterification products of a polyol with at least one member of the group consisting of glycerides, vegetable oils, hydrogenated vegetable oils, fatty acids and sterols; oil-soluble vitamins/vitamin derivatives; and mixtures thereof. Within this group, preferred lipophilic surfactants include glycerol fatty acid esters, propylene glycol fatty acid esters, and mixtures thereof, or are hydrophobic transesterification products of a polyol with at least one member of the group consisting of vegetable oils, hydrogenated vegetable oils, and triglycerides.

In one embodiment, the composition may include a solubilizer to ensure good solubilization and/or dissolution of the compound of the present invention and to minimize precipitation of the compound of the present invention. This can be especially important for compositions for non-oral use, e.g., compositions for injection. A solubilizer may also be added to increase the solubility of the hydrophilic drug and/or other components, such as surfactants, or to maintain the composition as a stable or homogeneous solution or dispersion.

Examples of suitable solubilizers include, but are not limited to, the following: alcohols and polyols, such as ethanol, isopropanol, butanol, benzyl alcohol, ethylene glycol, propylene glycol, butanediols and isomers thereof, glycerol, pentaerythritol, sorbitol, mannitol, transcutol, dimethyl isosorbide, polyethylene glycol, polypropylene glycol, polyvinylalcohol, hydroxypropyl methylcellulose and other cellulose derivatives, cyclodextrins and cyclodextrin derivatives; ethers of polyethylene glycols having an average molecular weight of about 200 to about 6000, such as tetrahydrofurfuryl alcohol PEG ether (glycofurol) or methoxy PEG; amides and other nitrogen-containing compounds such as 2-pyrrolidone, 2-piperidone, F-caprolactam, N-alkylpyrrolidone, N-hydroxyalkylpyrrolidone, N-alkylpiperidone, N-alkylcaprolactam, dimethylacetamide and polyvinylpyrrolidone; esters such as ethyl propionate, tributylcitrate, acetyl triethylcitrate, acetyl tributyl citrate, triethylcitrate, ethyl oleate, ethyl caprylate, ethyl butyrate, triacetin, propylene glycol monoacetate, propylene glycol diacetate, ε-caprolactone and isomers thereof, δ-valerolactone and isomers thereof, β-butyrolactone and isomers thereof, and other solubilizers known in the art, such as dimethyl acetamide, dimethyl isosorbide, N-methyl pyrrolidones, monooctanoin, diethylene glycol monoethyl ether, and water.

Mixtures of solubilizers may also be used. Examples include, but not limited to, triacetin, triethylcitrate, ethyl oleate, ethyl caprylate, dimethylacetamide, N-methylpyrrolidone, N-hydroxyethylpyrrolidone, polyvinylpyrrolidone, hydroxypropyl methylcellulose, hydroxypropyl cyclodextrins, ethanol, polyethylene glycol 200-100, glycofurol, transcutol, propylene glycol, and dimethyl isosorbide. Particularly preferred solubilizers include sorbitol, glycerol, triacetin, ethyl alcohol, PEG-400, glycofurol and propylene glycol.

The amount of solubilizer that can be included is not particularly limited. The amount of a given solubilizer may be limited to a bioacceptable amount, which may be readily determined by one of skill in the art. In some circumstances, it may be advantageous to include amounts of solubilizers far in excess of bioacceptable amounts, for example to maximize the concentration of the drug, with excess solubilizer removed prior to providing the composition to a subject using conventional techniques, such as distillation or evaporation. Thus, if present, the solubilizer can be in a weight ratio of 10%, 25%, 50%), 100%, or up to about 200%> by weight, based on the combined weight of the drug, and other excipients. If desired, very small amounts of solubilizer may also be used, such as 5%>, 2%>, 1%) or even less. Typically, the solubilizer may be present in an amount of about 1%> to about 100%, more typically about 5%> to about 25%> by weight.

The composition can further include one or more pharmaceutically acceptable additives and excipients. Such additives and excipients include, without limitation, detackifiers, anti-foaming agents, buffering agents, polymers, antioxidants, preservatives, chelating agents, viscomodulators, tonicifiers, flavorants, colorants, odorants, opacifiers, suspending agents, binders, fillers, plasticizers, lubricants, and mixtures thereof.

In addition, an acid or a base may be incorporated into the composition to facilitate processing, to enhance stability, or for other reasons. Examples of pharmaceutically acceptable bases include amino acids, amino acid esters, ammonium hydroxide, potassium hydroxide, sodium hydroxide, sodium hydrogen carbonate, aluminum hydroxide, calcium carbonate, magnesium hydroxide, magnesium aluminum silicate, synthetic aluminum silicate, synthetic hydrocalcite, magnesium aluminum hydroxide, diisopropylethylamine, ethanolamine, ethylenediamine, triethanolamine, triethylamine, triisopropanolamine, trimethylamine, tris(hydroxymethyl)aminomethane (TRIS) and the like. Also suitable are bases that are salts of a pharmaceutically acceptable acid, such as acetic acid, acrylic acid, adipic acid, alginic acid, alkanesulfonic acid, amino acids, ascorbic acid, benzoic acid, boric acid, butyric acid, carbonic acid, citric acid, fatty acids, formic acid, fumaric acid, gluconic acid, hydroquinosulfonic acid, isoascorbic acid, lactic acid, maleic acid, oxalic acid, para-bromophenylsulfonic acid, propionic acid, p-toluenesulfonic acid, salicylic acid, stearic acid, succinic acid, tannic acid, tartaric acid, thioglycolic acid, toluenesulfonic acid, uric acid, and the like. Salts of polyprotic acids, such as sodium phosphate, disodium hydrogen phosphate, and sodium dihydrogen phosphate can also be used. When the base is a salt, the cation can be any convenient and pharmaceutically acceptable cation, such as ammonium, alkali metals, alkaline earth metals, and the like. Example may include, but not limited to, sodium, potassium, lithium, magnesium, calcium and ammonium.

Suitable acids are pharmaceutically acceptable organic or inorganic acids. Examples of suitable inorganic acids include hydrochloric acid, hydrobromic acid, hydriodic acid, sulfuric acid, nitric acid, boric acid, phosphoric acid, and the like. Examples of suitable organic acids include acetic acid, acrylic acid, adipic acid, alginic acid, alkanesulfonic acids, amino acids, ascorbic acid, benzoic acid, boric acid, butyric acid, carbonic acid, citric acid, fatty acids, formic acid, fumaric acid, gluconic acid, hydroquinosulfonic acid, isoascorbic acid, lactic acid, maleic acid, methanesulfonic acid, oxalic acid, para-bromophenylsulfonic acid, propionic acid, p-toluenesulfonic acid, salicylic acid, stearic acid, succinic acid, tannic acid, tartaric acid, thioglycolic acid, toluenesulfonic acid, uric acid and the like.

### Pharmaceutical Compositions for Injection.

In some embodiments, the invention provides a pharmaceutical composition for injection containing a compound of the present invention and a pharmaceutical excipient suitable for injection. Components and amounts of agents in the compositions are as described herein.

The forms in which the novel compositions of the present invention may be incorporated for administration by injection include aqueous or oil suspensions, or emulsions, with sesame oil, corn oil, cottonseed oil, or peanut oil, as well as elixirs, mannitol, dextrose, or a sterile aqueous solution, and similar pharmaceutical vehicles.

Aqueous solutions in saline are also conventionally used for injection. Ethanol, glycerol, propylene glycol, liquid polyethylene glycol, and the like (and suitable mixtures thereof), cyclodextrin derivatives, and vegetable oils may also be employed. The proper fluidity can be maintained, for example, by the use of a coating, such as lecithin, for the maintenance of the required particle size in the case of dispersion and by the use of surfactants. The prevention of the action of microorganisms can be brought about by various antibacterial and antifungal agents, for example, parabens, chlorobutanol, phenol, sorbic acid, thimerosal, and the like.

Sterile injectable solutions are prepared by incorporating the compound of the present invention in the required amount in the appropriate solvent with various other ingredients as enumerated above, as required, followed by filtered sterilization. Generally, dispersions are prepared by incorporating the various sterilized active ingredients into a sterile vehicle which contains the basic dispersion medium and the required other ingredients from those enumerated above. In the case of sterile powders for the preparation of sterile injectable solutions, certain desirable methods of preparation are vacuum-drying and freeze-drying techniques which yield a powder of the active ingredient plus any additional desired ingredient from a previously sterile-filtered solution thereof.

Pharmaceutical Compositions for Topical (e.g. Transdermal) Delivery.

In some embodiments, the invention provides a pharmaceutical composition for transdermal delivery containing a compound of the present invention and a pharmaceutical excipient suitable for transdermal delivery.

Compositions of the present invention can be formulated into preparations in solid, semisolid, or liquid forms suitable for local or topical administration, such as gels, water soluble jellies, creams, lotions, suspensions, foams, powders, slurries, ointments, solutions, oils, pastes, suppositories, sprays, emulsions, saline solutions, dimethylsulfoxide (DMSO)-based solutions. In general, carriers with higher densities are capable of providing an area with a prolonged exposure to the active ingredients. In contrast, a solution formulation may provide more immediate exposure of the active ingredient to the chosen area.

The pharmaceutical compositions also may comprise suitable solid or gel phase carriers or excipients, which are compounds that allow increased penetration of, or assist in the delivery of, therapeutic molecules across the stratum corneum permeability barrier of the skin. There are many of these penetration-enhancing molecules known to those trained in the art of topical formulation.

Examples of such carriers and excipients include, but are not limited to, humectants (e.g., urea), glycols (e.g., propylene glycol), alcohols (e.g., ethanol), fatty acids (e.g., oleic acid), surfactants (e.g., isopropyl myristate and sodium lauryl sulfate), pyrrolidones, glycerol monolaurate, sulfoxides, terpenes (e.g., menthol), amines, amides, alkanes, alkanols, water, calcium carbonate, calcium phosphate, various sugars, starches, cellulose derivatives, gelatin, and polymers such as polyethylene glycols.

Another exemplary formulation for use in the methods of the present invention employs transdermal delivery devices (“patches”). Such transdermal patches may be used to provide continuous or discontinuous infusion of a compound of the present invention in controlled amounts, either with or without another agent.

The construction and use of transdermal patches for the delivery of pharmaceutical agents is well known in the art. See, e.g., U.S. Pat. Nos. 5,023,252, 4,992,445 and 5,001,139. Such patches may be constructed for continuous, pulsatile, or on demand delivery of pharmaceutical agents.

### Pharmaceutical Compositions for Inhalation.

Compositions for inhalation or insufflation include solutions and suspensions in pharmaceutically acceptable, aqueous or organic solvents, or mixtures thereof, and powders. The liquid or solid compositions may contain suitable pharmaceutically acceptable excipients as described supra. Preferably the compositions are administered by the oral or nasal respiratory route for local or systemic effect. Compositions in preferably pharmaceutically acceptable solvents may be nebulized by use of inert gases. Nebulized solutions may be inhaled directly from the nebulizing device or the nebulizing device may be attached to a face mask tent, or intermittent positive pressure breathing machine. Solution, suspension, or powder compositions may be administered, preferably orally or nasally, from devices that deliver the formulation in an appropriate manner.

### Other Pharmaceutical Compositions.

Pharmaceutical compositions may also be prepared from compositions described herein and one or more pharmaceutically acceptable excipients suitable for sublingual, buccal, rectal, intraosseous, intraocular, intranasal, epidural, or intraspinal administration, Preparations for such pharmaceutical compositions are well-known in the art. See, e.g., Anderson, Philip O.; Knoben, James E.; Troutman, William G, eds., Handbook of Clinical Drug Data, Tenth Edition, McGraw-Hill, 2002; Pratt and Taylor, eds., Principles of Drug Action, Third Edition, Churchill Livingston, N.Y., 1990; Katzung, ed., Basic and Clinical Pharmacology, Ninth Edition, McGraw Hill, 20037ybg; Goodman and Gilman, eds., The Pharmacological Basis of Therapeutics, Tenth Edition, McGraw Hill, 2001; Remingtons Pharmaceutical Sciences, 20th Ed., Lippincott Williams & Wilkins., 2000; Martindale, The Extra Pharmacopoeia, Thirty-Second Edition (The Pharmaceutical Press, London, 1999); all of which are incorporated by reference herein in their entirety.

Administration of the compounds or pharmaceutical composition of the present invention can be affected by any method that enables delivery of the compounds to the site of action. These methods include oral routes, intraduodenal routes, parenteral injection (including intravenous, intraarterial, subcutaneous, intramuscular, intravascular, intraperitoneal or infusion), topical (e.g. transdermal application), rectal administration, via local delivery by catheter or stent or through inhalation. Compounds can also be administered intraadiposally or intrathecally.

In some embodiments, the compounds or pharmaceutical composition of the present invention are administered by intravenous injection.

The amount of the compound administered will be dependent on the subject being treated, the severity of the disorder or condition, the rate of administration, the disposition of the compound and the discretion of the prescribing physician. However, an effective dosage is in the range of about 0.001 to about 100 mg per kg body weight per day, preferably about 1 to about 35 mg/kg/day, in single or divided doses. For a 70 kg human, this would amount to about 0.05 to 7 g/day, preferably about 0.05 to about 2.5 g/day. In some instances, dosage levels below the lower limit of the aforesaid range may be more than adequate, while in other cases still larger doses may be employed without causing any harmful side effect, e.g. by dividing such larger doses into several small doses for administration throughout the day.

In some embodiments, a compound of the invention is administered in a single dose.

Typically, such administration will be by injection, e.g., intravenous injection, in order to introduce the agent quickly. However, other routes may be used as appropriate. A single dose of a compound of the invention may also be used for treatment of an acute condition.

In some embodiments, a compound of the invention is administered in multiple doses. Dosing may be about once, twice, three times, four times, five times, six times, or more than six times per day. Dosing may be about once a month, once every two weeks, once a week, or once every other day. In another embodiment a compound of the invention and another agent are administered together about once per day to about 6 times per day. In another embodiment the administration of a compound of the invention and an agent continues for less than about 7 days. In yet another embodiment the administration continues for more than about 6, 10, 14, 28 days, two months, six months, or one year. In some cases, continuous dosing is achieved and maintained as long as necessary.

Administration of the compounds of the invention may continue as long as necessary. In some embodiments, a compound of the invention is administered for more than 1, 2, 3, 4, 5, 6, 7, 14, or 28 days. In some embodiments, a compound of the invention is administered for less than 28, 14, 7, 6, 5, 4, 3, 2, or 1 day. In some embodiments, a compound of the invention is administered chronically on an ongoing basis, e.g., for the treatment of chronic effects.

An effective amount of a compound of the invention may be administered in either single or multiple doses by any of the accepted modes of administration of agents having similar utilities, including rectal, buccal, intranasal and transdermal routes, by intra-arterial injection, intravenously, intraperitoneally, parenterally, intramuscularly, subcutaneously, orally, topically, or as an inhalant.

The compositions of the invention may also be delivered via an impregnated or coated device such as a stent, for example, or an artery-inserted cylindrical polymer. Such a method of administration may, for example, aid in the prevention or amelioration of restenosis following procedures such as balloon angioplasty. Without being bound by theory, compounds of the invention may slow or inhibit the migration and proliferation of smooth muscle cells in the arterial wall which contribute to restenosis. A compound of the invention may be administered, for example, by local delivery from the struts of a stent, from a stent graft, from grafts, or from the cover or sheath of a stent. In some embodiments, a compound of the invention is admixed with a matrix. Such a matrix may be a polymeric matrix and may serve to bond the compound to the stent. Polymeric matrices suitable for such use, include, for example, lactone-based polyesters or copolyesters such as polylactide, polycaprolactonglycolide, polyorthoesters, polyanhydrides, polyaminoacids, polysaccharides, polyphosphazenes, poly (ether-ester) copolymers (e.g. PEO-PLLA); polydimethylsiloxane, poly(ethylene-vinylacetate), acrylate-based polymers or copolymers (e.g. polyhydroxyethyl methylmethacrylate, polyvinyl pyrrolidinone), fluorinated polymers such as polytetrafluoroethylene and cellulose esters. Suitable matrices may be nondegrading or may degrade with time, releasing the compound or compounds. Compounds of the invention may be applied to the surface of the stent by various methods such as dip/spin coating, spray coating, dip-coating, and/or brush-coating. The compounds may be applied in a solvent and the solvent may be allowed to evaporate, thus forming a layer of compound onto the stent. Alternatively, the compound may be located in the body of the stent or graft, for example in microchannels or micropores. When implanted, the compound diffuses out of the body of the stent to contact the arterial wall. Such stents may be prepared by dipping a stent manufactured to contain such micropores or microchannels into a solution of the compound of the invention in a suitable solvent, followed by evaporation of the solvent. Excess drug on the surface of the stent may be removed via an additional brief solvent wash. In yet other embodiments, compounds of the invention may be covalently linked to a stent or graft. A covalent linker may be used which degrades in vivo, leading to the release of the compound of the invention. Any bio-labile linkage may be used for such a purpose, such as ester, amide or anhydride linkages. Compounds of the invention may additionally be administered intravascularly from a balloon used during angioplasty. Extravascular administration of the compounds via the pericard or via advential application of formulations of the invention may also be performed to decrease restenosis.

A variety of stent devices which may be used as described are disclosed, for example, in the following references, all of which are hereby incorporated by reference: U.S. Pat. Nos. 5,451,233; 5,040,548; 5,061,273; 5,496,346; 5,292,331; 5,674,278; 3,657,744; 4,739,762; 5,195,984; 5,292,331; 5,674,278; 5,879,382; 6,344,053.

The compounds of the invention may be administered in dosages. It is known in the art that due to intersubject variability in compound pharmacokinetics, individualization of dosing regimen is necessary for optimal therapy. Dosing for a compound of the invention may be found by routine experimentation in light of the instant disclosure.

When a compound of the invention is administered in a composition that comprises one or more agents, and the agent has a shorter half-life than the compound of the invention unit dose forms of the agent and the compound of the invention may be adjusted accordingly.

The subject pharmaceutical composition may, for example, be in a form suitable for oral administration as a tablet, capsule, pill, powder, sustained release formulations, solution, suspension, for parenteral injection as a sterile solution, suspension or emulsion, for topical administration as an ointment or cream or for rectal administration as a suppository. The pharmaceutical composition may be in unit dosage forms suitable for single administration of precise dosages. The pharmaceutical composition will include a conventional pharmaceutical carrier or excipient and a compound according to the invention as an active ingredient. In addition, it may include other medicinal or pharmaceutical agents, carriers, adjuvants, etc.

Exemplary parenteral administration forms include solutions or suspensions of active compound in sterile aqueous solutions, for example, aqueous propylene glycol or dextrose solutions. Such dosage forms can be suitably buffered, if desired.

### METHODS OF USE

The method typically comprises administering to a subject a therapeutically effective amount of a compound of the invention. The therapeutically effective amount of the subject combination of compounds may vary depending upon the intended application (in vitro or in vivo), or the subject and disease condition being treated, e.g., the weight and age of the subject, the severity of the disease condition, the manner of administration and the like, which can readily be determined by one of ordinary skill in the art. The term also applies to a dose that will induce a particular response in target cells, e.g., reduction of proliferation or downregulation of activity of a target protein. The specific dose will vary depending on the particular compounds chosen, the dosing regimen to be followed, whether it is administered in combination with other compounds, timing of administration, the tissue to which it is administered, and the physical delivery system in which it is carried.

As used herein, the term “IC₅₀” refers to the half maximal inhibitory concentration of an inhibitor in inhibiting biological or biochemical function. This quantitative measure indicates how much of a particular inhibitor is needed to inhibit a given biological process (or component of a process, i.e. an enzyme, cell, cell receptor or microorganism) by half. In other words, it is the half maximal (50%) inhibitory concentration (IC) of a substance (50% IC, or IC50). EC50 refers to the plasma concentration required for obtaining 50%> of a maximum effect in vivo.

In some embodiments, the subject methods utilize a MCL-1 inhibitor with an IC50 value of about or less than a predetermined value, as ascertained in an in vitro assay. In some embodiments, the MCL-1 inhibitor inhibits MCL-1 a with an IC50 value of about 1 nM or less, 2 nM or less, 5 nM or less, 7 nM or less, 10 nM or less, 20 nM or less, 30 nM or less, 40 nM or less, 50 nM or less, 60 nM or less, 70 nM or less, 80 nM or less, 90 nM or less, 100 nM or less, 120 nM or less, 140 nM or less, 150 nM or less, 160 nM or less, 170 nM or less, 180 nM or less, 190 nM or less, 200 nM or less, 225 nM or less, 250 nM or less, 275 nM or less, 300 nM or less, 325 nM or less, 350 nM or less, 375 nM or less, 400 nM or less, 425 nM or less, 450 nM or less, 475 nM or less, 500 nM or less, 550 nM or less, 600 nM or less, 650 nM or less, 700 nM or less, 750 nM or less, 800 nM or less, 850 nM or less, 900 nM or less, 950 nM or less, 1 μM or less, 1.1 μM or less, 1.2 μM or less, 1.3 μM or less, 1.4 μM or less, 1.5 μM or less, 1.6 μM or less, 1.7 μM or less, 1.8 μM or less, 1.9 μM or less, 2 μM or less, 5 μM or less, 10 μM or less, 15 μM or less, 20 μM or less, 25 μM or less, 30 μM or less, 40 μM or less, 50 μM, 60 μM, 70 μM, 80 μM, 90 μM, 100 μM, 200 μM, 300 μM, 400 μM, or 500 μM, or less, (or a number in the range defined by and including any two numbers above).

In some embodiments, the MCL-1 inhibitor selectively inhibits MCL-1 a with an IC50 value that is at least 2, 3, 4, 5, 6, 7, 8, 9, 10, 15, 20, 25, 30, 35, 40, 45, 50, 100, or 1000 times less (or a number in the range defined by and including any two numbers above) than its IC50 value against one, two, or three other MCL-1s.

In some embodiments, the MCL-1 inhibitor selectively inhibits MCL-1 a with an IC50 value that is less than about 1 nM, 2 nM, 5 nM, 7 nM, 10 nM, 20 nM, 30 nM, 40 nM, 50 nM, 60 nM, 70 nM, 80 nM, 90 nM, 100 nM, 120 nM, 140 nM, 150 nM, 160 nM, 170 nM, 180 nM, 190 nM, 200 nM, 225 nM, 250 nM, 275 nM, 300 nM, 325 nM, 350 nM, 375 nM, 400 nM, 425 nM, 450 nM, 475 nM, 500 nM, 550 nM, 600 nM, 650 nM, 700 nM, 750 nM, 800 nM, 850 nM, 900 nM, 950 nM, 1 μM, 1.1 M, 1.2 μM, 1.3 μM, 1.4 μM, 1.5 μM, 1.6 μM, 1.7 μM, 1.8 μM, 1.9 μM, 2 μM, 5 μM, 10 μM, 15 μM, 20 M, 25 μM, 30 μM, 40 μM, 50 μM, 60 μM, 70 μM, 80 μM, 90 μM, 100 μM, 200 μM, 300 μM, 400 μM, or 500 μM (or in the range defined by and including any two numbers above), and said IC50 value is at least 2, 3, 4, 5, 6, 7, 8, 9, 10, 15, 20, 25, 30, 35, 40, 45, 50, 100, or 1000 times less (or a number in the range defined by and including any two numbers above) than its IC50 value against one, two or three other MCL-1s.

The subject methods are useful for treating a disease condition associated with MCL-1. Any disease condition that results directly or indirectly from an abnormal activity or expression level of MCL-1 can be an intended disease condition.

Different disease conditions associated with MCL-1 have been reported. MCL-1 has been implicated, for example, auto-immune diseases, neurodegeneration (such as Parkinson's disease, Alzheimer's disease and ischaemia), inflammatory diseases, viral infections and cancer such as, for example, colon cancer, breast cancer, small-cell lung cancer, non-small-cell lung cancer, bladder cancer, ovarian cancer, prostate cancer, chronic lymphoid leukemia, lymphoma, myeloma, acute myeloid leukemia, or pancreatic cancer.

Non-limiting examples of such conditions include but are not limited to Acanthoma, Acinic cell carcinoma, Acoustic neuroma, Acral lentiginous melanoma, Acrospiroma, Acute eosinophilic leukemia, Acute lymphoblastic leukemia, Acute lymphocytic leukemia, Acute megakaryoblastic leukemia, Acute monocytic leukemia, Acute myeloblasts leukemia with maturation, Acute myeloid dendritic cell leukemia, Acute myeloid leukemia, Acute myelogenous leukemia, Acute promyelocytic leukemia, Adamantinoma, Adenocarcinoma, Adenoid cystic carcinoma, Adenoma, Adenomatoid odontogenic tumor, Adrenocortical carcinoma, Adult T-cell leukemia, Aggressive NK-cell leukemia, AIDS-Related Cancers, AIDS-related lymphoma, Alveolar soft part sarcoma, Ameloblastic fibroma, Anal cancer, Anaplastic large cell lymphoma, Anaplastic thyroid cancer, Angioimmunoblastic T-cell lymphoma, Angiomyolipoma, Angiosarcoma, Appendix cancer, Astrocytoma, Atypical teratoid rhabdoid tumor, Basal cell carcinoma, Basal-like carcinoma, B-cell leukemia, B-cell lymphoma, Bellini duct carcinoma, Biliary tract cancer, Bladder cancer, Blastoma, Bone Cancer, Bone tumor, Brain Stem Glioma, Brain Tumor, Breast Cancer, Brenner tumor, Bronchial Tumor, Bronchioloalveolar carcinoma, Brown tumor, Burkitt's lymphoma, Cancer of Unknown Primary Site, Carcinoid Tumor, Carcinoma, Carcinoma in situ, Carcinoma of the penis, Carcinoma of Unknown Primary Site, Carcinosarcoma, Castleman's Disease, Central Nervous System Embryonal Tumor, Cerebellar Astrocytoma, Cerebral Astrocytoma, Cervical Cancer, Cholangiocarcinoma, Chondroma, Chondrosarcoma, Chordoma, Choriocarcinoma, Choroid plexus papilloma, Chronic Lymphocytic Leukemia, Chronic monocytic leukemia, Chronic myelogenous leukemia, Chronic Myeloproliferative Disorder, Chronic neutrophilic leukemia, Clear-cell tumor, Colon Cancer, Colorectal cancer, Craniopharyngioma, Cutaneous T-cell lymphoma, Degos disease, Dermatofibrosarcoma protuberans, Dermoid cyst, Desmoplastic small round cell tumor, Diffuse large B cell lymphoma, Dysembryoplastic neuroepithelial tumor, Embryonal carcinoma, Endodermal sinus tumor, Endometrial cancer, Endometrial Uterine Cancer, Endometrioid tumor, Enteropathy-associated T-cell lymphoma, Ependymoblastoma, Ependymoma, Epidermoid cancer, Epithelioid sarcoma, Erythroleukemia, Esophageal cancer, Esthesioneuroblastoma, Ewing Family of Tumor, Ewing Family Sarcoma, Ewing's sarcoma, Extracranial Germ Cell Tumor, Extragonadal Germ Cell Tumor, Extrahepatic Bile Duct Cancer, Extramammary Paget's disease, Fallopian tube cancer, Fetus in fetu, Fibroma, Fibrosarcoma, Follicular lymphoma, Follicular thyroid cancer, Gallbladder Cancer, Gallbladder cancer, Ganglioglioma, Ganglioneuroma, Gastric Cancer, Gastric lymphoma, Gastrointestinal cancer, Gastrointestinal Carcinoid Tumor, Gastrointestinal Stromal Tumor, Gastrointestinal stromal tumor, Germ cell tumor, Germinoma, Gestational choriocarcinoma, Gestational Trophoblastic Tumor, Giant cell tumor of bone, Glioblastoma multiforme, Glioma, Gliomatosis cerebri, Glomus tumor, Glucagonoma, Gonadoblastoma, Granulosa cell tumor, Hairy Cell Leukemia, Head and Neck Cancer, Head and neck cancer, Heart cancer, Hemoglobinopathies such as b-thalassemia and sickle cell disease (SCD), Hemangioblastoma, Hemangiopericytoma, Hemangiosarcoma, Hematological malignancy, Hepatocellular carcinoma, Hepatosplenic T-cell lymphoma, Hereditary breast-ovarian cancer syndrome, Hodgkin Lymphoma, Hodgkin's lymphoma, Hypopharyngeal Cancer, Hypothalamic Glioma, Inflammatory breast cancer, Intraocular Melanoma, Islet cell carcinoma, Islet Cell Tumor, Juvenile myelomonocytic leukemia, Kaposi Sarcoma, Kaposi's sarcoma, Kidney Cancer, Klatskin tumor, Krukenberg tumor, Laryngeal Cancer, Laryngeal cancer, Lentigo maligna melanoma, Leukemia, Lip and Oral Cavity Cancer, Liposarcoma, Lung cancer, Luteoma, Lymphangioma, Lymphangiosarcoma, Lymphoepithelioma, Lymphoid leukemia, Lymphoma, Macroglobulinemia, Malignant Fibrous Histiocytoma, Malignant fibrous histiocytoma, Malignant Fibrous Histiocytoma of Bone, Malignant Glioma, Malignant Mesothelioma, Malignant peripheral nerve sheath tumor, Malignant rhabdoid tumor, Malignant triton tumor, MALT lymphoma, Mantle cell lymphoma, Mast cell leukemia, Mastocytosis, Mediastinal germ cell tumor, Mediastinal tumor, Medullary thyroid cancer, Medulloblastoma, Medulloblastoma, Medulloepithelioma, Melanoma, Melanoma, Meningioma, Merkel Cell Carcinoma, Mesothelioma, Mesothelioma, Metastatic Squamous Neck Cancer with Occult Primary, Metastatic urothelial carcinoma, Mixed Mullerian tumor, Monocytic leukemia, Mouth Cancer, Mucinous tumor, Multiple Endocrine Neoplasia Syndrome, Multiple Myeloma, Multiple myeloma, Mycosis Fungoides, Mycosis fungoides, Myelodysplasia Disease, Myelodysplasia Syndromes, Myeloid leukemia, Myeloid sarcoma, Myeloproliferative Disease, Myxoma, Nasal Cavity Cancer, Nasopharyngeal Cancer, Nasopharyngeal carcinoma, Neoplasm, Neurinoma, Neuroblastoma, Neuroblastoma, Neurofibroma, Neuroma, Nodular melanoma, Non-Hodgkin Lymphoma, Non-Hodgkin lymphoma, Nonmelanoma Skin Cancer, Non-Small Cell Lung Cancer, Ocular oncology, Oligoastrocytoma, Oligodendroglioma, Oncocytoma, Optic nerve sheath meningioma, Oral Cancer, Oral cancer, Oropharyngeal Cancer, Osteosarcoma, Osteosarcoma, Ovarian Cancer, Ovarian cancer, Ovarian Epithelial Cancer, Ovarian Germ Cell Tumor, Ovarian Low Malignant Potential Tumor, Paget's disease of the breast, Pancoast tumor, Pancreatic Cancer, Pancreatic cancer, Papillary thyroid cancer, Papillomatosis, Paraganglioma, Paranasal Sinus Cancer, Parathyroid Cancer, Penile Cancer, Perivascular epithelioid cell tumor, Pharyngeal Cancer, Pheochromocytoma, Pineal Parenchymal Tumor of Intermediate Differentiation, Pineoblastoma, Pituicytoma, Pituitary adenoma, Pituitary tumor, Plasma Cell Neoplasm, Pleuropulmonary blastoma, Polyembryoma, Precursor T-lymphoblastic lymphoma, Primary central nervous system lymphoma, Primary effusion lymphoma, Primary Hepatocellular Cancer, Primary Liver Cancer, Primary peritoneal cancer, Primitive neuroectodermal tumor, Prostate cancer, Pseudomyxoma peritonei, Rectal Cancer, Renal cell carcinoma, Respiratory Tract Carcinoma Involving the NUT Gene onChromosome 15, Retinoblastoma, Rhabdomyoma, Rhabdomyosarcoma, Richter's transformation, Sacrococcygeal teratoma, Salivary Gland Cancer, Sarcoma, Schwannomatosis, Sebaceous gland carcinoma, Secondary neoplasm, Seminoma, Serous tumor, Sertoli-Leydig cell tumor, Sex cord-stromal tumor, Sezary Syndrome, Signet ring cell carcinoma, Skin Cancer, Small blue round cell tumor, Small cell carcinoma, Small Cell Lung Cancer, Small cell lymphoma, Small intestine cancer, Soft tissue sarcoma, Somatostatinoma, Soot wart, Spinal Cord Tumor, Spinal tumor, Splenic marginal zone lymphoma, Squamous cell carcinoma, Stomach cancer, Superficial spreading melanoma, Supratentorial Primitive Neuroectodermal Tumor, Surface epithelial-stromal tumor, Synovial sarcoma, T-cell acute lymphoblastic leukemia, T-cell large granular lymphocyte leukemia, T-cell leukemia, T-cell lymphoma, T-cell prolymphocytic leukemia, Teratoma, Terminal lymphatic cancer, Testicular cancer, Thecoma, Throat Cancer, Thymic Carcinoma, Thymoma, Thyroid cancer, Transitional Cell Cancer of Renal Pelvis and Ureter, Transitional cell carcinoma, Urachal cancer, Urethral cancer, Urogenital neoplasm, Uterine sarcoma, Uveal melanoma, Vaginal Cancer, Verner Morrison syndrome, Verrucous carcinoma, Visual Pathway Glioma, Vulvar Cancer, Waldenstrom's macroglobulinemia, Warthin's tumor, Wilms' tumor, or any combination thereof.

In some embodiments, said method is for treating a disease selected from the group consisting of tumor angiogenesis, chronic inflammatory disease such as rheumatoid arthritis, atherosclerosis, inflammatory bowel disease, skin diseases such as psoriasis, eczema, and scleroderma, diabetes, diabetic retinopathy, retinopathy of prematurity, age-related macular degeneration, hemangioma, glioma, melanoma, Kaposi's sarcoma and ovarian, breast, lung, pancreatic, prostate, colon and epidermoid cancer.

In other embodiments, said method is for treating a disease selected from breast cancer, lung cancer, pancreatic cancer, prostate cancer, colon cancer, ovarian cancer, uterine cancer, or cervical cancer.

In other embodiments, said method is for treating a disease selected from leukemia such as acute myeloid leukemia (AML), acute lymphocytic leukemia, chronic lymphocytic leukemia, chronic myeloid leukemia, hairy cell leukemia, myelodysplasia, myeloproliferative disorders, acute myelogenous leukemia (AML), chronic myelogenous leukemia (CML), mastocytosis, chronic lymphocytic leukemia (CLL), multiple myeloma (MM), myelodysplastic syndrome (MDS) or epidermoid cancer.

Compounds of the disclosure, as well as pharmaceutical compositions comprising them, can be administered to treat any of the described diseases, alone or in combination with a medical therapy. Medical therapies include, for example, surgery and radiotherapy (e.g., gamma-radiation, neutron beam radiotherapy, electron beam radiotherapy, proton therapy, brachytherapy, systemic radioactive isotopes).

In other aspects, compounds of the disclosure, as well as pharmaceutical compositions comprising them, can be administered to treat any of the described diseases, alone or in combination with one or more other agents.

In other methods, the compounds of the disclosure, as well as pharmaceutical compositions comprising them, can be administered in combination with agonists of nuclear receptors agents.

In other methods, the compounds of the disclosure, as well as pharmaceutical compositions comprising them, can be administered in combination with antagonists of nuclear receptors agents.

In other methods, the compounds of the disclosure, as well as pharmaceutical compositions comprising them, can be administered in combination with an anti-proliferative agent.

### Combination Therapies

For treating cancer and other proliferative diseases, the compounds of the invention can be used in combination with chemotherapeutic agents, agonists or antagonists of nuclear receptors, or other anti-proliferative agents. The compounds of the invention can also be used in combination with a medical therapy such as surgery or radiotherapy, e.g., gamma-radiation, neutron beam radiotherapy, electron beam radiotherapy, proton therapy, brachytherapy, and systemic radioactive isotopes. Examples of suitable chemotherapeutic agents include any of: abarelix, aldesleukin, alemtuzumab, alitretinoin, allopurinol, all-trans retinoic acid, altretamine, anastrozole, arsenic trioxide, asparaginase, azacitidine, bendamustine, bevacizumab, bexarotene, bleomycin, bortezombi, bortezomib, busulfan intravenous, busulfan oral, calusterone, capecitabine, carboplatin, carmustine, cetuximab, chlorambucil, cisplatin, cladribine, clofarabine, cyclophosphamide, cytarabine, dacarbazine, dactinomycin, dalteparin sodium, dasatinib, daunorubicin, decitabine, denileukin, denileukin diftitox, dexrazoxane, docetaxel, doxorubicin, dromostanolone propionate, eculizumab, epirubicin, erlotinib, estramustine, etoposide phosphate, etoposide, exemestane, fentanyl citrate, filgrastim, floxuridine, fludarabine, fluorouracil, fulvestrant, gefitinib, gemcitabine, gemtuzumab ozogamicin, goserelin acetate, histrelin acetate, ibritumomab tiuxetan, idarubicin, ifosfamide, imatinib mesylate, interferon alfa 2a, irinotecan, lapatinib ditosylate, lenalidomide, letrozole, leucovorin, leuprolide acetate, levamisole, lomustine, meclorethamine, megestrol acetate, melphalan, mercaptopurine, methotrexate, methoxsalen, mitomycin C, mitotane, mitoxantrone, nandrolone phenpropionate, nelarabine, nofetumomab, oxaliplatin, paclitaxel, pamidronate, panobinostat, panitumumab, pegaspargase, pegfilgrastim, pemetrexed disodium, pentostatin, pipobroman, plicamycin, procarbazine, quinacrine, rasburicase, rituximab, ruxolitinib, sorafenib, streptozocin, sunitinib, sunitinib maleate, tamoxifen, temozolomide, teniposide, testolactone, thalidomide, thioguanine, thiotepa, topotecan, toremifene, tositumomab, trastuzumab, tretinoin, uracil mustard, valrubicin, vinblastine, vincristine, vinorelbine, vorinstat and zoledronate.

In some embodiments, the compounds of the invention can be used in combination with a therapeutic agent that targets an epigenetic regulator. Examples of epigenetic regulators include bromodomain inhibitors, the histone lysine methyltransferase inhibitors, histone arginine methyl transferase inhibitors, histone demethylase inhibitors, histone deacetylase inhibitors, histone acetylase inhibitors, and DNA methyltransferase inhibitors. Histone deacetylase inhibitors include, e.g., vorinostat. Histone arginine methyl transferase inhibitors include inhibitors of protein arginine methyltransferases (PRMTs) such as PRMT5, PRMT1 and PRMT4. DNA methyltransferase inhibitors include inhibitors of DNMT1 and DNMT3.

For treating cancer and other proliferative diseases, the compounds of the invention can be used in combination with targeted therapies, including JAK kinase inhibitors (e.g. Ruxolitinib), PI3 kinase inhibitors including PI3K-delta selective and broad spectrum PI3K inhibitors, MEK inhibitors, Cyclin Dependent kinase inhibitors, including CDK4/6 inhibitors and CDK9 inhibitors, BRAF inhibitors, mTOR inhibitors, proteasome inhibitors (e.g. Bortezomib, Carfilzomib), HDAC inhibitors (e.g. panobinostat, vorinostat), DNA methyl transferase inhibitors, dexamethasone, bromo and extra terminal family member (BET) inhibitors, BTK inhibitors (e.g. ibrutinib, acalabrutinib), BCL2 inhibitors (e.g. venetoclax), dual BCL2 family inhibitors (e.g. BCL2/BCLxL), PARP inhibitors, FLT3 inhibitors, or LSD1 inhibitors.

In some embodiments, the inhibitor of an immune checkpoint molecule is an inhibitor of PD-1, e.g., an anti-PD-1 monoclonal antibody. In some embodiments, the anti-PD-1 monoclonal antibody is nivolumab, pembrolizumab (also known as MK-3475), or PDR001. In some embodiments, the anti-PD-1 monoclonal antibody is nivolumab or pembrolizumab. In some embodiments, the anti-PD1 antibody is pembrolizumab. In some embodiments, the inhibitor of an immune checkpoint molecule is an inhibitor of PD-L1, e.g., an anti-PD-L1 monoclonal antibody. In some embodiments, the anti-PD-L1 monoclonal antibody is atezolizumab, durvalumab, or BMS-935559. In some embodiments, the inhibitor of an immune checkpoint molecule is an inhibitor of CTLA-4, e.g., an anti-CTLA-4 antibody. In some embodiments, the anti-CTLA-4 antibody is ipilimumab.

In some embodiments, the agent is an alkylating agent, a proteasome inhibitor, a corticosteroid, or an immunomodulatory agent. Examples of an alkylating agent include cyclophosphamide (CY), melphalan (MEL), and bendamustine. In some embodiments, the proteasome inhibitor is carfilzomib. In some embodiments, the corticosteroid is dexamethasone (DEX). In some embodiments, the immunomodulatory agent is lenalidomide (LEN) or pomalidomide (POM).

For treating autoimmune or inflammatory conditions, the compound of the invention can be administered in combination with a corticosteroid such as triamcinolone, dexamethasone, fluocinolone, cortisone, prednisolone, or flumetholone.

For treating autoimmune or inflammatory conditions, the compound of the invention can be administered in combination with an immune suppressant such as fluocinolone acetonide (Retisert®), rimexolone (AL-2178, Vexol, Alcon), or cyclosporine (Restasis®).

### Synthesis

Compounds of the invention can be prepared according to numerous synthetic methodologies and schemes known in the literature. The Schemes below provide general guidance in connection with preparing the compounds of the invention. One skilled in the art would understand that the preparations shown in the Schemes can be modified or optimized using general knowledge of organic chemistry to prepare various compounds of the invention. Example synthetic methods for preparing compounds of the invention are provided in the Schemes below.

A series of macrocyclic sulfonimidamide derivatives of formula 1-8 and 1-9 can be prepared by the methods outlined in Scheme 1. Reductive amination of the sulfonamide 1-1 with the appropriate aldehyde or ketone 1-2 (R^{13A}=optional substituent) under suitable conditions (e.g., imine formation followed by treatment with a suitable reducing agent, such as NaBH₃(CN) or NaBH(OAc)₃) can afford the corresponding N-alkylation product 1-3 which can be transformed into the sulfonamide 1-4 by the removal of the protecting groups (e.g., PMB) in 1-3 under acid conditions (e.g., TFA, HCl) or hydrogenation conditions (e.g., palladium catalyzed hydrogenation Pd/C with H₂) following by treatment with TBSCl in the presence of suitable base such as TEA, Hunig's base or other organic base. Sulfonimidamide 1-5 can be obtained by reaction of the sulfonamide 1-4 with (Ph)₃PCl₂ under basic conditions following by treatment with NH₃ (g). The amide coupling of the sulfonamide 1-5 with the suitable acid 1-6 using a coupling agent (e.g., EDC, DCC, or HATU) in the presence of a suitable base (e.g., TEA or DIEA) can yield the macrocyclic precursor 1-7 which can be then be transformed into the corresponding macrocyclic-sulfonamide derivative 1-8 using a suitable ring closing metathesis (RCM) catalyst (e.g., Grubbs catalyst, Grubbs-II catalyst, Grubbs-Ill catalyst, Hoveyda-Grubbs catalyst or Zhan catalyst 1B) under standard reaction conditions. Macrocyclic-sulfonamide derivative 1-9 can be obtained from macrocyclic-sulfonamide derivative 1-8 using hydrogenation conditions (e.g., palladium catalyzed hydrogenation Pd/C with H₂).

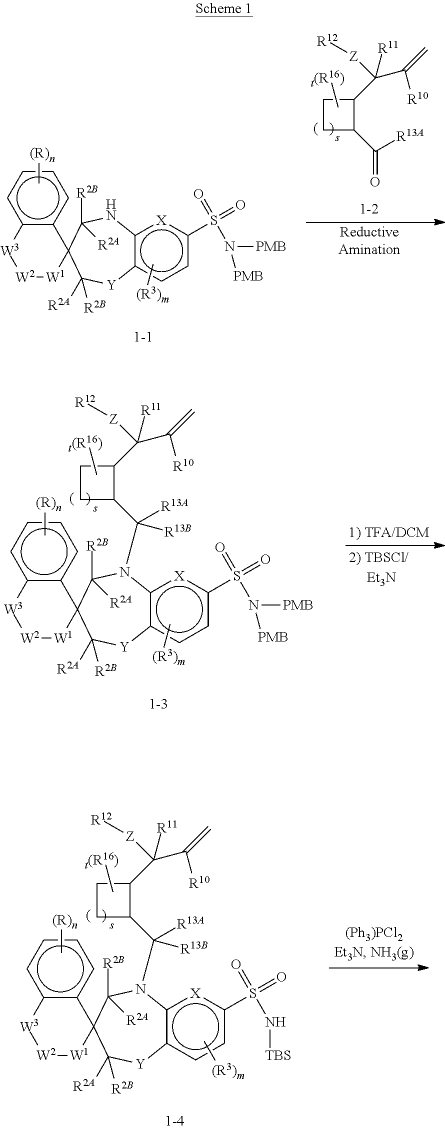

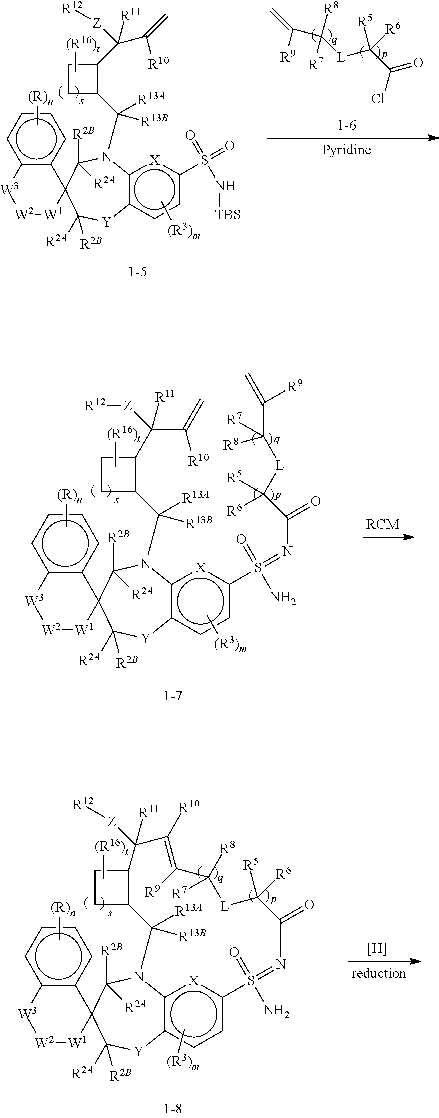

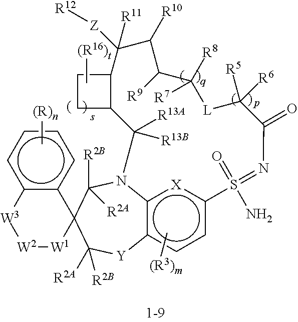

A series of urea derivatives of formula 2-3 can be prepared by the methods outlined in Scheme 2. Macrocyclic sulfonimidamide 2-1 can be transformed into the desired urea derivative 2-3 by reaction with a suitable dialkylcarbamoyl chloride 2-2, or by reaction with diphenyl carbonate or CDI followed by treatment with suitable amine R^{4C}R^{4D}NH.

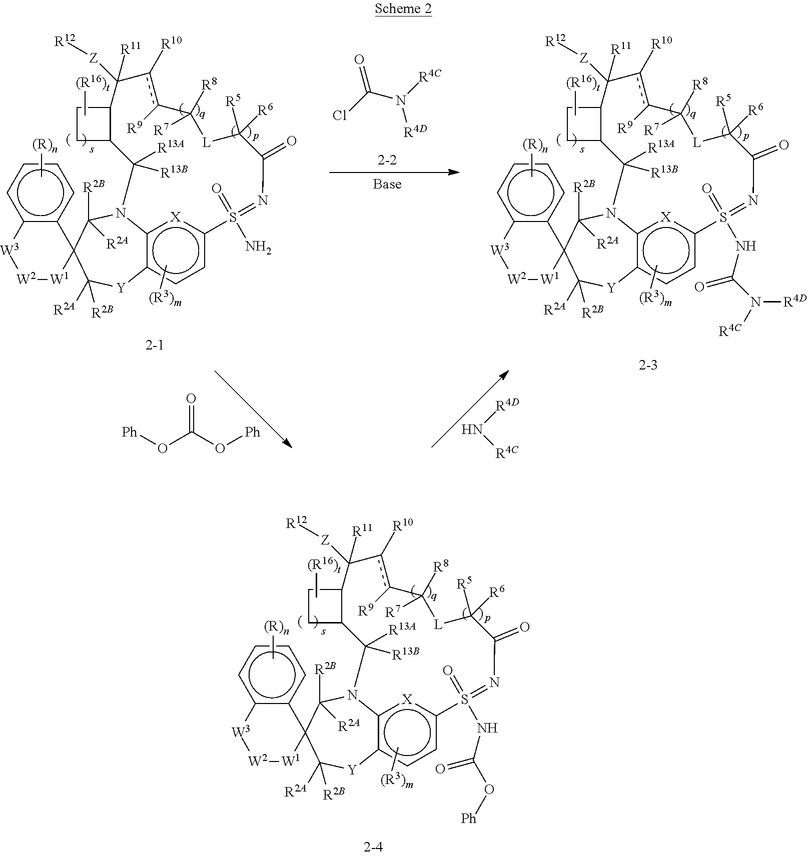

Similarly, various macrocyclic sulfonimidamide derivatives of formula 3-2 to 3-7 can be prepared by the methods outlined in Scheme 3. Reaction of the macrocyclic sulfonimidamide 3-1 with appropriate isocyanate R^{4C}N═C═O 3-8, chloroformate derivative Cl—C(O)OR^{4A} 3-9, acyl chloride Cl—C(O)R^{4B} 3-10, sulfonyl chloride Cl—S(O)₂R^{4B} 3-11, sulfuramidous chloride Cl —S(O)NR^{4C}R^{4D} 3-12, or sulfamoyl chloride Cl—S(O)₂NR^{4C}R^{4D} 3-13 in the presence of a suitable base (e.g., DIEA, TEA, or pyridine) to produce the corresponding macrocyclic sulfonimidamide derivatives of formula 3-2 to 3-7, respectively.

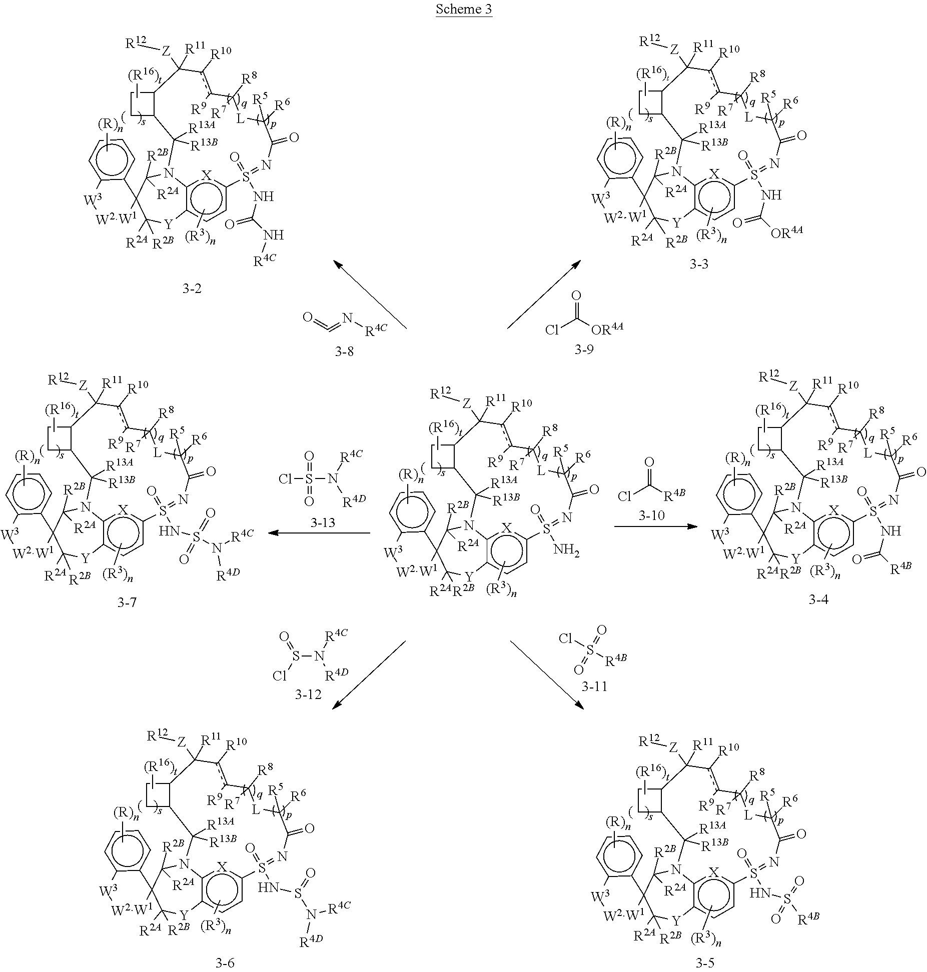

Alternatively, various macrocyclic sulfonimidamide derivatives of formula 4-3 can be prepared similar methods outlined in Scheme 4. The sulfonimidamide 4-1 can be transformed into the corresponding product 4-2 by reaction with appropriate R⁴Cl[ClC(O)OR^{4A}, ClC(O)R^{4B}, ClC(O)NR^{4C}R^{4D}, CIS(O)R^{4B}, ClS(O)₂R^{4B}, ClS(O)NR^{4C}R^{4D}, ClS(O)₂NR^{4C}R^{4D}] or isocyanate R^{4C}N═C═O in the presence of a suitable base (e.g., DIEA, TEA, or pyridine). RCM of sulfonimidamide derivatives 4-3 can afford a pair of diastereomers of the corresponding macrocyclic sulfonimidamide derivatives 4-3 which can be separated into single diastereomers 4-3A and 4-3B by flash chromatography on a normal phase column, a reverse phase column or a chiral column if necessary.

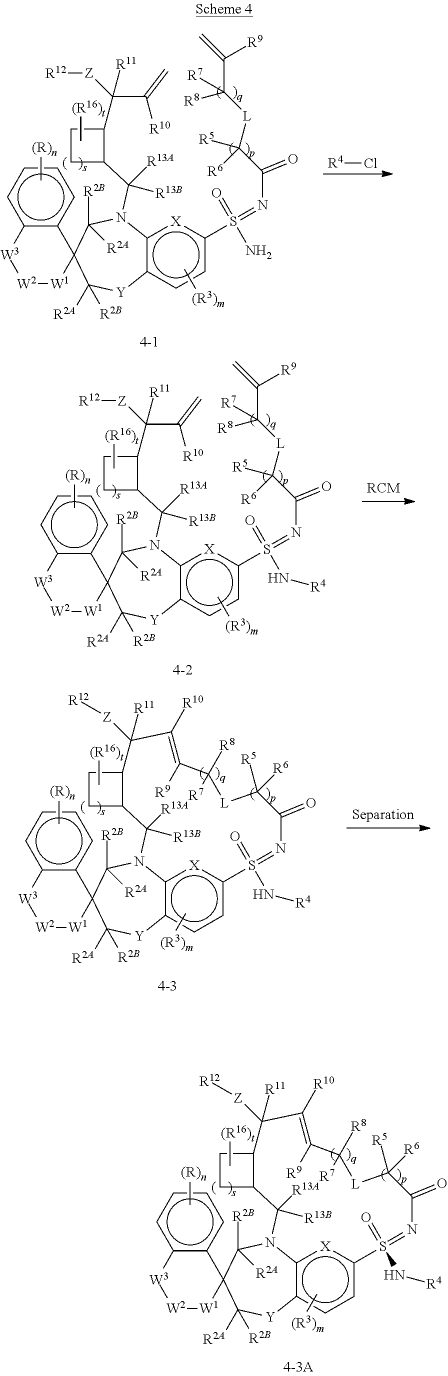

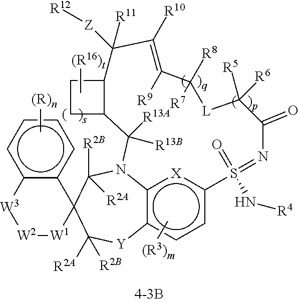

A series of 1,3-diol derivatives of formula 5-4 (n=0, 1, 2) can be prepared by the methods outlined in Scheme 5. Johnson-Corey-Chaykovsky reaction of the substituted ketone 5-1 with sulfur ylide can form the corresponding epoxide 5-2 which can be transformed into the aldehyde 5-3 under Lewis acid (e.g., boron trifluoride diethyl etherate or TiCl₄). The aldehyde 5-3 can be transformed to the 1,3-diol derivative 5-4 by treatment with formaldehyde under basic conditions (e.g., KOH).

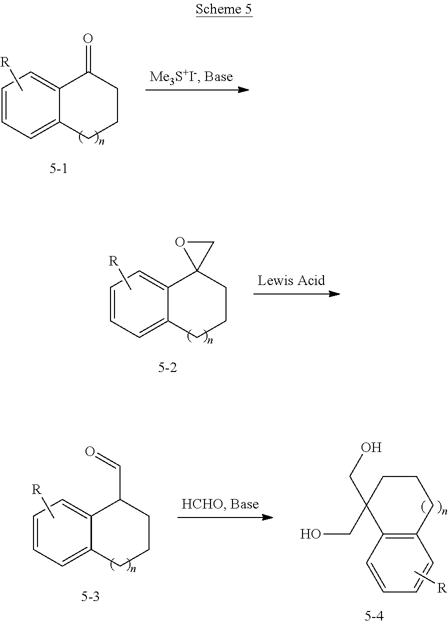

A series of 1,3-diol derivatives of formula 6-8 (n=0, 1, 2) can be prepared by the methods outlined in Scheme 6. Compound 6-3 can be obtained by protecting the alcohol group (e.g., TBS or other silyl group) in 6-2 obtained from the reduction of the acid 6-1 using a suitable reductive reagent including, (e.g., BH3, NaBH₄, LiBH₄, LiAlH₄). Lithiation of compound 6-3 with a suitable agent (e.g., BuLi or another lithium reagent) at low temperature and subsequent reaction of the intermediate with the ketone 6-4 can afford the alcohol derivative 6-5. Removal of the protecting group (e.g., TBAF removal of TBS group) of 6-5 followed by intra-molecular Mitsunobo reaction (e.g., DEAD and triphenylphospine) can yield the cyclic ether 6-7. Removal of the protecting group by treatment of 6-7 with acid (e.g. pTSA or HCl) can provide the 1,3-diol derivative 6-8.

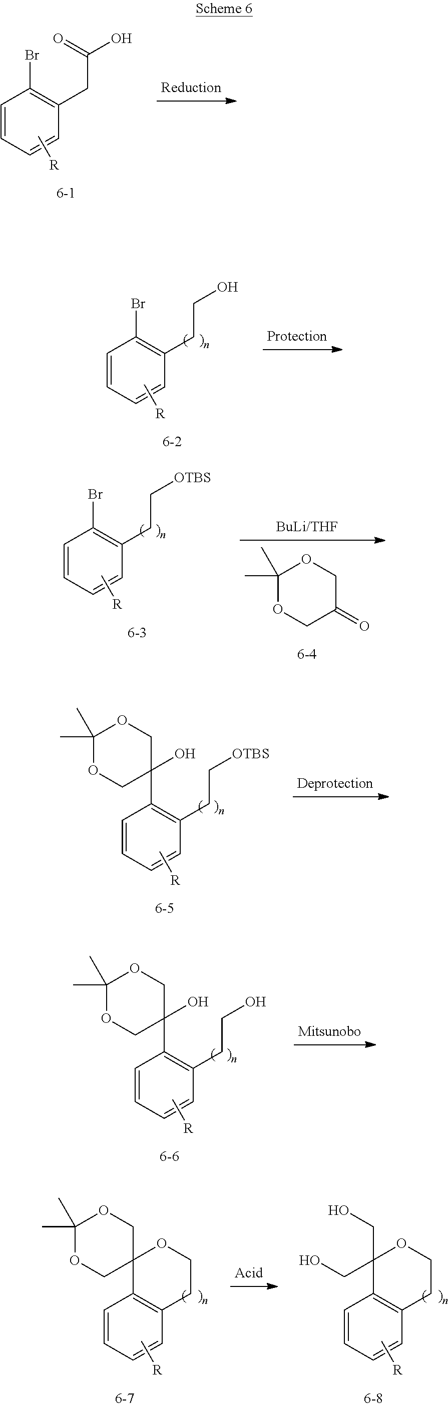

A series of 1,3-diol derivatives of formula 7-7 (n=0, 1, 2) can be prepared by the methods outlined in Scheme 7. Alkylation of phenol compound 7-1 with a bromide 7-2 can give the corresponding ether 7-3 in the presence of a base (e.g., NaH or NaOH) which can react with diethyl malonate 7-4 or other dialkyl malonate in the presence of a suitable palladium catalyst (e.g., Pd(PtButyl)₃ or other Pd catalyst) to afford the malonate derivative 7-5. 1,3-diol derivative 7-7 can be obtained by reduction (e.g., DIBAL, LAH, or NaBH₄) of the diethyl ester 7-6 provided by treatment of the malonate derivative 7-5 with 4-nitrobenzenesulfonyl fluoride in the presence of a base (e.g., DBU or DIEA).

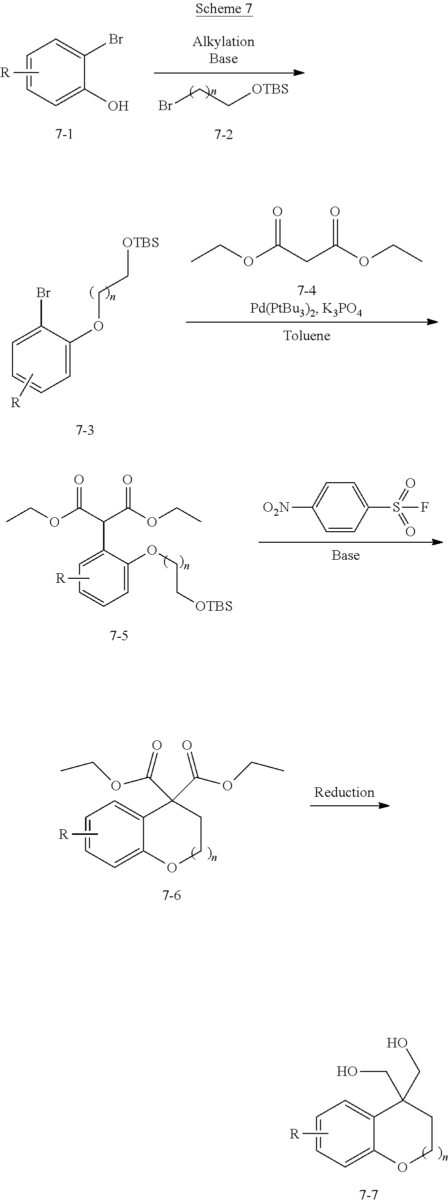

A series of spiro-cyclic sulfonamide derivatives of formula 8-9 can be prepared by the methods outlined in Scheme 8. Asymmetric mono-protecting of the OH-group 8-2 can be achieved by reaction of 8-1 with substituted benzoyl chloride in the presence of a suitable chiral catalyst, such as (R,R)-Kang Catalyst. Dess-Martin oxidation or Swern oxidation of the hydroxyl group of 8-2 can afford the corresponding aldehyde 8-3 which can be transformed to compound 8-4 by reaction with trimethyl orthoformate in the presence of acid (e.g., p-TsOH in methanol). Hydrolysis of compound 8-4 can yield the alcohol 8-5. Reaction of the alcohol 8-5 with 4-fluoro-3-nitrobenzenesulfonamide 8-6 in the presence of base (e.g., potassium tert-butoxide, sodium tert-butoxide, LiHMDS or NaLiHMDS) can afford the corresponding compound 8-7. De-protection of the acetal group in 8-7 to the aldehyde 8-8 can be achieved under acid conditions (e.g., Amberlyst, p-TsOH, HCl in dioxane or TFA). Reduction of the nitro group in 8-8 by using a suitable reducing agent (e.g., iron in acetic acid, iron or zinc and NH₄Cl in ethanol) followed by further reduction of the imine formed in situ with suitable reductive reagent (e.g., NaBH₄ or NaBH(OAc)) can afford the spiro-cyclic sulfonamide derivatives of formula 8-9.

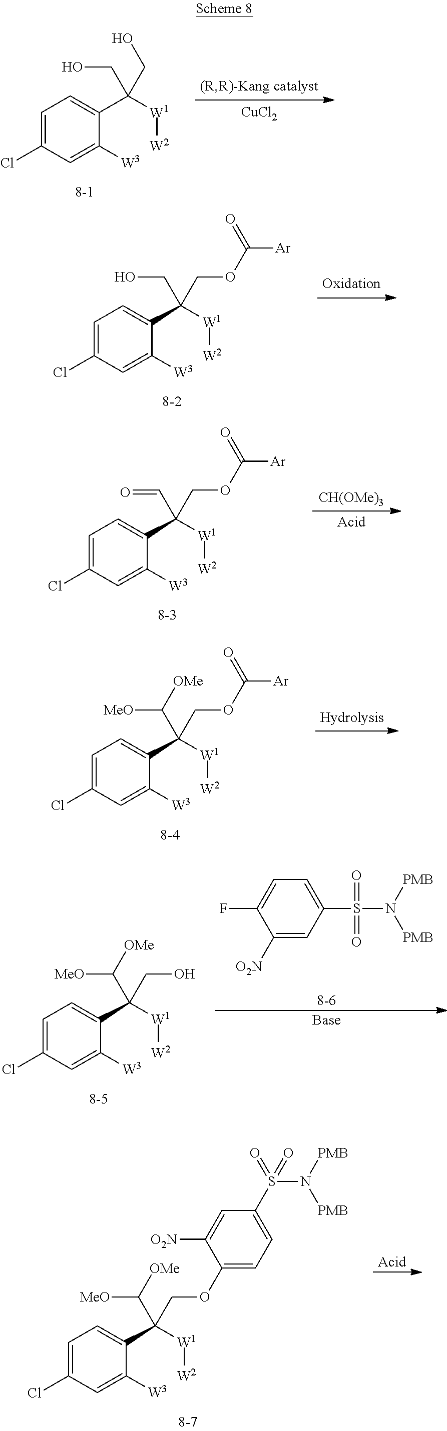

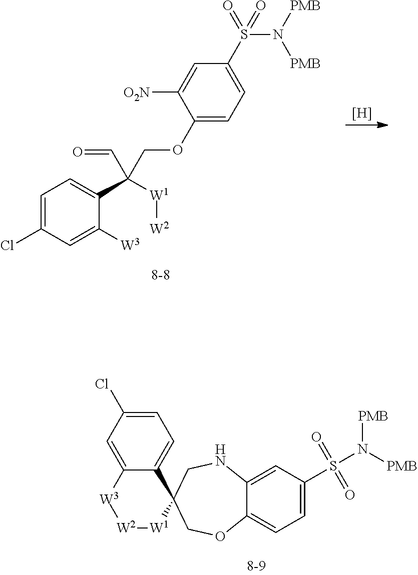

A series of allyl alcohol derivatives of formula 9-8 or 9-9 can be prepared by the methods outlined in Scheme 9. Reductive amination of the spiro-sulfonamide 9-1 with aldehyde 9-2 using reductive agent (e.g., NaBH₄, NaBH(OAc)₃, or NaBCNH3) can afford the corresponding product 9-3 which can be transformed into the alcohol 9-4 under saponification conditions (e.g. acid or base).

Swern oxidation or Dess-Martin oxidation of the alcohol 9-4 can afford the aldehyde 9-5. Alkylation of 9-5 with a suitable reagent (e.g., vinylmagnesium bromide or vinyllithium) can yield a mixture of the allyl alcohol 9-6 and 9-7. The diastereomers can be separated by chromatography (e.g., silica gel column, or preparative HPLC on a C18 column, or by SFC using a suitable column) using suitable conditions. 9-6 or 9-7 can be transformed into the corresponding 9-8 or 9-9, respectively, by reaction with a suitable reagent R¹²X (X=Cl, Br, I or other leaving group such as OMs, OTs, or OMTf) in the presence of base (NaH, Hunig's base, pyridine, etc. . . . ).

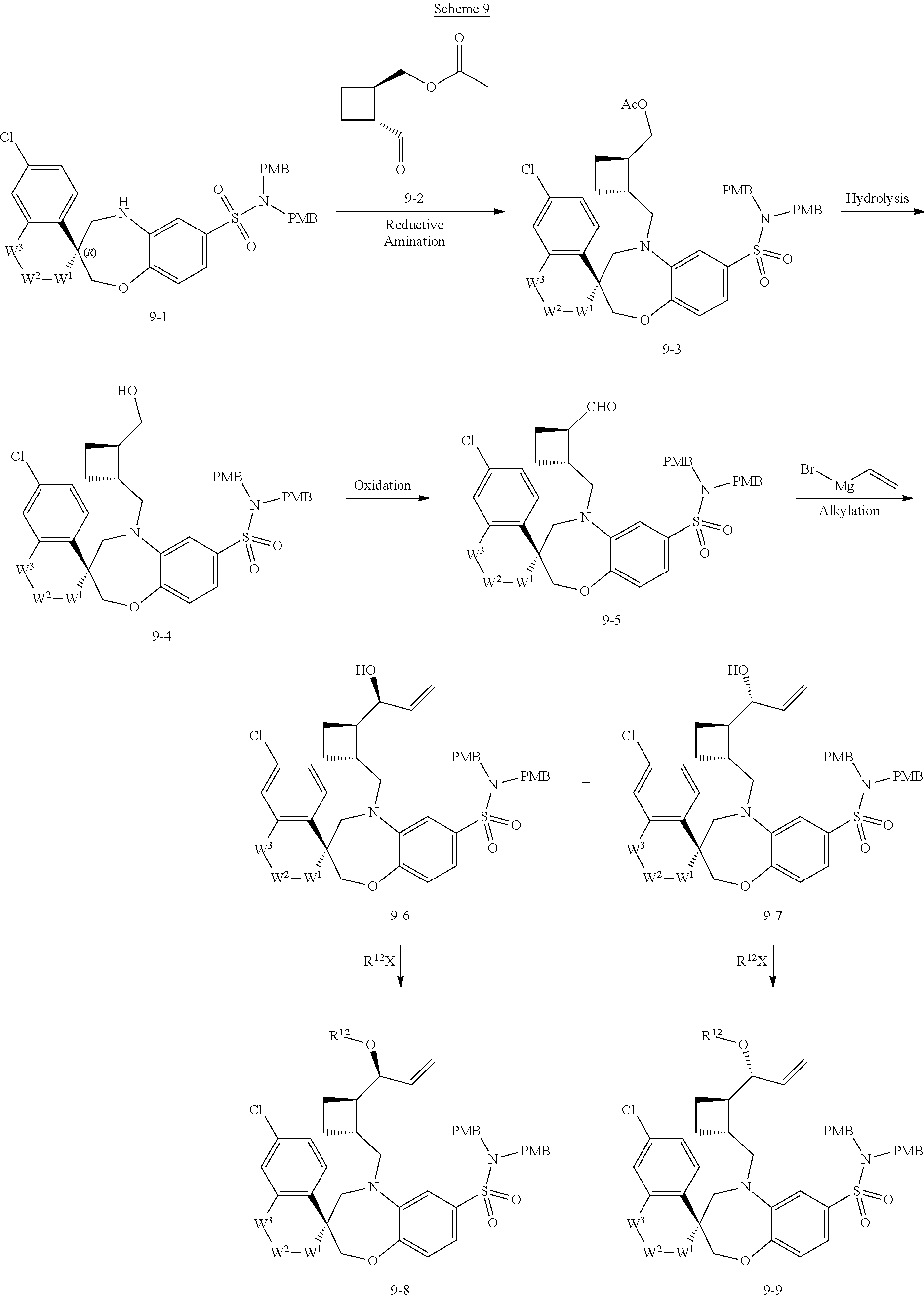

Alternatively, the allyl alcohol derivatives of formula 9-6 or 9-7 can be prepared by the methods outlined in Scheme 10 via reductive amination of the spiro-sulfonamide 10-1 with the aldehyde 10-2 or 10-3 by methods described in Scheme 9.

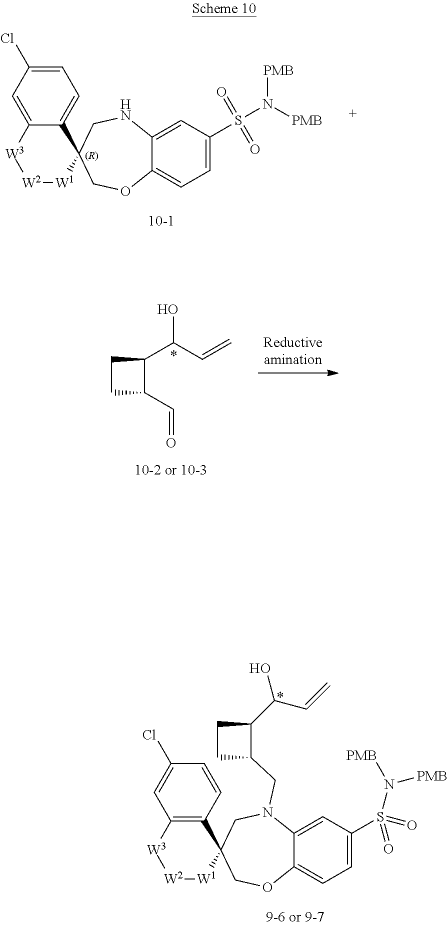

The aldehyde 10-2 or 10-3 can be prepared by the methods outlined in Scheme 11. Alkylation of 11-1 with a suitable agent (e.g., vinylmagnesium bromide or vinyllithium) can produce a mixture of the allyl alcohol 11-2 or 11-3 that can be separated by chromatographic methods as described above. Oxidation of 11-2 or 11-3 under suitable conditions (e.g., Dess-Martin or Swern conditions) can afford the 10-2 or 10-3, respectively.

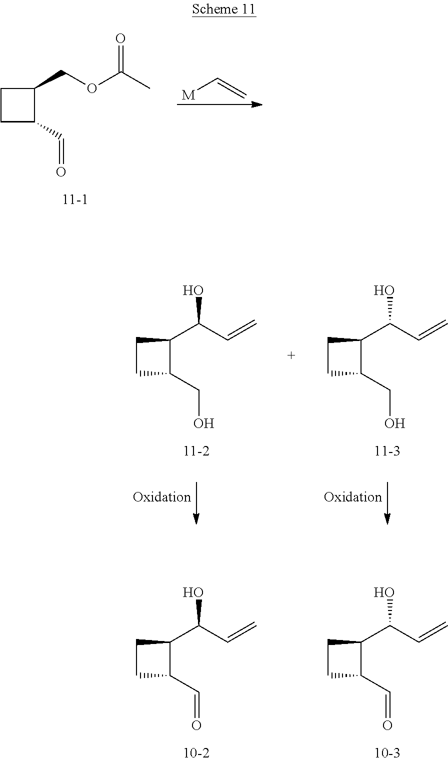

A series of macrocyclic sulfonimidamide derivatives of formula 12-9 can be prepared by the methods outlined in Scheme 12. Alkylation of 12-1 can afford the ether 12-2. Compound 12-4 can be obtained by treatment of 12-2 with TFA followed by reaction with TBSCl. 12-4 can be transferred to 12-5 by reaction with Ph₃PCl₂ and followed the treatment with NH₃. Reaction of 12-5 with acyl chloride 12-6 can yield sulfonimidamide 12-7 which can be transformed into the corresponding product 12-8 by treatment with an appropriate R⁴Cl[ClC(O)OR^{4A}, ClC(O)R^{4B}, ClC(O)NR^{4C}R^{4D}, ClS(O)R^{4B}, ClS(O)₂R^{4B}, ClS(O)NR^{4C}R^{4D}, ClS(O)₂NR^{4C}R^{4D}] or isocyanate R^{4C}N═C═O in the presence of a suitable base (e.g. DIEA, TEA, or pyridine). RCM of sulfonimidamide derivatives 12-8 can afford a pair of diastereomers of the corresponding macrocyclic sulfonimidamide derivatives 12-9 which can be separated into single diastereomers 12-9A and 12-9B by flash chromatography on a normal phase column, a reverse phase column or a chiral column if necessary.

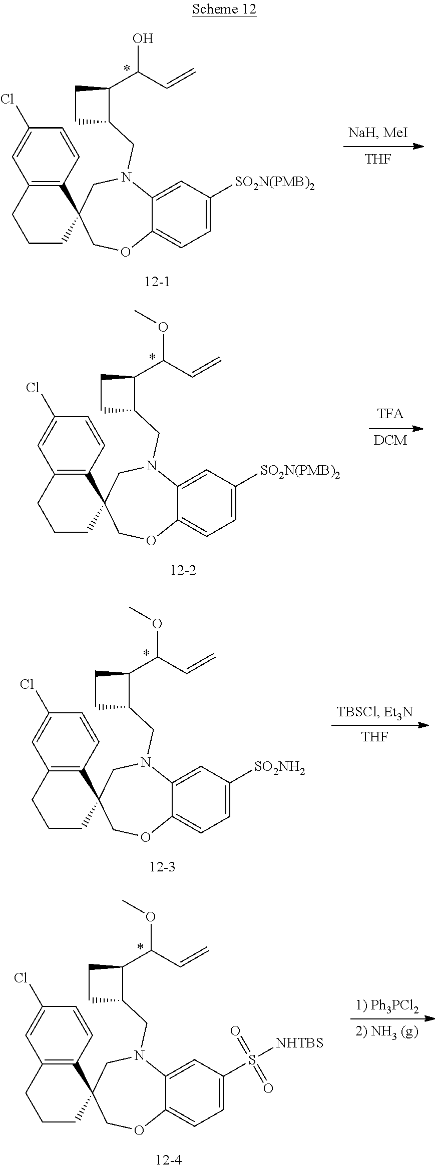

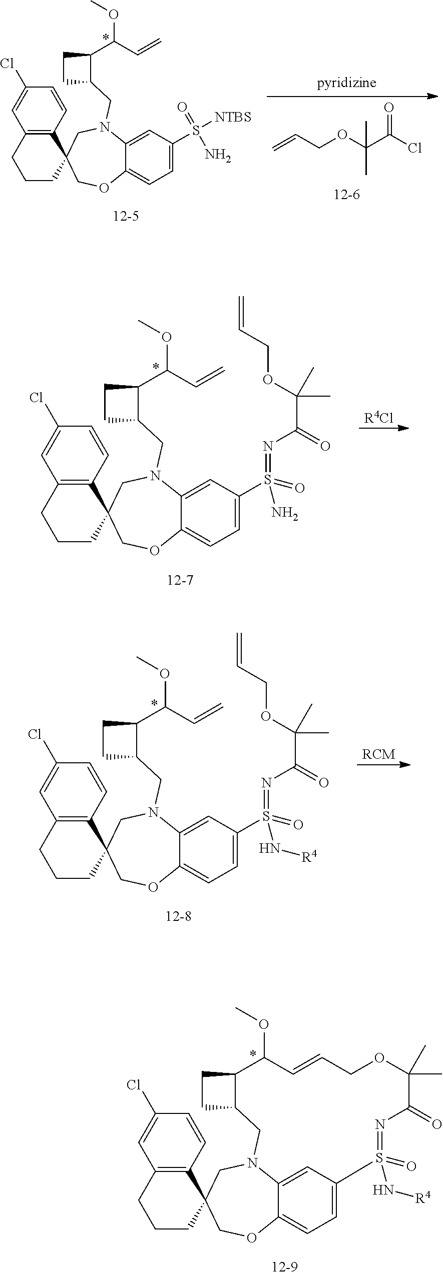

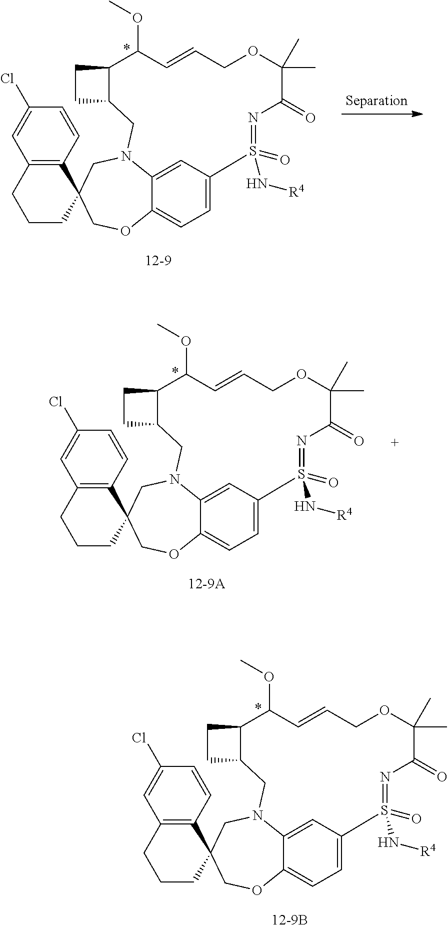

Similarly, a series of macrocyclic sulfonimidamide derivatives of formula 13-10 can be prepared by the methods outlined in Scheme 13. Compound 13-3 can be obtained by treatment of 13-1 with TFA followed by reaction with TBSCl. Protection of the OH group in 13-3 to give THP protecting product 13-4 can be achieved by reaction with THP in the present of acid such as TsOH. Compound 13-4 can be transformed into 13-9 by the similar consequences described in Scheme 12 for 12-4 to 12-9. Removal of the protecting THP group under acidic conditions (e.g., TsOH, HCl or TFA) of 13-9 can give the alcohol 13-10.

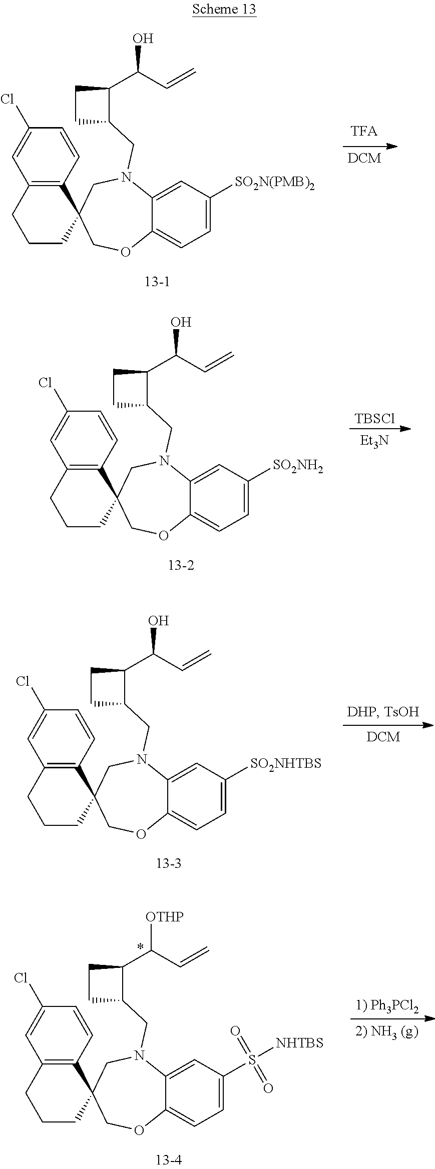

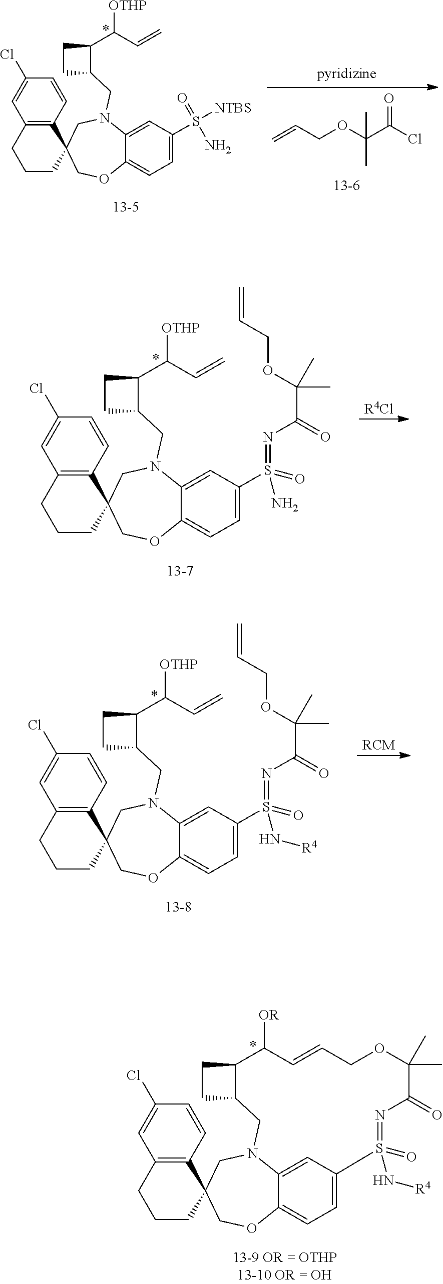

A series of carbamate derivatives of formula 14-3 can be prepared by the methods outlined in Scheme 14, The alcohol 14-1 can be transformed into the desired carbamate derivative 14-3 by reaction with a suitable dialkylcarbamoyl chloride 14-2, or by reaction with diphenyl carbonate or CDI followed by treatment with suitable amine R^{c}R^{d}NH. The carbamate derivative 14-3 can be separated into single diastereomers 14-3A and 14-3B by flash chromatography on a normal phase column, a reverse phase column or a chiral column if necessary.

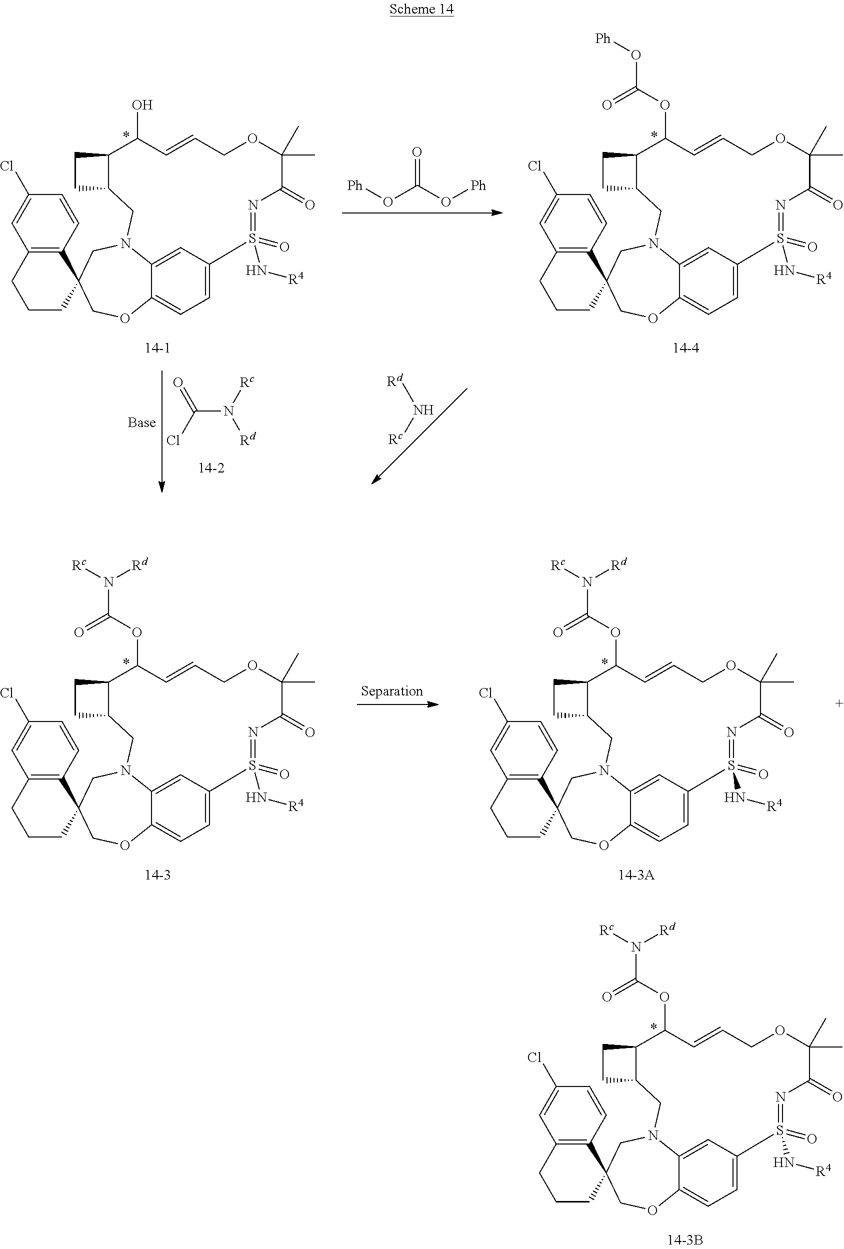

A series of 2-aminoethylene ether of formula 15-5 can be prepared by the methods outlined in Scheme 15. Epoxide opening of compound 15-2 (R^{os}=optional substituent) with macrocyclic alcohol 15-1 can give the corresponding alcohol 15-3 which can be transformed into the ether 15-5 by a two-step process of activation of the alcohol (e.g., mesylation or tosylation) to yield 15-4 with a leaving group (Lg) and then displacement of the Lg with an amine R^{c}R^{d}NH to afford 15-5.

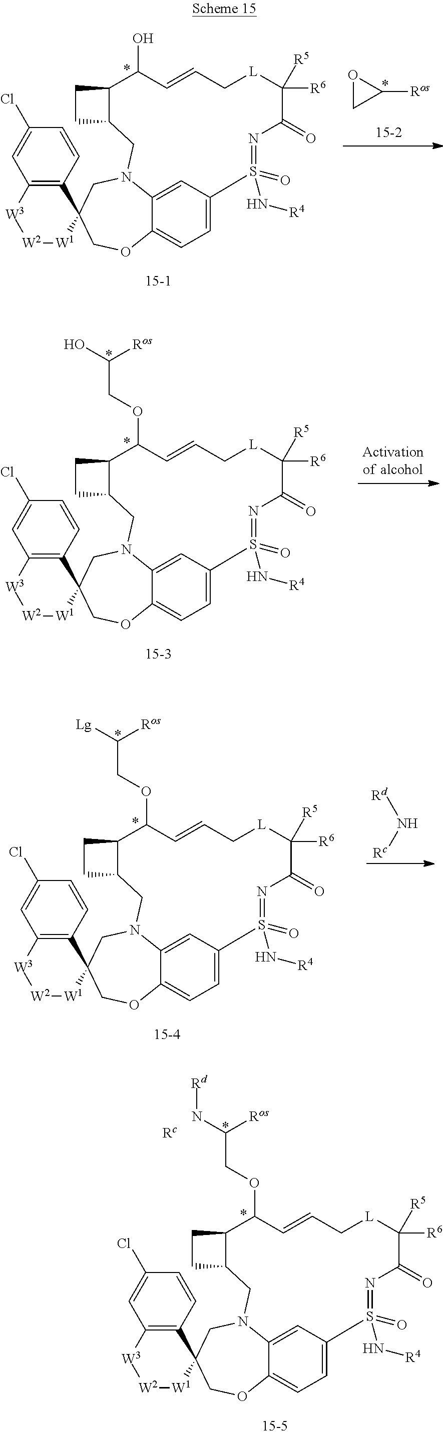

In a similar manner, a series of aminoalkylene ether of formula 16-4 can be prepared by the methods outlined in Scheme 16. Reaction of the macrocyclic alcohol 16-1 with bromoalkyl trifluoromethanesulfonate 16-2 (R^{os}=optional substituent, n=1-5) can give the bromide 16-3 which can be converted to the aminoalkylene ether 16-4 by reaction with a suitable amine R^{c}R^{d}NH.

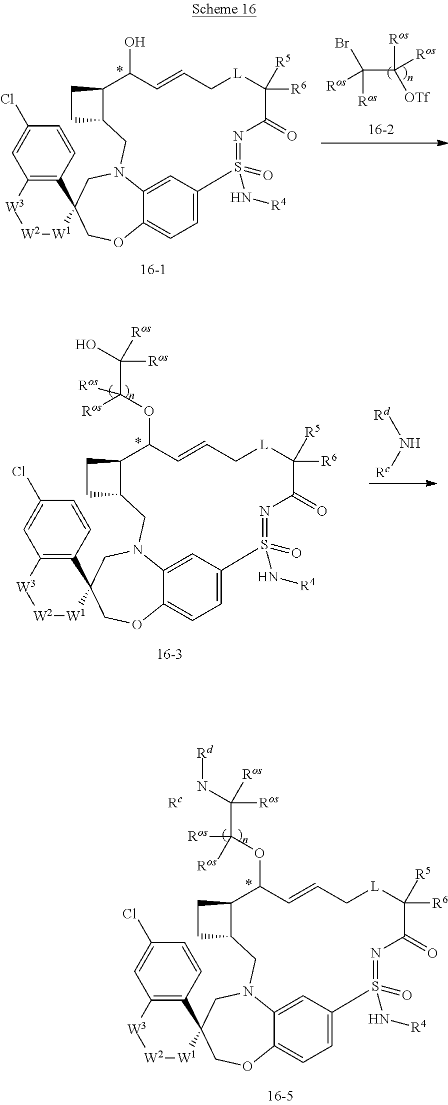

A series of 2-aminoethylene ether of formula 17-6 and amide derivative of formula 17-7 can be prepared by the methods outlined in Scheme 17. Reaction of the macrocyclic alcohol 17-1 with substituted 2-bromoacetic acid 17-2 (R^{os}=optional substituent) using a suitable base (e.g. NaH or DBU) can give the acid 17-3 which can be converted to the isobutyl carbonic anhydride 17-4 by treatment with isobutyl chloroformate. Reduction of the anhydride 17-4 with a suitable reducing agent (e.g., DIBAL or NaBH₄) at low temperature can provide the aldehyde 17-5 which can be transformed into the 2-aminoethylene ether 17-6 by reductive amination with R^{c}R^{d}NH using reductive agent (e.g., NaBH₄, NaBH(OAc)₃, or NaBCNH3). Alternatively, reaction of the anhydride 17-4 with amine R^{c}R^{d}NH can yield the corresponding amide 17-7.

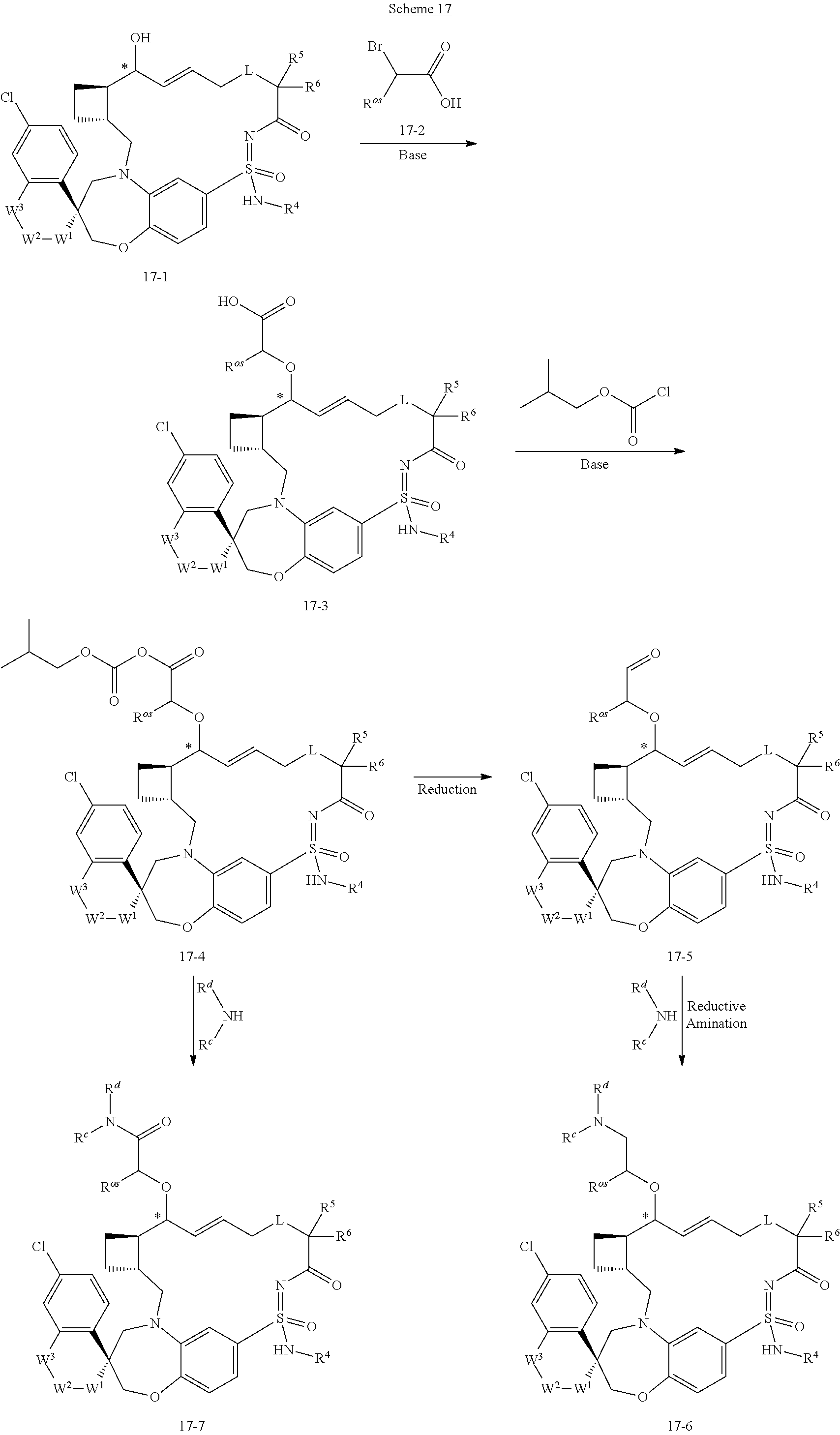

A series of macrocyclic sulfonimidamide derivatives of formula 18-6 can be prepared by the methods outlined in Scheme 18. Reaction of the sulfonimidamide derivative 18-1 with 3-allyl-oxazolidine-2,5-dione 18-2 in the presence of a suitable base (e.g., DBU or DIEA) can afford the amide 18-3 which can undergo reductive amination with an aldehyde RCHO by using a suitable reducing agent (e.g., NaBH₃(CN) or NaBH(OAc)₃) can afford 18-4. Transformation of 18-4 to the corresponding 18-5 can be achieved by reaction with an appropriate R⁴Cl[ClC(O)OR^{4A}, CLC(O)R^{4B}, ClC(O)NR^{4C}R^{4D}, ClS(O)R^{4B}, ClS(O)₂R^{4B}, ClS(O)NR^{4C}R^{4D}, ClS(O)₂NR^{4C}R^{4D}] or isocyanate R^{4C}N═C═O in the presence of a suitable base (e.g. DIEA, TEA, or pyridine). RCM of sulfonimidamide derivatives 18-5 can afford a pair of diastereomers of the corresponding macrocyclic sulfonimidamide derivatives 18-6 which can be separated into single diastereomers 18-6A and 18-6B by flash chromatography on a normal phase column, a reverse phase column or a chiral column if necessary.

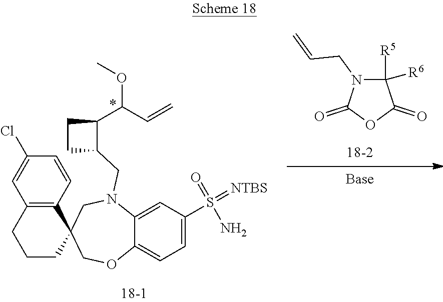

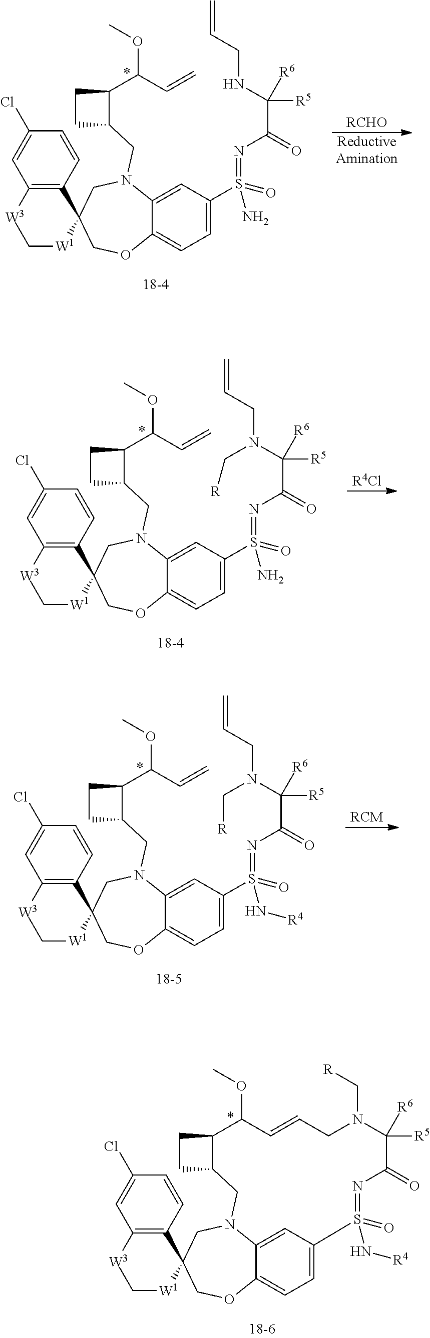

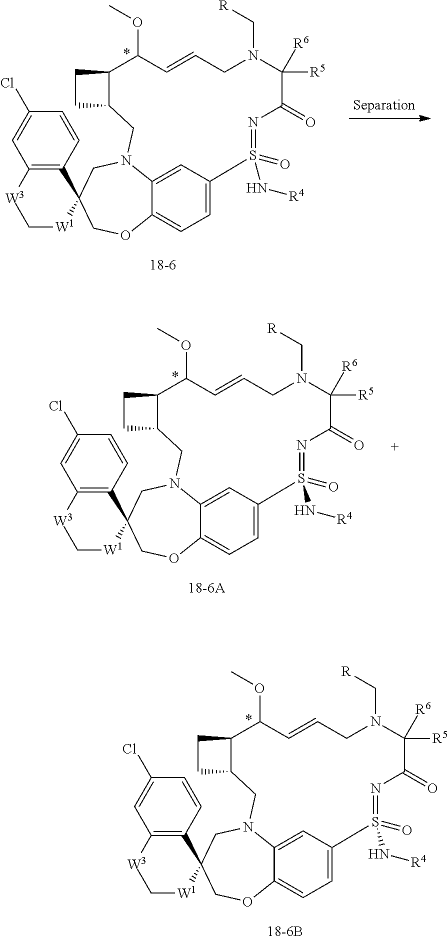

### Intermediate 1 N-[Amino-oxo-[(4S)-7-chloro-5′-[[(1R,2R)-2-[(1S)-1-methoxyprop-2-enyl]cyclobutyl]methyl]spiro[2,3-dihydro-1H-naphthalene-4,3′-2,4-dihydro-1,5-benzoxazepine]-7′-yl]-sulfanylidene]-2-methyl-2-prop-2-enoxypropanamide

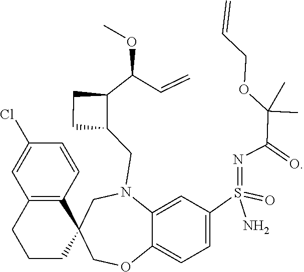 [SMILES: C=CCOC(C)(C)C(=O)N=S(N)(=O)c1ccc2c(c1)N(C[C@@H]1CC[C@H]1[C@H](C=C)OC)C[C@@]1(CCCc3cc(Cl)ccc31)CO2]

### Step 1: 6-chloro-3,4-dihydro-2H-spiro[naphthalene-1,2′-oxirane]

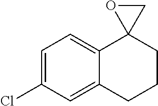 [SMILES: Clc1ccc2c(c1)CCCC21CO1]

To a solution of 6-chlorotetralin-1-one (208 g, 1.15 mol, 1 eq.) in DMSO (2080 mL) was added trimethylsulfonium iodide (246 g, 1.27 mol, 1.1 eq.) and potassium hydroxide (123 g, pellets, 2.3 mol, 2 eq.) portionwise. After addition, the mixture was stirred at 25° C. for 24 hours. TLC (PE:EA=10:1, R_{f}=0.5) showed the SM consumed, a new main spot was detected. The mixture was slowly poured into crushed ice (˜1600 g), and the flask was rinsed with MTBE (1 L). The resulting mixture was stirred for 5 min, and the layers were separated. The aqueous layer was extracted with MTBE (3×1 L). The combined organic layers were washed with brine (2×1 L). To the organic layer was added neutral Al₂O₃ (˜600 g). The resulting suspension was stirred for 5 min at r.t. and filtered. The filter cake was washed with MTBE (1 L). The filtrate was concentrated under reduced pressure to give 6-chloro-3,4-dihydro-2H-spiro[naphthalene-1,2′-oxirane] as a dark red oil (189 g) which was used for the next step without future purification.

### Step 2: 6-chloro-1,2,3,4-tetrahydronaphthalene-1-carbaldehyde

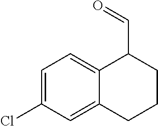 [SMILES: O=CC1CCCc2cc(Cl)ccc21]

To a solution of 6-chloro-3,4-dihydro-2H-spiro[naphthalene-1,2′-oxirane] (189 g, 0.97 mol, 1 eq.) in THF (1890 mL) was added boron trifluoride etherate (4.32 mL, 48.5 mmol, 0.05 eq.) dropwise over 3 min at −8° C. in a dry ice/IPA bath (the internal temperature raised to 10° C. instantly). The solution was then cooled down and stirred at −8° C. for another 10 mins. TLC (PE:EA=10:1) showed the reaction was completed. The reaction was then quenched with sat. NaHCO₃ (˜500 mL) at −8° C., and diluted by MTBE (1 mL). The layers were separated, and the aqueous layer was discarded (with some white suspension). The organic phase was then washed with brine (500 mL), dried over Na₂SO₄, filtered and concentrated under reduced pressure to give 6-chloro-1,2,3,4-tetrahydronaphthalene-1-carbaldehyde (136 g), which was used for the next step without further purification.

### Step 3: (6-chloro-1,2,3,4-tetrahydronaphthalene-1,1-diyl)dimethanol

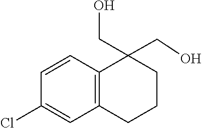 [SMILES: OCC1(CO)CCCc2cc(Cl)ccc21]

To a solution of 6-chloro-1,2,3,4-tetrahydronaphthalene-1-carbaldehyde (136 g, 0.7 mol, 1 eq.) in diethylene glycol (1360 mL) was added formaldehyde solution (37% wt in H₂O, 210.3 g, 521 mL, 7.0 mol, 10 eq.). The resulting mixture was cooled to 5° C. with an ice/salt bath. Potassium hydroxide (45% aqueous solution, 588 g solid, 15 eq.) was then added dropwise at such a rate to keep the reaction temperature below 20° C. After addition, the reaction mixture was heated to 45° C. and stirred for 1 h. GC analysis indicated that the reaction went completion. The reaction mixture was diluted with brine (500 mL), and the mixture was then extracted with DCM (7×1 L) until very small amount of product left in the aqueous layer. The combined organic layers were concentrated under reduced pressure and purified by silica gel column chromatography (10% to 80% EA in heptane) to give (6-chloro-1,2,3,4-tetrahydronaphthalene-1,1-diyl)dimethanol (81 g, 51%) as a white solid. ¹H NMR (400 MHz, CDCl₃): δ 7.31-7.34 (m, 2H), 7.11-7.14 (m, 2H), 3.87-3.91 (m, 2H), 3.72-3.76 (m, 2H), 2.73-2.76 (m, 2H), 2.11-2.15 (m, 2H), 1.89-1.92 (m, 2H), 1.79-1.83 (m, 2H).

### Step 4: (S)-(6-chloro-1-(hydroxymethyl)-1,2,3,4-tetrahydronaphthalen-1-yl)methyl 4-bromobenzoate

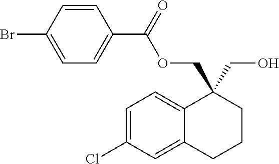 [SMILES: O=C(OC[C@@]1(CO)CCCc2cc(Cl)ccc21)c1ccc(Br)cc1]

To a solution of 2,6-bis((R)-5,5-dibutyl-4-phenyl-4,5-dihydrooxazol-2-yl)pyridine (R,R-Kang catalyst, 7.74 g, 13.0 mmol) in dry DCM (2220 mL), copper (II) chloride (1.75 g, 13.0 mmol) was added under N₂ and the resulting green colored solution was stirred at r.t. for 1 h. The solution was transferred via cannula to a solution of (6-chloro-1,2,3,4-tetrahydronaphthalene-1,1-diyl)dimethanol (148 g, 0.65 mol, 1 eq.) in dry DCM (4000 mL). The resulting mixture was cooled to −78° C. and a light green precipitate was observed. A solution of 4-bromobenzoyl chloride (171.4 g, 0.78 mol, 1.2 eq.) in dry DCM (2000 mL) was added dropwise, followed by the addition of DIPEA dropwise (98.6 g, 0.78 mol, 1.2 eq.). The mixture was stirred at −78° C. for 3 h and monitored by HPLC. Upon completion, the reaction was quenched by pH 3 phosphate buffer (5 L), and warmed to ambient temperature with vigorous stirring. The layers were separated, and the aqueous layer was extracted with DCM (3×2 L). The combined organic layers were then washed with pH 3 phosphate buffer (2 L), sat. NaHCO₃ (2 L) and brine (2 L), dried over Na₂SO₄, filtered and concentrated under reduced pressure. The residue was purified by a silica gel column chromatography (0%-30% EA in heptane) to afford (S)-(6-chloro-1-(hydroxymethyl)-1,2,3,4-tetrahydronaphthalen-1-yl)methyl 4-bromobenzoate as a colorless oil (211 g, 79.0% yield). ¹H NMR (400 MHz, CDCl₃): δ 7.85 (d, 2H), 7.58 (d, 2H), 7.40 (m, 1H), 7.11 (m, 2H), 4.48 (s, 2H), 3.79 (d, 1H), 3.82 (d, 1H), 2.80 (m, 2H), 1.83-1.98 (m, 4H).

### Step 5: (R)-(6-chloro-1-formyl-1,2,3,4-tetrahydronaphthalen-1-yl)methyl 4-bromobenzoate

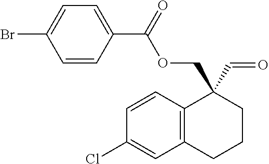 [SMILES: O=C[C@]1(COC(=O)c2ccc(Br)cc2)CCCc2cc(Cl)ccc21]

To a solution (S)-(6-chloro-1-(hydroxymethyl)-1,2,3,4-tetrahydronaphthalen-1-yl)methyl 4-bromobenzoate (250 g, 0.61 mol, 1 eq.) in DCM (2500 mL) was added Dess-Martin periodinane (310.5 g, 0.73 mol, 1.2 eq.) at 10° C. The mixture was stirred at 25° C. for 3 h. The reaction was monitored by LC-MS. Upon completion, H₂O (22.5 mL) was added and the resulting mixture was stirred at r.t. for 30 min. The mixture was then cooled to 0° C. and quenched with a 1:1 mixture of 10% Na₂S₂O₃/sat. NaHCO₃ solution (4 L). The mixture was extracted with EA (2×2 L). The combined organic layers were washed with brine (1 L), dried over Na₂SO₄, filtered and concentrated under reduced pressure. The residue was purified by silica gel column chromatography (20%-80% EA in heptane) to give (R)-(6-chloro-1-formyl-1,2,3,4-tetrahydronaphthalen-1-yl)methyl 4-bromobenzoate (200 g, 80.0% yield) as a creamy white solid. ¹H NMR (300 MHz, DMSO-d₆): δ 9.70 (s, 1H), 7.80 (m, 2H), 7.75 (m, 2H), 7.27-7.37 (m, 3H), 4.77 (d, J=11.6 Hz, 1H), 4.53 (d, J=11.6 Hz, 1H), 2.77-2.83 (m, 2H), 2.22-2.27 (m, 1H), 1.93-1.99 (m, 1H), 1.80-1.89 (m, 1H), 1.72-1.80 (m, 1H).

### Step 6: (R)-(6-chloro-1-(dimethoxymethyl)-1,2,3,4-tetrahydronaphthalen-1-yl)methanol

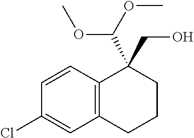 [SMILES: COC(OC)[C@]1(CO)CCCc2cc(Cl)ccc21]

To a solution of (R)-(6-chloro-1-formyl-1,2,3,4-tetrahydronaphthalen-1-yl)methyl 4-bromobenzoate (174 g, 0.43 mol, 1 eq.) in methanol (1700 mL) were added trimethyl orthoformate (135.9 g, 1.29 mol, 3 eq.) and p-TsOH H₂O (3.96 g, 21.5 mmol, 0.05 eq.). The mixture was stirred at 70° C. for 1 h. LC-MS analysis indicated that the reaction went completion. The mixture was then concentrated to 50% volume and diluted with THF (2.6 L) and 1 N NaOH (2.6 L). The resulting reaction mixture was stirred at 40° C. for overnight and monitored by LC-MS. Upon completion, the mixture was extracted with EA (3×3 L), and the combined organic layers were washed with 1 N NaOH (2 L) and brine (2 L), dried over Na₂SO₄, and concentrated under reduced pressure. The residue was purified by silica gel column chromatography (20%-80% EA in heptane) to give (R)-(6-chloro-1-(dimethoxymethyl)-1,2,3,4-tetrahydronaphthalen-1-yl)methanol (100 g, 86.0% yield) as a pale yellow oil. ¹H NMR (300 MHz, CDCl₃): δ 7.47 (d, J=8.4 Hz, 1H), 7.10-7.15 (m, 2H), 4.50 (s, 1H), 3.91 (dd, J=3.8, 11.2 Hz, 1H), 3.57 (dd, J=8.4, 11.2 Hz, 1H), 3.47 (s, 3H), 3.35 (s, 3H), 2.70-2.82 (m, 2H), 2.48 (m, 1H), 2.00-2.07 (m, 1H), 1.92-1.96 (m, 1H), 1.83-1.90 (m, 1H), 1.69-1.76 (m, 1H).

### Step 7: 4-fluoro-N,N-bis[(4-methoxyphenyl)methyl]-3-nitro-benzenesulfonamide

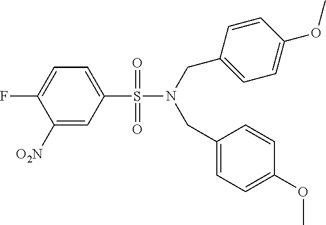 [SMILES: COc1ccc(CN(Cc2ccc(OC)cc2)S(=O)(=O)c2ccc(F)c([N+](=O)[O-])c2)cc1]

To a cooled (−35° C.) solution of 4-Fluoro-3-nitrobenzenesulfonyl chloride (4.89 g, 20.42 mmol) in THF (50 mL) was added Triethylamine (3.13 mL, 22.46 mmol), followed by addition of bis-(4-methoxybenzyl)amine (4.97 mL, 20.73 mmol) in THF (50 mL) solution over 30 min. while the temperature was kept at −35° C. After completion of the addition, the temperature was allowed slowly to warm to 0° C. over 1 h., and the mixture was stirred at 0° C. for an additional hour, TLC (PE:EA=5:1, R_{f}=0.5)). The mixture was neutralized with 1 N HCl to pH about 4-5 and diluted with EtOAc (100 mL). The organic layer was separated, washed with 1N HCl (10 mL), 7.5% NaHCO₃ aqueous solution (20 mL), and brine, dried over Na₂SO₄, filtered, and concentrated under reduced pressure. The residue was treated with DCM (30 mL), and hexane was added to the suspension until it became cloudy. The resulting suspension was sonicated for 2 min. and left at r.t. for 1 h. The mixture was filtered. and washed with hexane to afford the desired title product (6.85 g) without further purification. The mother liquid was concentrated under reduced pressure. The residue was treated with DCM (5 mL) and hexane was added as the procedures mentioned above to afford the additional 0.51 g of the title product. Total product 4-fluoro-N,N-bis[(4-methoxyphenyl)methyl]-3-nitro-benzenesulfonamide obtained is 7.36 g (78%). ¹H NMR (400 MHz, DMSO-d₆): δ 8.18-8.23 (m, 2H), 7.75-7.79 (q, 1H), 7.08 (d, 4H), 6.81 (d, 4H), 4.31 (s, 4H), 3.71 (s, 6H). ¹⁹F NMR (376 MHz, DMSO-d₆): δ −112.54 (s, 1F). LCMS calc. for C₂₂H₂₂FN₂O₆S (M+H)⁺: m/z=461.11; Found: 461.1.

### Step 8: (R)-4-((6-chloro-1-(dimethoxymethyl)-1,2,3,4-tetrahydronaphthalen-1-yl)methoxy)-N,N-bis(4-methoxybenzyl)-3-nitrobenzenesulfonamide

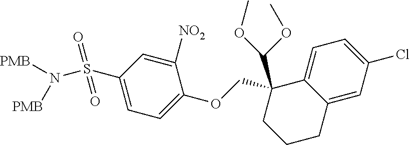

To a solution of (R)-(6-chloro-1-(dimethoxymethyl)-1,2,3,4-tetrahydronaphthalen-1-yl)methanol (100 g, 0.37 mol, 1 eq.) in THF (1600 mL) was added LiHMDS dropwise (20% in THF, 1.3 mol/L, 512 mL, 0.67 mol, 1.8 eq.) under N₂ atmosphere at −40° C., the solution was stirred at −40° C. for 5 mins, then a solution of 4-fluoro-N,N-bis[(4-methoxyphenyl)methyl]-3-nitro-benzenesulfonamide (187.42 g, 0.41 mol, 1.1 eq) in THF (500 mL) was added dropwise. The resulting mixture was stirred another 5 mins under −40° C., and then allowed to warm to RT and stirred for 1 hour. HPLC indicated that the reaction went completion. The reaction was cooled with ice-water bath and quenched with sat. NH₄Cl aqueous solution (2 L). The mixture was extracted with EtOAc (3×2 L). The combined organic layers were washed with sat. NH₄Cl solution (1 L) and brine (1 L), dried over Na₂SO₄, filtered and concentrated under reduced pressure. The residue was purified by silica gel column chromatography (10%-80% EA in heptane) to give N,N-bis[(4-methoxyphenyl)methyl]-3-nitro-4-[[(1R)-6-chloro-1-(dimethoxymethyl)tetralin-1-yl]methoxy]benzenesulfonamide (180 g, 63% yield) as a yellow solid. ¹H NMR (400 MHz, DMSO-d₆): δ 8.06-8.07 (m, 1H), 7.97-8.00 (m, 1H), 7.60-7.62 (m, 1H), 7.49-7.51 (m, 1H), 7.14-7.17 (m, 2H), 6.99-7.07 (m, 4H), 6.77-6.79 (m, 4H), 4.62 (s, 1H), 4.27-4.36 (m, 2H), 4.24 (s, 4H), 3.70 (s, 6H), 3.39 (s, 3H), 3.30 (s, 3H), 2.68-2.71 (m, 2H), 1.98-2.00 (m, 1H), 1.81-1.85 (m, 2H), 1.71-1.73 (m, 1H).

### Step 9: (R)-4-((6-chloro-1-formyl-1,2,3,4-tetrahydronaphthalen-1-yl)methoxy)-N,N-bis(4-methoxybenzyl)-3-nitrobenzenesulfonamide

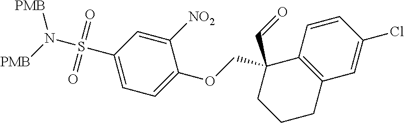

To a solution of (R)-4-((6-chloro-1-(dimethoxymethyl)-1,2,3,4-tetrahydronaphthalen-1-yl)methoxy)-N,N-bis(4-methoxybenzyl)-3-nitrobenzenesulfonamide (140 g, 0.197 mol, 1 eq.) in acetone (1.3 L) was added p-TsOH·H₂O (64 g, 0.331 mol, 1.7 eq.). The mixture was stirred at r.t for 20 hrs. TLC (PE:EA=4:1, R_{f}=0.3) showed the reaction was completed. The acetone was removed under reduced pressure and the residue was diluted with EA (0 L) and cooled to 0° C. Cold sat. NaHCO₃ (0.5 L) was added slowly, and the layers were separated. The organic phase was dried over Na₂SO₄, filtered and concentrated under reduced pressure to give (R)-4-((6-chloro-1-formyl-1,2,3,4-tetrahydronaphthalen-1-yl)methoxy)-N,N-bis(4-methoxybenzyl)-3-nitrobenzenesulfonamide as yellow oil (124 g, 95% yield), which was directly used for the next step without further purification.

### Step 10: (S)-6′-chloro-N,N-bis(4-methoxybenzyl)-3′,4,4′,5-tetrahydro-2H,2′H-spiro[benzo[b][1,4]oxazepine-3,1′-naphthalene]-7-sulfonamide

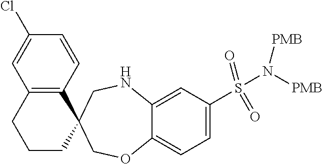

A) A solution of (R)-4-((6-chloro-1-formyl-1,2,3,4-tetrahydronaphthalen-1-yl)methoxy)-N,N-bis(4-methoxybenzyl)-3-nitrobenzenesulfonamide (124 g, 0.186 mol, 1 eq.) in AcOH (1.8 L) was heated to 70° C. and iron powder (94 g, 1.68 mol, 9 eq.) was added portion-wise. After addition, the reaction mixture was heated for 3 hours at 75° C. LC-MS showed that all starting material was consumed. The acetic acid was then removed under reduced pressure and the residue was poured into a 12 L, 3-neck round bottom flask charged with 1,2-dichloroethane (2.5 L). Then NaBH(OAc)₃ (18 g, 0.556 mol, 1 eq.) was added portion-wise over 30 min. and the reaction mixture was stirred at ambient temperature for 1 h. The reaction was monitored by LC-MS. Upon complete, the reaction was quenched with H₂O (500 mL), followed by addition of 10% aqueous citric acid (1 L). The layers were separated, and the aqueous phase was extracted with DCE (1 L). The combined organic layers were washed with half brine (2 L), and dried over Na₂SO₄, and concentrated under reduced pressure. The crude product was purified by column chromatography (silica gel 80-200 mesh, eluted with 10%-80% EA in heptane) to afford (S)-6′-chloro-N,N-bis(4-methoxybenzyl)-3′,4,4′,5-tetrahydro-2H,2′H-spiro[benzo[b][1,4]oxazepine-3,1′-naphthalene]-7-sulfonamide as white solid (93 g, 82% yield, 74% ee).

B) To a solution of (S)-6′-chloro-N,N-bis(4-methoxybenzyl)-3,4,4′,5-tetrahydro-H,2′H-spiro[benzo[b][1,4]oxazepine-3,1′-naphthalene]-7-sulfonamide (69.6 g, 0.112 mol, 1.0 eq., ee: 74%) in ethyl acetate (EA, 290 mL) was added 1S-(+)-CSA (31.4 g, 0.135 mol, 1.2 eq.) at 20° C. After stirred for 5-10 min., the suspension was turn to clear solution. Then slowly white precipitate was formed. The resulting suspension was stirred for 12 h. The product was filtered, and filter cake was washed with EA/heptane (40 mL/160 mL) and dried in vacuum to obtain (S)-7-(N,N-bis(4-methoxybenzyl)sulfamoyl)-6′-chloro-3′,4,4′,5-tetrahydro-2H,2′H-spiro[benzo[b][1,4]oxazepine-3,1′-naphthalen]-5-ium ((1S)-7,7-dimethyl-2-oxobicyclo[2.2.1]heptan-1-yl)methanesulfonate (69.9 g, yield 73%, purity: 98.4%, ee: 98.3%) as white solid. LCMS calc. for C₃₄H₃₆ClN₂O₅S (M+H)⁺: m/z=619.2; Found: 619.3. ¹H NMR (400 MHz, DMSO-d₆): δ 7.81-7.83 (m, 1H), 7.24-7.28 (m, 2H), 7.17-7.18 (m, 1H), 6.95-7.06 (m, 6H), 6.78-6.80 (m, 4H), 6.20 (s, 1H), 4.15 (m, 4H), 4.08-4.14 (m, 2H), 3.68 (s, 6H), 3.30-3.36 (m, 1H), 3.23-3.27 (m, 1H), 2.71-2.75 (m, 2H), 1.76-1.86 (m, 3H), 1.56-1.61 (m, 1H).

### Step 11: [(1R,2R)-2-[[(3S)-7-[bis[(4-methoxyphenyl)methyl]sulfamoyl]-6′-chloro-spiro[2,4-dihydro-1,5-benzoxazepine-3,1′-tetralin]-5-yl]methyl]cyclobutyl]methyl acetate

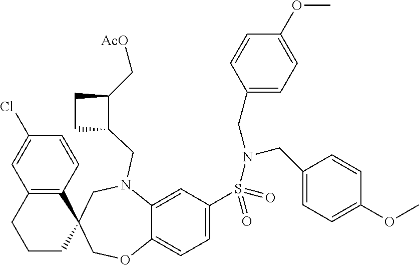 [SMILES: COc1ccc(CN(Cc2ccc(OC)cc2)S(=O)(=O)c2ccc3c(c2)N(C[C@@H]2CC[C@H]2COC(C)=O)C[C@@]2(CCCc4cc(Cl)ccc42)CO3)cc1]

To a stirred solution of sodium borohydride (3.48 g, 92.06 mmol) in DCM (200 mL) was dropwise added 2,2,2-trifluoroacetic acid (7.04 mL, 92.06 mmol). The resulting mixture was stirred at 0° C. for 10 min. A solution of (S)-6′-chloro-N,N-bis(4-methoxybenzyl)-3′,4,4′,5-tetrahydro-2H,2′H-spiro[benzo[b][1,4]oxazepine-3,1′-naphthalene]-7-sulfonamide (28.5 g, 46 mmol) and [(1R,2R)-2-formylcyclobutyl]methyl acetate (8.63 g, 55.24 mmol) in DCM (200 mL) was then dropwise added at 0° C. The resulting mixture was stirred at r.t. overnight. The reaction was monitored by LC-MS. Sodium borohydride (3.48 g, 92.06 mmol, 2 eq.) and 2,2,2-trifluoroacetic acid (7.04 mL, 92.06 mmol, 2 eq.) were added to the mixture, and stirred for 3 h. The reaction was quenched with methanol (30 mL), followed by saturated NaHCO₃ solution (300 mL). The mixture was extracted with DCM (300 mL×3). The combined organic layers were dried over Na₂SO₄, filtered and concentrated under reduced pressure. The residue was purified by flash chromatography on a silica gel column using EtOAc/Heptanes (5-40%) to afford the desired product [(1R,2R)-2-[[(3S)-7-[bis[(4-methoxyphenyl)methyl]sulfamoyl]-6′-chloro-spiro[2,4-dihydro-1,5-benzoxazepine-3,1′-tetralin]-5-yl]methyl]cyclobutyl]methyl acetate (34.5 g, 98.7% yield) as a white solid. LC-MS calc. for C₄₂H₄₈ClN₂O₇S [M+H]⁺. m/z=759.28/761.28; Found 759.7/761.6.

### Step 12: (3S)-6′-chloro-N,N-bis[(4-methoxyphenyl)methyl]-5-[[(1R,2R)-2-(hydroxymethyl)cyclobutyl]methyl]spiro[2,4-dihydro-1,5-benzoxazepine-3,1′-tetralin]-7-sulfonamide

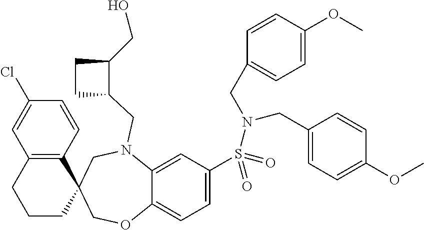 [SMILES: COc1ccc(CN(Cc2ccc(OC)cc2)S(=O)(=O)c2ccc3c(c2)N(C[C@@H]2CC[C@H]2CO)C[C@@]2(CCCc4cc(Cl)ccc42)CO3)cc1]

To a solution of [(1R,2R)-2-[[(3S)-7-[bis[(4-methoxyphenyl)methyl]sulfamoyl]-6′-chloro-spiro[2,4-dihydro-1,5-benzoxazepine-3,1′-tetralin]-5-yl]methyl]cyclobutyl]methyl acetate (54.0 g, 71.11 mmol) in THF (500 mL), methanol (500 mL) and water (500 mL) was added lithium monohydroxide (14.921 g, 355.57 mmol). The mixture was stirred at r.t. overnight. The solvent was then removed under reduced pressure, and the aqueous layer was extracted with DCM (100 mL×3). The combined organic layers were dried over Na₂SO₄, filtered and concentrated under reduced pressure to afford the crude product (3S)-6′-chloro-N,N-bis[(4-methoxyphenyl)methyl]-5-[[(1R,2R)-2-(hydroxymethyl)cyclobutyl]methyl]spiro[2,4-dihydro-1,5-benzoxazepine-3,1′-tetralin]-7-sulfonamide as white solid (52 g, quantitively yield) which was directly used in the next step without further purification. LC-MS calc. for C₄₀H₄₆ClN₂O₆S [M+H]⁺: m/z=717.27/719.27; Found 717.6/719.6.

### Step 13: (3S)-6′-chloro-N,N-bis[(4-methoxyphenyl)methyl]-5-[[(1R,2R)-2-formylcyclobutyl]methyl]spiro[2,4-dihydro-1,5-benzoxazepine-3,1′-tetralin]-7-sulfonamide

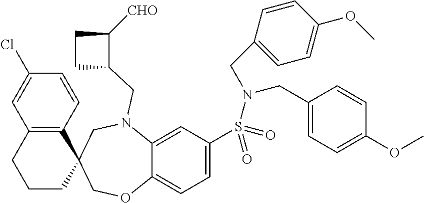 [SMILES: COc1ccc(CN(Cc2ccc(OC)cc2)S(=O)(=O)c2ccc3c(c2)N(C[C@@H]2CC[C@H]2C=O)C[C@@]2(CCCc4cc(Cl)ccc42)CO3)cc1]

DMSO (20.58 mL, 289.97 mmol) was slowly added to a cooled (−78° C.) solution of oxalyl chloride (12.43 mL, 144.99 mmol) in DCM (1000 mL). Gas was produced during this addition. The mixture was stirred at −78° C. for 30 min. Then a solution of (3S)-6′-chloro-N,N-bis[(4-methoxyphenyl)methyl]-5-[[(1R,2R)-2-(hydroxymethyl)cyclobutyl]methyl]spiro[2,4-dihydro-1,5-benzoxazepine-3,1′-tetralin]-7-sulfonamide (52.0 g, 72.49 mmol) in DCM (50 mL) was added to the cooled (−78° C.) solution over 5 min. The resulting mixture was stirred at −78° C. for 40 min. Triethylamine (101.04 mL, 724.93 mmol) was added. The solution was stirred at −78° C. for 10 min, and then slowly warmed up to 0° C. LC-MS showed that the starting material was consumed. Water (150 mL) was added and the layers were separated. The aqueous layer was extracted with DCM (300 mL×3). The combined organic layers were dried over Na₂SO₄, filtered and concentrated under reduced pressure. The residue was purified by flash chromatography on a silica gel column with EtOAc/Heptanes (5-50%) to afford (3S)-6′-chloro-N,N-bis[(4-methoxyphenyl)methyl]-5-[[(1R,2R)-2-formylcyclobutyl]methyl]spiro[2,4-dihydro-1,5-benzoxazepine-3,1′-tetralin]-7-sulfonamide (43 g, 82.9% yield) as a white solid. LC-MS calc. for C₄₀H₄₄ClN₂O₆S [M+H]⁺: m/z=715.25/717.26; Found 715.7/717.7.

### Step 14: (3S)-6′-chloro-N,N-bis[(4-methoxyphenyl)methyl]-5-[[(1R,2R)-2-[(1S)-1-hydroxyallyl]cyclobutyl]methyl]spiro[2,4-dihydro-1,5-benzoxazepine-3,1′-tetralin]-7-sulfonamide and (3S)-6′-chloro-N,N-bis[(4-methoxyphenyl)methyl]-5-[[(1R,2R)-2-[(1R)-1-hydroxyallyl]cyclobutyl]methyl]spiro[2,4-dihydro-1,5-benzoxazepine-3,1′-tetralin]-7-sulfonamide

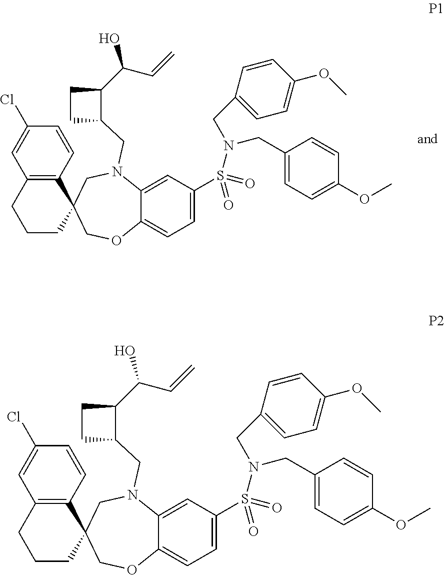 [SMILES: C=C[C@H](O)[C@@H]1CC[C@H]1CN1C[C@@]2(CCCc3cc(Cl)ccc32)COc2ccc(S(=O)(=O)N(Cc3ccc(OC)cc3)Cc3ccc(OC)cc3)cc21 ; C=C[C@@H](O)[C@@H]1CC[C@H]1CN1C[C@@]2(CCCc3cc(Cl)ccc32)COc2ccc(S(=O)(=O)N(Cc3ccc(OC)cc3)Cc3ccc(OC)cc3)cc21]

Vinylmagnesium bromide (1.0M solution in THF, 300 mL, 300 mmol) was diluted with THF (200 mL) in a 3 necked round bottom flack under nitrogen. (3S)-6′-chloro-N,N-bis[(4-methoxyphenyl)methyl]-5-[[(1R,2R)-2-formylcyclobutyl]methyl]spiro[2,4-dihydro-1,5-benzoxazepine-3,1′-tetralin]-7-sulfonamide (43.0 g, 60.1 mmol) dissolved in THF (400 mL) was introduced dropwise through a dropping funnel over 2 hours at room temperature. The reaction was monitored by LC-MS. After the starting material was consumed, the reaction was then quenched by addition of sat. aqueous solution NH₄Cl (300 mL) at 0° C. The organic layer was then separated, and the aqueous layer was extracted with ethyl acetate (300 mL×2). The combined organic layers were dried over Na₂SO₄, filtered and concentrated under reduced pressure. The residue was purified by flash chromatography on a silica gel column using EtOAc/Heptanes (5-40%) to afford two products: P1 (the earlier eluted product: 24.3 g, 40.5%) and P2 (the latter eluted product: 20 g, 33.3%).

P1 was assigned as (3S)-6′-chloro-N,N-bis[(4-methoxyphenyl)methyl]-5-[[(1R,2R)-2-[(1S)-1-hydroxyallyl]cyclobutyl]methyl]spiro[2,4-dihydro-1,5-benzoxazepine-3,1′-tetralin]-7-sulfonamide (Rt=4.43 min from LC-MS). LCMS calc. for C₄₂H₄₈ClN₂O₆S [M+H]⁺: m/z=743.28/744.29; Found 743.76/744.78. ¹H NMR (300 MHz, CDCl₃) δ 7.76 (t, J=7.2 Hz, 1H), 7.53 (d, J=1.9 Hz, 1H), 7.24-7.14 (m, 2H), 7.12 (d, J=2.0 Hz, 1H), 7.03-6.97 (m, 5H), 6.79 (t, J=5.7 Hz, 4H), 5.84-5.69 (m, 1H), 5.16 (d, J=17.2 Hz, 1H), 5.05 (d, J=10.4 Hz, 1H), 4.26 (t, J=5.6 Hz, 4H), 4.13 (s, 2H), 3.97 (d, J=4.4 Hz, 1H), 3.80 (d, J=1.8 Hz, 6H), 3.74 (d, J=6.2 Hz, 1H), 3.26 (d, J=14.2 Hz, 1H), 3.09 (dd, J=15.0, 9.3 Hz, 1H), 2.93 (d, J=4.2 Hz, 1H), 2.83-2.75 (m, 2H), 2.48-2.35 (m, 1H), 2.10-1.92 (m, 4H), 1.82 (m, 3H), 1.50 (m, 2H).

P2 was assigned as (3S)-6′-chloro-N,N-bis[(4-methoxyphenyl)methyl]-5-[[(1R,2R)-2-[(1R)-1-hydroxyallyl]cyclobutyl]methyl]spiro[2,4-dihydro-1,5-benzoxazepine-3,1′-tetralin]-7-sulfonamide (Rt=4.13 min from LC-MS). LCMS calc. for C₄₂H₄₈ClN₂O₆S [M+H]⁺: m/z=743.28/745.29; Found 743.8/745.8. ¹H NMR (300 MHz, CDCl₃) δ 7.75-7.68 (m, 1H), 7.24-7.14 (m, 3H), 7.12 (d, J=2.0 Hz, 1H), 7.01 (t, J=8.3 Hz, 5H), 6.79 (d, J=8.7 Hz, 4H), 5.85 (ddd, J=17.0, 10.4, 6.4 Hz, 1H), 5.29 (dd, J=17.2, 1.2 Hz, 1H), 5.17-5.08 (m, 1H), 4.26 (d, J=8.4 Hz, 4H), 4.14 (d, J=8.0 Hz, 3H), 3.81 (s, 6H), 3.69 (d, J=14.3 Hz, 11H), 3.59 (d, J=12.9 Hz, 1H), 3.31 (d, J=14.3 Hz, 1H), 3.15 (dd, J=14.9, 9.0 Hz, 1H), 2.84-2.76 (m, 2H), 2.67-2.56 (m, 1H), 2.23
2.09 (m, 2H), 2.03 (m, 2H), 1.86-1.73 (m, 3H), 1.59-1.46 (m, 2H).

### Step 15: (3S)-6′-chloro-N,N-bis[(4-methoxyphenyl)methyl]-5-[[(1R,2R)-2-[(1S)-1-methoxyallyl]cyclobutyl]methyl]spiro[2,4-dihydro-1,5-benzoxazepine-3,1′-tetralin]-7-sulfonamide

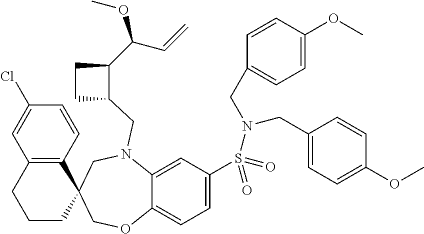 [SMILES: C=C[C@H](OC)[C@@H]1CC[C@H]1CN1C[C@@]2(CCCc3cc(Cl)ccc32)COc2ccc(S(=O)(=O)N(Cc3ccc(OC)cc3)Cc3ccc(OC)cc3)cc21]

To a cooled (ice-water bath) solution of (3S)-6′-chloro-N,N-bis[(4-methoxyphenyl)methyl]-5-[[(1R,2R)-2-[(1S)-1-hydroxyallyl]cyclobutyl]methyl]spiro[2,4-dihydro-1,5-benzoxazepine-3,1′-tetralin]-7-sulfonamide (3.2 g, 4.3 mmol, Step 14, P1) in THF (70 mL) was added sodium hydride (0.52 g, 12.91 mmol). The mixture was stirred for 5 min., then iodomethane (0.8 mL, 12.91 mmol) was added. The mixture was stirred at 50° C. overnight. The reaction was quenched by addition of saturated aqueous ammonium chloride (70 mL) and water (70 mL). The mixture was extracted with EtOAc (70 mL×3). The combined organic layers were washed with brine, dried over sodium sulfate, filtered and concentrated under reduced pressure to afford the crude product (3S)-6′-chloro-N,N-bis[(4-methoxyphenyl)methyl]-5-[[(1R,2R)-2-[(1S)-1-methoxyallyl]cyclobutyl]methyl]spiro[2,4-dihydro-1,5-benzoxazepine-3,1′-tetralin]-7-sulfonamide (3.1 g, 95.1% yield) which was directly used in next step directly without further purification. LCMS calc. for C₄₃H₅₀ClN₂O₆S [M+H]⁺. m/z=757.3/759.3. Found: 757.0/759.4.

### Step 16: (3S)-6′-chloro-5-[[(1R,2R)-2-[(1S)-1-methoxyallyl]cyclobutyl]methyl]spiro[2,4-dihydro-1,5-benzoxazepine-3,1′-tetralin]-7-sulfonamide

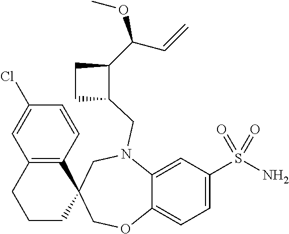 [SMILES: C=C[C@H](OC)[C@@H]1CC[C@H]1CN1C[C@@]2(CCCc3cc(Cl)ccc32)COc2ccc(S(N)(=O)=O)cc21]

To a stirred solution of (3S)-6′-chloro-N,N-bis[(4-methoxyphenyl)methyl]-5-[[(1R,2R)-2-[(1S)-1-methoxyallyl]cyclobutyl]methyl]spiro[2,4-dihydro-1,5-benzoxazepine-3,1′-tetralin]-7-sulfonamide (3.1 g, 4.1 mmol) in DCM (20 mL) was added TFA (25 mL) dropwise. The reaction was stirred at 40° C. overnight. The reaction was cooled to r.t. and slowly poured into a 120 mL of saturated K₂CO₃ solution under an ice bath. The mixture was extracted with DCM (30 mL×3). The combined organic layers were dried over sodium sulfate and concentrated under reduced pressure.

The crude product was purified by flash chromatography on a silica gel column (12 g) using EtOAc/Hetpanes (2% to 50%) to afford (3S)-6′-chloro-5-[[(1R,2R)-2-[(1S)-1-methoxyallyl]cyclobutyl]methyl]spiro[2,4-dihydro-1,5-benzoxazepine-3,1′-tetralin]-7-sulfonamide (1.7 g, 80%). LCMS calc. for C₂₇H₃₄ClN₂O₄S [M+H]⁺: m/z=517.2/519.2. Found: 516.8/518.7.

### Step 17: (4S)—N-[tert-butyl(dimethyl)silyl]-7-chloro-5′-[[(1R,2R)-2-[(1S)-1-methoxyprop-2-enyl]cyclobutyl]methyl]spiro[2,3-dihydro-1H-naphthalene-4,3′-2,4-dihydro-1,5-benzoxazepine]-7′-sulfonamide

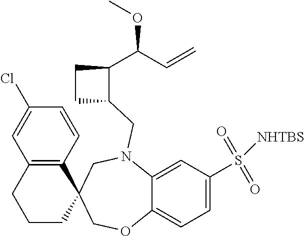

To a solution of (3S)-6′-chloro-5-[[(1R,2R)-2-[(1S)-1-methoxyallyl]cyclobutyl]methyl]spiro[2,4-dihydro-1,5-benzoxazepine-3,1′-tetralin]-7-sulfonamide (1.65 g, 3.19 mmol) in THF (30 mL) was added tert-butyl dimethylchlorosilane (0.58 g, 3.83 mmol) followed by triethylamine (0.89 mL, 6.38 mmol). The resulting mixture was stirred at 40° C. for 4 days. LC-MS indicated the consumption of starting material and the formation of desired product.

The solution was concentrated under reduced pressure. The residue was purified by flash chromatography on a silica gel column (12 g) eluting with EtOAc/heptane (5% to 60%) to afford (4S)—N-[tert-butyl(dimethyl)silyl]-7-chloro-5′-[[(1R,2R)-2-[(1S)-1-methoxyprop-2-enyl]cyclobutyl]methyl]spiro[2,3-dihydro-1H-naphthalene-4,3′-2,4-dihydro-1,5-benzoxazepine]-7′-sulfonamide (1.74 g, 86.4% yield) as a white solid. LC-MS calc. for C₃₃H₄₈ClN₂O₄SSi [M+H]⁺: m/z=631.3/633.3; Found: 631.2/633.5.

### Step 18: (4S)-7′-[S-amino-N-[tert-butyl(dimethyl)silyl]sulfonimidoyl]-7-chloro-5′-[[(1R,2R)-2-[(1S)-1-methoxyprop-2-enyl]cyclobutyl]methyl]spiro[2,3-dihydro-1H-naphthalene-4,3′-2,4-dihydro-1,5-benzoxazepine]

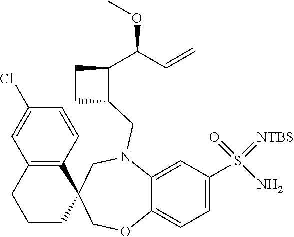

To a heating gun dried 50 mL round bottom flask was added (4S)—N-[tert-butyl(dimethyl)silyl]-7-chloro-5′-[[(1R,2R)-2-[(1S)-1-methoxyprop-2-enyl]cyclobutyl]methyl]spiro[2,3-dihydro-1H-naphthalene-4,3′-2,4-dihydro-1,5-benzoxazepine]-7′-sulfonamide (1.26 g, 2 mmol), followed by DCM (25 mL), triphenylphosphine (0.79 g, 2.99 mmol), hexachloroethane (0.71 g, 2.99 mmol) and triethylamine (1.11 mL, 7.98 mmol). The resulting mixture was stirred at 35° C. for 3 h. Then the solution was bubbled with ammonia gas for 5 min.

White solids precipitated during this process. Then the reaction was stirred at r.t. for an additional 30 min. The solution was concentrated under reduced pressure. The residue was purified by flash chromatography on a silica gel column (12 g) eluting EtOAc/heptane (5% to 80%) to afford (4S)-7′-[S-amino-N-[tert-butyl(dimethyl)silyl]sulfonimidoyl]-7-chloro-5′-[[(1R,2R)-2-[(1S)-1-methoxyprop-2-enyl]cyclobutyl]methyl]spiro[2,3-dihydro-1H-naphthalene-4,3′-2,4-dihydro-1,5-benzoxazepine] (770 mg, 61.2% yield) as a light brown oil. LC-MS calc. for C₃₃H₄₉ClN₃O₃SSi [M+H]⁺: m/z=630.3/632.3; Found: 630.0/632.4.

### Step 19: 2-methyl-2-prop-2-enoxypropanoyl chloride

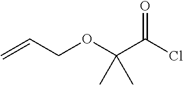

To a solution of 2-methyl-2-prop-2-enoxypropanoic acid (1.0 g, 6.94 mmol) in DCM (20 mL) was added DMF (0.01 mL, 0.06 mmol) followed by thionyl chloride (1.01 mL, 13.87 mmol). The resulting mixture was stirred at r.t. for 6 h. The mixture was concentrated under reduced pressure and then dried on high vacuum for 20 min. to remove all the excess thionyl chloride. The crude product 2-methyl-2-prop-2-enoxypropanoyl chloride (390 mg, 34.6% yield) was used in next step without further purification.

### Step 20: N-[amino-oxo-[(4S)-7-chloro-5′-[[(1R,2R)-2-[(S)-1-methoxyprop-2-enyl]cyclobutyl]methyl]spiro[2,3-dihydro-1H-naphthalene-4,3′-2,4-dihydro-1,5-benzoxazepine]-7′-yl]-sulfanylidene]-2-methyl-2-prop-2-enoxypropanamide

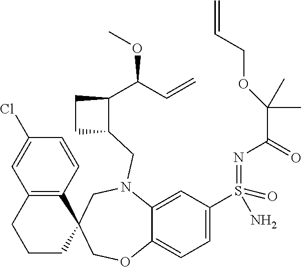 [SMILES: C=CCOC(C)(C)C(=O)N=S(N)(=O)c1ccc2c(c1)N(C[C@@H]1CC[C@H]1[C@H](C=C)OC)C[C@@]1(CCCc3cc(Cl)ccc31)CO2]

To a solution of (4S)-7′-[S-amino-N-[tert-butyl(dimethyl)silyl]sulfonimidoyl]-7-chloro-5′-[[(1R,2R)-2-[(1S)-1-methoxyprop-2-enyl]cyclobutyl]methyl]spiro[2,3-dihydro-1H-naphthalene-4,3′-2,4-dihydro-1,5-benzoxazepine] (130.0 mg, 0.21 mmol) in MeCN (3 mL) was added pyridazine (0.02 mL, 0.31 mmol) followed by 2-methyl-2-prop-2-enoxypropanoyl chloride (50.3 mg, 0.31 mmol). The resulting mixture was stirred at r.t. overnight, and then concentrated under reduced pressure. The residue was purified by flash chromatography on a silica gel column (12 g) with EtOAc/heptane (20% to 100%) to afford N-[amino-oxo-[(4S)-7-chloro-5′-[[(1R,2R)-2-[(1S)-1-methoxyprop-2-enyl]cyclobutyl]methyl]spiro[2,3-dihydro-1H-naphthalene-4,3′-2,4-dihydro-1,5-benzoxazepine]-7′-yl]-sulfanylidene]-2-methyl-2-prop-2-enoxypropanamide (127 mg, 95.9% yield) as a light yellow oil. LC-MS calc. for C₃₄H₄₄ClN₃O₅S [M+H]⁺: m/z=642.3/644.3; Found: 641.9/644.3. 1H NMR (300 MHz, CDCl₃) δ 7.68 (d, J=8.5 Hz, 1H), 7.30 (d, J=2.0 Hz, 1H), 7.26-7.21 (m, 1H), 7.21-7.15 (m, 1H), 7.10 (d, J=1.9 Hz, 1H), 6.95 (dd, J=8.3, 2.3 Hz, 1H), 5.97 (ddd, J=22.7, 10.8, 5.6 Hz, 1H), 5.58 (dtd, J=18.2, 10.6, 7.8 Hz, 1H), 5.34-5.09 (m, 4H), 4.11 (s, 2H), 4.03-3.94 (m, 2H), 3.68 (d, J=14.3 Hz, 2H), 3.53 (dd, J=18.9, 8.0 Hz, 1H), 3.35 (d, J=15.1 Hz, 1H), 3.30 (s, 3H), 3.21 (dd, J=15.7, 7.4 Hz, 1H), 2.78 (dd, J=8.8, 5.1 Hz, 2H), 2.59-2.44 (m, 1H), 2.18-2.08 (m, 1H), 2.06-1.52 (m, 8H), 1.46 (s, 6H).

### Intermediate 2 N-[amino-oxo-[(4S)-7-chloro-5′-[[(1R,2R)-2-[(1R)-1-methoxyprop-2-enyl]cyclobutyl]methyl]spiro[2,3-dihydro-1H-naphthalene-4,3′-2,4-dihydro-1,5-benzoxazepine]-7′-yl]-sulfanylidene]-2-methyl-2-prop-2-enoxypropanamide

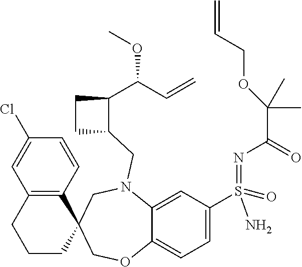 [SMILES: C=CCOC(C)(C)C(=O)N=S(N)(=O)c1ccc2c(c1)N(C[C@@H]1CC[C@H]1[C@@H](C=C)OC)C[C@@]1(CCCc3cc(Cl)ccc31)CO2]

This compound was prepared using procedures analogous to those described for Intermediate 1 Step 15-20 using (3S)-6′-chloro-N,N-bis[(4-methoxyphenyl)methyl]-5-[[(1R,2R)-2-[(1R)-1-hydroxyallyl]cyclobutyl]methyl]spiro[2,4-dihydro-1,5-benzoxazepine-3,1′-tetralin]-7-sulfonamide (P2, Intermediate 1 Step 14) in Step 15. LC-MS calc. for C₃₄H₄₄ClN₃O₅S [M+H]⁺. m/z=642.3/644.3; Found: 641.9/644.3. ¹H NMR (300 MHz, CDCl₃) δ 7.65 (dd, J=8.5, 2.1 Hz, 1H), 7.26 (dd, J=8.3, 3.8 Hz, 2H), 7.15 (d, J=8.5 Hz, 1H), 7.08 (s, 1H), 6.97-6.91 (m, 1H), 6.32 (s, 2H), 5.94 (ddd, J=22.5, 10.7, 5.6 Hz, 1H), 5.72-5.55 (m, 1H), 5.38-5.17 (m, 5H), 5.11 (dd, J=10.3, 0.7 Hz, 1H), 4.12-4.03 (m, 2H), 3.96 (t, J=6.3 Hz, 2H), 3.64 (dd, J=14.2, 2.4 Hz, 1H), 3.56-3.45 (m, 2H), 3.34-3.27 (m, 1H), 3.25 (d, J=6.5 Hz, 3H), 2.77 (s, 2H), 2.64-2.48 (m, 1H), 2.19 (dd, J=12.9, 5.3 Hz, 1H), 2.05-1.69 (m, 7H), 1.43 (s, 6H).

### Example 1 and Example 2 N-[(3R,6R,7S,8E,15R,22S)-7′-Chloro-7-methoxy-12,12-dimethyl-13,15-dioxospiro[11,20-dioxa-15-thia-1,14-diazatetracyclo[14.7.2.03,6.019,24]pentacosa-8,14,16,18,24-pentaene-22,4′-2,3-dihydro-1H-naphthalene]-15-yl]-2-methylpropanamide and N-[(3R,6R,7S,8E,15S,22S)-7′-chloro-7-methoxy-12,12-dimethyl-13,15-dioxospiro[11,20-dioxa-15-thia-1,14-diazatetracyclo[14.7.2.03,6.019,24]pentacosa-8,14,16,18,24-pentaene-22,4′-2,3-dihydro-1H-naphthalene]-15-yl]-2-methylpropanamide

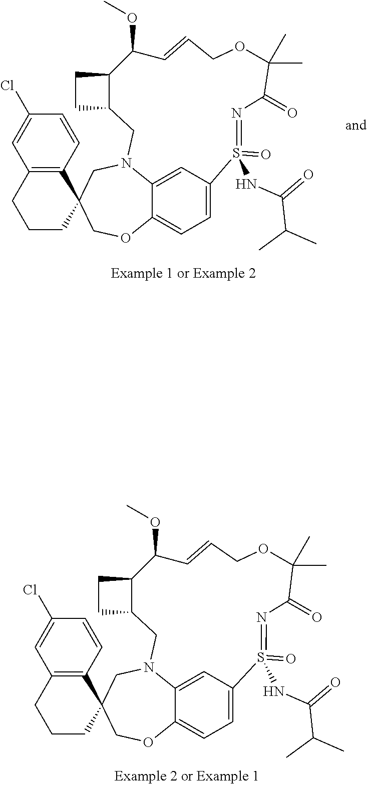 [SMILES: CO[C@H]1/C=C/COC(C)(C)C(=O)N=[S@@](=O)(NC(=O)C(C)C)c2ccc3c(c2)N(C[C@@H]2CC[C@H]21)C[C@@]1(CCCc2cc(Cl)ccc21)CO3 ; CO[C@H]1/C=C/COC(C)(C)C(=O)N=[S@](=O)(NC(=O)C(C)C)c2ccc3c(c2)N(C[C@@H]2CC[C@H]21)C[C@@]1(CCCc2cc(Cl)ccc21)CO3]

### Step 1: N-[[(4S)-7-chloro-5′-[[(1R,2R)-2-[(1S)-1-methoxyprop-2-enyl]cyclobutyl]methyl]spiro[2,3-dihydro-1H-naphthalene-4,3′-2,4-dihydro-1,5-benzoxazepine]-7′-yl]-(2-methylpropanoylamino)-oxo-sulfanylidene]-2-methyl-2-prop-2-enoxypropanamide

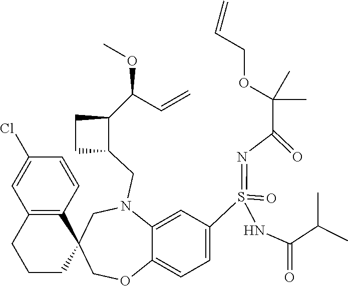 [SMILES: C=CCOC(C)(C)C(=O)N=S(=O)(NC(=O)C(C)C)c1ccc2c(c1)N(C[C@@H]1CC[C@H]1[C@H](C=C)OC)C[C@@]1(CCCc3cc(Cl)ccc31)CO2]

To a solution of N-[amino-oxo-[(4S)-7-chloro-5′-[[(1R,2R)-2-[(1S)-1-methoxyprop-2-enyl]cyclobutyl]methyl]spiro[2,3-dihydro-1H-naphthalene-4,3′-2,4-dihydro-1,5-benzoxazepine]-7′-yl]-sulfanylidene]-2-methyl-2-prop-2-enoxypropanamide (50.0 mg, 0.08 mmol, Intermediate 1) in DCM (1 mL) was added triethylamine (0.02 mL, 0.16 mmol) followed by isobutyryl chloride (0.01 mL, 0.12 mmol). The resulting mixture was stirred at r.t. for 30 min. The mixture was concentrated under reduced pressure. The residue was purified by flash chromatography on a silica gel column (12 g) with EtOAc/heptane (20% to 100% to afford N-[[(4S)-7-chloro-5′-[[(1R,2R)-2-[(1S)-1-methoxyprop-2-enyl]cyclobutyl]methyl]spiro[2,3-dihydro-1H-naphthalene-4,3′-2,4-dihydro-1,5-benzoxazepine]-7′-yl]-(2-methylpropanoylamino)-oxo-sulfanylidene]-2-methyl-2-prop-2-enoxypropanamide (42 mg, 75.8% yield) as a white solid. LC-MS calc. for C₃₈H₅ClN₃O₆S [M+H]⁺: m/z=712.3/714.3; Found: 712.2/714.6.

### Step 2: N-[(3R,6R,7S,8E,15R,22S)-7′-chloro-7-methoxy-12,12-dimethyl-13,15-dioxospiro[11,20-dioxa-15-thia-1,14-diazatetracyclo[14.7.2.03,6.019,24]pentacosa-8,14,16,18,24-pentaene-22,4′-2,3-dihydro-1H-naphthalene]-15-yl]-2-methylpropanamide and N-[(3R,6R,7S,8E,15S,22S)-7′-chloro-7-methoxy-12,12-dimethyl-13,15-dioxospiro[11,20-dioxa-15-thia-1,14-diazatetracyclo[14.7.2.03,6.019,24]pentacosa-8,14,16,18,24-pentaene-22,4′-2,3-dihydro-1H-naphthalene]-15-yl]-2-methylpropanamide

To a solution of N-[[(4S)-7-chloro-5′-[[(1R,2R)-2-[(1S)-1-methoxyprop-2-enyl]cyclobutyl]methyl]spiro[2,3-dihydro-1H-naphthalene-4,3′-2,4-dihydro-1,5-benzoxazepine]-7′-yl]-(2-methylpropanoylamino)-oxo-sulfanylidene]-2-methyl-2-prop-2-enoxypropanamide (42.0 mg, 0.06 mmol) in DCE (60 mL) was bubbled with nitrogen for 5 min. Then Hoveyda-Grubbs II catalyst (7.39 mg, 0.01 mmol) was added under nitrogen. The resulting mixture further bubbled with nitrogen for 5 min. and stirred at 70° C. under nitrogen overnight. The mixture was cooled to r.t. and stirred under air for 30 min. to deactivate the catalyst. The mixture was concentrated under reduced pressure. The residue was purified by prep-HPLC on C18 column (30×250 mm, 10 μm) with MeCN/H₂O (w/0.1% TFA, 20% to 100%) to afford P1 (the earlier eluted product) (4.7 mg, 10.3% yield) as a light yellow solid, and P2 (the latter eluted product) (5.3 mg, 11.8% yield) as a light yellow solid.

P1 was assigned to Example 1 or Example 2: LC-MS calc. for C₃₆H₄₇ClN₃O₆S [M+H]⁺: m/z=684.3/686.3; Found: 684.1/686.3. HPLC: C18 column (4.6×100 mm, 5 μm); flow rate=1 mL/min; mobile phase: 50% MeCN/H₂O (with 0.1% TFA) to 95% 4 min, 95% 6 min; λ=220 nm. tR=6.27 min. ¹H NMR (300 MHz, CDCl₃) δ 7.66 (d, J=8.6 Hz, 1H), 7.55-7.49 (m, 1H), 7.22-7.17 (m, 1H), 7.10 (d, J=2.3 Hz, 1H), 7.06-6.98 (m, 2H), 5.76 (dt, J=15.6, 3.5 Hz, 1H), 5.63 (dd, J=15.8, 7.1 Hz, 1H), 5.36 (s, 1H), 4.18 (d, J=4.5 Hz, 1H), 4.12 (s, 2H), 3.86 (dd, J=13.9, 5.8 Hz, 1H), 3.72 (d, J=14.4 Hz, 1H), 3.60-3.54 (m, 1H), 3.47-3.40 (m, 1H), 3.37 (d, J=3.2 Hz, 1H), 3.31 (d, J=4.6 Hz, 1H), 3.26 (d, J=1.1 Hz, 3H), 2.69-2.60 (m, 1H), 2.51-2.44 (m, 1H), 2.28 (t, J=7.6 Hz, 1H), 2.02 (d, J=12.1 Hz, 3H), 1.80 (t, J=8.5 Hz, 2H), 1.70-1.57 (m, 2H), 1.46 (s, 3H), 1.41 (d, J=2.8 Hz, 3H), 1.31-1.22 (m, 9H).

P2 was assigned to Example 2 or Example 1: LC-MS calc. for C₃₆H₄₇ClN₃O₆S [M+H]⁺: m/z=684.3/686.3; Found: 684.2/686.0. HPLC: C18 column (4.6×100 mm, 5 μm); flow rate=1 mL/min; mobile phase: 50% MeCN/H₂O (with 0.1% TFA) to 95% 4 min, 95% 6 min; λ=220 nm. tR=6.55 min. ¹H NMR (300 MHz, CDCl₃) δ 7.68 (d, J=8.5 Hz, 1H), 7.52 (dd, J=8.4, 2.2 Hz, 1H), 7.19 (dd, J=8.6, 2.4 Hz, 1H), 7.09 (d, J=2.4 Hz, 1H), 6.99 (d, J=8.4 Hz, 1H), 6.81 (d, J=2.3 Hz, 1H), 6.01 (dt, J=15.9, 3.0 Hz, 1H), 5.63 (dd, J=15.9, 5.2 Hz, 1H), 4.27-4.03 (m, 3H), 3.73 (t, J=14.2 Hz, 2H), 3.61 (s, 1H), 3.38 (s, 3H), 3.33-3.27 (m, 1H), 3.06 (dd, J=15.0, 10.5 Hz, 1H), 2.79 (d, J=4.5 Hz, 2H), 2.69-2.60 (m, 1H), 2.13-1.84 (m, 6H), 1.81-1.72 (m, 1H), 1.62 (q, J=8.7 Hz, 2H), 1.51 (s, 3H), 1.38 (s, 3H), 1.34-1.22 (m, 9H).

### Example 3 and Example 4 N-[(3R,6R,7S,8E,15R,22S)-7′-Chloro-7-methoxy-12,12-dimethyl-13,15-dioxospiro[11,20-dioxa-15-thia-1,14-diazatetracyclo[14.7.2.03,6.019,24]pentacosa-8,14,16,18,24-pentaene-22,4′-2,3-dihydro-1H-naphthalene]-15-yl]acetamide and N-[(3R,6R,7S,8E,15S,22S)-7′-chloro-7-methoxy-12,12-dimethyl-13,15-dioxospiro[11,20-dioxa-15-thia-1,14-diazatetracyclo[14.7.2.03,6.019,24]pentacosa-8,14,16,18,24-pentaene-22,4′-2,3-dihydro-1H-naphthalene]-15-yl]acetamide

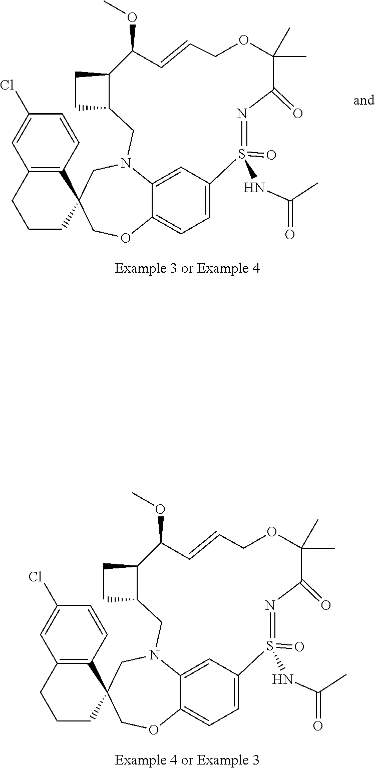 [SMILES: CO[C@H]1/C=C/COC(C)(C)C(=O)N=[S@@](=O)(NC(C)=O)c2ccc3c(c2)N(C[C@@H]2CC[C@H]21)C[C@@]1(CCCc2cc(Cl)ccc21)CO3 ; CO[C@H]1/C=C/COC(C)(C)C(=O)N=[S@](=O)(NC(C)=O)c2ccc3c(c2)N(C[C@@H]2CC[C@H]21)C[C@@]1(CCCc2cc(Cl)ccc21)CO3]

This compound was prepared using procedures analogous to those described for Example 1 and 2 Step 1-2 using acetyl chloride to replace isobutyryl chloride in Step 1.

P1 was assigned to Example 3 or Example 4. LC-MS calc. for C₃₄H₄₃ClN₃O₆S [M+H]⁺: m/z=656.3/658.3; Found: 656.2/657.9. HPLC: C18 column (4.6×100 mm, 5 μm); flow rate=1 mL/min; mobile phase: MeCN/H₂O (with 0.1% TFA) 50% to 95% 3 min, 95% 7 min; λ=220 nm. tR=5.448. ¹H NMR (300 MHz, CDCl₃) δ 7.66 (dd, J=8.6, 1.5 Hz, 1H), 7.50 (dd, J=8.4, 2.2 Hz, 1H), 7.41-7.34 (m, 11H), 7.19 (d, J=6.5 Hz, 1H), 7.10 (d, J=2.3 Hz, 1H), 7.07 (d, J=2.3 Hz, 1H), 5.81-5.64 (m, 2H), 4.11 (d, J=2.1 Hz, 5H), 3.81-3.54 (m, 5H), 3.42-3.33 (m, 4H), 3.31 (s, 1H), 3.29 (s, 3H), 2.80 (s, 4H), 2.26 (s, 3H), 2.19 (d, J=3.5 Hz, 1H), 2.12 (s, 1H), 1.45 (s, 3H), 1.44-1.42 (s, 3H).

P2 was obtained assigned to Example 4 or Example 3. LC-MS calc. for C₃₄H₄₃ClN₃O₆S [M+H]⁺: m/z=656.3/658.3; Found: 656.0/658.3. HPLC: C18 column (4.6×100 mm, 5 μm); flow rate=1 mL/min; mobile phase: MeCN/H₂O (with 0.1% TFA) 50% to 95% 3 min, 95% 7 min; λ=220 nm. tR=5.770. ¹H NMR (300 MHz, CDCl₃) δ 7.67 (d, J=8.5 Hz, 1H), 7.51 (dd, J=8.4, 2.3 Hz, 1H), 7.19 (dd, J=8.4, 2.3 Hz, 1H), 7.09 (d, J=2.4 Hz, 1H), 7.00 (d, J=5.1 Hz, 1H), 6.85 (d, J=2.3 Hz, 1H), 6.07-5.96 (m, 1H), 5.66 (dd, J=16.0, 4.9 Hz, 1H), 4.20-4.06 (m, 3H), 3.75 (s, 1H), 3.70 (s, 1H), 3.60 (d, J=5.3 Hz, 1H), 3.40 (d, J=2.7 Hz, 3H), 3.37-3.33 (m, 1H), 3.06 (dd, J=15.0, 10.6 Hz, 1H), 2.78 (d, J=10.8 Hz, 3H), 2.25 (s, 3H), 2.14-2.06 (m, 2H), 2.03-1.63 (m, 8H), 1.51 (s, 3H), 1.38 (s, 3H).

### Example 5 and Example 6 N-[(3R,6R,7S,8E,15R,22S)-7′-Chloro-7-methoxy-12,12-dimethyl-13,15-dioxospiro[11,20-dioxa-15-thia-1,14-diazatetracyclo[14.7.2.03,6.019,24]pentacosa-8,14,16,18,24-pentaene-22,4′-2,3-dihydro-1H-naphthalene]-15-yl]propenamide and N-[(3R,6R,7S,8E,15S,22S)-7′-chloro-7-methoxy-12,12-dimethyl-13,15-dioxospiro[11,20-dioxa-15-thia-1,14-diazatetracyclo[14.7.2.03,6.019,24]pentacosa-8,14,16,18,24-pentaene-22,4′-2,3-dihydro-1H-naphthalene]-15-yl]propenamide

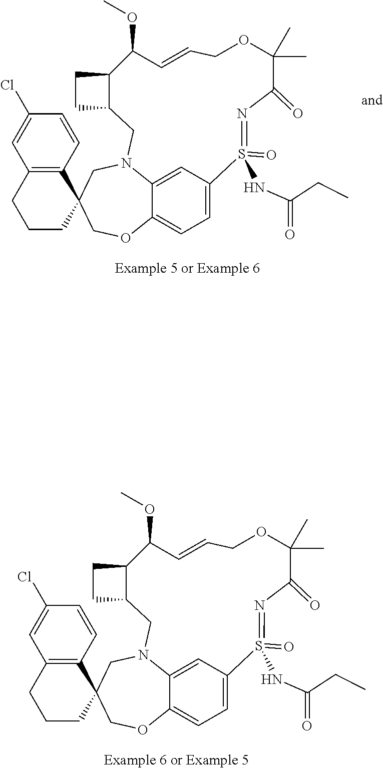 [SMILES: CCC(=O)N[S@]1(=O)=NC(=O)C(C)(C)OC/C=C/[C@H](OC)[C@@H]2CC[C@H]2CN2C[C@@]3(CCCc4cc(Cl)ccc43)COc3ccc1cc32 ; CCC(=O)N[S@@]1(=O)=NC(=O)C(C)(C)OC/C=C/[C@H](OC)[C@@H]2CC[C@H]2CN2C[C@@]3(CCCc4cc(Cl)ccc43)COc3ccc1cc32]

This compound was prepared using procedures analogous to those described for Example 1 and 2 Step 1-2 using propionyl chloride to replace isobutyryl chloride in Step 1.

P1 was assigned to Example 5 or Example 6. LC-MS calc. for C₃₅H₄₅ClN₃O₆S [M+H]⁺: m/z=670.3/672.3; Found: 670.2/672.2. HPLC: C18 column (4.6×100 mm, 5 μm); flow rate=1 mL/min; mobile phase: MeCN/H₂O (with 0.1% TFA) 50% to 95% 3 min, 95% 7 min; λ=220 nm. tR=5.856. ¹H NMR (300 MHz, CDCl₃) δ 7.66 (dd, J=8.5, 1.8 Hz, 1H), 7.51 (dd, J=8.4, 2.3 Hz, 1H), 7.19 (dd, J=8.4, 2.3 Hz, 1H), 7.10 (d, J=2.5 Hz, 1H), 7.07-6.93 (m, 2H), 5.70 (tq, J=15.8, 5.7, 5.3 Hz, 2H), 4.11 (d, J=2.4 Hz, 3H), 3.83-3.54 (m, 3H), 3.41-3.31 (m, 3H), 3.28 (s, 2H), 2.78 (d, J=10.8 Hz, 3H), 2.56-2.38 (m, 3H), 2.04-1.87 (m, 4H), 1.52-1.34 (m, 8H), 1.32-1.10 (m, 6H).

P2 was assigned to Example 6 or Example 5. LC-MS calc. for C₃₅H₄₅ClN₃O₆S [M+H]⁺: m/z=670.3/672.3; Found: 670.1/672.6. HPLC: C18 column (4.6×100 mm, 5 μm); flow rate=1 mL/min; mobile phase: MeCN/H₂O (with 0.1% TFA) 50% to 95% 3 min, 95% 7 min; λ=220 nm. tR=6.181. ¹H NMR (300 MHz, CDCl₃) δ 7.67 (d, J=8.5 Hz, 1H), 7.52 (dd, J=8.4, 2.2 Hz, 1H), 7.19 (dd, J=8.5, 2.3 Hz, 1H), 7.09 (d, J=2.4 Hz, 1H), 6.99 (dd, J=8.5, 0.7 Hz, 1H), 6.84 (d, J=2.4 Hz, 1H), 6.01 (dd, J=15.8, 6.2 Hz, 1H), 5.65 (dd, J=15.7, 4.9 Hz, 1H), 4.20-4.05 (m, 3H), 3.73 (d, J=13.8 Hz, 2H), 3.60 (d, J=5.3 Hz, 1H), 3.44-3.34 (m, 5H), 3.06 (dd, J=15.0, 10.5 Hz, 1H), 2.78 (d, J=10.4 Hz, 3H), 2.55-2.43 (m, 2H), 2.10-1.95 (m, 4H), 1.49 (d, J=8.5 Hz, 4H), 1.38 (s, 4H), 1.28-1.16 (m, 6H).

### Example 7 and Example 8 N-[(3R,6R,7S,8E,15R,22S)-7′-Chloro-7-methoxy-12,12-dimethyl-13,15-dioxospiro[11,20-dioxa-15-thia-1,14-diazatetracyclo[14.7.2.03,6.019,24]pentacosa-8,14,16,18,24-pentaene-22,4′-2,3-dihydro-1H-naphthalene]-15-yl]-2-cyclopropylacetamide and N-[(3R,6R,7S,8E,15S,22S)-7′-chloro-7-methoxy-12,12-dimethyl-13,15-dioxospiro[11,20-dioxa-15-thia-1,14-diazatetracyclo[14.7.2.03,6.019,24]pentacosa-8,14,16,18,24-pentaene-22,4′-2,3-dihydro-1H-naphthalene]-15-yl]-2-cyclopropylacetamide

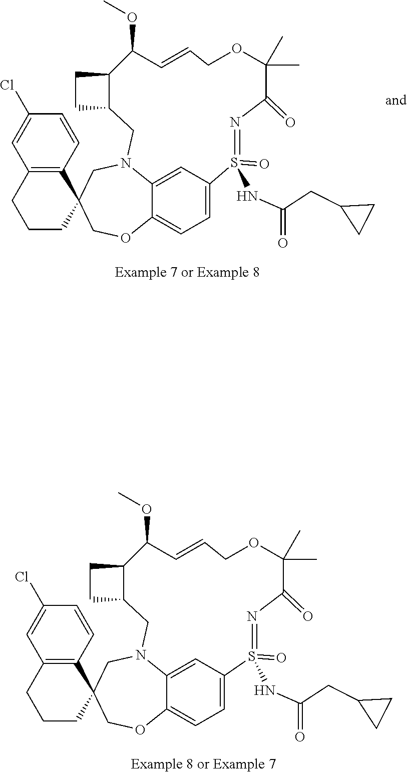 [SMILES: CO[C@H]1/C=C/COC(C)(C)C(=O)N=[S@@](=O)(NC(=O)CC2CC2)c2ccc3c(c2)N(C[C@@H]2CC[C@H]21)C[C@@]1(CCCc2cc(Cl)ccc21)CO3 ; CO[C@H]1/C=C/COC(C)(C)C(=O)N=[S@](=O)(NC(=O)CC2CC2)c2ccc3c(c2)N(C[C@@H]2CC[C@H]21)C[C@@]1(CCCc2cc(Cl)ccc21)CO3]

This compound was prepared using procedures analogous to those described for Example 1 and 2 Step 1-2 using 2-cyclopropylacetyl chloride to replace isobutyryl chloride in Step 1.

P1 was assigned to Example 7 or Example 8. LC-MS calc. for C₃₇H₄₇ClN₃O₆S [M+H]⁺: m/z=696.3/671.3; Found: 696.3/671.3. HPLC: C18 column (4.6×100 mm, 5 μm); flow rate=1 mL/min; mobile phase: MeCN/H₂O (with 0.1% TFA) 50% to 95% 3 min, 95% 7 min; λ=220 nm. tR=6.149. ¹H NMR (300 MHz, CDCl₃) δ 7.66 (d, J=8.5 Hz, 1H), 7.51 (dd, J=8.4, 2.2 Hz, 1H), 7.19 (dd, J=8.5, 2.3 Hz, 1H), 7.10 (d, J=2.3 Hz, 1H), 7.06 (d, J=2.3 Hz, 1H), 7.01 (d, J=8.4 Hz, 1H), 5.80-5.60 (m, 2H), 4.11 (s, 4H), 3.92 (dd, J=14.0, 5.3 Hz, 1H), 3.72 (d, J=14.6 Hz, 1H), 3.59 (dd, J=6.7, 3.5 Hz, 1H), 3.45 (dd, J=14.9, 5.2 Hz, 1H), 3.36 (d, J=3.7 Hz, 1H), 3.33-3.30 (m, 1H), 3.26 (s, 3H), 2.78 (d, J=10.1 Hz, 3H), 2.38 (d, J=7.1 Hz, 2H), 2.07-1.75 (m, 8H), 1.46 (s, 3H), 1.43 (s, 3H), 0.93-0.85 (m, 1H), 0.60 (dt, J=9.1, 3.0 Hz, 2H), 0.25 (d, J=4.9 Hz, 2H).

P2 was assigned to Example 8 or Example 7. LC-MS calc. for C₃₇H₄₇ClN₃O₆S [M+H]⁺: m/z=696.3/671.3; Found: 696.3/671.3. HPLC: C18 column (4.6×100 mm, 5 μm); flow rate=1 mL/min; mobile phase: MeCN/H₂O (with 0.1% TFA) 50% to 95% 3 min, 95% 7 min; λ=220 nm. tR=6.332. ¹H NMR (300 MHz, CDCl₃) δ 7.68 (d, J=8.4 Hz, 1H), 7.53 (dd, J=8.4, 2.2 Hz, 1H), 7.19 (dd, J=8.5, 2.3 Hz, 1H), 7.09 (d, J=2.5 Hz, 1H), 7.00 (d, J=8.4 Hz, 1H), 6.86 (d, J=2.3 Hz, 1H), 6.07-5.96 (m, 1H), 5.71-5.59 (m, 1H), 4.24-4.02 (m, 4H), 3.81-3.68 (m, 2H), 3.60 (d, J=4.3 Hz, 1H), 3.44-3.27 (m, 6H), 3.07 (dd, J=15.0, 10.4 Hz, 1H), 2.84-2.70 (m, 3H), 2.41-2.29 (m, 2H), 2.08-1.77 (m, 7H), 1.51 (s, 3H), 1.38 (s, 3H), 0.92-0.87 (m, 1H), 0.59-0.52 (m, 2H), 0.21 (dd, J=6.8, 5.1 Hz, 2H).

### Example 9 and Example 10 N-[(3R,6R,7S,8E,15R,22S)-7′-Chloro-7-methoxy-12,12-dimethyl-13,15-dioxospiro[11,20-dioxa-15-thia-1,14-diazatetracyclo[14.7.2.03,6.019,24]pentacosa-8,14,16,18,24-pentaene-22,4′-2,3-dihydro-1H-naphthalene]-15-yl]cyclobutanecarboxamide and N-[(3R,6R,7S,8E,15S,22S)-7′-chloro-7-methoxy-12,12-dimethyl-13,15-dioxospiro[11,20-dioxa-15-thia-1,14-diazatetracyclo[14.7.2.03,6.019,24]pentacosa-8,14,16,18,24-pentaene-22,4′-2,3-dihydro-1H-naphthalene]-15-yl]cyclobutanecarboxamide

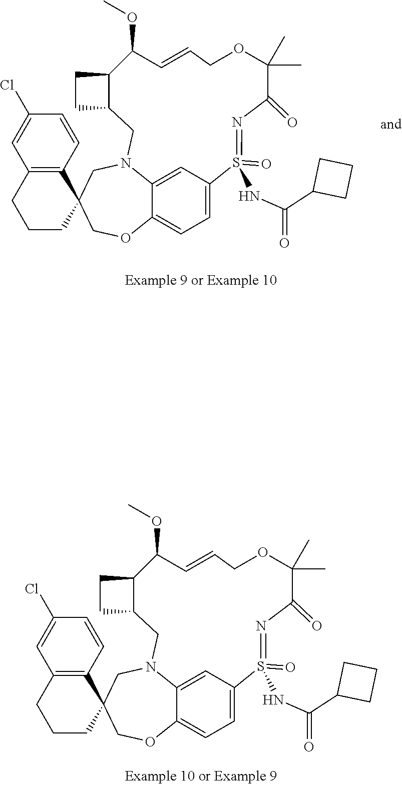 [SMILES: CO[C@H]1/C=C/COC(C)(C)C(=O)N=[S@@](=O)(NC(=O)C2CCC2)c2ccc3c(c2)N(C[C@@H]2CC[C@H]21)C[C@@]1(CCCc2cc(Cl)ccc21)CO3 ; CO[C@H]1/C=C/COC(C)(C)C(=O)N=[S@](=O)(NC(=O)C2CCC2)c2ccc3c(c2)N(C[C@@H]2CC[C@H]21)C[C@@]1(CCCc2cc(Cl)ccc21)CO3]

This compound was prepared using procedures analogous to those described for Example 1 and 2 Step 1-2 using cyclobutanecarbonyl chloride to replace isobutyryl chloride in Step 1.

P1 as a light yellow solid was assigned to Example 9 or Example 10. LC-MS calc. for C₃₇H₄₇ClN₃O₆S [M+H]⁺: m/z=696.3/698.3; Found: 696.0/698.4. HPLC: C18 column (4.6×100 mm, 5 am); flow rate=1 mL/min; mobile phase: 50% MeCN/H₂O (with 0.1% TFA) to 95% 4 min, 95% 6 min; λ=220 nm. tR=6.33 min. ¹H NMR (300 MHz, CDCl₃) δ 7.66 (d, J=8.6 Hz, 1H), 7.50 (dd, J=8.3, 2.2 Hz, 1H), 7.19 (dd, J=8.7, 2.3 Hz, 1H), 7.10 (d, J=2.4 Hz, 1H), 7.05-6.98 (m, 2H), 5.83-5.70 (m, 1H), 5.63 (dd, J=15.8, 7.2 Hz, 1H), 5.37 (br, 1H), 4.14-4.09 (m, 3H), 3.86 (dd, J=13.9, 5.6 Hz, 1H), 3.72 (d, J=14.4 Hz, 1H), 3.59-3.53 (m, 1H), 3.44 (d, J=9.5 Hz, 1H), 3.31 (t, J=4.4 Hz, 1H), 3.26 (s, 3H), 2.81-2.78 (m, 2H), 2.51-2.33 (m, 4H), 2.27 (t, J=7.7 Hz, 2H), 2.04-1.93 (m, 5H), 1.85-1.78 (m, 2H), 1.46 (s, 3H), 1.42 (s, 3H), 1.34-1.28 (m, 5H).

P2 as a light yellow solid was assigned to Example 10 or Example 9. LC-MS calc. for C₃₇H₄₇ClN₃O₆S [M+H]⁺: m/z=696.3/698.3; Found: 696.1/698.1. HPLC: C18 column (4.6×100 mm, 5 μm); flow rate=1 mL/min; mobile phase: 50% MeCN/H₂O (with 0.1% TFA) to 95% 4 min, 95% 6 min; λ=220 nm. tR=6.70 min. ¹H NMR (300 MHz, CDCl₃) δ 7.68 (d, J=8.5 Hz, 1H), 7.52 (dd, J=8.4, 2.2 Hz, 1H), 7.19 (dd, J=8.7, 2.4 Hz, 1H), 7.09 (d, J=2.3 Hz, 1H), 6.99 (d, J=8.2 Hz, 1H), 6.82 (d, J=2.3 Hz, 1H), 6.04-5.99 (m, 1H), 5.63 (dd, J=15.7, 5.0 Hz, 1H), 5.34 (br, 1H), 4.21-4.05 (m, 3H), 3.70 (d, J=11.0 Hz, 2H), 3.60 (s, 1H), 3.38 (s, 3H), 3.34-3.25 (m, 3H), 3.06 (dd, J=15.1, 10.5 Hz, 2H), 2.79 (d, J=4.6 Hz, 2H), 2.44-2.39 (m, 2H), 2.29-2.19 (m, 2H), 2.11-1.85 (m, 7H), 1.51 (s, 3H), 1.38 (s, 3H), 1.33-1.28 (m, 5H).

### Example 11 N-[(3R,6R,7S,8E,22S)-7′-Chloro-7-methoxy-12,12-dimethyl-13,15-dioxospiro[11,20-dioxa-15-thia-1,14-diazatetracyclo[14.7.2.03,6.019,24]pentacosa-8,14,16,18,24-pentaene-22,4′-2,3-dihydro-1H-naphthalene]-15-yl]oxane-4-carboxamide

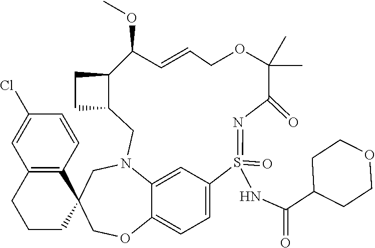 [SMILES: CO[C@H]1/C=C/COC(C)(C)C(=O)N=S(=O)(NC(=O)C2CCOCC2)c2ccc3c(c2)N(C[C@@H]2CC[C@H]21)C[C@@]1(CCCc2cc(Cl)ccc21)CO3]

This compound was prepared as a white solid using procedures analogous to those described for Example 1 and 2 Step 1-2 using oxane-4-carbonyl chloride to replace isobutyryl chloride in Step 1. A mixture of two diastereomers was obtained as a light-yellow solid. LC-MS calc. for C₃₈H₄₉ClN₃O₇S [M+H]⁺: m/z=726.3/728.3; Found: 726.2/728.3. HPLC: C18 column (4.6×100 mm, 5 μm); flow rate=1 mL/min; mobile phase: 50% MeCN/H₂O (with 0.1% TFA) to 95% 4 min, 95% 6 min; λ=220 nm. tR=5.77 min. ¹H NMR (300 MHz, CDCl₃) (1:1 diastereomers) δ 7.67 (dd, J=8.6, 4.5 Hz, 1H), 7.54-7.50 (m, 1H), 7.22-7.18 (m, 1H), 7.12-7.07 (m, 1H), 7.01 (dd, J=8.4, 6.1 Hz, 2H), 6.80 (d, J=2.3 Hz, 1H), 5.99 (dd, J=16.4, 6.7 Hz, 0.5H), 5.84-5.61 (m, 1.5H), 4.24-3.95 (m, 6H), 3.74 (t, J=12.4 Hz, 2H), 3.58 (d, J=12.1 Hz, 1H), 3.53-3.42 (m, 3H), 3.38 (s, 1.5H), 3.27 (s, 1.5H), 3.08-3.05 (m, 1H), 2.78 (d, J=10.3 Hz, 2H), 2.68-2.59 (m, 1H), 2.07-1.72 (m, 12H), 1.64 (d, J=9.4 Hz, 2H), 1.50 (s, 1.5H), 1.46 (s, 1.5H), 1.41 (s, 1.5H), 1.38 (s, 1.5H).

### Example 12 N-[(3R,6R,7S,8E,22S)-7′-Chloro-7-methoxy-12,12-dimethyl-13,15-dioxospiro[11,20-dioxa-15-thia-1,14-diazatetracyclo[14.7.2.03,6.019,24]pentacosa-8,14,16,18,24-pentaene-22,4′-2,3-dihydro-1H-naphthalene]-15-yl]benzamide

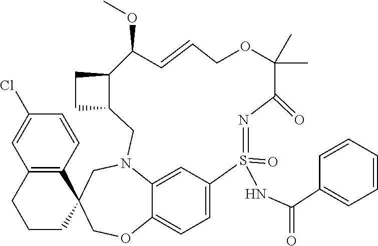 [SMILES: CO[C@H]1/C=C/COC(C)(C)C(=O)N=S(=O)(NC(=O)c2ccccc2)c2ccc3c(c2)N(C[C@@H]2CC[C@H]21)C[C@@]1(CCCc2cc(Cl)ccc21)CO3]

This compound was prepared as a white solid using procedures analogous to those described for Example 1 and 2 Step 1-2 using benzoyl chloride to replace isobutyryl chloride in Step 1. A mixture of two diastereomers was obtained as a light-yellow solid. LC-MS calc. for C₃₉H₄₅ClN₃O₆S [M+H]⁺: m/z=718.3/720.3; Found: 718.0/720.3. HPLC: C18 column (4.6×100 mm, 5 μm); flow rate=1 mL/min; mobile phase: 50% MeCN/H₂O (with 0.1% TFA) to 95% 4 min, 95% 6 min; λ=220 nm. tR=9.36 min and 9.62 min. ¹H NMR (300 MHz, CDCl₃) (1:1 diastereomers) δ 8.27-8.17 (m, 2H), 7.69-7.67 (m, 1H), 7.60-7.39 (m, 4H), 7.21-7.13 (m, 1H), 7.10-6.94 (m, 3H), 6.02 (dd, J=17.0, 5.4 Hz, 0.5H), 5.76-5.64 (m, 1.5H), 4.27-4.05 (m, 3H), 3.87-3.67 (m, 2H), 3.54-3.50 (m, 1H), 3.42-3.29 (m, 2H), 3.22 (s, 3H), 3.09-3.03 (m, 1H), 2.82-2.76 (m, 2H), 2.56-2.46 (m, 1H), 2.37-2.32 (m, 1H), 2.12-1.79 (m, 6H), 1.73-1.67 (m, 2H), 1.55 (s, 1.5H), 1.51 (s, 1.5H), 1.49-1.47 (m, 1H), 1.44 (s, 1.5H), 1.41 (s, 1.5H).

### Example 13 and Example 14 4-Bromo-N-[(3R,6R,7S,8E,15R,22S)-7′-chloro-7-methoxy-12,12-dimethyl-13,15-dioxospiro[11,20-dioxa-15-thia-1,14-diazatetracyclo[14.7.2.03,6.019,24]pentacosa-8,14,16,18,24-pentaene-22,4′-2,3-dihydro-1H-naphthalene]-15-yl]benzamide and 4-Bromo-N-[(3R,6R,7S,8E,15S,22S)-7′-chloro-7-methoxy-12,12-dimethyl-13,15-dioxospiro[11,20-dioxa-15-thia-1,14-diazatetracyclo[14.7.2.03,6.019,24]pentacosa-8,14,16,18,24-pentaene-22,4′-2,3-dihydro-1H-naphthalene]-15-yl]benzamide

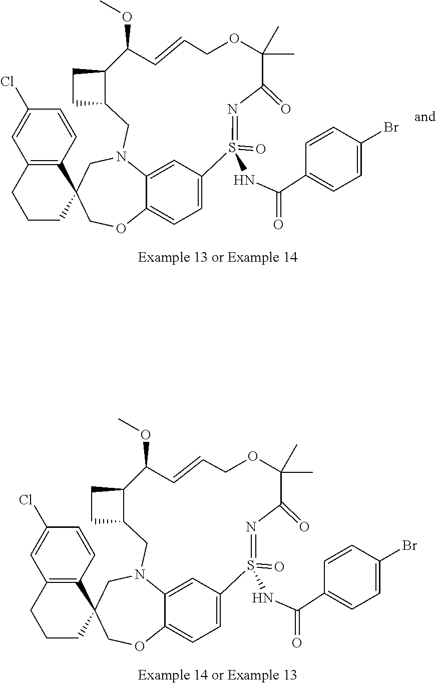 [SMILES: CO[C@H]1/C=C/COC(C)(C)C(=O)N=[S@@](=O)(NC(=O)c2ccc(Br)cc2)c2ccc3c(c2)N(C[C@@H]2CC[C@H]21)C[C@@]1(CCCc2cc(Cl)ccc21)CO3 ; CO[C@H]1/C=C/COC(C)(C)C(=O)N=[S@](=O)(NC(=O)c2ccc(Br)cc2)c2ccc3c(c2)N(C[C@@H]2CC[C@H]21)C[C@@]1(CCCc2cc(Cl)ccc21)CO3]

This compound was prepared as a white solid using procedures analogous to those described for Example 1 and 2 Step 1-2 using 4-bromobenzoyl chloride to replace isobutyryl chloride in Step 1.

P1 was assigned to Example 13 or Example 14. LC-MS calc. for C₃₉H₄₄BrClN₃O₆S [M+H]⁺: m/z=796.2/798.2; Found: 796.2/797.8. HPLC: C18 column (4.6×100 mm, 5 μm); flow rate=1 mL/min; mobile phase: MeCN/H₂O (with 0.1% TFA) 50% to 95% 3 min, 95% 7 min; λ=220 nm. tR=8.296. ¹H NMR (300 MHz, CDCl₃) δ 8.09 (d, J=8.5 Hz, 2H), 7.66 (d, J=8.5 Hz, 1H), 7.63-7.55 (m, 3H), 7.19 (dd, J=8.5, 2.3 Hz, 1H), 7.10 (dd, J=4.1, 2.3 Hz, 2H), 7.04 (d, J=8.4 Hz, 1H), 5.82 (dt, J=16.0, 5.1 Hz, 1H), 5.68 (dd, J=15.7, 6.7 Hz, 1H), 4.21 (dd, J=14.0, 4.1 Hz, 1H), 4.11 (s, 2H), 3.88 (dd, J=13.9, 6.0 Hz, 1H), 3.73 (d, J=14.6 Hz, 1H), 3.54 (dd, J=6.7, 3.6 Hz, 1H), 3.42-3.30 (m, 2H), 3.24 (s, 4H), 2.78 (q, J=5.4 Hz, 2H), 2.52 (p, J=8.2 Hz, 1H), 2.36 (qd, J=9.7, 9.2, 3.8 Hz, 1H), 2.11-1.55 (m, 8H), 1.49 (s, 3H), 1.44 (s, 3H).

P2 as a light yellow solid was assigned to Example 14 or Example 13. LC-MS calc. for C₃₉H₄₄BrClN₃O₆S [M+H]⁺: m/z=796.2/798.2; Found: 796.0/798.2. HPLC: C18 column (4.6×100 mm, 5 μm); flow rate=1 mL/min; mobile phase: MeCN/H₂O (with 0.1% TFA) 50% to 95% 3 min, 95% 7 min; λ=220 nm. tR=9.011. ¹H NMR (300 MHz, CDCl₃) δ 8.08-8.02 (m, 2H), 7.67 (d, J=8.5 Hz, 1H), 7.60-7.53 (m, 3H), 7.18 (dd, J=8.5, 2.4 Hz, 1H), 7.08 (d, J=2.3 Hz, 1H), 7.03 (d, J=8.4 Hz, 1H), 6.94 (d, J=2.3 Hz, 1H), 5.97 (dd, J=6.6, 2.8 Hz, 1H), 5.75 (dd, J=15.8, 5.3 Hz, 1H), 4.29-4.04 (m, 3H), 3.74 (dd, J=14.2, 9.8 Hz, 2H), 3.56 (t, J=4.0 Hz, 1H), 3.44-3.29 (m, 2H), 3.23 (s, 3H), 3.07 (dd, J=15.0, 10.3 Hz, 1H), 2.89-2.53 (m, 4H), 2.18-1.78 (m, 7H), 1.72 (q, J=8.2, 7.7 Hz, 1H), 1.55 (s, 3H), 1.41 (s, 3H).

### Example 15 N-[(3R,6R,7S,8E,22S)-7′-Chloro-7-methoxy-12,12-dimethyl-13,15-dioxospiro[11,20-dioxa-15-thia-1,14-diazatetracyclo[14.7.2.03,6.019,24]pentacosa-8,14,16,18,24-pentaene-22,4′-2,3-dihydro-1H-naphthalene]-15-yl]-1-methylpyrazole-4-carboxamide

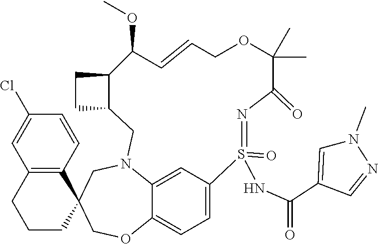 [SMILES: CO[C@H]1/C=C/COC(C)(C)C(=O)N=S(=O)(NC(=O)c2cnn(C)c2)c2ccc3c(c2)N(C[C@@H]2CC[C@H]21)C[C@@]1(CCCc2cc(Cl)ccc21)CO3]

This compound was prepared as a white solid using procedures analogous to those described for Example 1 and 2 Step 1-2 using 1-methylpyrazole-4-carbonyl chloride to replace isobutyryl chloride in Step 1. A mixture of two diastereomers was obtained as a light-yellow solid. LC-MS calc. for C₃₇H₄₅ClN₅O₆S [M+H]⁺: m/z=722.3/724.3; Found: 722.0/724.4. HPLC: C18 column (4.6×100 mm, 5 μm); flow rate=1 mL/min; mobile phase: 50% MeCN/H₂O (with 0.1% TFA) to 95% 4 min, 95% 6 min; λ=220 nm. tR=5.44 min. ¹H NMR (300 MHz, CDCl₃) (1:1 diastereomers) δ 8.04-8.00 (m, 1H), 7.98-7.92 (m, 1H), 7.68-7.62 (m, 1H), 7.58-7.51 (m, 1H), 7.23-7.16 (m, 1H), 7.13-7.08 (m, 1H), 7.03 (d, J=4.9 Hz, 0.5H), 7.00 (d, J=4.8 Hz, 0.5H), 6.94-6.88 (m, 1H), 6.06-5.96 (m, 0.5H), 5.79-5.64 (m, 1.5H), 5.40-5.31 (m, 1H), 4.25-4.08 (m, 3H), 3.96 (s, 1.5H), 3.94 (s, 1.5H), 3.74-3.70 (m, 2H), 3.59-3.53 (m, 1H), 3.42-3.37 (m, 1H), 3.29 (s, 1.5H), 3.24 (s, 1.5H), 3.12-3.02 (m, 1H), 2.79-2.72 (m, 4H), 2.53 (d, J=7.7 Hz, 0.5H), 2.41 (d, J=6.9 Hz, 0.5H), 2.31-2.26 (m, 1H), 2.11-1.90 (m, 5H), 1.78-1.73 (m, 2H), 1.53 (s, 15H), 1.48 (s, 1.5H), 1.44 (s, 1.5H), 1.39 (s, 15H).

### Example 16 N-[(3R,6R,7S,8E,22S)-7′-Chloro-7-methoxy-12,12-dimethyl-13,15-dioxospiro[11,20-dioxa-15-thia-1,14-diazatetracyclo[14.7.2.03,6.019,24]pentacosa-8,14,16,18,24-pentaene-22,4′-2,3-dihydro-1H-naphthalene]-15-yl]-3-methoxy-1-methylpyrazole-4-carboxamide

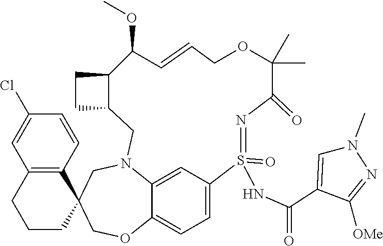 [SMILES: COc1nn(C)cc1C(=O)NS1(=O)=NC(=O)C(C)(C)OC/C=C/[C@H](OC)[C@@H]2CC[C@H]2CN2C[C@@]3(CCCc4cc(Cl)ccc43)COc3ccc1cc32]

This compound was prepared as a white solid using procedures analogous to those described for Example 1 and 2 Step 1-2 using 3-methoxy-1-methylpyrazole-4-carbonyl chloride to replace isobutyryl chloride in Step 1. A mixture of two diastereomers was obtained as a light yellow solid. LC-MS calc. for C₃₈H₄₇ClN₅O₇S [M+H]⁺: m/z=752.3/754.3; Found: 752.1/754.2. HPLC: C18 column (4.6×100 mm, 5 μm); flow rate=1 mL/min; mobile phase: 50% MeCN/H₂O (with 0.1% TFA) to 95% 4 min, 95% 6 min; λ=220 nm. tR=5.58 min. ¹H NMR (300 MHz, CDCl₃) δ 7.82-7.75 (m, 1H), 7.68-7.61 (m, 1H), 7.54 (dd, J=8.4, 2.2 Hz, 0.5H), 7.46 (dd, J=8.4, 2.2 Hz, 0.5H), 7.22-7.16 (m, 1H), 7.12-7.05 (m, 1H), 7.04-6.95 (m, 2H), 6.93 (br, 1H), 6.05-5.94 (m, 0.5H), 5.70-5.60 (m, 1.5H), 4.13-4.08 (m, 2H), 4.05 (s, 1.5H), 4.01 (s, 1.5H), 3.78 (s, 1.5H), 3.76 (s, 1.5H), 3.71-3.64 (m, 1H), 3.5-3.53 (m, 1H), 3.39-3.32 (m, 2H), 3.28 (s, 1.5H), 3.24 (s, 1.5H), 3.15-2.98 (m, 1H), 2.81-2.74 (m, 3H), 2.61 (d, J=8.1 Hz, 0.5H), 2.45 (dd, J=9.1, 3.6 Hz, 0.5H), 2.19-2.14 (m, 1H), 2.07-1.67 (m, 7H), 1.66-1.53 (m, 2H), 1.50 (s, 1.5H), 1.46-1.44 (m, 3H), 1.39 (s, 1.5H).

### Example 17 N-[(3R,6R,7S,8E,22S)-7′-Chloro-7-methoxy-12,12-dimethyl-13,15-dioxospiro[11,20-dioxa-15-thia-1,14-diazatetracyclo[14.7.2.03,6.019,24]pentacosa-8,14,16,18,24-pentaene-22,4′-2,3-dihydro-1H-naphthalene]-15-yl]-1-methylpyrazole-4-carboxamide

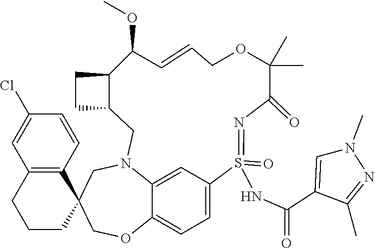 [SMILES: CO[C@H]1/C=C/COC(C)(C)C(=O)N=S(=O)(NC(=O)c2cn(C)nc2C)c2ccc3c(c2)N(C[C@@H]2CC[C@H]21)C[C@@]1(CCCc2cc(Cl)ccc21)CO3]

### Step 1: 1,3-dimethylpyrazole-4-carbonyl chloride

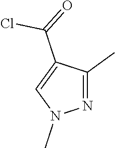

To a solution of 1,3-dimethylpyrazole-4-carboxylic acid (200.0 mg, 1.43 mmol) in DCM was added thionyl chloride (0.21 mL, 2.85 mmol) followed by one drop of DMF. The resulting mixture was stirred at r.t. overnight. The solution was concentrated under reduced pressure and further dried over high vacuum for 1 h to remove all the excess thionyl chloride to afford 1,3-dimethylpyrazole-4-carbonyl chloride (210 mg, 92.8% yield) as a yellow solid. The crude product was used in next step without further purification.

### Step 2: N-[(3R,6R,7S,8E,22S)-7′-chloro-7-methoxy-12,12-dimethyl-13,15-dioxospiro[11,20-dioxa-15-thia-1,14-diazatetracyclo[14.7.2.03,6.019,24]pentacosa-8,14,16,18,24-pentaene-22,4′-2,3-dihydro-1H-naphthalene]-15-yl]-1,3-dimethylpyrazole-4-carboxamide

This compound was prepared as a white solid using procedures analogous to those described for Example 1 and 2 Step 1-2 using 1,3-dimethylpyrazole-4-carbonyl chloride to replace isobutyryl chloride in Step 1. A mixture of two diastereomers was obtained as a light-yellow solid. LC-MS calc. for C₃₈H₄₇ClN₅O₆S [M+H]⁺: m/z=736.3/738.3; Found: 736.0/738.1. HPLC: C18 column (4.6×100 mm, 5 μm); flow rate=1 mL/min; mobile phase: 50% MeCN/H₂O (with 0.1% TFA) to 95% 4 min, 95% 6 min; λ=220 nm. tR=5.68 min. ¹H NMR (300 MHz, CDCl₃) δ 7.94-7.91 (m, 1H), 7.69-7.64 (m, 1H), 7.58-7.51 (m, 1H), 7.23-7.15 (m, 2H), 7.13-7.06 (m, 1H), 7.05-6.93 (m, 2H), 6.00 (dd, J=16.2, 4.7 Hz, 0.5H), 5.87-5.64 (m, 1.5H), 4.26-4.10 (m, 3H), 3.92 (s, 1.5H), 3.90 (s, 1.5H), 3.76-3.72 (m, 1H), 3.63-3.58 (m, 1H), 3.44-3.31 (m, 2H), 3.27 (s, 1.5H), 3.24 (s, 1.5H), 3.14-3.10 (m, 1H), 2.81-2.75 (m, 2H), 2.56 (s, 1.5H), 2.55 (s, 1.5H), 2.46-2.35 (m, 1H), 2.13-2.10 (m, 1H), 2.06-1.86 (m, 4H), 1.79-1.73 (m, 2H), 1.63-1.58 (m, 1H), 1.53 (s, 1.5H), 1.48 (s, 1.5H), 1.43 (s, 1.5H), 1.40 (s, 1.5H), 1.33-1.27 (m, 2H).

### Example 18 N-[(3R,6R,7S,8E,22S)-7′-Chloro-7-methoxy-12,12-dimethyl-13,15-dioxospiro[11,20-dioxa-15-thia-1,14-diazatetracyclo[14.7.2.03,6.019,24]pentacosa-8,14,16,18,24-pentaene-22,4′-2,3-dihydro-1H-naphthalene]-15-yl]-1,5-dimethylpyrazole-4-carboxamide

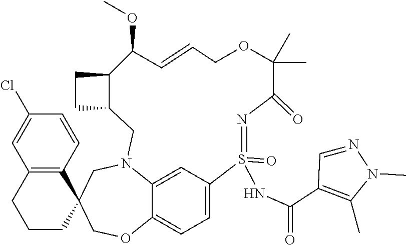 [SMILES: CO[C@H]1/C=C/COC(C)(C)C(=O)N=S(=O)(NC(=O)c2cnn(C)c2C)c2ccc3c(c2)N(C[C@@H]2CC[C@H]21)C[C@@]1(CCCc2cc(Cl)ccc21)CO3]

This compound was prepared as a white solid using procedures analogous to those described for Example 17 Step 1-2 using 1,5-dimethylpyrazole-4-carboxylic acid to replace 1,3-dimethylpyrazole-4-carboxylic acid in Step 1. A mixture of two diastereomers was obtained. LC-MS calc. for C₃₈H₄₇ClN₅O₆S [M+H]⁺: m/z=736.3/738.3; Found: 736.2/738.5. HPLC: C18 column (4.6×100 mm, 5 μm); flow rate=1 mL/min; mobile phase: MeCN/H₂O (with 0.1% TFA) 50% to 95% 3 min, 95% 7 min; =220 nm. tR=5.700. ¹H NMR (300 MHz, CDCl₃) δ 9.65 (s, 1H), 8.10 (d, J=5.3 Hz, 1H), 7.66 (dd, J=8.5, 3.5 Hz, 1H), 7.56 (dt, J=8.4, 2.1 Hz, 1H), 7.18 (dt, J=8.5, 2.7 Hz, 1H), 7.12-7.08 (m, 1H), 7.02 (dd, J=8.4, 4.8 Hz, 1H), 6.98-6.92 (m, 1H), 6.02-5.67 (m, 2H), 4.25-4.05 (m, 4H), 3.88 (d, J=4.1 Hz, 3H), 3.75 (dd, J=14.4, 8.4 Hz, 2H), 3.59 (q, J=4.8, 3.8 Hz, 1H), 3.34 (dt, J=17.4, 4.5 Hz, 2H), 3.27 (d, J=4.1 Hz, 3H), 2.84-2.69 (m, 3H), 2.62 (d, J=9.6 Hz, 4H), 2.05-1.85 (m, 5H), 1.54-1.38 (m, 9H).

### Example 19 N-[(3R,6R,7S,8E,22S)-7′-Chloro-7-methoxy-12,12-dimethyl-13,15-dioxospiro[11,20-dioxa-15-thia-1,14-diazatetracyclo[14.7.2.03,6.019,24]pentacosa-8,14,16,18,24-pentaene-22,4′-2,3-dihydro-1H-naphthalene]-15-yl]-1,5-dimethylpyrazole-3-carboxamide

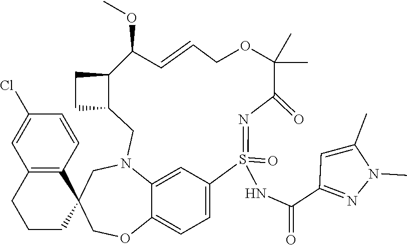 [SMILES: CO[C@H]1/C=C/COC(C)(C)C(=O)N=S(=O)(NC(=O)c2cc(C)n(C)n2)c2ccc3c(c2)N(C[C@@H]2CC[C@H]21)C[C@@]1(CCCc2cc(Cl)ccc21)CO3]

This compound was prepared as a white solid using procedures analogous to those described for Example 17 Step 1-2 using 1,5-dimethylpyrazole-3-carboxylic acid to replace 1,3-dimethylpyrazole-4-carboxylic acid in Step 1. A mixture of two diastereomers was obtained. LC-MS calc. for C₃₈H₄₇ClN₅O₆S [M+H]⁺: m/z=736.3/738.3; Found: 736.2/738.2. HPLC: C18 column (4.6×100 mm, 5 μm); flow rate=1 mL/min; mobile phase: MeCN/H₂O (with 0.1% TFA) 50% to 95% 3 min, 95% 7 min; λ=220 nm. tR=5.576. ¹H NMR (300 MHz, CDCl₃) δ 7.66 (dd, J=8.6, 4.5 Hz, 1H), 7.59-7.49 (m, 1H), 7.18 (dd, J=8.8, 2.3 Hz, 1H), 7.09 (dd, J=3.9, 2.3 Hz, 1H), 7.05-6.92 (m, 2H), 6.63 (d, J=8.3 Hz, 1H), 6.03 (dd, J=15.6, 4.2 Hz, 1H), 5.76-5.62 (m, 1H), 4.24-3.99 (m, 4H), 3.87 (d, J=8.7 Hz, 3H), 3.81-3.65 (m, 2H), 3.61-3.54 (m, 1H), 3.40-3.34 (m, 1H), 3.31 (d, J=2.9 Hz, 2H), 3.24 (s, 2H), 2.78 (s, 3H), 2.31 (d, J=5.8 Hz, 4H), 2.05-1.85 (m, 5H), 1.46 (dd, J=22.5, 14.3 Hz, 9H).

### Example 20 1-[(3R,6R,7S,8E,22S)-7′-Chloro-7-methoxy-12,12-dimethyl-13,15-dioxospiro[11,20-dioxa-15-thia-1,14-diazatetracyclo[14.7.2.03,6.019,24]pentacosa-8,14,16,18,24-pentaene-22,4′-2,3-dihydro-1H-naphthalene]-15-yl]-3-ethylurea

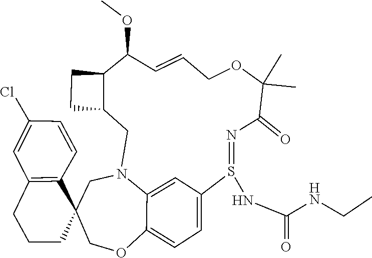 [SMILES: CCNC(=O)N/S1=N\C(=O)C(C)(C)OC/C=C/[C@H](OC)[C@@H]2CC[C@H]2CN2C[C@@]3(CCCc4cc(Cl)ccc43)COc3ccc1cc32]

### Step 1: N-[[(4S)-7-chloro-5′-[[(1R,2R)-2-[(1S)-1-methoxyprop-2-enyl]cyclobutyl]methyl]spiro[2,3-dihydro-1H-naphthalene-4,3′-2,4-dihydro-1,5-benzoxazepine]-7′-yl]-(ethylcarbamoylamino)-oxo-sulfanylidene]-2-methyl-2-prop-2-enoxypropanamide

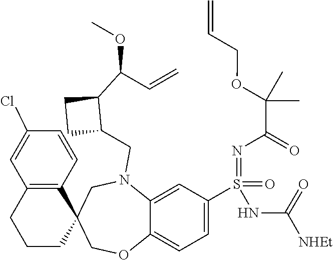

To a solution of N-[amino-oxo-[(4S)-7-chloro-5′-[[(1R,2R)-2-[(1S)-1-methoxyprop-2-enyl]cyclobutyl]methyl]spiro[2,3-dihydro-1H-naphthalene-4,3′-2,4-dihydro-1,5-benzoxazepine]-7′-yl]-sulfanylidene]-2-methyl-2-prop-2-enoxypropanamide (50.0 mg, 0.08 mmol, Intermediate 1) in DCM (4 mL) was added triethylamine (0.04 mL, 0.31 mmol) followed by phenyl chloroformate (0.02 mL, 0.16 mmol). The resulting mixture was stirred at r.t. for 30 min., then ethylamine (2.0 M in THF 0.93 mL, 1.86 mmol) was added. The resulting mixture was stirred at r.t. overnight. The mixture was concentrated under reduced pressure. The residue was purified by prep-HPLC on C18 column (30×250 mm, 10 μm) with MeCN/H₂O (w/0.1% TFA, 20-100%) to afford N-[[(4S)-7-chloro-5′-[[(1R,2R)-2-[(1S)-1-methoxyprop-2-enyl]cyclobutyl]methyl]spiro[2,3-dihydro-1H-naphthalene-4,3′-2,4-dihydro-1,5-benzoxazepine]-7′-yl]-(ethylcarbamoylamino)-oxo-sulfanylidene]-2-methyl-2-prop-2-enoxypropanamide (16 mg, 24.1% yield) as a white solid. LC-MS calc. for C₃₇H₅₀ClN₄O₆S [M+H]⁺: m/z=713.3/715.3; Found: 713.3/715.7.

### Step 2: 1-[(3R,6R,7S,8E,22S)-7′-chloro-7-methoxy-12,12-dimethyl-13,15-dioxospiro[1,20-dioxa-15-thia-1,14-diazatetracyclo[14.7.2.03,6.019,24]pentacosa-8,14,16,18,24-pentaene-22,4′-2,3-dihydro-1H-naphthalene]-15-yl]-3-ethylurea

This compound was prepared as a white solid using procedures analogous to those described for Example 1 and 2 using N-[[(4S)-7-chloro-5′-[[(1R,2R)-2-[(1S)-1-methoxyprop-2-enyl]cyclobutyl]methyl]spiro[2,3-dihydro-1H-naphthalene-4,3′-2,4-dihydro-1,5-benzoxazepine]-7′-yl]-(ethylcarbamoylamino)-oxo-sulfanylidene]-2-methyl-2-prop-2-enoxypropanamide in Step 2. A mixture of two inseparable diastereomers as a light yellow solid was obtained. LC-MS calc. for C₃₅H₄₆ClN₄O₆S [M+H]⁺: m/z=685.3/687.3; Found: 685.2/687.5. HPLC: C18 column (4.6×100 mm, 5 μm); flow rate=1 mL/min; mobile phase: 50% MeCN/H₂O (with 0.1% TFA) to 95% 4 min, 95% 6 min; λ=220 nm. tR=5.51 min and 5.75 min.

### Example 21 and Example 22 1-[(3R,6R,7S,8E,15R,22S)-7′-Chloro-7-methoxy-12,12-dimethyl-13,15-dioxospiro[11,20-dioxa-15-thia-1,14-diazatetracyclo[14.7.2.03,6.019,24]pentacosa-8,14,16,18,24-pentaene-22,4′-2,3-dihydro-1H-naphthalene]-15-yl]-3-propan-2-ylurea and 1-[(3R,6R,7S,8E,15S,22S)-7′-Chloro-7-methoxy-12,12-dimethyl-13,15-dioxospiro[11,20-dioxa-15-thia-1,14-diazatetracyclo[14.7.2.03,6.019,24]pentacosa-8,14,16,18,24-pentaene-22,4′-2,3-dihydro-1H-naphthalene]-15-yl]-3-propan-2-ylurea

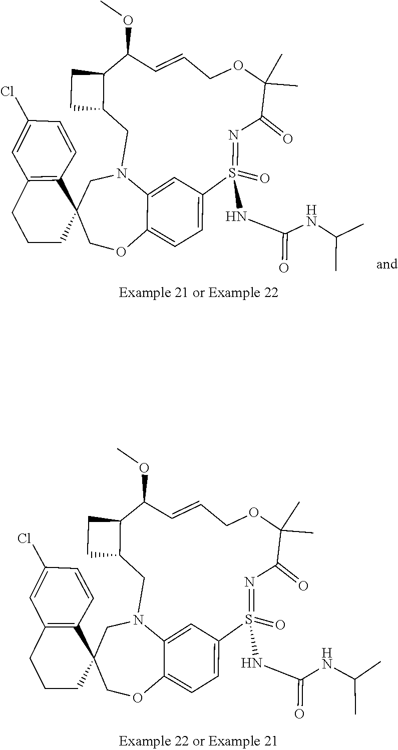 [SMILES: CO[C@H]1/C=C/COC(C)(C)C(=O)N=[S@@](=O)(NC(=O)NC(C)C)c2ccc3c(c2)N(C[C@@H]2CC[C@H]21)C[C@@]1(CCCc2cc(Cl)ccc21)CO3 ; CO[C@H]1/C=C/COC(C)(C)C(=O)N=[S@](=O)(NC(=O)NC(C)C)c2ccc3c(c2)N(C[C@@H]2CC[C@H]21)C[C@@]1(CCCc2cc(Cl)ccc21)CO3]

This compound was prepared as a white solid using procedures analogous to those described for Example 20 Step 1-2 using 2-aminopropane to replace ethylamine (2.0 M in THF) in Step 1.

P1 as a yellow solid was assigned to Example 11 or Example 12. LC-MS calc. for C₃₆H₄₈ClN₄O₆S [M+H]⁺: m/z=699.3/701.3; Found: 699.2/701.5. HPLC: C18 column (4.6×100 mm, 5 μm); flow rate=1 mL/min; mobile phase: 50% MeCN/H₂O (with 0.1% TFA) to 95% 4 min, 95% 6 min; λ=220 nm. tR=5.80 min. ¹H NMR (300 MHz, CDCl₃) δ 7.72-7.65 (m, 1H), 7.52 (dd, J=8.3, 2.3 Hz, 1H), 7.19 (dd, J=8.7, 2.4 Hz, 1H), 7.10 (d, J=2.4 Hz, 1H), 7.04-6.98 (m, 2H), 5.82-5.76 (m, 1H), 5.63 (d, J=16.1 Hz, 1H), 5.34 (br, 1H), 5.15 (d, J=10.2 Hz, 1H), 4.14-4.10 (m, 3H), 4.00 (d, J=6.3 Hz, 1H), 3.93-3.85 (m, 1H), 3.72-3.69 (m, 1H), 3.59-3.55 (m, 1H), 3.38-3.29 (m, 2H), 3.27 (s, 3H), 2.81-2.76 (m, 2H), 2.48-2.44 (m, 1H), 2.02-1.94 (m, 3H), 1.80-1.76 (m, 2H), 1.65-1.60 (m, 1H), 1.47 (s, 3H), 1.43 (s, 3H), 1.33-1.25 (m, 4H), 1.20 (d, J=6.4 Hz, 6H).

P2 as a yellow solid was assigned to Example 12 or Example 11. LC-MS calc. for C₃₆H₄₈ClN₄O₆S [M+H]⁺: m/z=699.3/701.3; Found: 699.0/701.1. HPLC: C18 column (4.6×100 mm, 5 μm); flow rate=1 mL/min; mobile phase: 50% MeCN/H₂O (with 0.1% TFA) to 95% 4 min, 95% 6 min; λ=220 nm. tR=6.11 min. ¹H NMR (300 MHz, CDCl₃) δ 7.68 (d, J=8.6 Hz, 1H), 7.50 (d, J=8.4 Hz, 1H), 7.19 (dd, J=8.7, 2.4 Hz, 1H), 7.10 (s, 1H), 7.01-6.95 (m, 1H), 6.92 (d, J=5.3 Hz, 1H), 6.19-5.92 (m, 1H), 5.66 (d, J=17.6 Hz, 1H), 5.36 (br, 1H), 5.16 (br, 1H), 4.20-4.07 (m, 3H), 4.03-3.98 (m, 1H), 3.69-3.65 (m, 2H), 3.38 (s, 3H), 3.34-3.29 (m, 2H), 3.10 (d, J=10.4 Hz, 1H), 2.77-2.74 (m, 2H), 2.09-1.93 (m, 4H), 1.75 (d, J=8.6 Hz, 1H), 1.62 (d, J=9.6 Hz, 1H), 1.49 (s, 3H), 1.38 (s, 3H), 1.35-1.33 (m, 2H), 1.30-1.25 (m, 3H), 1.20 (d, J=6.6 Hz, 6H).

### Example 23 and Example 24 1-[(3R,6R,7S,8E,15R,22S)-7′-Chloro-7-methoxy-12,12-dimethyl-13,15-dioxospiro[11,20-dioxa-15-thia-1,14-diazatetracyclo[14.7.2.03,6.019,24]pentacosa-8,14,16,18,24-pentaene-22,4′-2,3-dihydro-1H-naphthalene]-15-yl]-3-cyclopropylurea and 1-[(3R,6R,7S,8E,15S,22S)-7′-chloro-7-methoxy-12,12-dimethyl-13,15-dioxospiro[11,20-dioxa-15-thia-1,14-diazatetracyclo[14.7.2.03,6.019,24]pentacosa-8,14,16,18,24-pentaene-22,4′-2,3-dihydro-1H-naphthalene]-15-yl]-3-cyclopropylurea

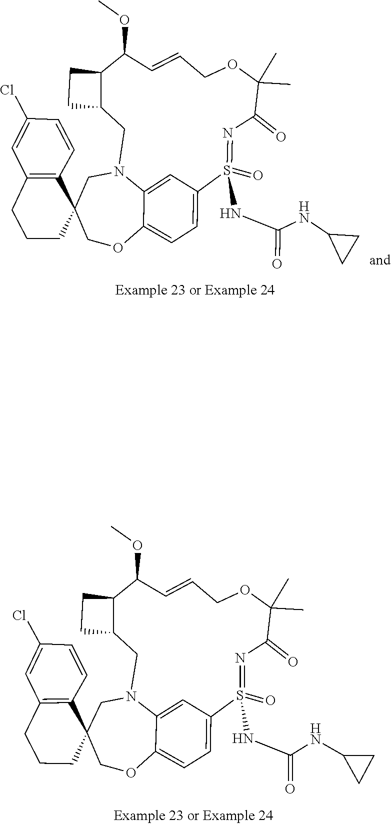 [SMILES: CO[C@H]1/C=C/COC(C)(C)C(=O)N=[S@@](=O)(NC(=O)NC2CC2)c2ccc3c(c2)N(C[C@@H]2CC[C@H]21)C[C@@]1(CCCc2cc(Cl)ccc21)CO3 ; CO[C@H]1/C=C/COC(C)(C)C(=O)N=[S@](=O)(NC(=O)NC2CC2)c2ccc3c(c2)N(C[C@@H]2CC[C@H]21)C[C@@]1(CCCc2cc(Cl)ccc21)CO3]

This compound was prepared as a white solid using procedures analogous to those described for Example 20 Step 1-2 using cyclopropylamine to replace ethylamine (2.0 M in THF) in Step 1.

P1 as a light brown solid was assigned to Example 13 or Example 14. LC-MS calc. for C₃₆H₄₆ClN₄O₆S [M+H]⁺: m/z=697.3/699.3; Found: 697.1/699.2. HPLC: C18 column (4.6×100 mm, 5 μm); flow rate=1 mL/min; mobile phase: 50% MeCN/H₂O (with 0.1% TFA) to 95% 4 min, 95% 6 min; λ=220 nm. tR=5.50 min. ¹H NMR (300 MHz, CDCl₃) δ 7.66 (d, J=8.5 Hz, 1H), 7.50 (d, J=8.3 Hz, 1H), 7.19 (dd, J=8.5, 2.3 Hz, 1H), 7.10 (d, J=2.3 Hz, 1H), 7.05 (s, 1H), 7.00 (d, J=8.4 Hz, 1H), 5.88-5.79 (m, 1H), 5.65 (dd, J=15.7, 7.1 Hz, 1H), 5.42 (br, 1H), 4.12-4.04 (m, 3H), 3.89 (dd, J=13.9, 6.0 Hz, 1H), 3.72 (d, J=14.5 Hz, 1H), 3.58 (dd, J=7.1, 3.1 Hz, 1H), 3.49 (d, J=14.5 Hz, 1H), 3.35 (d, J=4.5 Hz, 1H), 3.27 (s, 3H), 2.78-2.70 (m, 3H), 2.49-2.45 (m, 2H), 2.01-1.78 (m, 6H), 1.69-1.52 (m, 2H), 1.47 (s, 3H), 1.43 (s, 3H), 1.28-1.25 (m, 2H), 0.81-0.77 (m, 2H), 0.59-0.54 (m, 2H).

P2 as a light brown solid was assigned to Example 14 or Example 13. LC-MS calc. for C₃₆H₄₆ClN₄O₆S [M+H]⁺: m/z=697.3/699.3; Found: 697.1/699.3. HPLC: C18 column (4.6×100 mm, 5 μm); flow rate=1 mL/min; mobile phase: 50% MeCN/H₂O (with 0.1% TFA) to 95% 4 min, 95% 6 min; λ=220 nm. tR=5.71 min. ¹H NMR (300 MHz, CDCl₃) δ 7.67 (d, J=8.4 Hz, 1H), 7.50 (d, J=8.7 Hz, 1H), 7.19 (dd, J=8.5, 2.4 Hz, 1H), 7.09 (d, J=2.2 Hz, 1H), 7.01-6.89 (m, 2H), 6.12-6.08 (m, 1H), 5.65 (dd, J=16.1, 5.9 Hz, 1H), 5.47 (br, 1H), 4.18-4.11 (m, 3H), 3.70 (d, J=15.0 Hz, 2H), 3.61 (s, 1H), 3.39 (s, 3H), 3.32-3.27 (m, 1H), 3.09 (t, J=12.8 Hz, 1H), 2.78-2.72 (m, 3H), 2.15-1.93 (m, 6H), 1.76 (d, J=8.7 Hz, 1H), 1.61 (t, J=8.6 Hz, 1H), 1.51 (s, 3H), 1.44-1.42 (m, 1H), 1.38 (s, 3H), 1.35-1.24 (m, 3H), 0.78-0.73 (m, 2H), 0.62-0.53 (m, 2H).

### Example 25 and Example 26 1-[(3R,6R,7S,8E,15R,22S)-7′-Chloro-7-methoxy-12,12-dimethyl-13,15-dioxospiro[11,20-dioxa-15-thia-1,14-diazatetracyclo[14.7.2.03,6.019,24]pentacosa-8,14,16,18,24-pentaene-22,4′-2,3-dihydro-1H-naphthalene]-15-yl]-3-cyclobutylurea and 1-[(3R,6R,7S,8E,15S,22S)-7′-chloro-7-methoxy-12,12-dimethyl-13,15-dioxospiro[11,20-dioxa-15-thia-1,14-diazatetracyclo[14.7.2.03,6.019,24]pentacosa-8,14,16,18,24-pentaene-22,4′-2,3-dihydro-1H-naphthalene]-15-yl]-3-cyclobutylurea

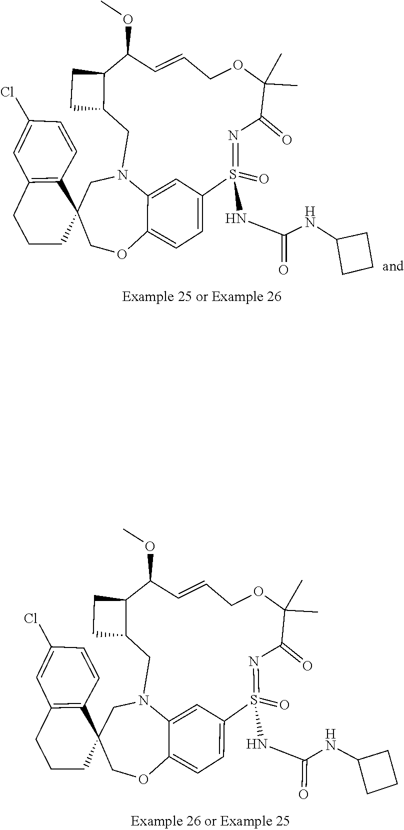 [SMILES: CO[C@H]1/C=C/COC(C)(C)C(=O)N=[S@@](=O)(NC(=O)NC2CCC2)c2ccc3c(c2)N(C[C@@H]2CC[C@H]21)C[C@@]1(CCCc2cc(Cl)ccc21)CO3 ; CO[C@H]1/C=C/COC(C)(C)C(=O)N=[S@](=O)(NC(=O)NC2CCC2)c2ccc3c(c2)N(C[C@@H]2CC[C@H]21)C[C@@]1(CCCc2cc(Cl)ccc21)CO3]

This compound was prepared as a white solid using procedures analogous to those described for Example 20 Step 1-2 using cyclobutanamine to replace ethylamine (2.0 M in THF) in Step 1.

P1 as a light brown solid was assigned to Example 15 or Example 16. LC-MS calc. for C₃₇H₄₈ClN₄O₆S [M+H]⁺: m/z=711.3/713.3; Found: 711.2/713.5. HPLC: C18 column (4.6×100 mm, 5 μm); flow rate=1 mL/min; mobile phase: 50% MeCN/H₂O (with 0.1% TFA) to 95% 4 min, 95% 6 min; λ=220 nm. tR=5.96 min. ¹H NMR (300 MHz, CDCl₃) δ 7.66 (d, J=8.6 Hz, 1H), 7.51 (d, J=8.4 Hz, 1H), 7.22-7.17 (m, 1H), 7.10 (s, 1H), 7.07-6.97 (m, 2H), 5.88-5.80 (m, 1H), 5.64 (dd, J=15.9, 7.3 Hz, 1H), 5.39 (br, 1H), 4.32 (d, J=7.9 Hz, 1H), 4.14-4.10 (m, 2H), 3.88 (dd, J=14.0, 6.3 Hz, 1H), 3.72 (d, J=14.2 Hz, 1H), 3.57 (s, 1H), 3.48-3.41 (m, 1H), 3.36 (t, J=1.8 Hz, 1H), 3.27 (s, 3H), 2.81-2.78 (m, 2H), 2.40-2.35 (m, 2H), 2.28 (t, J=7.6 Hz, 1H), 1.96-1.90 (m, 5H), 1.80-1.63 (m, 6H), 1.47 (s, 3H), 1.42 (s, 3H), 1.30-1.24 (m, 5H).

P2 as a light brown solid was assigned to Example 16 or Example 15. LC-MS calc. for C₃₇H₄₈ClN₄O₆S [M+H]⁺: m/z=711.3/713.3; Found: 711.2/713.5. HPLC: C18 column (4.6×100 mm, 5 μm); flow rate=1 mL/min; mobile phase: 50% MeCN/H₂O (with 0.1% TFA) to 95% 4 min, 95% 6 min; λ=220 nm. tR=6.31 min. ¹H NMR (300 MHz, CDCl₃) δ 7.67 (d, J=8.4 Hz, 1H), 7.50 (d, J=8.5 Hz, 1H), 7.19 (dd, J=8.7, 2.4 Hz, 1H), 7.09 (s, 1H), 6.97 (d, J=8.2 Hz, 1H), 6.90 (s, 1H), 6.08 (br, 1H), 5.70-5.64 (m, 1H), 5.51-5.33 (m, 1H), 4.35 (d, J=8.2 Hz, 1H), 4.20-4.14 (m, 2H), 3.68 (d, J=14.1 Hz, 2H), 3.60 (s, 1H), 3.37 (s, 3H), 3.33 (s, 1H), 3.07 (t, J=12.8 Hz, 1H), 2.81-2.77 (m, 2H), 2.29 (d, J=7.8 Hz, 2H), 2.11-1.81 (m, 8H), 1.70-1.61 (m, 4H), 1.50 (d, J=7.6 Hz, 3H), 1.38 (d, J=8.5 Hz, 3H), 1.30 (d, J=15.7 Hz, 5H).

### Example 27 and Example 28 N-[(3R,6R,7S,8E,15R,22S)-7′-Chloro-7-methoxy-12,12-dimethyl-13,15-dioxospiro[11,20-dioxa-15-thia-1,14-diazatetracyclo[14.7.2.03,6.019,24]pentacosa-8,14,16,18,24-pentaene-22,4′-2,3-dihydro-1H-naphthalene]-15-yl]azetidine-1-carboxamide and N-[(3R,6R,7S,8E,15S,22S)-7′-chloro-7-methoxy-12,12-dimethyl-13,15-dioxospiro[11,20-dioxa-15-thia-1,14-diazatetracyclo[14.7.2.03,6.019,24]pentacosa-8,14,16,18,24-pentaene-22,4′-2,3-dihydro-1H-naphthalene]-15-yl]azetidine-1-carboxamide

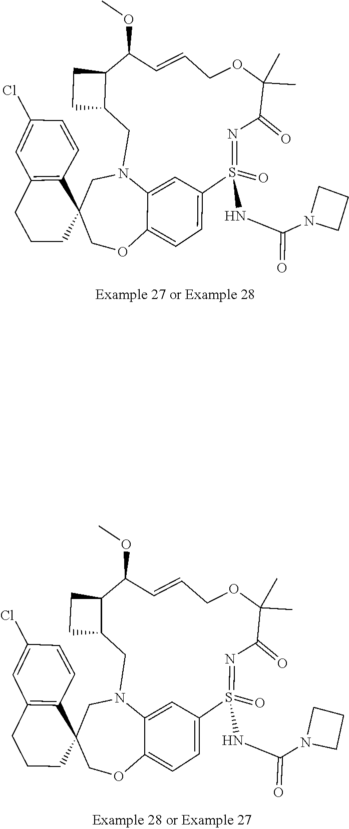 [SMILES: CO[C@H]1/C=C/COC(C)(C)C(=O)N=[S@@](=O)(NC(=O)N2CCC2)c2ccc3c(c2)N(C[C@@H]2CC[C@H]21)C[C@@]1(CCCc2cc(Cl)ccc21)CO3 ; CO[C@H]1/C=C/COC(C)(C)C(=O)N=[S@](=O)(NC(=O)N2CCC2)c2ccc3c(c2)N(C[C@@H]2CC[C@H]21)C[C@@]1(CCCc2cc(Cl)ccc21)CO3]

This compound was prepared as a white solid using procedures analogous to those described for Example 20 Step 1-2 using triethylamine and azetidine hydrochloride to replace ethylamine (2.0 M in THF) in Step 1.

P1 as a light brown solid was assigned to Example 17 or Example 18. LC-MS calc. for C₃₆H₄₆ClN₄O₆S [M+H]⁺: m/z=697.3/699.3; Found: 697.0/699.2. HPLC: C18 column (4.6×100 mm, 5 μm); flow rate=1 mL/min; mobile phase: 50% MeCN/H₂O (with 0.1% TFA) to 95% 4 min, 95% 6 min; λ=220 nm. tR=5.82 min. ¹H NMR (300 MHz, CDCl₃) δ 7.65 (d, J=8.5 Hz, 1H), 7.50 (dd, J=8.5, 2.2 Hz, 1H), 7.19 (d, J=9.5 Hz, 1H), 7.14-7.08 (m, 2H), 7.00 (dd, J=8.3, 1.1 Hz, 1H), 5.78-5.71 (m, 1H), 5.60 (dd, J=15.7, 7.2 Hz, 1H), 5.36 (br, 1H), 4.15-4.07 (m, 4H), 3.84 (dd, J=14.0, 6.3 Hz, 1H), 3.70 (d, J=14.3 Hz, 1H), 3.52 (d, J=7.1 Hz, 1H), 3.34 (d, J=14.2 Hz, 1H), 3.24 (s, 3H), 2.86-2.80 (m, 2H), 2.57-2.47 (m, 2H), 2.26 (t, J=7.7 Hz, 2H), 1.89-1.78 (m, 10H), 1.66-1.57 (m, 3H), 1.46 (s, 3H), 1.42 (s, 3H).

P2 as a light brown solid was assigned to Example 18 or Example 17. LC-MS calc. for C₃₆H₄₆ClN₄O₆S [M+H]⁺: m/z=697.3/699.3; Found: 697.0/699.2. HPLC: C18 column (4.6×100 mm, 5 μm); flow rate=1 mL/min; mobile phase: 50% MeCN/H₂O (with 0.1% TFA) to 95% 4 min, 95% 6 min; λ=220 nm. tR=6.18 min. ¹H NMR (300 MHz, CDCl₃) δ 7.68 (d, J=8.4 Hz, 1H), 7.50 (dd, J=8.4, 2.2 Hz, 1H), 7.21-7.17 (m, 1H), 7.09 (d, J=2.3 Hz, 1H), 6.97 (d, J=8.4 Hz, 1H), 6.90 (d, J=2.4 Hz, 1H), 6.09-6.02 (m, 1H), 5.66 (dd, J=15.9, 5.0 Hz, 1H), 5.36 (s, 1H), 4.15-4.06 (m, 4H), 3.64 (d, J=15.8 Hz, 2H), 3.38 (s, 3H), 3.34-3.30 (m, 1H), 3.10 (dd, J=15.0, 10.2 Hz, 1H), 2.81-2.74 (m, 2H), 2.29-2.22 (m, 3H), 2.06-1.99 (m, 4H), 1.87-1.68 (m, 7H), 1.65-1.62 (m, 3H), 1.50 (s, 3H), 1.36 (s, 3H).

### Example 29 and Example 30 N-[(3R,6R,7S,8E,15R,22S)-7′-Chloro-7-methoxy-12,12-dimethyl-13,15-dioxospiro[11,20-dioxa-15-thia-1,14-diazatetracyclo[14.7.2.03,6.019,24]pentacosa-8,14,16,18,24-pentaene-22,4′-2,3-dihydro-1H-naphthalene]-15-yl]-3-hydroxyazetidine-1-carboxamide and N-[(3R,6R,7S,8E,15S,22S)-7′-chloro-7-methoxy-12,12-dimethyl-13,15-dioxospiro[11,20-dioxa-15-thia-1,14-diazatetracyclo[14.7.2.03,6.019,24]pentacosa-8,14,16,18,24-pentaene-22,4′-2,3-dihydro-1H-naphthalene]-15-yl]-3-hydroxyazetidine-1-carboxamide

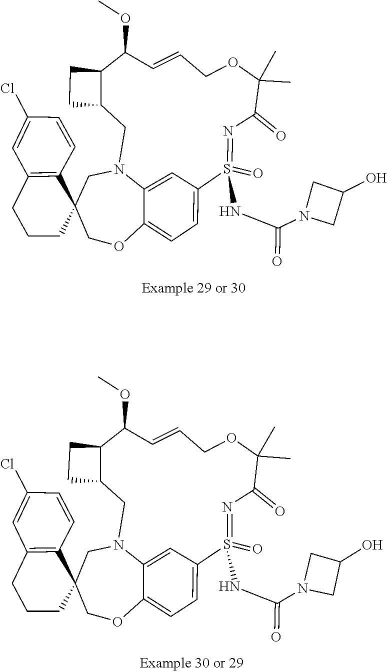 [SMILES: CO[C@H]1/C=C/COC(C)(C)C(=O)N=[S@@](=O)(NC(=O)N2CC(O)C2)c2ccc3c(c2)N(C[C@@H]2CC[C@H]21)C[C@@]1(CCCc2cc(Cl)ccc21)CO3 ; CO[C@H]1/C=C/COC(C)(C)C(=O)N=[S@](=O)(NC(=O)N2CC(O)C2)c2ccc3c(c2)N(C[C@@H]2CC[C@H]21)C[C@@]1(CCCc2cc(Cl)ccc21)CO3]

This compound was prepared as a white solid using procedures analogous to those described for Example 20 Step 1-2 using triethylamine and azetidin-3-ol hydrochloride to replace ethylamine (2.0 M in THF) in Step 1.

P1 as a light yellow solid was assigned to Example 19 or Example 20. LC-MS calc. for C₃₆H₄₆ClN₄O₇S [M+H]⁺: m/z=713.3/715.3; Found: 713.1/715.5. HPLC: C18 column (4.6×100 mm, 5 μm); flow rate=1 mL/min; mobile phase: 50% MeCN/H₂O (with 0.1% TFA) to 95% 4 min, 95% 6 min; λ=220 nm. tR=4.92 min. ¹H NMR (300 MHz, CDCl₃) δ 7.65 (d, J=8.5 Hz, 1H), 7.50 (dd, J=8.5, 2.2 Hz, 1H), 7.19 (dd, J=8.5, 2.3 Hz, 1H), 7.13-7.09 (m, 2H), 7.00 (d, J=8.4 Hz, 1H), 5.79-5.71 (m, 1H), 5.61 (dd, J=15.8, 7.2 Hz, 1H), 5.36 (br, 1H), 4.68 (br, 1H), 4.36-4.29 (m, 1H), 4.18 (dd, J=14.4, 4.3 Hz, 1H), 4.11 (d, J=3.6 Hz, 1H), 3.99-3.94 (m, 1H), 3.84 (dd, J=13.9, 5.9 Hz, 1H), 3.71 (d, J=14.5 Hz, 1H), 3.54 (d, J=7.0 Hz, 1H), 3.45 (d, J=13.4 Hz, 1H), 3.34 (d, J=14.3 Hz, 2H), 3.24 (s, 3H), 2.78 (d, J=10.3 Hz, 2H), 2.54-2.51 (m, 2H), 2.25 (t, J=7.6 Hz, 1H), 2.02-1.98 (m, 3H), 1.85-1.71 (m, 8H), 1.46 (s, 3H), 1.42 (s, 3H).

P2 as a light yellow solid was assigned to Example 20 or Example 19. LC-MS calc. for C₃₆H₄₆ClN₄O₇S [M+H]⁺: m/z=713.3/715.3; Found: 713.0/715.2. HPLC: C18 column (4.6×100 mm, 5 μm); flow rate=1 mL/min; mobile phase: 50% MeCN/H₂O (with 0.1% TFA) to 95% 4 min, 95% 6 min; λ=220 nm. tR=5.00 min. ¹H NMR (300 MHz, CDCl₃) δ 7.73-7.65 (m, 1H), 7.50 (dt, J=8.3, 2.3 Hz, 1H), 7.19 (dd, J=8.7, 2.5 Hz, 1H), 7.14-7.07 (m, 1H), 6.98 (dd, J=8.4, 2.2 Hz, 1H), 6.87-6.83 (m, 1H), 6.12-5.96 (m, 1H), 5.66 (dd, J=15.5, 5.1 Hz, 1H), 5.34 (br, 1H), 4.63 (br, 1H), 4.31 (t, J=8.6 Hz, 1H), 4.15 (dd, J=11.9, 9.5 Hz, 2H), 3.94 (d, J=5.5 Hz, 1H), 3.73-3.67 (m, 3H), 3.40 (s, 3H), 3.34-3.23 (m, 2H), 3.10 (dd, J=15.0, 10.3 Hz, 1H), 2.83-2.76 (m, 3H), 2.27 (t, J=7.8 Hz, 2H), 2.17-1.88 (m, 7H), 1.80-1.75 (m, 2H), 1.70-1.59 (m, 2H), 1.49 (s, 3H), 1.38 (m, 3H).

### Example 31 and Example 32 N-[(3R,6R,7S,8E,15R,22S)-7′-Chloro-7-methoxy-12,12-dimethyl-13,15-dioxospiro[11,20-dioxa-15-thia-1,14-diazatetracyclo[14.7.2.03,6.019,24]pentacosa-8,14,16,18,24-pentaene-22,4′-2,3-dihydro-1H-naphthalene]-15-yl]-3-methoxyazetidine-1-carboxamide and N-[(3R,6R,7S,8E,15S,22S)-7′-chloro-7-methoxy-12,12-dimethyl-13,15-dioxospiro[11,20-dioxa-15-thia-1,14-diazatetracyclo[14.7.2.03,6.019,24]pentacosa-8,14,16,18,24-pentaene-22,4′-2,3-dihydro-1H-naphthalene]-15-yl]-3-methoxyazetidine-1-carboxamide

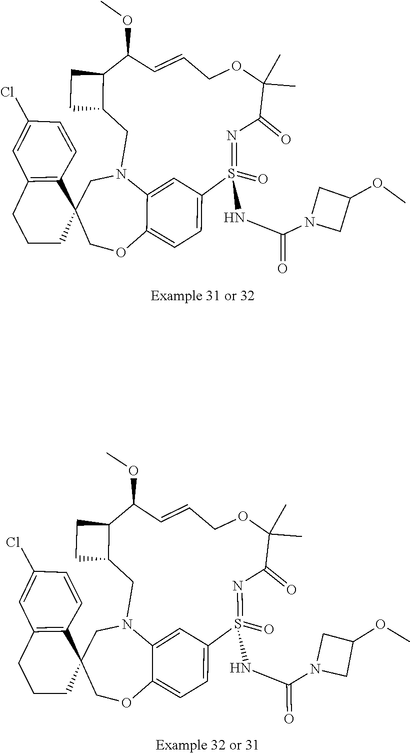 [SMILES: COC1CN(C(=O)N[S@]2(=O)=NC(=O)C(C)(C)OC/C=C/[C@H](OC)[C@@H]3CC[C@H]3CN3C[C@@]4(CCCc5cc(Cl)ccc54)COc4ccc2cc43)C1 ; COC1CN(C(=O)N[S@@]2(=O)=NC(=O)C(C)(C)OC/C=C/[C@H](OC)[C@@H]3CC[C@H]3CN3C[C@@]4(CCCc5cc(Cl)ccc54)COc4ccc2cc43)C1]

This compound was prepared as a white solid using procedures analogous to those described for Example 20 Step 1-2 using triethylamine and 3-methoxyazetidine hydrochloride to replace ethylamine (2.0 M in THF) in Step 1.

P1 as a light yellow solid was assigned to Example 21 or Example 22. LC-MS calc. for C₃₇H₄₈ClN₄O₇S [M+H]⁺: m/z=727.3/729.3; Found: 726.9/729.2. HPLC: C18 column (4.6×100 mm, 5 μm); flow rate=1 mL/min; mobile phase: 50% MeCN/H₂O (with 0.1% TFA) to 95% 4 min, 95% 6 min; λ=220 nm. tR=5.64 min. ¹H NMR (300 MHz, CDCl₃) δ 7.66 (d, J=8.5 Hz, 1H), 7.50 (dd, J=8.2, 2.2 Hz, 1H), 7.19 (dd, J=8.6, 2.5 Hz, 1H), 7.13-7.08 (m, 2H), 7.04-6.96 (m, 1H), 5.78-5.70 (m, 1H), 5.61 (dd, J=15.6, 7.1 Hz, 1H), 5.36 (br, 1H), 4.30-4.08 (m, 5H), 3.98 (d, J=7.0 Hz, 2H), 3.82-3.77 (m, 1H), 3.71 (d, J=14.5 Hz, 1H), 3.54 (d, J=6.3 Hz, 1H), 3.32 (s, 3H), 3.24 (s, 3H), 2.80-2.75 (m, 2H), 2.50-2.44 (m, 2H), 2.28 (t, J=7.6 Hz, 1H), 2.09-1.92 (m, 5H), 1.81 (t, J=8.8 Hz, 2H), 1.67-1.63 (m, 2H), 1.46 (s, 3H), 1.42 (s, 3H), 1.34-1.30 (m, 2H).

P2 as a light yellow solid was assigned to Example 22 or Example 21. LC-MS calc. for C₃₇H₄₈ClN₄O₇S [M+H]⁺: m/z=727.3/729.3; Found: 727.0/729.2. HPLC: C18 column (4.6×100 mm, 5 μm); flow rate=1 mL/min; mobile phase: 50% MeCN/H₂O (with 0.1% TFA) to 95% 4 min, 95% 6 min; λ=220 nm. tR=5.84 min. ¹H NMR (300 MHz, CDCl₃) δ 7.68 (d, J=8.4 Hz, 1H), 7.50 (dd, J=8.4, 2.2 Hz, 1H), 7.22-7.16 (m, 1H), 7.09 (d, J=2.3 Hz, 1H), 7.01-6.95 (m, 1H), 6.89 (d, J=2.3 Hz, 1H), 6.10-5.94 (m, 1H), 5.66 (dd, J=15.7, 5.3 Hz, 1H), 5.31 (br, 1H), 4.24-4.06 (m, 5H), 3.96 (d, J=7.2 Hz, 1H), 3.77-3.70 (m, 2H), 3.62-3.57 (m, 1H), 3.37 (s, 3H), 3.30 (s, 3H), 3.10 (dd, J=14.9, 9.8 Hz, 1H), 2.80-2.74 (m, 2H), 2.28 (t, J=7.6 Hz, 1H), 2.12-2.07 (m, 2H), 2.06-1.92 (m, 5H), 1.80-1.75 (m, 2H), 1.66-1.61 (m, 2H), 1.50 (s, 3H), 1.38 (s, 3H), 1.33-1.30 (m, 2H).

### Example 33 N-[(3R,6R,7S,8E,22S)-7′-Chloro-7-methoxy-12,12-dimethyl-13,15-dioxospiro[11,20-dioxa-15-thia-1,14-diazatetracyclo[14.7.2.03,6.019,24]pentacosa-8,14,16,18,24-pentaene-22,4′-2,3-dihydro-1H-naphthalene]-15-yl]-3-(dimethylamino)azetidine-1-carboxamide

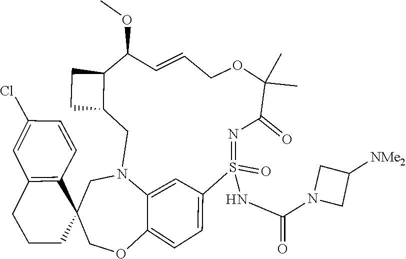 [SMILES: CO[C@H]1/C=C/COC(C)(C)C(=O)N=S(=O)(NC(=O)N2CC(N(C)C)C2)c2ccc3c(c2)N(C[C@@H]2CC[C@H]21)C[C@@]1(CCCc2cc(Cl)ccc21)CO3]

This compound was prepared as a white solid using procedures analogous to those described for Example 20 Step 1-2 using triethylamine and N,N-dimethylazetidin-3-amine dihydrochloride to replace ethylamine (2.0 M in THF) in Step 1. A mixture of two diastereomers was obtained as a light yellow solid was assigned to Example 19 or Example 20. as a yellow solid. LC-MS calc. for C₃₈H₅₁ClN₅O₆S [M+H]⁺: m/z=740.3/742.3; Found: 740.1/742.2. HPLC: C18 column (4.6×100 mm, 5 μm); flow rate=1 mL/min; mobile phase: 50% MeCN/H₂O (with 0.1% TFA) to 95% 4 min, 95% 6 min; μ=220 nm; tR=6.54 min. ¹H NMR (300 MHz, CDCl₃) (1:1 diastereomers) δ 7.68-7.63 (m, 1H), 7.50-7.43 (m, 1H), 7.20-7.15 (m, 1H), 7.12-7.08 (m, 1H), 7.02-6.97 (m, 1H), 6.89-6.85 (m, 1H), 5.94 (dd, J=15.4, 5.9 Hz, 0.5H), 5.74-5.68 (m, 1.5H), 4.37-4.30 (m, 3H), 4.16-4.11 (m, 2H), 4.01-3.94 (m, 1H), 3.76-3.59 (m, 2H), 3.34 (s, 1.5H), 3.27 (s, 1.5H), 3.16-3.13 (m, 1H), 2.83 (s, 6H), 2.79-2.58 (m, 4H), 2.49 (s, 1H), 2.24-2.21 (m, 1H), 2.12-1.87 (m, 4H), 1.79-1.75 (m, 2H), 1.70-1.54 (m, 2H), 1.49 (s, 1.5H), 1.44 (s, 1.511), 1.42 (s, 1.5H), 1.37 (s, 1.5H), 1.33-1.20 (m, 3H).

### Example 34 and Example 35 3-[(3R,6R,7S,8E,15R,22S)-7′-Chloro-7-methoxy-12,12-dimethyl-13,15-dioxospiro[11,20-dioxa-15 λ6-thia-1,14-diazatetracyclo[14.7.2.03,6.019,24]pentacosa-8,14,16,18,24-pentaene-22,4′-2,3-dihydro-1H-naphthalene]-15-yl]-1,1-dimethylurea and 3-[(3R,6R,7S,8E,15S,22S)-7′-chloro-7-methoxy-12,12-dimethyl-13,15-dioxospiro[11,20-dioxa-15 λ6-thia-1,14-diazatetracyclo[14.7.2.03,6.019,24]pentacosa-8,14,16,18,24-pentaene-22,4′-2,3-dihydro-1H-naphthalene]-15-yl]-1,1-dimethylurea

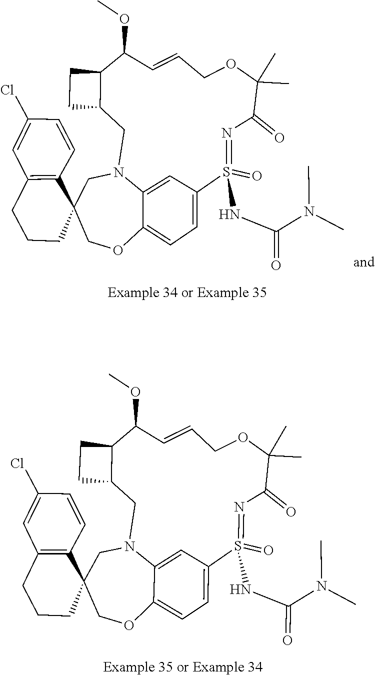 [SMILES: CO[C@H]1/C=C/COC(C)(C)C(=O)N=[S@@](=O)(NC(=O)N(C)C)c2ccc3c(c2)N(C[C@@H]2CC[C@H]21)C[C@@]1(CCCc2cc(Cl)ccc21)CO3 ; CO[C@H]1/C=C/COC(C)(C)C(=O)N=[S@](=O)(NC(=O)N(C)C)c2ccc3c(c2)N(C[C@@H]2CC[C@H]21)C[C@@]1(CCCc2cc(Cl)ccc21)CO3]

This compound was prepared as a white solid using procedures analogous to those described for Example 20 Step 1-2 using dimethylamine (2.0 M in THF) to replace ethylamine (2.0 M in THF) in Step 1.

P1 as a light yellow solid was assigned to Example 34 or Example 35. LC-MS calc. for C₃₇H₄₈ClN₄O₇S [M+H]⁺: m/z=. HPLC: C18 column (4.6×100 mm, 5 μm); flow rate=1 mL/min; mobile phase: 95% MeCN/120 (with 0.1% HCO₂H) 10 min; λ=220 nm. tR=5.64 min. ¹H NMR (300 MHz, CDCl₃) δ 7.65 (d, J=8.5 Hz, 1H), 7.51 (dd, J=8.4, 2.2 Hz, 1H), 7.21-7.12 (m, 2H), 7.10 (d, J=2.4 Hz, 1H), 7.00 (d, J=8.3 Hz, 1H), 5.77 (d, J=15.9 Hz, 1H), 5.62 (dd, J=15.9, 7.1 Hz, 1H), 4.13 (dd, J=11.9, 6.8 Hz, 3H), 3.86 (dd, J=13.8, 6.2 Hz, 1H), 3.70 (d, J=14.6 Hz, 1H), 3.56-3.49 (m, 1H), 3.35 (dt, J=21.6, 6.3 Hz, 3H), 3.25 (s, 3H), 3.07 (d, J=17.3 Hz, 6H), 2.78 (d, J=10.1 Hz, 3H), 2.59-2.41 (m, 3H), 2.00-1.76 (m, 6H), 1.46 (s, 3H), 1.42 (s, 3H).

P2 as a light yellow solid was assigned to Example 35 or Example 34. LC-MS calc. for C₃₇H₄₈ClN₄O₇S [M+H]⁺: m/z=. HPLC: C18 column (4.6×100 mm, 5 μm); flow rate=1 mL/min; mobile phase: 95% MeCN/H₂O (with 0.1% HCO₂H) 10 min; λ=220 nm. tR=5.64 min.

¹H NMR (300 MHz, CDCl₃) δ 7.68 (d, J=8.4 Hz, 1H), 7.51 (dd, J=8.4, 2.2 Hz, 1H), 7.18 (dd, J=8.5, 2.3 Hz, 1H), 7.09 (d, J=2.3 Hz, 1H), 6.97 (dd, J=8.4, 0.8 Hz, 1H), 6.92 (d, J=2.3 Hz, 1H), 6.07 (dd, J=16.3, 5.4 Hz, 1H), 5.74-5.64 (m, 1H), 4.21-4.07 (m, 3H), 3.78-3.55 (m, 4H), 3.42 (s, 1H), 3.36 (d, J=0.8 Hz, 3H), 3.31 (d, J=5.1 Hz, 1H), 3.04 (s, 6H), 2.79 (s, 3H), 2.12-1.81 (m, 9H), 1.51 (s, 3H), 1.37 (s, 3H).

### Example 36 and Example 37 1-[(3R,6R,7S,8E,15R,22S)-7′-Chloro-7-methoxy-12,12-dimethyl-13,15-dioxospiro[11,20-dioxa-15-thia-1,14-diazatetracyclo[14.7.2.03,6.019,24]pentacosa-8,14,16,18,24-pentaene-22,4′-2,3-dihydro-1H-naphthalene]-15-yl]-3-(4-(trans)-methoxycyclohexyl)urea and 1-[(3R,6R,7S,8E,15S,22S)-7′-chloro-7-methoxy-12,12-dimethyl-13,15-dioxospiro[11,20-dioxa-15-thia-1,14-diazatetracyclo[14.7.2.03,6.019,24]pentacosa-8,14,16,18,24-pentaene-22,4′-2,3-dihydro-1H-naphthalene]-15-yl]-3-(4-(trans)-methoxycyclohexyl)urea

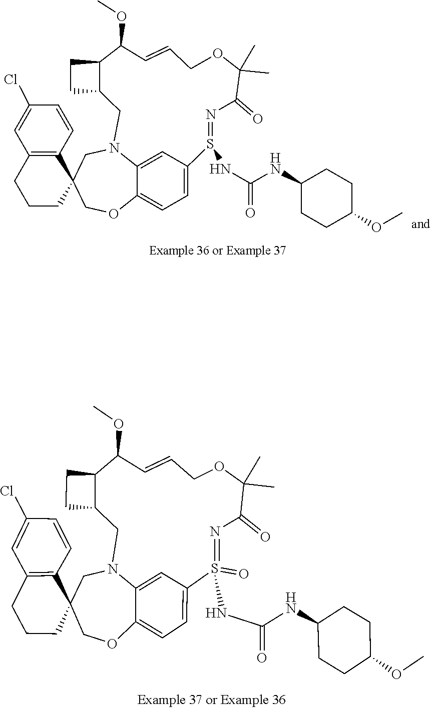 [SMILES: CO[C@H]1/C=C/COC(C)(C)C(=O)/N=[S@@](\NC(=O)N[C@H]2CC[C@H](OC)CC2)c2ccc3c(c2)N(C[C@@H]2CC[C@H]21)C[C@@]1(CCCc2cc(Cl)ccc21)CO3 ; CO[C@H]1/C=C/COC(C)(C)C(=O)N=[S@](=O)(NC(=O)N[C@H]2CC[C@H](OC)CC2)c2ccc3c(c2)N(C[C@@H]2CC[C@H]21)C[C@@]1(CCCc2cc(Cl)ccc21)CO3]

This compound was prepared as a white solid using procedures analogous to those described for Example 20 Step 1-2 using (trans)-4-methoxycyclohexan-1-amine to replace ethylamine (2.0 M in THF) in Step 1.

P1 was assigned to Example 36 or Example 37. LC-MS calc. for C₄₀H₅₄ClN₄O₇S [M+H]⁺: m/z=769.3/771.3; Found: 769.3/771.6. HPLC: C18 column (4.6×100 mm, 5 μm); flow rate=1 mL/min; mobile phase: 50% MeCN/H₂O (with 0.1% TFA) to 95% 5 min, 95% 5 min; λ=220 nm. tR=8.059 min. ¹H NMR (300 MHz, CDCl₃) δ 7.66 (d, J=8.5 Hz, 1H), 7.51 (dd, J=8.5, 2.2 Hz, 1H), 7.19 (dd, J=8.7, 2.3 Hz, 1H), 7.10 (d, J=2.4 Hz, 1H), 7.06-6.92 (m, 2H), 5.89-5.51 (m, 2H), 5.12 (d, J=8.0 Hz, 1H), 4.25-4.02 (m, 3H), 3.86 (dd, J=13.9, 5.9 Hz, 1H), 3.79-3.43 (m, 5H), 3.36 (d, J=0.8 Hz, 3H), 3.25 (d, J=0.9 Hz, 3H), 3.16 (t, J=10.4 Hz, 2H), 2.78 (d, J=10.2 Hz, 3H), 2.47 (s, 2H), 2.13-1.79 (m, 9H), 1.48 (s, 3H), 1.42 (s, 3H), 1.36-1.17 (m, 6H).

P2 was assigned to Example 37 or Example 36. LC-MS calc. for C₄₀H₅₄ClN₄O₇S [M+H]⁺: m/z=769.3/771.3; Found: 769.3/771.6. HPLC: C18 column (4.6×100 mm, 5 μm); flow rate=1 mL/min; mobile phase: 50% MeCN/H₂O (with 0.1% TFA) to 95% 5 min, 95% 5 min; =220 nm. tR=8.345 min. ¹H NMR (300 MHz, CDCl₃) δ 7.67 (d, J=8.6 Hz, 1H), 7.48 (dd, J=8.4, 2.2 Hz, 1H), 7.19 (dd, J=8.6, 2.4 Hz, 1H), 7.09 (d, J=2.3 Hz, 1H), 6.97 (d, J=8.5 Hz, 1H), 6.88 (d, J=2.3 Hz, 1H), 6.15 (dd, J=15.6, 6.7 Hz, 1H), 5.66 (dd, J=15.8, 4.8 Hz, 1H), 5.09 (d, J=8.4 Hz, 1H), 4.21-4.02 (m, 3H), 3.69 (d, J=14.5 Hz, 3H), 3.60 (s, 2H), 3.43 (d, J=2.0 Hz, 1H), 3.38 (s, 3H), 3.35 (s, 3H), 3.32 (d, J=3.6 Hz, 2H), 3.18-3.01 (m, 2H), 2.79 (s, 3H), 2.16-1.92 (m, 9H), 1.52 (s, 3H), 1.37 (d, J=2.5 Hz, 3H), 1.24 (d, J=16.5 Hz, 6H).

### Example 38 1-[(3R,6R,7S,8E,22S)-7′-Chloro-7-methoxy-12,12-dimethyl-13,15-dioxospiro[11,20-dioxa-15-thia-1,14-diazatetracyclo[14.7.2.03,6.019,24]pentacosa-8,14,16,18,24-pentaene-22,4′-2,3-dihydro-1H-naphthalene]-15-yl]-3-(oxan-4-yl)urea

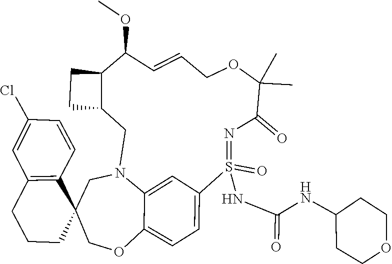 [SMILES: CO[C@H]1/C=C/COC(C)(C)C(=O)N=S(=O)(NC(=O)NC2CCOCC2)c2ccc3c(c2)N(C[C@@H]2CC[C@H]21)C[C@@]1(CCCc2cc(Cl)ccc21)CO3]

This compound was prepared as a white solid using procedures analogous to those described for Example 20 Step 1-2 using 4-aminotetrahydropyran to replace ethylamine (2.0 M in THF) in Step 1. A mixture of two diastereomers was obtained. LC-MS calc. for C₃₈H₅₀ClN₄O₇S [M+H]⁺: m/z=741.3/743.3.3; Found: 741.2/743.6. HPLC: C18 column (4.6×100 mm, 5 μm); flow rate=1 mL/min; mobile phase: 50% MeCN/H₂O (with 0.1% TFA) to 95% 5 min, 95% 5 min; λ=220 nm. tR=7.782 min. ¹H NMR (300 MHz, CDCl₃) δ 7.67 (d, J=8.5 Hz, 1H), 7.50 (d, J=8.8 Hz, 1H), 7.19 (d, J=8.7 Hz, 1H), 7.10 (s, 1H), 6.98 (d, J=8.5 Hz, 1H), 6.90 (s, 1H), 6.12 (d, J=15.2 Hz, 1H), 5.67 (d, J=15.2 Hz, 1H), 5.22 (d, J=9.0 Hz, 1H), 4.14 (ddd, J=26.2, 17.2, 9.9 Hz, 3H), 3.97 (d, J=12.2 Hz, 3H), 3.79 (d, J=2.5 Hz, 1H), 3.76-3.55 (m, 4H), 3.54-3.23 (m, 9H), 3.13-3.02 (m, 1H), 2.79 (s, 3H), 1.97 (s, 9H), 1.51 (s, 3H), 1.38 (s, 3H).

### Example 39 and Example 40 1-[(3R,6R,7S,8E,15R,22S)-7′-Chloro-7-methoxy-12,12-dimethyl-13,15-dioxospiro[11,20-dioxa-15-thia-1,14-diazatetracyclo[14.7.2.03,6.019,24]pentacosa-8,14,16,18,24-pentaene-22,4′-2,3-dihydro-1H-naphthalene]-15-yl]-3-(1-methylpyrazol-4-yl)urea and 1-[(3R,6R,7S,8E,15S,22S)-7′-chloro-7-methoxy-12,12-dimethyl-13,15-dioxospiro[11,20-dioxa-15-thia-1,14-diazatetracyclo[14.7.2.03,6.019,24]pentacosa-8,14,16,18,24-pentaene-22,4′-2,3-dihydro-1H-naphthalene]-15-yl]-3-(1-methylpyrazol-4-yl)urea

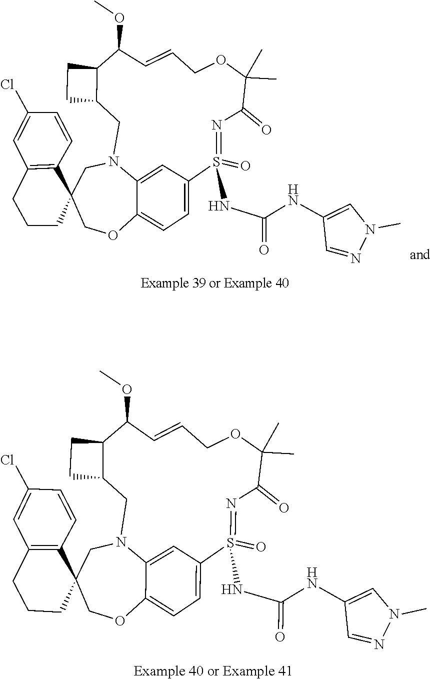 [SMILES: CO[C@H]1/C=C/COC(C)(C)C(=O)N=[S@@](=O)(NC(=O)Nc2cnn(C)c2)c2ccc3c(c2)N(C[C@@H]2CC[C@H]21)C[C@@]1(CCCc2cc(Cl)ccc21)CO3 ; CO[C@H]1/C=C/COC(C)(C)C(=O)N=[S@](=O)(NC(=O)Nc2cnn(C)c2)c2ccc3c(c2)N(C[C@@H]2CC[C@H]21)C[C@@]1(CCCc2cc(Cl)ccc21)CO3]

This compound was prepared as a white solid using procedures analogous to those described for Example 20 Step 1-2 using 1-methylpyrazol-4-amine to replace ethylamine (2.0 M in THF) in Step 1.

P1 was assigned to Example 39 or Example 40. LC-MS calc. for C₃₇H₄₆ClN₆O₆S [M+H]⁺: m/z=737.3/739.3; Found: 737.2/739.5. HPLC: C18 column (4.6×100 mm, 5 μm); flow rate=1 mL/min; mobile phase: 50% MeCN/H₂O (with 0.1% TFA) to 95% 5 min, 95% 5 min; λ=220 nm. tR=6.963 min. ¹H NMR (300 MHz, CDCl₃) δ 7.77 (s, 1H), 7.66 (d, J=8.5 Hz, 1H), 7.54 (dd, J=8.4, 2.2 Hz, 1H), 7.47 (s, 1H), 7.19 (dd, J=8.5, 2.3 Hz, 1H), 7.10 (d, J=2.3 Hz, 2H), 7.01 (d, J=8.1 Hz, 2H), 5.90-5.78 (m, 1H), 5.71 (dd, J=15.7, 6.6 Hz, 1H), 4.23-4.03 (m, 4H), 3.90 (s, 3H), 3.73 (d, J=14.7 Hz, 1H), 3.59 (t, J=5.1 Hz, 1H), 3.54-3.44 (m, 1H), 3.42-3.30 (m, 2H), 3.25 (s, 3H), 2.77 (d, J=10.5 Hz, 3H), 2.58-2.37 (m, 2H), 2.05-1.71 (m, 7H), 1.48 (s, 3H), 1.43 (s, 3H).

P2 was assigned to Example 40 or Example 39. LC-MS calc. for C₃₇H₄₆ClN₆O₆S [M+H]⁺: m/z=737.3/739.3; Found: 737.2/739.5. HPLC: C18 column (4.6×100 mm, 5 μm); flow rate=1 mL/min; mobile phase: 50% MeCN/H₂O (with 0.1% TFA) to 95% 5 min, 95% 5 min; =220 nm. tR=7.143 min. 1H NMR (300 MHz, CDCl₃) δ 7.94 (s, 1H), 7.67 (d, J=8.5 Hz, 1H), 7.53-7.44 (m, 2H), 7.18 (dd, J=8.5, 2.4 Hz, 1H), 7.13-7.06 (m, 2H), 7.03-6.95 (m, 2H), 6.22-6.04 (m, 1H), 5.76 (dd, J=15.7, 5.1 Hz, 1H), 4.30-3.99 (m, 4H), 3.90 (s, 3H), 3.74 (t, J=13.4 Hz, 2H), 3.63 (d, J=5.0 Hz, 1H), 3.41 (s, 1H), 3.36 (s, 1H), 3.27 (d, J=7.7 Hz, 4H), 3.07 (dd, J=15.0, 10.8 Hz, 1H), 2.88-2.72 (m, 3H), 2.15-1.76 (m, 7H), 1.53 (s, 3H), 1.39 (s, 3H).

### Example 41 and Example 42 N-[(3R,6R,7R,8E,15R,22S)-7′-Chloro-7-methoxy-12,12-dimethyl-13,15-dioxospiro[11,20-dioxa-15-thia-1,14-diazatetracyclo[14.7.2.03,6.019,24]pentacosa-8,14,16,18,24-pentaene-22,4′-2,3-dihydro-1H-naphthalene]-15-yl]acetamide and N-[(3R,6R,7R,8E,15S,22S)-7′-chloro-7-methoxy-12,12-dimethyl-13,15-dioxospiro[11,20-dioxa-15-thia-1,14-diazatetracyclo[14.7.2.03,6.019,24]pentacosa-8,14,16,18,24-pentaene-22,4′-2,3-dihydro-1H-naphthalene]-15-yl]acetamide

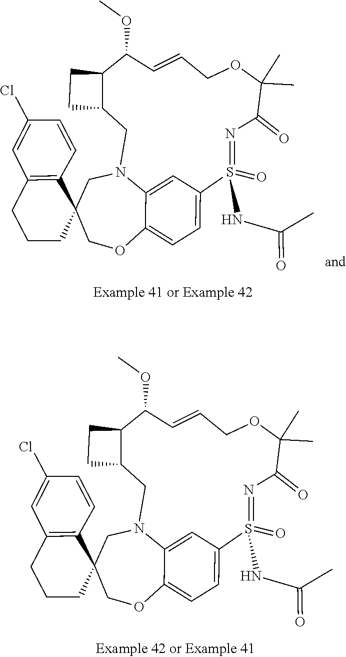 [SMILES: CO[C@@H]1/C=C/COC(C)(C)C(=O)N=[S@@](=O)(NC(C)=O)c2ccc3c(c2)N(C[C@@H]2CC[C@H]21)C[C@@]1(CCCc2cc(Cl)ccc21)CO3 ; CO[C@@H]1/C=C/COC(C)(C)C(=O)N=[S@](=O)(NC(C)=O)c2ccc3c(c2)N(C[C@@H]2CC[C@H]21)C[C@@]1(CCCc2cc(Cl)ccc21)CO3]

This compound was prepared using procedures analogous to those described for Example 1 and 2 Step 1-2 using N-[amino-oxo-[(4S)-7-chloro-5′-[[(1S,2S)-2-[(1R)-1-methoxyprop-2-enyl]cyclobutyl]methyl]spiro[2,3-dihydro-1H-naphthalene-4,3′-2,4-dihydro-1,5-benzoxazepine]-7′-yl]-sulfanylidene]-2-methyl-2-prop-2-enoxypropanamide (Intermediate 2) and acetyl chloride in Step 1.

P1 was assigned to Example 41 or Example 42. LC-MS calc. for C₃₄H₄₃ClN₃O₆S [M+H]⁺: m/z=656.3/658.3; Found: 656.1/658.3. HPLC: C18 column (4.6×100 mm, 5 μm); flow rate=1 mL/min; mobile phase: MeCN/H₂O (with 0.1% TFA) 50% to 95% 5 min, 95% 5 min; λ=220 nm. tR=6.957. ¹H NMR (300 MHz, CDCl₃) δ 7.67 (d, J=8.5 Hz, 1H), 7.50 (dd, J=8.4, 2.2 Hz, 1H), 7.19 (dd, J=8.5, 2.3 Hz, 1H), 7.09 (d, J=2.3 Hz, 1H), 7.01 (d, J=8.4 Hz, 1H), 6.83 (d, J=2.3 Hz, 1H), 6.18 (ddd, J=15.9, 7.3, 3.1 Hz, 1H), 5.56 (dd, J=16.0, 8.1 Hz, 1H), 4.34-4.01 (m, 4H), 3.82-3.70 (m, 2H), 3.55 (t, J=8.5 Hz, 1H), 3.42 (dd, J=15.0, 4.3 Hz, 2H), 3.33 (s, 3H), 3.04 (dd, J=15.1, 11.0 Hz, 1H), 2.79 (dd, J=10.3, 5.1 Hz, 3H), 2.42 (d, J=7.6 Hz, 1H), 2.23 (s, 3H), 2.07-1.94 (m, 5H), 1.75-1.65 (m, 2H), 1.51 (s, 3H), 1.39 (s, 3H).

P2 was assigned to Example 42 or Example 41. LC-MS calc. for C₃₄H₄₃ClN₃O₆S [M+H]⁺: m/z=656.3/658.3; Found: 656.2/658.4. HPLC: C18 column (4.6×100 mm, 5 μm); flow rate=1 mL/min; mobile phase: MeCN/H₂O (with 0.1% TFA) 50% to 95% 5 min, 95% 5 min; λ=220 nm. tR=7.828. ¹H NMR (300 MHz, CDCl₃) δ 7.69 (d, J=8.5 Hz, 1H), 7.55 (dd, J=8.4, 2.2 Hz, 1H), 7.19 (dd, J=8.5, 2.4 Hz, 1H), 7.09 (d, J=2.3 Hz, 1H), 7.03-6.95 (m, 2H), 5.91-5.69 (m, 2H), 4.19-4.03 (m, 4H), 3.86-3.69 (m, 2H), 3.58 (dd, J=8.0, 4.7 Hz, 1H), 3.32 (s, 4H), 3.03 (dd, J=15.2, 10.8 Hz, 1H), 2.87-2.68 (m, 3H), 2.49 (q, J=10.7, 10.1 Hz, 1H), 2.31 (dq, J=8.9, 4.5, 3.8 Hz, 1H), 2.02-1.80 (m, 5H), 1.74-1.58 (m, 2H), 1.44 (s, 3H), 1.40 (s, 3H).

### Example 43 and Example 44 N-[(3R,6R,7R,8E,15R,22S)-7′-Chloro-7-methoxy-12,12-dimethyl-13,15-dioxospiro[11,20-dioxa-15-thia-1,14-diazatetracyclo[14.7.2.03,6.019,24]pentacosa-8,14,16,18,24-pentaene-22,4′-2,3-dihydro-1H-naphthalene]-15-yl]propanamide and N-[(3R,6R,7R,8E,15S,22S)-7′-chloro-7-methoxy-12,12-dimethyl-13,15-dioxospiro[11,20-dioxa-15-thia-1,14-diazatetracyclo[14.7.2.03,6.019,24]pentacosa-8,14,16,18,24-pentaene-22,4′-2,3-dihydro-1H-naphthalene]-15-yl]propenamide

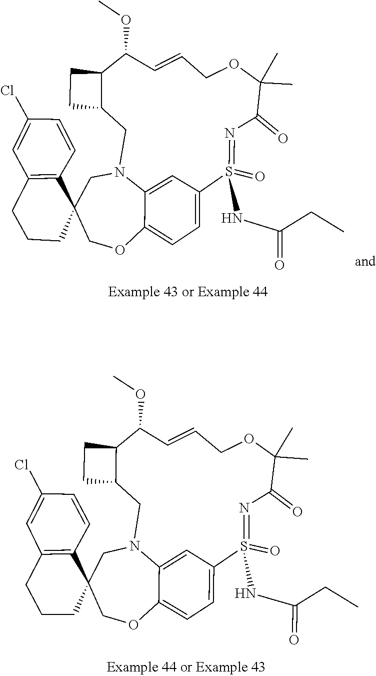 [SMILES: CCC(=O)N[S@]1(=O)=NC(=O)C(C)(C)OC/C=C/[C@@H](OC)[C@@H]2CC[C@H]2CN2C[C@@]3(CCCc4cc(Cl)ccc43)COc3ccc1cc32 ; CCC(=O)N[S@@]1(=O)=NC(=O)C(C)(C)OC/C=C/[C@@H](OC)[C@@H]2CC[C@H]2CN2C[C@@]3(CCCc4cc(Cl)ccc43)COc3ccc1cc32]

This compound was prepared using procedures analogous to those described for Example 1 and 2 Step 1-2 using N-[amino-oxo-[(4S)-7-chloro-5′-[[(1S,2S)-2-[(1R)-1-methoxyprop-2-enyl]cyclobutyl]methyl]spiro[2,3-dihydro-1H-naphthalene-4,3′-2,4-dihydro-1,5-benzoxazepine]-7′-yl]-sulfanylidene]-2-methyl-2-prop-2-enoxypropanamide (Intermediate 2) and propionyl chloride in Step 1.

P1 was assigned to Example 43 or Example 44. LC-MS calc. for C₃₅H₄₅ClN₃O₆S [M+H]⁺: m/z=670.3/672.3; Found: 670.1/672.1. HPLC: C18 column (4.6×100 mm, 5 μm); flow rate=1 mL/min; mobile phase: MeCN/H₂O (with 0.1% TFA) 50% to 95% 5 min, 95% 5 min; λ=220 nm. tR=7.437. 1H NMR (300 MHz, CDCl₃) δ 7.67 (d, J=8.5 Hz, 1H), 7.50 (dd, J=8.4, 2.2 Hz, 1H), 7.19 (dd, J=8.5, 2.3 Hz, 1H), 7.09 (d, J=2.3 Hz, 1H), 7.01 (d, J=8.4 Hz, 1H), 6.82 (d, J=2.3 Hz, 1H), 6.21 (ddd, J=15.9, 7.5, 3.1 Hz, 1H), 5.54 (dd, J=16.1, 8.3 Hz, 1H), 4.32-4.22 (m, 1H), 4.19-4.02 (m, 2H), 3.83-3.67 (m, 2H), 3.53 (t, J=8.6 Hz, 1H), 3.41 (dd, J=15.1, 3.9 Hz, 2H), 3.32 (s, 3H), 3.03 (dd, J=15.1, 11.1 Hz, 1H), 2.79 (dd, J=10.1, 5.2 Hz, 2H), 2.48 (qd, J=7.6, 1.7 Hz, 3H), 2.01 (ddt, J=17.6, 12.8, 7.5 Hz, 7H), 1.74-1.62 (m, 2H), 1.52 (s, 3H), 1.39 (s, 3H), 1.21 (t, J=7.5 Hz, 3H).

P2 was assigned to Example 44 or Example 43. LC-MS calc. for C₃₅H₄₅ClN₃O₆S [M+H]⁺: m/z=670.3/672.3; Found: 670.2/672.2. HPLC: C18 column (4.6×100 mm, 5 μm); flow rate=1 mL/min; mobile phase: MeCN/H₂O (with 0.1% TFA) 50% to 95% 5 min, 95% 5 min; λ=220 nm, tR=8.357. 1H NMR (300 MHz, CDCl₃) δ 7.69 (d, J=8.5 Hz, 1H), 7.55 (dd, J=8.4, 2.2 Hz, 1H), 7.19 (dd, J=8.5, 2.4 Hz, 1H), 7.09 (d, J=2.4 Hz, 1H), 7.03-6.93 (m, 2H), 5.91-5.72 (m, 2H), 4.21-4.03 (m, 4H), 3.83-3.66 (m, 2H), 3.56 (dd, J=7.8, 4.6 Hz, 1H), 3.36-3.24 (m, 4H), 3.03 (dd, J=15.1, 10.7 Hz, 1H), 2.79 (t, J=5.2 Hz, 2H), 2.48 (q, J=7.5 Hz, 3H), 2.36-2.23 (m, 1H), 2.07-1.77 (m, 6H), 1.74-1.58 (m, 2H), 1.44 (s, 3H), 1.40 (s, 3H), 1.18 (t, J=7.5 Hz, 3H).

### Example 45 and Example 46 N-[(3R,6R,7R,8E,15R,22S)-7′-Chloro-7-methoxy-12,12-dimethyl-13,15-dioxospiro[11,20-dioxa-15-thia-1,14-diazatetracyclo[14.7.2.03,6.019,24]pentacosa-8,14,16,18,24-pentaene-22,4′-2,3-dihydro-1H-naphthalene]-15-yl]-2-methylpropanamide and N-[(3R,6R,7R,8E,15S,22S)-7′-chloro-7-methoxy-12,12-dimethyl-13,15-dioxospiro[11,20-dioxa-15-thia-1,14-diazatetracyclo[14.7.2.03,6.019,24]pentacosa-8,14,16,18,24-pentaene-22,4′-2,3-dihydro-1H-naphthalene]-15-yl]-2-methylpropanamide

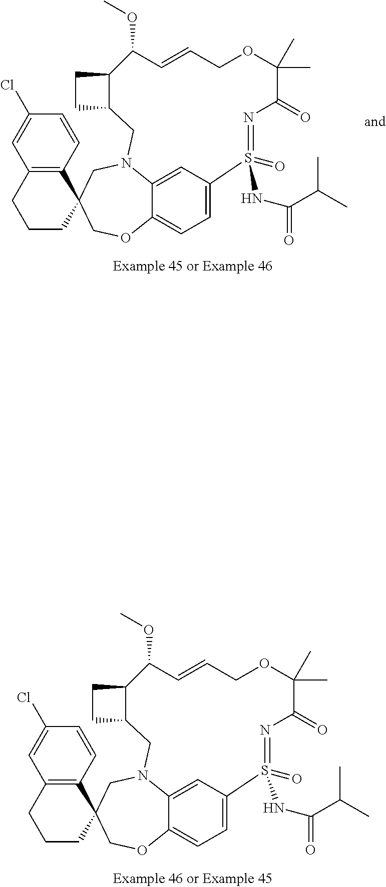 [SMILES: CO[C@@H]1/C=C/COC(C)(C)C(=O)N=[S@@](=O)(NC(=O)C(C)C)c2ccc3c(c2)N(C[C@@H]2CC[C@H]21)C[C@@]1(CCCc2cc(Cl)ccc21)CO3 ; CO[C@@H]1/C=C/COC(C)(C)C(=O)N=[S@](=O)(NC(=O)C(C)C)c2ccc3c(c2)N(C[C@@H]2CC[C@H]21)C[C@@]1(CCCc2cc(Cl)ccc21)CO3]

This compound was prepared using procedures analogous to those described for Example 1 and 2 Step 1-2 using N-[amino-oxo-[(4S)-7-chloro-5′-[[(1S,2S)-2-[(1R)-1-methoxyprop-2-enyl]cyclobutyl]methyl]spiro[2,3-dihydro-1H-naphthalene-4,3′-2,4-dihydro-1,5-benzoxazepine]-7′-yl]-sulfanylidene]-2-methyl-2-prop-2-enoxypropanamide (Intermediate 2) and isobutyryl chloride in Step 1.

P1 was assigned to Example 45 or Example 46. LC-MS calc. for C₃₆H₄₇ClN₃O₆S [M+H]⁺: m/z=684.3/686.3; Found: 684.2/686.5. HPLC: C18 column (4.6×100 mm, 5 μm); flow rate=1 mL/min; mobile phase: MeCN/H₂O (with 0.1% TFA) 50% to 95% 5 min, 95% 5 min; λ=220 nm. tR=7.964. ¹H NMR (300 MHz, CDCl₃) δ 7.68 (d, J=8.5 Hz, 1H), 7.51 (dd, J=8.4, 2.2 Hz, 1H), 7.19 (dd, J=8.5, 2.3 Hz, 1H), 7.09 (d, J=2.3 Hz, 1H), 7.01 (d, J=8.4 Hz, 1H), 6.77 (d, J=2.3 Hz, 1H), 6.21 (ddd, J=15.9, 7.7, 3.0 Hz, 1H), 5.50 (dd, J=16.0, 8.3 Hz, 1H), 4.34-4.22 (m, 1H), 4.12 (q, J=12.1 Hz, 2H), 3.86-3.65 (m, 2H), 3.53 (t, J=8.8 Hz, 1H), 3.39 (dd, J=14.9, 4.7 Hz, 2H), 3.32 (s, 3H), 3.02 (dd, J=15.1, 11.1 Hz, 1H), 2.79 (dd, J=10.0, 5.2 Hz, 2H), 2.64 (p, J=6.9 Hz, 1H), 2.41 (d, J=8.6 Hz, 1H), 2.18-1.77 (m, 7H), 1.75-1.61 (m, 2H), 1.52 (s, 3H), 1.39 (s, 3H), 1.23 (dd, J=6.9, 6.0 Hz, 6H).

P2 was assigned to Example 46 or Example 45. LC-MS calc. for C₃₆H₄₇ClN₃O₆S [M+H]⁺: m/z=684.2/686.4; Found: 684.2/686.5. HPLC: C18 column (4.6×100 mm, 5 μm); flow rate=1 mL/min; mobile phase: MeCN/H₂O (with 0.1% TFA) 50% to 95% 5 min, 95% 5 min; λ=220 nm. tR=8.943. ¹H NMR (300 MHz, CDCl₃) δ 7.69 (d, J=8.5 Hz, 1H), 7.55 (dd, J=8.4, 2.2 Hz, 1H), 7.19 (dd, J=8.4, 2.3 Hz, 1H), 7.09 (d, J=2.3 Hz, 1H), 7.00 (d, J=8.4 Hz, 1H), 6.94 (d, J=2.3 Hz, 1H), 5.87-5.70 (m, 2H), 4.12 (d, J=14.0 Hz, 4H), 3.79 (d, J=14.5 Hz, 1H), 3.73-3.67 (m, 1H), 3.53 (dd, J=7.5, 4.5 Hz, 1H), 3.30 (s, 3H), 3.04 (dd, J=15.2, 10.6 Hz, 1H), 2.79 (t, J=5.2 Hz, 2H), 2.69-2.48 (m, 2H), 2.37-2.23 (m, 1H), 1.92 (dtd, J=43.4, 17.6, 15.8, 9.2 Hz, 7H), 1.73-1.54 (m, 2H), 1.44 (s, 3H), 1.39 (s, 3H), 1.22 (dd, J=6.9, 3.4 Hz, 6H).

### Example 47 and Example 48 N-[(3R,6R,7R,8E,15R,22S)-7′-Chloro-7-methoxy-12,12-dimethyl-13,15-dioxospiro[11,20-dioxa-15-thia-1,14-diazatetracyclo[14.7.2.03,6.019,24]pentacosa-8,14,16,18,24-pentaene-22,4′-2,3-dihydro-1H-naphthalene]-15-yl]-1,5-dimethylpyrazole-4-carboxamide and N-[(3R,6R,7R,8E,15S,22S)-7′-chloro-7-methoxy-12,12-dimethyl-13,15-dioxospiro[11,20-dioxa-15-thia-1,14-diazatetracyclo[14.7.2.03,6.019,24]pentacosa-8,14,16,18,24-pentaene-22,4′-2,3-dihydro-1H-naphthalene]-15-yl]-1,5-dimethylpyrazole-4-carboxamide

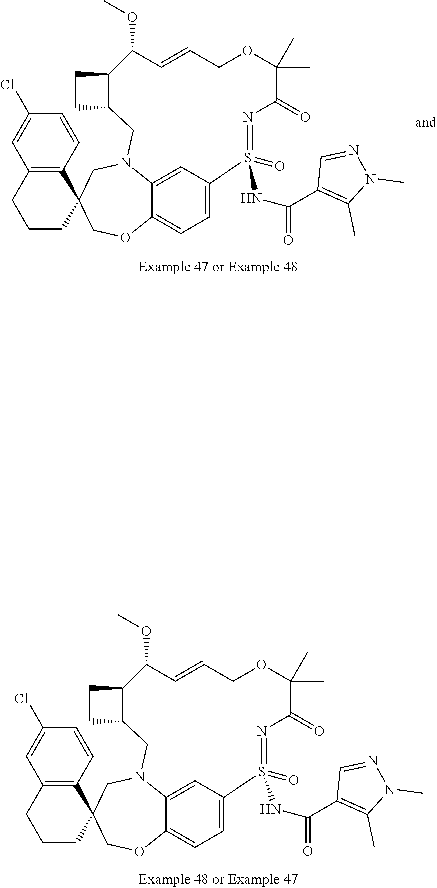 [SMILES: CO[C@@H]1/C=C/COC(C)(C)C(=O)N=[S@@](=O)(NC(=O)c2cnn(C)c2C)c2ccc3c(c2)N(C[C@@H]2CC[C@H]21)C[C@@]1(CCCc2cc(Cl)ccc21)CO3 ; CO[C@@H]1/C=C/COC(C)(C)C(=O)N=[S@](=O)(NC(=O)c2cnn(C)c2C)c2ccc3c(c2)N(C[C@@H]2CC[C@H]21)C[C@@]1(CCCc2cc(Cl)ccc21)CO3]

This compound was prepared using procedures analogous to those described for Example 1 and 2 Step 1-2 using N-[amino-oxo-[(4S)-7-chloro-5′-[[(1S,2S)-2-[(1R)-1-methoxyprop-2-enyl]cyclobutyl]methyl]spiro[2,3-dihydro-1H-naphthalene-4,3′-2,4-dihydro-1,5-benzoxazepine]-7′-yl]-sulfanylidene]-2-methyl-2-prop-2-enoxypropanamide (Intermediate 2) and 1,5-dimethylpyrazole-4-carbonyl chloride in Step 1.

P1 was assigned to Example 47 or Example 48. LC-MS calc. for C₃₈H₄₇ClN₅O₆S [M+H]⁺: m/z=736.3/738.3; Found: 736.3/738.5. HPLC: C18 column (4.6×100 mm, 5 μm); flow rate=1 mL/min; mobile phase: MeCN/H₂O (with 0.1% TFA) 50% to 95% 5 min, 95% 5 min; λ=220 nm. tR=7.514. ¹H NMR (300 MHz, CDCl₃) δ 7.95 (s, 1H), 7.67 (d, J=8.5 Hz, 1H), 7.53 (dd, J=8.5, 2.3 Hz, 1H), 7.18 (dd, J=8.5, 2.3 Hz, 1H), 7.09 (d, J=2.3 Hz, 1H), 7.01 (dd, J=8.4, 3.2 Hz, 1H), 6.95 (d, J=2.3 Hz, 1H), 6.42-6.21 (m, 1H), 5.57 (dd, J=16.0, 8.0 Hz, 1H), 4.29 (dd, J=13.5, 7.6 Hz, 1H), 4.12 (q, J=12.1 Hz, 2H), 3.78 (d, J=22.6 Hz, 5H), 3.42 (dd, J=15.0, 11.6 Hz, 3H), 3.28 (d, J=8.2 Hz, 3H), 3.00 (dd, J=15.1, 11.0 Hz, 1H), 2.78 (d, J=10.4 Hz, 2H), 2.61 (s, 3H), 2.41 (d, J=8.6 Hz, 1H), 2.25 (t, J=7.6 Hz, 1H), 1.98-1.80 (m, 6H), 1.68-1.59 (m, 2H), 1.54 (s, 3H), 1.42 (d, J=5.0 Hz, 3H).

P2 was assigned to Example 48 or Example 47. LC-MS calc. for C₃₈H₄₇ClN₅O₆S [M+H]⁺: m/z=736.3/738.3; Found: 736.3/738.5. HPLC: C18 column (4.6×100 mm, 5 μm); flow rate=1 mL/min; mobile phase: MeCN/H₂O (with 0.1% TFA) 50% to 95% 5 min, 95% 5 min; λ=220 nm. tR=7.846. ¹H NMR (300 MHz, CDCl₃) δ 7.99 (s, 1H), 7.70 (d, J=8.5 Hz, 1H), 7.61 (dd, J=8.4, 2.2 Hz, 1H), 7.20 (dd, J=8.5, 2.4 Hz, 1H), 7.08 (dd, J=4.4, 2.3 Hz, 2H), 7.02 (d, J=8.4 Hz, 1H), 5.83 (d, J=4.3 Hz, 2H), 4.14 (d, J=24.5 Hz, 4H), 3.82 (s, 3H), 3.78-3.64 (m, 2H), 3.52 (q, J=4.1 Hz, 1H), 3.35-3.26 (m, 1H), 3.21 (s, 3H), 3.00 (dd, J=15.2, 10.5 Hz, 1H), 2.77 (q, J=5.5 Hz, 2H), 2.57 (s, 3H), 2.52 (s, 1H), 2.33-2.22 (m, 1H), 1.89 (ddq, J=43.6, 17.5, 9.1, 8.1 Hz, 6H), 1.63 (q, J=9.1, 8.5 Hz, 2H), 1.46 (s, 3H), 1.41 (s, 3H).

### Example 49 and Example 50 N-[(3R,6R,7S,8E,15R,22S)-7′-Chloro-7-hydroxy-12,12-dimethyl-13,15-dioxospiro[11,20-dioxa-15-thia-1,14-diazatetracyclo[14.7.2.03,6.019,24]pentacosa-8,14,16,18,24-pentaene-22,4′-2,3-dihydro-1H-naphthalene]-15-yl]-1,3-dimethylpyrazole-4-carboxamide and N-[(3R,6R,7S,8E,15S,22S)-7′-Chloro-7-hydroxy-12,12-dimethyl-13,15-dioxospiro[11,20-dioxa-15-thia-1,14-diazatetracyclo[14.7.2.03,6.019,24]pentacosa-8,14,16,18,24-pentaene-22,4′-2,3-dihydro-1H-naphthalene]-15-yl]-1,3-dimethylpyrazole-4-carboxamide

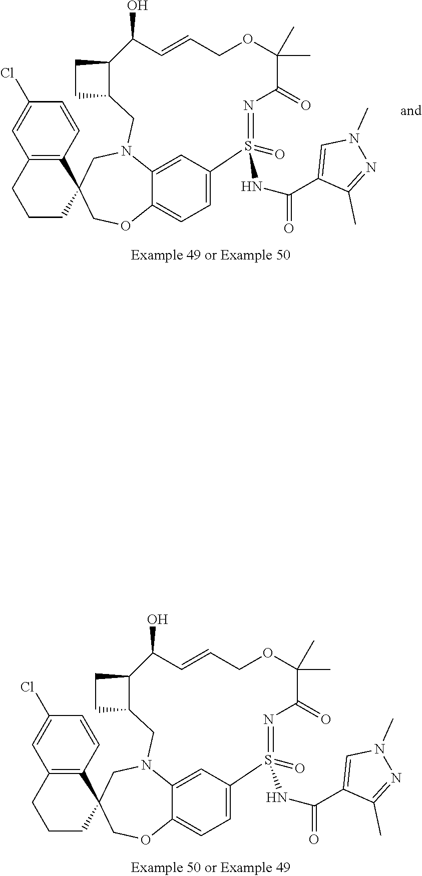 [SMILES: Cc1nn(C)cc1C(=O)N[S@]1(=O)=NC(=O)C(C)(C)OC/C=C/[C@H](O)[C@@H]2CC[C@H]2CN2C[C@@]3(CCCc4cc(Cl)ccc43)COc3ccc1cc32 ; Cc1nn(C)cc1C(=O)N[S@@]1(=O)=NC(=O)C(C)(C)OC/C=C/[C@H](O)[C@@H]2CC[C@H]2CN2C[C@@]3(CCCc4cc(Cl)ccc43)COc3ccc1cc32]

### Step 1: (4S)-7-chloro-5′-[[(1R,2R)-2-[(1S)-1-hydroxyprop-2-enyl]cyclobutyl]methyl]spiro[2,3-dihydro-1H-naphthalene-4,3′-2,4-dihydro-1,5-benzoxazepine]-7′-sulfonamide

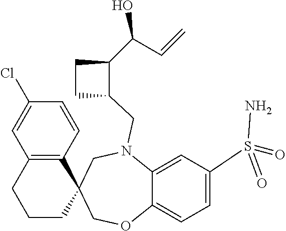 [SMILES: C=C[C@H](O)[C@@H]1CC[C@H]1CN1C[C@@]2(CCCc3cc(Cl)ccc32)COc2ccc(S(N)(=O)=O)cc21]

To a solution of (3S)-6′-chloro-N,N-bis[(4-methoxyphenyl)methyl]-5-[[(1R,2R)-2-[(1S)-1-hydroxyallyl]cyclobutyl]methyl]spiro[2,4-dihydro-1,5-benzoxazepine-3,1′-tetralin]-7-sulfonamide (24.3 g, 32.7 mmol, P1, Intermediate 1 Step 14) and anisole (23.7 mL, 218.1 mmol) in DCM (240 mL) was added 2,2,2-trifluoroacetic acid (243.0 mL). The mixture was stirred overnight. The reaction was monitored by LC-MS. All starting material was consumed from LC-MS. Solvents were removed under reduced pressure. The residue was diluted with DCM (200 mL). The mixture was washed with saturated aqueous NaHCO₃ solution (200 mL×3) and brine, dried over Na₂SO₄, filtered and concentrated under reduced pressure. The residue was purified by flash chromatography on a silica gel column with EA/heptane (5%-70%) to afford (4S)-7-chloro-5′-[[(1R,2R)-2-[(1S)-1-hydroxyprop-2-enyl]cyclobutyl]methyl]spiro[2,3-dihydro-1H-naphthalene-4,3′-2,4-dihydro-1,5-benzoxazepine]-7′-sulfonamide (15.7 g, 31.21 mmol, 95.47% yield) as a pale white solid. LCMS calc. for C₂₆H₃₂ClN₂O₄S [M+H]r: m/z=503.17/505.17; Found 503.5/505.5; ¹H NMR (300 MHz, CDCl₃) δ 7.74 (d, J=8.5 Hz, 1H), 7.55 (d, J=1.8 Hz, 1H), 7.21 (dd, J=11.4, 4.2 Hz, 2H), 7.12-7.08 (m, 2H), 6.97-6.94 (m, 1H), 6.85 (d, J=8.6 Hz, 1H), 5.90-5.76 (m, 1H), 5.25 (d, J=17.2 Hz, 1H), 5.16-5.08 (m, 1H), 4.11 (s, 2H), 3.88 (d, J=5.1 Hz, 1H), 3.81 (s, 2H), 3.27 (d, J=14.3 Hz, 1H), 3.14 (m, 1H), 2.84-2.75 (m, 2H), 2.51 (dd, J=16.9, 8.5 Hz, 1H), 2.08 (m, 3H), 1.90 (dd, J=15.8, 5.6 Hz, 2H), 1.63 (m, 3H), 1.45 (t, J=12.1 Hz, 1H).

### Step 2: (4S)—N-[tert-butyl(dimethyl)silyl]-7-chloro-5′-[[(1R,2R)-2-[(S)-1-hydroxyprop-2-enyl]cyclobutyl]methyl]spiro[2,3-dihydro-1H-naphthalene-4,3′-2,4-dihydro-1,5-benzoxazepine]-7′-sulfonamide

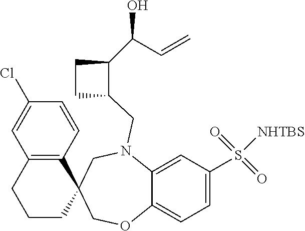

To a stirred solution of (4S)-7-chloro-5′-[[(1R,2R)-2-[(1S)-1-hydroxyprop-2-enyl]cyclobutyl]methyl]spiro[2,3-dihydro-1H-naphthalene-4,3′-2,4-dihydro-1,5-benzoxazepine]-7′-sulfonamide (200.0 mg, 0.40 mmol) in THF (4 mL) was added tert-butyl dimethylchlorosilane (239.69 mg, 1.59 mmol) followed by triethylamine (0.44 mL, 3.18 mmol). The resulting mixture was stirred at r.t. for 4 days. LC-MS indicated the consumption of starting material and the formation of desired product. The solution was concentrated under reduced pressure. The residue was purified by flash column chromatography on a silica gel column with EtOAc/heptane (5% to 60%) to afford (4S)—N-[tert-butyl(dimethyl)silyl]-7-chloro-5′-[[(1R,2R)-2-[(1S)-1-hydroxyprop-2-enyl]cyclobutyl]methyl]spiro[2,3-dihydro-1H-naphthalene-4,3′-2,4-dihydro-1,5-benzoxazepine]-7′-sulfonamide (0.217 g, 88.4% yield) as a white solid. LC-MS calc. for C₃₃H₄₈ClN₂O₄SSi [M+H]⁺: m/z=617.3/619.3; Found: 617.2/619.6.

### Step 3: (4S)—N-[tert-butyl(dimethyl)silyl]-7-chloro-5′-[[(1R,2R)-2-[(S)-1-(oxan-2-yloxy)prop-2-enyl]cyclobutyl]methyl]spiro[2,3-dihydro-1H-naphthalene-4,3′-2,4-dihydro-1,5-benzoxazepine]-7′-sulfonamide

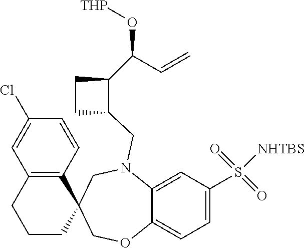

To a solution of (4S)—N-[tert-butyl(dimethyl)silyl]-7-chloro-5′-[[(1R,2R)-2-[(1S)-1-hydroxyprop-2-enyl]cyclobutyl]methyl]spiro[2,3-dihydro-1H-naphthalene-4,3′-2,4-dihydro-1,5-benzoxazepine]-7′-sulfonamide (217.0 mg, 0.35 mmol) in DCM (5 mL) was added 3,4-dihydro-2H-pyran (0.04 mL, 0.39 mmol) followed by p-toluenesulfonic acid monohydrate (6.69 mg, 0.04 mmol). The mixture was stirred at r.t. for 2 h. LC-MS showed the consumption of starting material and the formation of desired product. The solution was concentrated under reduced pressure. The residue was purified by flash chromatography on a silica gel column using 5% to 80% EtOAc/heptane (with 0.1% trimethylamine). The desired fractions were collected to afford (4S)—N-[tert-butyl(dimethyl)silyl]-7-chloro-5′-[[(1R,2R)-2-[(1S)-1-(oxan-2-yloxy)prop-2-enyl]cyclobutyl]methyl]spiro[2,3-dihydro-1H-naphthalene-4,3′-2,4-dihydro-1,5-benzoxazepine]-7′-sulfonamide (130 mg, 52.7% yield) as a light brown solid. LC-MS calc. for C₃₇H₅₄ClN₂O₅SSi [M+H]⁺: m/z=701.3/703.3; Found: 701.4/703.5.

### Step 4: (4S)-7′-[S-amino-N-[tert-butyl(dimethyl)silyl]sulfonimidoyl]-7-chloro-5′-[[(1R,2R)-2-[(S)-1-(oxan-2-yloxy)prop-2-enyl]cyclobutyl]methyl]spiro[2,3-dihydro-1H-naphthalene-4,3′-2,4-dihydro-1,5-benzoxazepine]

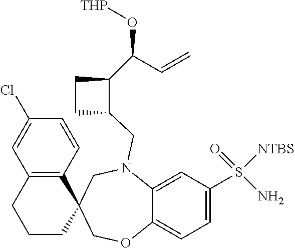

To a solution of (4S)—N-[tert-butyl(dimethyl)silyl]-7-chloro-5′-[[(1R,2R)-2-[(1S)-1-(oxan-2-yloxy)prop-2-enyl]cyclobutyl]methyl]spiro[2,3-dihydro-1H-naphthalene-4,3′-2,4-dihydro-1,5-benzoxazepine]-7′-sulfonamide (1.14 g, 1.63 mmol) in DCM (3 mL) was added hexachloroethane (0.58 g, 2.44 mmol), triphenylphosphine (0.64 g, 2.44 mmol) and triethylamine (0.9 mL, 6.5 mmol). The resulting mixture was stirred at 35° C. for 2 h. The reaction was then bubbled with ammonia gas for 5 min., and stirred at r.t. for an additional 30 min. with a sealed cap. LC-MS indicated the consumption of starting material and the formation of desired product. The reaction mixture was filtered. The filtrate was concentrated under reduced pressure. The residue was purified by flash chromatography on a silica gel column with EtOAc/heptane (5% to 100% with 0.1% triethylamine) to afford (4S)-7′-[S-amino-N-[tert-butyl(dimethyl)silyl]sulfonimidoyl]-7-chloro-5′-[[(1R,2R)-2-[(1S)-1-(oxan-2-yloxy)prop-2-enyl]cyclobutyl]methyl]spiro[2,3-dihydro-1H-naphthalene-4,3′-2,4-dihydro-1,5-benzoxazepine] (1.04 g, 91.3% yield) as a light brown solid. LC-MS calc. for C₃₇H₅₅ClN₃O₄SSi [M+H]+. m/z=700.3/702.3; Found: 700.1/702.3.

### Step 5: N-[amino-oxo-[(4S)-7-chloro-5′-[[(1R,2R)-2-[(1S)-1-methoxyprop-2-enyl]cyclobutyl]methyl]spiro[2,3-dihydro-1H-naphthalene-4,3′-2,4-dihydro-1,5-benzoxazepine]-7′-yl]-sulfanylidene]-2-methyl-2-prop-2-enoxypropanamide

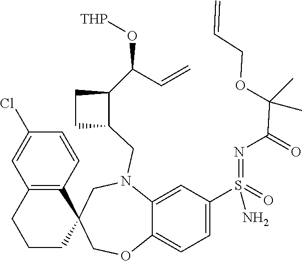

To a solution of (4S)-7′-[S-amino-N-[tert-butyl(dimethyl)silyl]sulfonimidoyl]-7-chloro-5′-[[(1R,2R)-2-[(1S)-1-(oxan-2-yloxy)prop-2-enyl]cyclobutyl]methyl]spiro[2,3-dihydro-1H-naphthalene-4,3′-2,4-dihydro-1,5-benzoxazepine] (1.04 g, 1.48 mmol) in DCM (2 mL) was added pyridazine (0.16 mL, 2.23 mmol) followed by 2-methyl-2-prop-2-enoxypropanoyl chloride (362.16 mg, 2.23 mmol). The resulting mixture was stirred at r.t. for 2 h. LC-MS indicated the consumption of starting material and the formation of desired product. The crude solution was concentrated under reduced pressure while the temperature of the water bath of the rotary evaporator remained below 20° C. The crude product was purified by flash chromatography on a silica gel column with EtOAc/heptane (2% to 80% with 0.1% triethylamine) to afford N-[amino-[(4S)-7-chloro-5′-[[(1R,2R)-2-[(1S)-1-(oxan-2-yloxy)prop-2-enyl]cyclobutyl]methyl]spiro[2,3-dihydro-1H-naphthalene-4,3′-2,4-dihydro-1,5-benzoxazepine]-7′-yl]-oxo-sulfanylidene]-2-methyl-2-prop-2-enoxypropanamide (580 mg, 54.8% yield) as a light brown solid. LC-MS calc. for C₃₄H₄₄ClN₃O₅S [M+H]⁺: m/z=712.3/714.3; Found: 712.5/714.3.

### Step 6: N—[S-[(4S)-7-chloro-5′-[[(1R,2R)-2-[(1S)-1-(oxan-2-yloxy)prop-2-enyl]cyclobutyl]methyl]spiro[2,3-dihydro-1H-naphthalene-4,3′-2,4-dihydro-1,5-benzoxazepine]-7′-yl]-N-(2-methyl-2-prop-2-enoxypropanoyl)sulfonimidoyl]-1,3-dimethylpyrazole-4-carboxamide

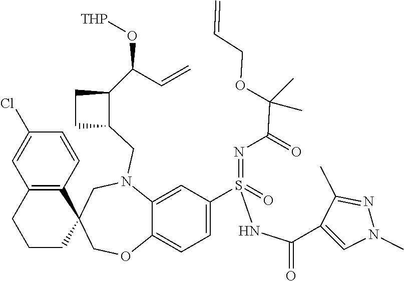

To a solution of N-[amino-[(4S)-7-chloro-5′-[[(1R,2R)-2-[(1S)-1-(oxan-2-yloxy)prop-2-enyl]cyclobutyl]methyl]spiro[2,3-dihydro-1H-naphthalene-4,3′-2,4-dihydro-1,5-benzoxazepine]-7′-yl]-oxo-sulfanylidene]-2-methyl-2-prop-2-enoxypropanamide (220.0 mg, 0.31 mmol) in DCM (1 mL) was added triethylamine (0.09 mL, 0.62 mmol) followed by 1,3-dimethylpyrazole-4-carbonyl chloride (53.88 mg, 0.34 mmol). The resulting mixture was stirred at r.t. for 2 h. LC-MS indicated the consumption of starting material and the formation of desired product. The crude product was concentrated under reduced pressure. The residue was purified by flash chromatography on a silica gel column with EtOAc/heptane (0% to 50% with 0.1% triethylamine) to afford N—[S-[(4S)-7-chloro-5′-[[(1R,2R)-2-[(1S)-1-(oxan-2-yloxy)prop-2-enyl]cyclobutyl]methyl]spiro[2,3-dihydro-1H-naphthalene-4,3′-2,4-dihydro-1,5-benzoxazepine]-7′-yl]-N-(2-methyl-2-prop-2-enoxypropanoyl)sulfonimidoyl]-1,3-dimethylpyrazole-4-carboxamide (190 mg, 73.7% yield) as a light yellow oil. LC-MS calc. for C₄₄H₅₇ClN₅O₇S [M+H]⁺: m/z=834.4/836.4; Found: 834.1/836.5.

### Step 7: N-[(3R,6R,7S,8E,22S)-7′-chloro-12,12-dimethyl-7-(oxan-2-yloxy)-13,15-dioxospiro[11,20-dioxa-15-thia-1,14-diazatetracyclo[14.7.2.03,6.019,24]pentacosa-8,14,16,18,24-pentaene-22,4′-2,3-dihydro-1H-naphthalene]-15-yl]-1,3-dimethylpyrazole-4-carboxamide

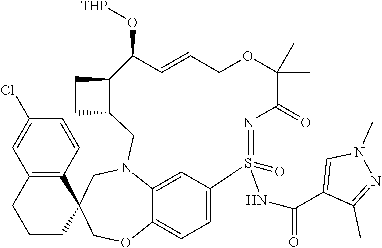

To a stirred solution of N—[S-[(4S)-7-chloro-5′-[[(1R,2R)-2-[(1S)-1-(oxan-2-yloxy)prop-2-enyl]cyclobutyl]methyl]spiro[2,3-dihydro-1H-naphthalene-4,3′-2,4-dihydro-1,5-benzoxazepine]-7′-yl]-N-(2-methyl-2-prop-2-enoxypropanoyl)sulfonimidoyl]-1,3-dimethylpyrazole-4-carboxamide (190.0 mg, 0.23 mmol) in DCE (220 mL) was bubbled with nitrogen for 5 min. Then Hoveyda-Grubbs II catalyst (28.54 mg, 0.05 mmol) was added under nitrogen. The resulting mixture further bubbled with nitrogen for 5 min. The solution was stirred at 70° C. under nitrogen overnight. LC-MS indicated the consumption of starting material and the formation of desired product. The solution was cooled to r.t. and stirred under air for 30 min. to deactivate the catalyst. The solution was concentrated under reduced pressure and the crude product N-[(3R,6R,7S,8E,22S)-7′-chloro-12,12-dimethyl-7-(oxan-2-yloxy)-13,15-dioxospiro[11,20-dioxa-15-thia-1,14-diazatetracyclo[14.7.2.03,6.019,24]pentacosa-8,14,16,18,24-pentaene-22,4′-2,3-dihydro-1H-naphthalene]-15-yl]-1,3-dimethylpyrazole-4-carboxamide (150 mg, 81.7% yield) was directly used in next step without further purification. LC-MS calc. for C₄₂H₅₃ClN₅O₇S [M+H]⁺: m/z=806.3/808.3; Found: 806.5/808.2.

### Step 8: N-[(3R,6R,7S,8E,15R,22S)-7′-chloro-7-hydroxy-12,12-dimethyl-13,15-dioxospiro[11,20-dioxa-15-thia-1,14-diazatetracyclo[14.7.2.03,6.019,24]pentacosa-8,14,16,18,24-pentaene-22,4′-2,3-dihydro-1H-naphthalene]-15-yl]-1,3-dimethylpyrazole-4-carboxamide and N-[(3R,6R,7S,8E,15S,22S)-7′-chloro-7-hydroxy-12,12-dimethyl-13,15-dioxospiro[11,20-dioxa-15-thia-1,14-diazatetracyclo[14.7.2.03,6.019,24]pentacosa-8,14,16,18,24-pentaene-22,4′-2,3-dihydro-1H-naphthalene]-15-yl]-1,3-dimethylpyrazole-4-carboxamide

To a stirred solution of N-[(3R,6R,7S,8E,22S)-7′-chloro-12,12-dimethyl-7-(oxan-2-yloxy)-13,15-dioxospiro[11,20-dioxa-15-thia-1,14-diazatetracyclo[14.7.2.03,6.019,24]pentacosa-8,14,16,18,24-pentaene-22,4′-2,3-dihydro-1H-naphthalene]-15-yl]-1,3-dimethylpyrazole-4-carboxamide (130.0 mg, 0.16 mmol) in DCM (3 mL) was added TFA (0.13 mL, 1.7 mmol). The resulting mixture was stirred at r.t. for 1 h. LC-MS indicated the consumption of starting material and the formation of desired product. Water (1 mL) was added to the reaction mixture. The solution was concentrated under reduced pressure. The residue was purified by prep-HPLC on C18 column (30×250 mm, 10 μm) with MeCN/H₂O (20% to 100% w/0.1% TFA) to afford P1 (the earlier eluted product) (10 mg), and P2 (the latter eluted product) (10 mg, 11.8% yield), and a mixture of P1 and P2 (70 mg). LC-MS calc. for C₃₇H₄₅ClN₅O₆S [M+H]⁺: m/z=722.3/724.3; Found: 722.3/724.6.

P1 was assigned to Example 49 or Example 50: ¹H NMR (300 MHz, CDCl₃) δ 7.90 (s, 1H), 7.68 (d, J=8.5 Hz, 1H), 7.55 (d, J=8.7 Hz, 1H), 7.21 (s, 1H), 7.20-7.17 (m, 1H), 7.10 (s, 1H), 7.01 (d, J=8.4 Hz, 1H), 6.04-5.94 (m, 1H), 5.88-5.79 (m, 1H), 5.31 (br, 1H), 4.22-4.13 (m, 3H), 3.98-3.90 (m, 1H), 3.85 (s, 3H), 3.75 (d, J=14.4 Hz, 1H), 3.50-3.44 (m, 1H), 3.34 (d, J=14.7 Hz, 1H), 3.23-3.13 (m, 1H), 2.80 (d, J=5.1 Hz, 1H), 2.52 (s, 3H), 2.24 (t, J=7.6 Hz, 2H), 2.08-1.97 (m, 3H), 1.68-1.59 (m, 8H), 1.47 (s, 3H), 1.43 (s, 3H).

P2 was assigned to Example 50 or Example 49: ¹H NMR (300 MHz, CDCl₃) δ 7.89 (s, 1H), 7.68 (d, J=8.5 Hz, 1H), 7.43 (dd, J=8.4, 2.2 Hz, 1H), 7.23-7.18 (m, 2H), 7.09 (s, 1H), 7.00 (d, J=8.4 Hz, 1H), 6.31 (dd, J=15.9, 7.1 Hz, 1H), 6.06-5.99 (m, 1H), 5.33 (br, 1H), 4.23 (d, J=12.4 Hz, 1H), 4.10 (d, J=2.9 Hz, 2H), 3.96 (d, J=13.4 Hz, 1H), 3.82 (s, 3H), 3.58 (d, J=14.8 Hz, 1H), 3.42 (d, J=14.7 Hz, 1H), 3.19-3.07 (m, 1H), 2.78 (d, J=11.8 Hz, 2H), 2.49 (s, 3H), 2.25 (t, J=7.7 Hz, 2H), 2.06-1.98 (m, 3H), 1.70-1.60 (m, 8H), 1.51 (s, 3H), 1.43 (s, 3H).

### Example 51 [(3R,6R,7S,8E,22S)-7′-Chloro-15-[(1,3-dimethylpyrazole-4-carbonyl)amino]-12,12-dimethyl-13,15-dioxospiro[11,20-dioxa-15-thia-1,14-diazatetracyclo[14.7.2.03,6.019,24]pentacosa-8,14,16,18,24-pentaene-22,4′-2,3-dihydro-1H-naphthalene]-7-yl] N,N-dimethylcarbamate

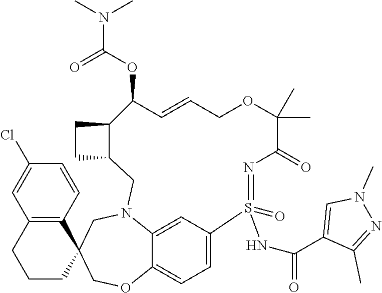 [SMILES: Cc1nn(C)cc1C(=O)NS1(=O)=NC(=O)C(C)(C)OC/C=C/[C@H](OC(=O)N(C)C)[C@@H]2CC[C@H]2CN2C[C@@]3(CCCc4cc(Cl)ccc43)COc3ccc1cc32]

To a mixture of N-[(3R,6R,7S,8E,15R,22S)-7′-chloro-7-hydroxy-12,12-dimethyl-13,15-dioxospiro[11,20-dioxa-15-thia-1,14-diazatetracyclo[14.7.2.03,6.019,24]pentacosa-8,14,16,18,24-pentaene-22,4′-2,3-dihydro-1H-naphthalene]-15-yl]-1,3-dimethylpyrazole-4-carboxamide and N-[(3R,6R,7S,8E,15S,22S)-7′-chloro-7-hydroxy-12,12-dimethyl-13,15-dioxospiro[11,20-dioxa-15-thia-1,14-diazatetracyclo[14.7.2.03,6.019,24]pentacosa-8,14,16,18,24-pentaene-22,4′-2,3-dihydro-1H-naphthalene]-15-yl]-1,3-dimethylpyrazole-4-carboxamide (50.0 mg, 0.07 mmol, Example 49 and 50) in THF (1 mL) was added 1,1′-carbonyldiimidazole (56.12 mg, 0.35 mmol, Example 49). The reaction was stirred at 50° C. overnight. LC-MS indicated the consumption of starting material. Then dimethylamine in THF solution (1.0 mL, 2.0 mmol, 2.0 M) was added. The resulting mixture was stirred at r.t. overnight. LC-MS indicated the consumption of intermediate and the formation of desired product. The crude product was directly purified by prep-HPLC on C18 column (30×250 mm, 10 μm) with MeCN/H₂O (20% to 100% w/0.1% TFA) to afford [(3R,6R,7S,8E,22S)-7′-chloro-15-[(1,3-dimethylpyrazole-4-carbonyl)amino]-12,12-dimethyl-13,15-dioxospiro[11,20-dioxa-15-thia-1,14-diazatetracyclo[14.7.2.03,6.019,24]pentacosa-8,14,16,18,24-pentaene-22,4′-2,3-dihydro-1H-naphthalene]-7-yl] N,N-dimethylcarbamate (9.6 mg, 15.9% yield) as a white solid as a mixture of two inseparable diastereomers. LC-MS calc. for C₄₀H₅₀ClN₆O₇S [M+H]⁺: m/z=793.3/795.3; Found: 793.2/795.7. HPLC: C18 column (4.6×100 mm, 5 μm); flow rate=1 mL/min; mobile phase: 50% MeCN/H₂O (with 0.1% TFA) to 95% 4 min, 95% 6 min; λ=220 nm. tR=7.27 min. and 7.53 min. ¹H NMR (300 MHz, CDCl₃) δ 7.96-7.90 (m, 1H), 7.69-7.64 (m, 1H), 7.60-7.56 (m, 1H), 7.24-7.14 (m, 2H), 7.11-7.07 (m, 1H), 7.05-6.98 (m, 1H), 5.99 (dd, J=15.8, 4.7 Hz, 0.5H), 5.92-5.75 (m, 15H), 5.35 (br, 1H), 4.18-4.03 (m, 3H), 3.90 (s, 15H), 3.86 (s, 1.5H), 3.76-3.71 (m, 1H), 3.59-3.49 (m, 1H), 3.42-3.26 (m, 1H), 3.16-3.13 (m, 1H), 2.94-2.92 (m, 3H), 2.88-2.78 (m, 3H), 2.67-2.60 (m, 2H), 2.54 (s, 1.5H), 2.52 (s, 1.5H), 2.34-2.25 (m, 1H), 2.04-1.89 (m, 3H), 1.87-1.71 (m, 3H), 1.70-1.56 (m, 2H), 1.52 (s, 1.5H), 1.48-1.40 (m, 4H), 1.38 (s, 1.5H), 1.34-1.28 (m, 2H).

### Example 52 N-[(3R,6R,7S,8E,22S)-7′-Chloro-7-hydroxy-12,12-dimethyl-13,15-dioxospiro[11,20-dioxa-15-thia-1,14-diazatetracyclo[14.7.2.03,6.019,24]pentacosa-8,14,16,18,24-pentaene-22,4′-2,3-dihydro-1H-naphthalene]-15-yl]-1-methylpyrazole-4-carboxamide

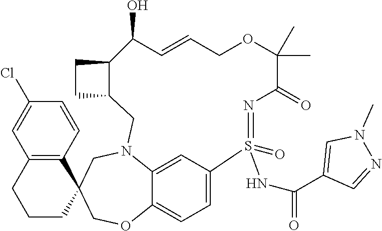 [SMILES: Cn1cc(C(=O)NS2(=O)=NC(=O)C(C)(C)OC/C=C/[C@H](O)[C@@H]3CC[C@H]3CN3C[C@@]4(CCCc5cc(Cl)ccc54)COc4ccc2cc43)cn1]

This compound was prepared using procedures analogous to those described for Example 49 and 50 Step 6-8 using N-[amino-[(4S)-7-chloro-5′-[[(1R,2R)-2-[(1S)-1-(oxan-2-yloxy)prop-2-enyl]cyclobutyl]methyl]spiro[2,3-dihydro-1H-naphthalene-4,3′-2,4-dihydro-1,5-benzoxazepine]-7′-yl]-oxo-sulfanylidene]-2-methyl-2-prop-2-enoxypropanamide (Example 49 Step 5) and 1-methylpyrazole-4-carbonyl chloride in Step 6. LC-MS calc. for C₃₆H₄₃ClN₅O₆S [M+H]⁺: m/z=708.3/710.3; Found: 708.3/710.5. ¹H NMR (300 MHz, CDCl₃) δ 7.80-7.92 (m, 2H), 7.69-7.64 (m, 1H), 7.45-7.41 (m, 1H), 7.34-7.21 (m, 1H), 7.20-7.14 (m, 1H), 7.10-7.06 (m, 1H), 7.01-6.99 (m, 1H), 6.42-6.32 (m, 1H), 6.05-5.92 (m, 1H), 4.66-4.60 (m, 1H), 4.27-4.04 (m, 4H), 4.02-3.91 (m, 1H), 3.92-3.89 (m, 3H), 3.79-3.75 (m, 1H), 3.62-3.55 (m, 2H), 3.43-3.40 (m, 1H), 3.20-3.06 (m, 1H), 2.85-2.76 (m, 3H), 2.26-2.19 (m, 3H), 2.04-1.95 (m, 4H), 1.65-1.60 (m, 2H), 1.53-1.38 (m, 6H).

### Example 53 [(3R,6R,7S,8E,22S)-7′-Chloro-12,12-dimethyl-15-[(1-methylpyrazole-4-carbonyl)amino]-13,15-dioxospiro[11,20-dioxa-15-thia-1,14-diazatetracyclo[14.7.2.03,6.019,24]pentacosa-8,14,16,18,24-pentaene-22,4′-2,3-dihydro-1H-naphthalene]-7-yl] N,N-dimethylcarbamate

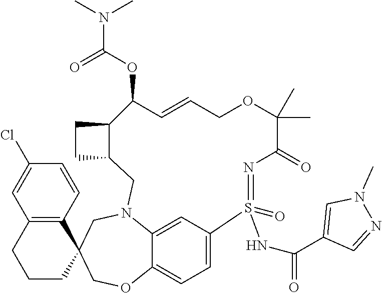 [SMILES: CN(C)C(=O)O[C@H]1/C=C/COC(C)(C)C(=O)N=S(=O)(NC(=O)c2cnn(C)c2)c2ccc3c(c2)N(C[C@@H]2CC[C@H]21)C[C@@]1(CCCc2cc(Cl)ccc21)CO3]

This compound was prepared using procedures analogous to those described for Example 51 using N-[(3R,6R,7S,8E,22S)-7′-chloro-7-hydroxy-12,12-dimethyl-13,15-dioxospiro[11,20-dioxa-15-thia-1,14-diazatetracyclo[14.7.2.03,6.019,24]pentacosa-8,14,16,18,24-pentaene-22,4′-2,3-dihydro-1H-naphthalene]-15-yl]-1-methylpyrazole-4-carboxamide (Example 52). LC-MS calc. for C₃₉H₄₈ClN₆O₇S [M+H]⁺: m/z=779.3/781.3; Found: 779.2/781.0. HPLC: C18 column (4.6×100 mm, 5 μm); flow rate=1 mL/min; mobile phase: 50% MeCN/H₂O (with 0.1% TFA) to 95% 4 min, 95% 6 min; λ=220 nm. tR1=7.64 min. ¹H NMR (300 MHz, CDCl₃) δ 7.95-7.90 (m, 2H), 7.67-7.62 (m, 1H), 7.56-7.51 (m, 1H), 7.19-7.15 (m, 1H), 7.09-7.06 (m, 1H), 7.02-6.98 (m, 1H), 6.94-6.89 (m, 1H), 5.89-5.74 (m, 2H), 5.24 (br, 1H), 4.18-4.10 (m, 3H), 3.95-3.88 (m, 3H), 3.68-3.65 (m, 1H), 3.53-3.47 (m, 1H), 3.44-3.42 (m, 1H), 3.27-3.21 (m, 1H), 2.88-2.86 (m, 3H), 2.79-2.75 (m, 2H), 2.74-2.67 (m, 3H), 2.38-2.32 (m, 1H), 2.28-2.23 (m, 3H), 1.96-1.76 (m, 8H), 1.50 (s, 1.5H), 1.44-1.42 (m, 3H), 1.36 (s, 1.5H).

### Example 54 N-[(3R,6R,7S,8E,22S)-7′-Chloro-12,12-dimethyl-13,15-dioxo-7-(2-pyrrolidin-1-ylethoxy)spiro[11,20-dioxa-15-thia-1,14-diazatetracyclo[14.7.2.03,6.019,24]pentacosa-8,14,16,18,24-pentaene-22,4′-2,3-dihydro-1H-naphthalene]-15-yl]-2-methylpropanamide

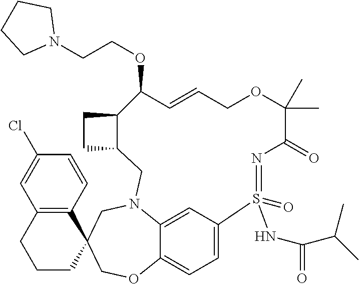 [SMILES: CC(C)C(=O)NS1(=O)=NC(=O)C(C)(C)OC/C=C/[C@H](OCCN2CCCC2)[C@@H]2CC[C@H]2CN2C[C@@]3(CCCc4cc(Cl)ccc43)COc3ccc1cc32]

### Step 1: N-[[(4S)-7-chloro-5′-[[(1R,2R)-2-[(1S)-1-(oxan-2-yloxy)prop-2-enyl]cyclobutyl]methyl]spiro[2,3-dihydro-1H-naphthalene-4,3′-2,4-dihydro-1,5-benzoxazepine]-7′-yl]-(2-methylpropanoylamino)-oxo-sulfanylidene]-2-methyl-2-prop-2-enoxypropanamide

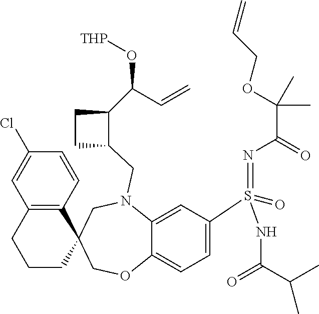

To a stirred solution of N-[amino-[(4S)-7-chloro-5′-[[(1R,2R)-2-[(1S)-1-(oxan-2-yloxy)prop-2-enyl]cyclobutyl]methyl]spiro[2,3-dihydro-1H-naphthalene-4,3′-2,4-dihydro-1,5-benzoxazepine]-7′-yl]-oxo-sulfanylidene]-2-methyl-2-prop-2-enoxypropanamide (500.0 mg, 0.70 mmol, Example 49 Step 5) in DCM (10 mL) was added triethylamine (0.2 mL, 1.4 mmol) followed by isobutyryl chloride (0.08 mL, 0.77 mmol). The resulting mixture was stirred at r.t. for 30 min. The solution was concentrated at 20° C. The residue was purified by flash chromatography on a silica gel column with EtOAc/heptane (20% to 100% with 0.1% triethylamine) then 0% to 50% MeOH/EtOAc (with 0.1 triethylamine) to afford N-[[(4S)-7-chloro-5′-[[(1R,2R)-2-[(1S)-1-(oxan-2-yloxy)prop-2-enyl]cyclobutyl]methyl]spiro[2,3-dihydro-1H-naphthalene-4,3′-2,4-dihydro-1,5-benzoxazepine]-7′-yl]-(2-methylpropanoylamino)-oxo-sulfanylidene]-2-methyl-2-prop-2-enoxypropanamide (300 mg, 54.6% yield) as a light brown solid. LC-MS calc. for C₄₂H₅₇ClN₃O₇S [M+H]⁺: m/z=782.4/784.4; Found: 782.2/784.5.

### Step 2: N-[(3R,6R,7S,8E,22S)-7′-chloro-7-hydroxy-12,12-dimethyl-13,15-dioxospiro[11,20-dioxa-15-thia-1,14-diazatetracyclo[14.7.2.03,6.019,24]pentacosa-8,14,16,18,24-pentaene-22,4′-2,3-dihydro-1H-naphthalene]-15-yl]-2-methylpropanamide

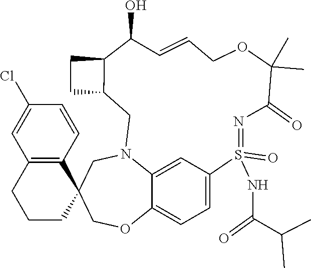 [SMILES: CC(C)C(=O)NS1(=O)=NC(=O)C(C)(C)OC/C=C/[C@H](O)[C@@H]2CC[C@H]2CN2C[C@@]3(CCCc4cc(Cl)ccc43)COc3ccc1cc32]

To a solution of N-[[(4S)-7-chloro-5′-[[(1R,2R)-2-[(1S)-1-(oxan-2-yloxy)prop-2-enyl]cyclobutyl]methyl]spiro[2,3-dihydro-1H-naphthalene-4,3′-2,4-dihydro-1,5-benzoxazepine]-7′-yl]-(2-methylpropanoylamino)-oxo-sulfanylidene]-2-methyl-2-prop-2-enoxypropanamide (310.0 mg, 0.40 mmol) in DCE (390 mL) was bubbled with nitrogen for 5 min. Then Hoveyda-Grubbs II catalyst (49.65 mg, 0.08 mmol) was added under nitrogen. The resulting mixture further bubbled with nitrogen for 5 min. Then the solution was stirred at 70° C. under nitrogen overnight. The solution was cooled to r.t. and bubbled with oxygen then stirred under air for 30 min. to deactivate the catalyst. The solution was concentrated under reduced pressure. The residue was purified by prep-HPLC on C18 column (30×250 mm, 10 μm) with MeCN/H₂O (20% to 100% w/0.1% TFA). The desired fractions were collected and concentrated to ˜5 mL. LC-MS indicated all the THP-protected product was consumed and the formation of desired product. Water (20 mL) was added. The mixture was extracted with DCM (20 mL×3). The combined organic layers were dried over sodium sulfated, filtered and concentrated under reduced pressure to afford N-[(3R,6R,7S,8E,22S)-7′-chloro-7-hydroxy-12,12-dimethyl-13,15-dioxospiro[11,20-dioxa-15-thia-1,14-diazatetracyclo[14.7.2.03,6.019,24]pentacosa-8,14,16,18,24-pentaene-22,4′-2,3-dihydro-1H-naphthalene]-15-yl]-2-methylpropanamide (150 mg, 56.4% yield) as a light brown solid. LC-MS calc. for C₃₅H₄₅ClN₃O₆S [M+H]⁺: m/z=670.3/672.3; Found: 670.4/672.1.

### Step 3: 2-bromoethyl trifluoromethanesulfonate

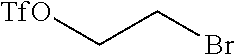 [SMILES: O=S(=O)(OCCBr)C(F)(F)F]

To a stirred solution of pyridine (1.42 mL, 17.61 mmol) in DCM (40 mL) at −20° C. was added triflic anhydride (2.69 mL, 16.01 mmol) dropwise. The resulting suspension was stirred for 10 min at −20° C. and then 2-bromoethanol (1.13 mL, 16.01 mmol) was slowly added. The reaction mixture was stirred for 10 min at −20° C. then 10 min at r.t. The resulting suspension was filtered and concentrated at 18° C. under reduced pressure. The crude product was purified by flash chromatography (10% MTBE in hexanes) to afford 2-bromoethyl trifluoromethanesulfonate (0.80 g, 19.4% yield) as a clear liquid.

### Step 4: N-[(3R,6R,7S,8E,22S)-7-(2-bromoethoxy)-7′-chloro-12,12-dimethyl-13,15-dioxospiro[11,20-dioxa-15-thia-1,14-diazatetracyclo[14.7.2.03,6.019,24]pentacosa-8,14,16,18,24-pentaene-22,4′-2,3-dihydro-1H-naphthalene]-15-yl]-2-methylpropanamide

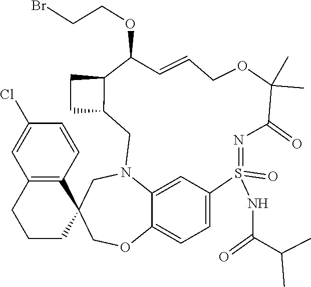 [SMILES: CC(C)C(=O)NS1(=O)=NC(=O)C(C)(C)OC/C=C/[C@H](OCCBr)[C@@H]2CC[C@H]2CN2C[C@@]3(CCCc4cc(Cl)ccc43)COc3ccc1cc32]

A solution of N-[(3R,6R,7S,8E,22S)-7′-chloro-7-hydroxy-12,12-dimethyl-13,15-dioxospiro[11,20-dioxa-15-thia-1,14-diazatetracyclo[14.7.2.03,6.019,24]pentacosa-8,14,16,18,24-pentaene-22,4′-2,3-dihydro-1H-naphthalene]-15-yl]-2-methylpropanamide (150.0 mg, 0.22 mmol), 2-bromoethyl trifluoromethanesulfonate (402.65 mg, 1.57 mmol) and 2,6-di-tert-butylpyridine (0.69 mL, 3.13 mmol) in chloroform (6 mL) was stirred at 90° C. in a sealed tube for 2 days. The reaction was cooled to r.t. and quenched with 1 M HCl (20 mL). The mixture was extracted with DCM (20 mL×3). The combined organic layers were dried over sodium sulfate, filtered and concentrated under reduced pressure. The residue was purified by prep-HPLC on C18 column (30×250 mm, 10 μm) with MeCN/H₂O (20% to 100% w/0.1% TFA) to afford N-[(3R,6R,7S,8E,22S)-7-(2-bromoethoxy)-7′-chloro-12,12-dimethyl-13,15-dioxospiro[11,20-dioxa-15-thia-1,14-diazatetracyclo[14.7.2.03,6.019,24]pentacosa-8,14,16,18,24-pentaene-22,4′-2,3-dihydro-1H-naphthalene]-15-yl]-2-methylpropanamide (45 mg, 25.9% yield) as a yellow solid. LC-MS calc. for C₃₇H₄₇BrClN₄O₆S [M+H]⁺: m/z=776.2/778.2; Found: 776.4/778.1.

### Step 5: N-[(3R,6R,7S,8E,22S)-7′-chloro-12,12-dimethyl-13,15-dioxo-7-(2-pyrrolidin-1-ylethoxy)spiro[11,20-dioxa-15-thia-1,14-diazatetracyclo[14.7.2.03,6.019,24]pentacosa-8,14,16,18,24-pentaene-22,4′-2,3-dihydro-1H-naphthalene]-15-yl]-2-methylpropanamide

To a stirred solution of N-[(3R,6R,7S,8E,22S)-7-(2-bromoethoxy)-7′-chloro-12,12-dimethyl-13,15-dioxospiro[11,20-dioxa-15-thia-1,14-diazatetracyclo[14.7.2.03,6.019,24]pentacosa-8,14,16,18,24-pentaene-22,4′-2,3-dihydro-1H-naphthalene]-15-yl]-2-methylpropanamide (30.0 mg, 0.04 mmol) in DMSO (2 mL) was added pyrrolidine (0.16 mL, 1.93 mmol). The resulting mixture was stirred at r.t. for 30 min. The crude product was directly purified by prep-HPLC on C18 column (30×250 mm, 10 μm) with MeCN/H₂O (20% to 100%) to afford N-[(3R,6R,7S,8E,22S)-7′-chloro-12,12-dimethyl-13,15-dioxo-7-(2-pyrrolidin-1-ylethoxy)spiro[11,20-dioxa-15-thia-1,14-diazatetracyclo[14.7.2.03,6.019,24]pentacosa-8,14,16,18,24-pentaene-22,4′-2,3-dihydro-1H-naphthalene]-15-yl]-2-methylpropanamide (8 mg, 25% yield) as a white solid. LC-MS calc. for C₄₁H₅₆ClN₄O₆S [M+H]⁺: m/z=767.4/769.4; Found: 767.3/769.5. HPLC: C18 column (4.6×100 mm, 5 μm); flow rate=1 mL/min; mobile phase: 50% MeCN/H₂O (with 0.1% TFA) to 95% 4 min, 95% 6 min; λ=220 nm. tR1=5.42 min. ¹H NMR (300 MHz, CDCl₃) δ 7.78-7.61 (m, 1.5H), 7.50 (d, J=8.5 Hz, 0.5H), 7.24-7.15 (m, 1H), 7.11-7.08 (m, 1H), 7.03-6.77 (m, 2H), 5.88-5.63 (m, 2H), 5.31-5.21 (m, 1H), 4.24-4.06 (m, 3H), 3.98-3.94 (m, 1H), 3.86-3.82 (m, 1H), 3.80-3.77 (m, 1H), 3.72-3.64 (m, 2H), 3.48-3.44 (m, 1H), 3.36-3.31 (m, 2H), 3.11-2.98 (m, 5H), 2.79-2.70 (m, 2H), 2.58-2.51 (m, 1H), 2.34-2.24 (m, 1H), 2.23-1.87 (m, 7H), 1.83-1.80 (m, 1H), 1.68-1.62 (m, 2H), 1.51 (s, 1.5H), 1.43 (s, 1.5H), 1.40-1.31 (m, 7H), 1.25-1.17 (m, 3H), 1.16 (d, J=6.5 Hz, 3H).

### Example 55 N-[(3R,6R,7S,8E,22S)-7′-Chloro-7-[2-(dimethylamino)ethoxy]-12,12-dimethyl-13,15-dioxospiro[11,20-dioxa-15-thia-1,14-diazatetracyclo[14.7.2.03,6.019,24]pentacosa-8,14,16,18,24-pentaene-22,4′-2,3-dihydro-1H-naphthalene]-15-yl]-2-methylpropanamide

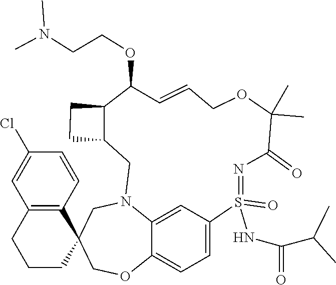 [SMILES: CC(C)C(=O)NS1(=O)=NC(=O)C(C)(C)OC/C=C/[C@H](OCCN(C)C)[C@@H]2CC[C@H]2CN2C[C@@]3(CCCc4cc(Cl)ccc43)COc3ccc1cc32]

This compound was prepared using procedures analogous to those described for Example 52 Step 5 using N-[(3R,6R,7S,8E,22S)-7-(2-bromoethoxy)-7′-chloro-12,12-dimethyl-13,15-dioxospiro[11,20-dioxa-15-thia-1,14-diazatetracyclo[14.7.2.03,6.019,24]pentacosa-8,14,16,18,24-pentaene-22,4′-2,3-dihydro-1H-naphthalene]-15-yl]-2-methylpropanamide and dimethylamine THF solution (2.0 M). LC-MS calc. for C₃₉H₅₄ClN₄O₆S [M+H]⁺: m/z=741.3/743.3; Found: 741.1/743.4. HPLC: C18 column (4.6×100 mm, 5 μm); flow rate=1 mL/min; mobile phase: 50% MeCN/H₂O (with 0.1% TFA) to 95% 4 min, 95% 6 min; λ=220 nm. tR1=5.16 min. ¹H NMR (300 MHz, CDCl₃) δ 7.74-7.57 (m, 1.5H), 7.48 (dd, J=8.4, 2.1 Hz, 0.5H), 7.19-7.16 (m, 1H), 7.10-7.06 (m, 1H), 7.01-6.90 (m, 1H), 6.87-6.77 (m, 1H), 5.91-5.60 (m, 2H), 5.12 (br, 1H), 4.11-4.08 (m, 1H), 3.99-3.96 (m, 1H), 3.85-3.81 (m, 1H), 3.73-3.69 (m, 2H), 3.58-3.48 (m, 2H), 3.43-3.36 (m, 1H), 3.32-3.28 (m, 1H), 3.20-3.09 (m, 1H), 2.92 (s, 3H), 2.75 (s, 3H), 2.60-2.51 (m, 1H), 2.24-2.21 (m, 1H), 2.03-1.79 (m, 12H), 1.65-1.60 (m, 2H), 1.47 (s, 1.5H), 1.40 (s, 15H), 1.36 (s, 1.5H), 1.33 (s, 1.5H), 1.32-1.28 (m, 3H), 1.18 (d, J=6.9, 3H).

### Biological Assays

### Cell Free Mcl-1:Bim Affinity Assay (Mcl-1 Bim)

The binding affinity of each compound was measured via a fluorescence polarization competition assay, in which the compound competes for the same binding site with the ligand, and thus leads to a dose-dependent anisotropy reduction. The tracer ligand utilized was a fluorescein isothiocyanate labelled peptide (FITC-ARIAQELRRIGDEFNETYTR) derived from Bim (GenScript).

The assay was carried out in black half-area 96-well NBS plate (Corning), containing 15 nM of MCL-1 (BPS Bioscience), 5 nM of FITC-Bim and 3-fold serial diluted test compounds in a total volume of 50 μL of assay buffer (20 mM HEPES, 50 mM NaCl, 0.002% Tween 20, 1 mM TCEP, and 1% DMSO). The reaction plate was incubated for 1 hour at room temperature. The change of anisotropy is measured with an Envision multimode plate reader (PerkinElmer) at emission wavelength 535 nm. Fluorescence polarization was calc. in mP unit and the percentage inhibition was calc. by % inhibition=100×(m_{PDMSO}−mP)/(m_{PDMSO}−mP_{PC}), in which m_{PDMSO} is the DMSO control, and mP_{PC} is the positive control. IC₅₀ values were determined from a 10-point dose response curve by fitting the percent inhibition against compound concentration using the GraphPad Prism software. The inhibition constant Kᵢ was subsequently calc. according to the Nikolovska-Coleska's equation (Anal. Biochem., 2004, 332, 261),

$${K}_{i} = \frac{{[ I ]}_{50}}{\frac{{[ L ]}_{50}}{{K}_{d}} + \frac{{[ P ]}_{0}}{{K}_{d}} + 1}$$

where [I]₅₀ is the concentration of the free inhibitor at 50% inhibition, [L]₅₀ is the concentration of the free labeled ligand at 50% inhibition, [P]₀ is the concentration of the free protein at 0% inhibition, and K_{d} is the dissociation constant of the protein-ligand complex. See Table 1.

### Caspase 3/7 Activity Assay

Dispense 10 μL aliquot of prepared H929 cells (1.1 ratio of cells:Trypan Blue (#1450013, Bio-Rad)) onto cell counting slide (#145-0011, Bio-Rad) and obtain cell density and cell viability using cell counter (TC20, Bio-Rad). Remove appropriate volume of resuspended cells from culture flask to accommodate 2000 cells/well @ 5 μL/well. Transfer H929 cells to 50 mL conical (#430290, Corning) for each of the FBS concentration to be assayed (10%, 0.1%). Spin down at 1000 rpm for 5 min. using tabletop centrifuge (SPINCHRON 15, Beckman). Discard supernatant and resuspend cell pellet in modified RPMI 1640 (#10-040-CV, Corning) cell culture media containing sodium pyruvate (100 mM) (#25-000-CL, Corning), HEPES buffer (1 M) (#25-060-CL, Corning) and glucose (200 g/L) (A24940-01, Gibco) with appropriate FBS (F2422-500ML, Sigma) concentration to a cell density of 400,000 cells/mL. Dispense 5 μL of resuspended H929 cells per well in 384-well small volume TC treated plate (#784080, Greiner Bio-one) using standard cassette (#50950372, Thermo Scientific) on Multidrop Combi (#5840310, Thermo Scientific) in laminar flow cabinet. Dispense compounds onto plates using digital liquid dispenser (D300E, Tecan). Incubate plates in humidified tissue culture incubator @ 37° C. for 4 hours. Add 5 μL of prepared Caspase-Glo® 3/7 detection buffer (G8093, Promega) to each well of 384-well plate using small tube cassette (#24073295, Thermo Scientific) on Combi multi-drop, incubate @ RT for 30-60 min. Read plates with microplate reader (PheraStar, BMG Labtech) using 384 well luminescence mode.

### Cell Viability Assay (H929 10 FBS)

Dispense 10 μL aliquot of prepared H929 cells (1:1 ratio of cells:Trypan Blue (#1450013, Bio-Rad)) onto cell counting slide (#145-0011, Bio-Rad) and obtain cell density and cell viability using cell counter (TC20, Bio-Rad). Remove appropriate volume of resuspended cells from culture flask to accommodate 4000 cells/well @ 10 μL/well. Transfer H929 cells to 50 mL conical (#430290, Corning). Spin down at 1000 rpm for 5 min using tabletop centrifuge (SPINCHRON 15, Beckman). Discard supernatant and resuspend cell pellet in modified RPMI 1640 (#10-040-CV, Corning) cell culture media containing 10% FBS (F2422-500 ML, Sigma), sodium pyruvate (100 mM) (#25-000-CL, Corning), HEPES buffer (1 M) (#25-060-CL, Corning) and glucose (200 g/L) (A24940-01, Gibco) to a cell density of 400,000 cells/mL. Dispense 10 μL of resuspended H929 cells per well in 384-well small volume TC treated plate (#784080, Greiner Bio-one) using standard cassette (#50950372, Thermo Scientific) on Multi-drop Combi (#5840310, Thermo Scientific) in laminar flow cabinet. Dispense compounds onto plates using digital liquid dispenser (D300E, Tecan). Incubate plates in humidified tissue culture incubator @ 37° C. for 24 hours. Add 10 μL of prepared CellTiTer-Glo® detection buffer (G7570, Promega) or ATPlite 1Step detection reagent (#6016731, Perkin Elmer) to each well of 384-well plate using small tube cassette (#24073295, Thermo Scientific) on Combi multi drop, incubate @ RT for 30-60 min. Read plates with microplate reader (PheraStar, BMG Labtech) using 384 well luminescence mode.

### Cytotoxicity Studies in NCI-H929 Cells

Cytotoxicity studies were conducted in NCI-H929 multiple myeloma cell line. Cells were maintained in RPMI 1640 (Corning Cellgro, Catalog #: 10-040-CV) supplemented with 10% v/v FBS (GE Healthcare, Catalog #: SH30910.03), 10 mM HEPES (Corning, Catalog #: 25-060-CI), 1 mM sodium pyruvate (Corning Cellgro, Catalog #: 25-000-CI and 2500 mg/L glucose (Gibco, Catalog #: A24940-01). Cells were seeded in 96-well plates at a density of 75000 cells/well. Compounds dissolved in DMSO were plated in duplicate using a digital dispenser (Tecan D300E) and tested on a 9-point 3-fold serial dilution. Cells were incubated for 24 hr in a 37° C. incubator at 5% CO₂. Cell viability was measured using the Cell Counting Kit-8 (CCK-8, Jojindo, CK04-13) as per manufacturer's instructions. Cells were incubated for 4 hours at 37° C. 5% CO₂ following addition of reagent and OD₄₅₀ values were measured with a microplate reader (iMark microplate reader, Bio-Rad). Background from media only wells were averaged and subtracted from all readings. OD₄₅₀ values were then normalized to DMSO controls to obtain percentage of viable cells, relative to DMSO vehicle control and plotted in Graphpad Prism ([Inhibitor] vs. normalized response−Variable slope; equation: Y=100/(1+(X\hat{ }HillSlope)/(IC₅₀\hat{ }HillSlope))) to determine IC₅₀ values (the concentration of compound inhibiting half of the maximal activity).

TABLE 1
Cell free Mcl-1: Bim affinity assay (Mcl-1 Bim) and
Cell viability assay (H929_10FBS)

| Ex | BIM_ | H929_10FBS |
|---|---|---|
| No. | Ki (nM) | IC₅₀ (nM) |
| 1 or 2 | ++ | ### |
| 2 or 1 | ++ | ## |
| 3 or 4 | ++ | NT |
| 4 or 3 | ++ | NT |
| 5 or 6 | ++ | NT |
| 6 or 5 | ++ | NT |
| 7 or 8 | ++ | NT |
| 8 or 7 | NT | NT |
| 9 or 10 | ++ | ### |
| 10 or 9 | + | ## |
| 11 | ++ | ### |
| 12 | ++ | ## |
| 13 or 14 | + | NT |
| 14 or 13 | + | NT |
| 15 | ++ | ### |
| 16 | ++ | ### |
| 17 | ++ | ### |
| 18 | ++ | NT |
| 19 | ++ | NT |
| 20 | ++ | ### |
| 21 or 22 | ++ | ### |
| 22 or 21 | ++ | ## |
| 23 or 24 | ++ | ### |
| 24 or 23 | ++ | ## |
| 25 or 26 | ++ | ### |
| 26 or 25 | ++ | # |
| 27 or 28 | ++ | ### |
| 28 or 27 | ++ | ## |
| 29 or 30 | ++ | ### |
| 30 or 29 | + | # |
| 31 or 32 | ++ | ### |
| 32 or 31 | ++ | ## |
| 33 | ++ | ### |
| 34 or 35 | ++ | NT |
| 35 or 34 | ++ | NT |
| 36 or 37 | ++ | NT |
| 37 or 36 | + | NT |
| 38 | + | NT |
| 39 or 40 | ++ | NT |
| 40 or 39 | ++ | NT |
| 41 or 42 | + | NT |
| 42 or 41 | ++ | NT |
| 43 or 44 | ++ | NT |
| 44 or 43 | ++ | NT |
| 45 or 46 | ++ | NT |
| 46 or 45 | ++ | NT |
| 47 or 48 | + | NT |
| 48 or 47 | ++ | NT |
| 49 or 50 | ++ | NT |
| 50 or 49 | + | NT |
| 51 | ++ | NT |
| 52 | + | NT |
| 53 | ++ | NT |
| 54 | ++ | NT |
| 55 | + | NT |

++ Ki <1 nM;
+ Ki = >1 nM;
### IC₅₀ <100 nM;
## 100 nM < IC₅₀ <500 nM;
# IC₅₀ >500 nM;
NT = not tested

In some embodiments, the disclosure is directed to the following aspects:
Aspect 1. A compound of Formula I:

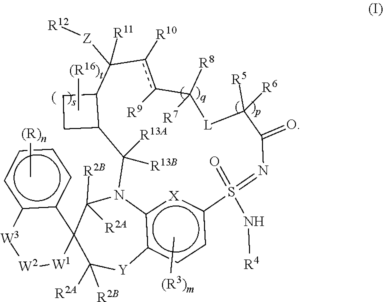

or a pharmaceutically acceptable salt or solvate thereof;

wherein
L is absent, —NR¹⁴—, —O—, —S—, —S(O)—, or —S(O)₂—, arylene, —O-arylene, cycloalkylene, —O— cycloalkylene, cycloalkenylene, spirocycloalkylene, heteroarylene, heterocycloalkylene, —O-heterocycloalkylene, heterocycloalkenylene, or spiroheterocycloalkylene wherein said arylene, cycloalkylene, cycloalkenylene, spirocycloalkylene, heteroarylene, heterocycloalkylene, heterocycloalkenylene, or spiroheterocycloalkylene is optionally substituted;
[symbol] is single bond or double bond;
X is CH or N;
Y is —O—, —S—, —S(O)—, or —S(O)₂—;
Z is —NR¹⁵—, —O—, or —S—;
the moiety —W¹—W²—W³ is —CR^{1A}R^{1B}—CR^{1C}R^{1D}—CR^{1A}R^{1B}—, —O—CR^{1C}R^{1D}—CR^{1A}R^{1B}—, —CR^{1A}R^{1B}—CR^{1C}R^{1D}—O—, —NR^{1C}—CR^{1C}R^{1D}—CR^{1A}R^{1B}—, —CR^{1A}R^{1B}—CR^{1C}R^{1D}—NR^{1C}—, —S—CR^{1C}R^{1D} CR^{1A}R^{1B}, or —CR^{1A}R^{1B}—CR^{1C}R^{1D}—S—;
each n is independently 0-3;
each m is independently 0-2;
each p is independently 0-4;
each q is independently 0-4;
each s is independently 0-3;
each t is independently 0-6;
each R is independently -D, -halo, —CN, —NO₂, —C₁-C₆alkyl, —C₂-C₆alkenyl, —C₁-C₆alkoxy, -cycloalkyl, —OR^{a}, —SR^{a}, —C(O)R^{b}, —C(O)OR^{b}, —NR^{c}R^{d}, —NR^{a}R^{c}, —C(O)NR^{c}R^{d}, —S(O)R^{b}, —S(O)₂R^{b}, wherein said —C₁-C₆alkyl, —C₂-C₆alkenyl, —C₁-C₆alkoxy, or -cycloalkyl is optionally substituted;
each R^{1A}, or R^{1B} is independently H, D, halo, optionally substituted C₁-C₆alkyl, or R^{1A} and R^{1B} that are attached to the same carbon atom may, together with the carbon atom to which they are both attached, form an optionally substituted cycloalkyl ring;
each R^{1C} and R^{1D} is independently H, D, fluoro, optionally substituted C₁-C₆alkyl, or R^{1C} and R^{1D} may, together with the carbon atom to which they are both attached, form an optionally substituted cycloalkyl ring;
or R^{1B} and R^{1C} that are attached to the adjacent carbon atoms may, together with the carbon atoms to which they are attached, form an optionally substituted cycloalkyl ring;
each R^{2A} and R^{2B} is independently H, D, fluoro, optionally substituted C₁-C₆alkyl, or R^{2A} and R^{2B} may, together with the carbon atom to which they are both attached, form an optionally substituted cycloalkyl ring;
each R³ is independently -D, -halo, —CN, —NO₂, —C₁-C₆alkyl, —C₂-C₆alkenyl, —C₂-C₆alkynyl, —OR^{a}, —SR^{a}, —NR^{c}R^{d}, —NR^{a}R^{c}, —C(O)R^{b}, —OC(O)R^{b}, —C(O)OR^{b}, —C(O)NR^{c}R^{d}, —S(O)₂R^{b}; -aryl, -heteroaryl, -cycloalkyl, or -heterocycloalkyl, wherein said —C₁-C₆alkyl, —C₂-C₆alkenyl, —C₂-C₆alkynyl, -cycloalkyl, -heterocycloalkyl, -aryl, or -heteroaryl is optionally substituted;
R⁴ is H, —C(O)OR^{4A}, —C(O)R^{4B}, —C(O)NR^{4C}R^{4D}, —S(O)R^{4B}, —S(O)₂R^{4B}, —S(O)NR^{4C}R^{4D} or —S(O)₂NR^{4C}R^{4D};
each R^{4A} is independently —C₁-C₁₀alkyl, —C₃-C₁₀ alkenyl, —C₃-C₁₀ alkynyl, aryl, cycloalkyl, C₁-C₆heteroalkyl, heteroaryl, heterocycloalkyl, or heterocycloalkenyl wherein said C₁-C₁₀ alkyl, C₃-C₁₀ alkenyl, C₃-C₁₀ alkynyl, aryl, cycloalkyl, heteroaryl, heterocycloalkyl, or heterocycloalkenyl is optionally substituted;
each R^{4B} is independently H, D, —C₁-C₆ alkyl, —C₂-C₆ alkenyl, —C₂-C₆ alkynyl, aryl, cycloalkyl, C₁-C₆heteroalkyl, heteroaryl, heterocycloalkyl, or heterocycloalkenyl wherein said —C₁-C₆ alkyl, —C₂-C₆ alkenyl, —C₂-C₆ alkynyl, aryl, cycloalkyl, heteroaryl, heterocycloalkyl, or heterocycloalkenyl is optionally substituted;
each R^{4C} or R^{4D} is independently H, D, —C₁-C₁₀ alkyl, —C₂-C₆ alkenyl, —C₂-C₆ alkynyl, —OC₁-C₆alkyl, —O-cycloalkyl, aryl, heteroaryl, cycloalkyl, C₁-C₆heteroalkyl, heterocycloalkyl, or heterocycloalkenyl, wherein said C₁-C₁₀ alkyl, C₂-C₆ alkenyl, C₂-C₆ alkynyl, —OC₁-C₆alkyl, —O-cycloalkyl, aryl, heteroaryl, cycloalkyl, heterocycloalkyl, or heterocycloalkenyl are each optionally substituted;
or R^{4C} and R^{4D}, together with the N atom to which they are both attached, form an optionally substituted monocyclic or multicyclic heterocycloalkyl, or optionally substituted monocyclic or multicyclic heterocycloalkenyl group;
each R⁵, R⁷ or R¹¹ is independently H, D, halo, —OH, —CN, —NO₂, —C₁-C₆alkyl, —C₂-C₆alkenyl, —C₂-C₆alkynyl, aryl, heteroaryl, cycloalkyl, heterocycloalkyl, heterocycloalkenyl, —OR^{a}, —SR^{a}, —NR^{c}R^{d}, —NR^{a}R^{c}, —C(O)R^{b}, —OC(O)R^{b}, —C(O)OR^{b}, —C(O)NR^{c}R^{d}, —S(O)R^{b}, or —S(O)₂R^{b}, wherein said C₁-C₆alkyl, —C₂-C₆alkenyl, —C₂-C₆alkynyl, aryl, heteroaryl, cycloalkyl, heterocycloalkenyl, or heterocycloalkyl is optionally substituted;
each R⁶ or R⁸ is independently H, D, halo, —C₁-C₆alkyl, —C₂-C₆alkenyl, —C₂-C₆alkynyl, —C(O)R^{b}, —C(O)OR^{a}, —C(O)NR^{c}R^{d}, aryl, heteroaryl, cycloalkyl, heterocycloalkyl, or heterocycloalkenyl, wherein said C₁-C₆alkyl, —C₂-C₆alkenyl, —C₂-C₆alkynyl, aryl, heteroaryl, cycloalkyl, heterocycloalkyl, or heterocycloalkenyl is optionally substituted;
or R⁵ and R⁶ together with the C atom to which they are attached form a cycloalkyl, heterocycloalkyl, or heterocycloalkenyl ring, each optionally substituted;
or an R⁵ and an R⁶ attached to adjacent carbon atoms, together with the C atoms to which they are attached, form a cycloalkyl, heterocycloalkyl, or heterocycloalkenyl ring, each optionally substituted;
or R⁷ and R⁸ together with the C atom to which they are both attached form a cycloalkyl, heterocycloalkyl, or heterocycloalkenyl ring, each optionally substituted, each optionally substituted;
or an R⁷ and an R⁸ attached to adjacent carbon atoms, together with the C atoms to which they are attached, form a cycloalkyl, heterocycloalkyl, or heterocycloalkenyl ring, each optionally substituted;
each R⁹ or R¹⁰ is independently H, D, -Me, CN, —CH₂CN, —CH₂F, —CHF₂, —CF₃ or —F;
each R¹² is H, D, —C(O)R^{b}, —C(O)OR^{c}, —C(O)NR^{c}R^{d}, P(OR^{c})₂, P(O)R^{c}R^{b}, P(O)OR^{c}OR^{b}, S(O)R^{b}, S(O)NR^{c}R^{d}, S(O)₂R^{b}, S(O)₂NR^{c}R^{d}, B(OR⁴)(OR^{b}), SiR^{b}₃, C₁-C₁₀alkyl, C₂-C₁₀ alkenyl, C₂-C₁₀ alkynyl, —(CH₂CH₂O)ₒR^{a} wherein o=1 to 10, aryl, cycloalkyl, heteroaryl, or heterocycloalkyl, wherein said C₁-C₁₀ alkyl, C₂-C₁₀alkenyl, C₂-C₁₀ alkynyl, aryl, cycloalkyl, heteroaryl, or heterocycloalkyl is optionally substituted;
or R¹² and R¹¹ form an optionally substituted monocyclic or multicyclic heterocycloalkyl, or optionally substituted monocyclic or multicyclic heterocycloalkenyl group;
each R^{13A} or R^{13B} is independently H, D, optionally substituted C₁-C₆alkyl;
or R^{13A} and R^{13B} that are attached to the same carbon atom may, together with the carbon atom to which they are both attached, form an optionally substituted cycloalkyl ring;
each R¹⁴ or R¹⁵ is independently H, D, —C₁-C₆alkyl, —C₂-C₆alkenyl, —C₂-C₆alkynyl, —OC₁-C₆alkyl, —C(O)R^{b}, —C(O)OR^{b}, —C(O)NR^{c}R^{d}, —S(O)R^{b} or —S(O)₂R^{b}, aryl, heteroaryl, cycloalkyl, heterocycloalkyl, or heterocycloalkenyl group, wherein said C₁-C₆alkyl, —C₂-C₆alkenyl, —C₂-C₆alkynyl, —OC₁-C₆alkyl, aryl, heteroaryl, cycloalkyl, heterocycloalkyl, or heterocycloalkenyl ring, is optionally substituted;
or R¹⁴ together with an R⁴, R⁵, R⁷ or an R⁸ form an optionally substituted monocyclic or multicyclic heterocycloalkyl, or optionally substituted monocyclic or multicyclic heterocycloalkenyl group;
each R¹⁶ is independently H, D, —OH, -Me, —CH₂F, —CHF₂, —CF₃ or —F;
each R^{a} is independently H, D, —C(O)R^{b}, —C(O)OR^{c}, —C(O)NR^{c}R^{d}, —P(OR^{c})₂, —P(O)R^{c}R^{b}, —P(O)OR^{c}OR^{b}, —S(O)R^{b}, —S(O)NR^{c}R^{d}, —S(O)₂R^{b}, —S(O)₂NR^{c}R^{d}, —B(OR^{c})(OR^{b}), SiR^{b}₃, —C₁-C₁₀alkyl, —C₂-C₁₀ alkenyl, —C₂-C₁₀ alkynyl, aryl, cycloalkyl, heteroaryl, heterocycloalkyl, or heterocycloalkenyl wherein said C₁-C₁₀ alkyl, C₂-C₁₀ alkenyl, C₂-C₁₀ alkynyl, aryl, cycloalkyl, heteroaryl, heterocycloalkyl, or heterocycloalkenyl is optionally substituted;
each R^{b}, is independently H, D, —C₁-C₆ alkyl, —C₂-C₆ alkenyl, —C₂-C₆ alkynyl, aryl, cycloalkyl, heteroaryl, heterocycloalkyl, or heterocycloalkenyl wherein said —C₁-C₆ alkyl, —C₂-C₆ alkenyl, —C₂-C₆ alkynyl, aryl, cycloalkyl, heteroaryl, heterocycloalkyl, or heterocycloalkenyl is optionally substituted;
each R^{c} or R^{d} is independently H, D, —C₁-C₁₀ alkyl, —C₂-C₆ alkenyl, —C₂-C₆ alkynyl, —OC₁-C₆alkyl, —O-cycloalkyl, aryl, heteroaryl, cycloalkyl, heterocycloalkyl, or heterocycloalkenyl, wherein said C₁-C₁₀ alkyl, C₂-C₆ alkenyl, C₂-C₆ alkynyl, —OC₁-C₆alkyl, —O-cycloalkyl, aryl, heteroaryl, cycloalkyl, heterocycloalkyl, or heterocycloalkenyl are each optionally substituted;

or R^{c} and R^{d}, together with the N atom to which they are both attached, form an optionally substituted monocyclic or multicyclic heterocycloalkyl, or optionally substituted monocyclic or multicyclic heterocycloalkenyl group.
Aspect 2. The compound of aspect 1, wherein s=1.
Aspect 3. The compound of aspect 2, wherein t=0; R^{2A} is H; R^{2B} is H; R^{13A} is H; and R^{13B} is H.
Aspect 4. The compound of aspect 3, wherein m=0.
Aspect 5. The compound according to any one of aspects 1 to 4, wherein the compound has the structure IA:

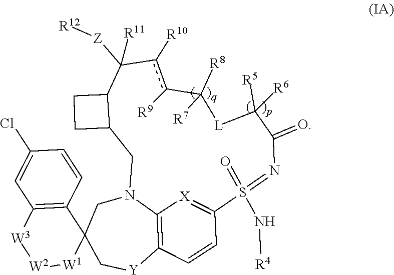

Aspect 6. The compound according to any one of aspects 1 to 5, wherein Y is O.
Aspect 7. The compound according to any one of aspects 1 to 6, wherein X is CH.
Aspect 8. The compound according to any one of aspects 1 to 7, wherein the compound has the structure IB:

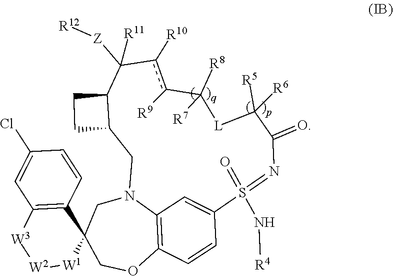

Aspect 9. The compound according to any one of aspects 1 to 8, wherein the moiety —W¹—W²—W³— is —CR^{1A}R^{1B}—CR^{1C}R^{1D}—O—.
Aspect 10. The compound according to aspect 9, wherein R^{1A} is H; R^{1B} is H; R^{1C} is H; and R^{1D} is H.
Aspect 11. The compound according to any one of aspects 1 to 8, wherein the moiety —W¹—W²—W³ is —O—CR^{1C}R^{1D}—CR^{1A}R^{1B}—.
Aspect 12. The compound according to aspect 11, wherein R^{1A} is H; R^{1B} is H; R^{1C} is H; and R^{1D} is H.
Aspect 13. The compound according to any one of aspects 1 to 8, wherein the moiety —W¹—W²—W³ is —CR^{1A}R^{1B}—CR^{1C}R^{1D}—CR^{1A}R^{1B}—.
Aspect 14. The compound according to aspect 13, wherein R^{1A} is H; R^{1B} is H; R^{1C} is H; and R^{1D} is H. [symbol]
Aspect 15. The compound according to any one of aspects 1 to 14, wherein [symbol] is a single bond.
Aspect 16. The compound according to aspect 15, wherein L is absent.
Aspect 17. The compound according to aspect 15, wherein p=0.
Aspect 18. The compound according to aspect 15, wherein q=0.
Aspect 19. The compound according to any one of aspects 1 to 14, wherein [symbol] is a double bond.
Aspect 20. The compound according to aspect 19, wherein L is absent.
Aspect 21. The compound according to aspect 19, wherein p=0.
Aspect 22. The compound according to aspect 19, wherein p=1.
Aspect 23. The compound according to aspect 22, wherein q=1.
Aspect 24. The compound according to any one of aspects 19, or 21-23, wherein L is NR¹.
Aspect 25. The compound according to aspect 24, wherein R¹⁴ is —C₁-C₆alkyl.
Aspect 26. The compound according to aspect 25, wherein R¹⁴ is —CH₃.
Aspect 27. The compound according to any one of aspects 1-26, wherein R⁹ is H and R¹⁰ is H.
Aspect 28. The compound according to aspect 8, wherein the compound of Formula IB has the structure IC:

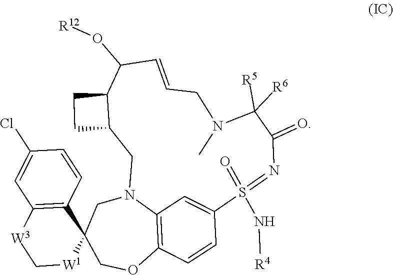

Aspect 29. The compound according to aspect 8, wherein the compound of Formula IB has the structure ID:

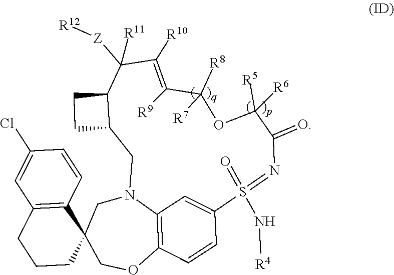

Aspect 30. The compound according to aspect 29, wherein R⁹ is H; R¹⁰ is H; R¹¹ is H; p=1; and q=1.
Aspect 31. The compound according to aspect 8, wherein the compound of Formula (IB) is a compound of Formula (IE):

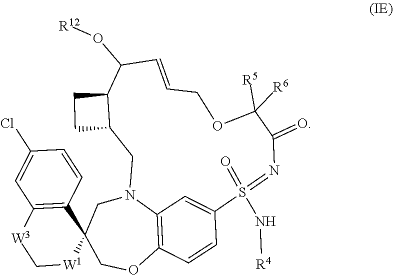

Aspect 32. The compound according to aspect 31, wherein the compound of Formula (IE) has the structure of Formula (IE-1):

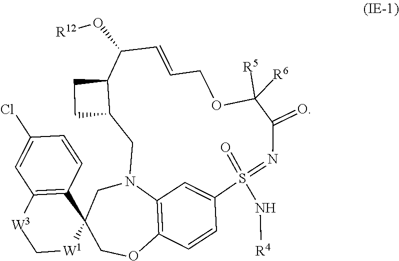

Aspect 33. The compound according to aspect 32, wherein both W³ and W¹ are —CH₂—.
Aspect 34. The compound according to any one of aspect 32 or 33, wherein R¹² is H, optionally substituted C₁-C₁₀alkyl, or —C(O)NR^{c}R^{d}.
Aspect 35. The compound according to aspect 34, wherein R¹² is H.
Aspect 36. The compound according to aspect 34, wherein R¹² is —C₁-C₁₀alkyl.
Aspect 37. The compound according to aspect 35, wherein R¹² is —CH₃.
Aspect 38. The compound according to aspect 24, wherein R¹² is C₁-C₁₀alkyl substituted with —NR^{c1}R^{d1}.
Aspect 39. The compound according to aspect 38, wherein R¹² is —CH₂CH₂NR^{c1}R^{d1}.
Aspect 40. The compound according to one of aspects 38 or 39, wherein R^{c1} and R^{d1} are independently C₁-C₁₀ alkyl.
Aspect 41. The compound according to any one of aspects 38-40, wherein R¹² is —CH₂CH₂—N(CH₃)₂.
Aspect 42. The compound according to aspect 34, wherein R¹² is C₁-C₁₀alkyl substituted with heterocycloalkyl.

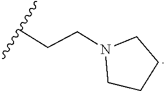 [SMILES: *CCN1CCCC1]

Aspect 43. The compound according to aspect 42, wherein R¹² is
Aspect 44. The compound according to aspect 34, wherein R¹² is C(O)NR^{c}R^{d}.
Aspect 45. The compound according to aspect 44, wherein R¹² is —C(O)NR^{c}R^{d} wherein R and R^{d} are each independently —C₁-C₁₀ alkyl.
Aspect 46. The compound according to aspect 45, wherein R¹² is —C(O)N(CH₃)₂.
Aspect 47. The compound according to any one of aspects 32-46, wherein the compound of Formula (IE-1) is a compound of Formula (IE-1-1):

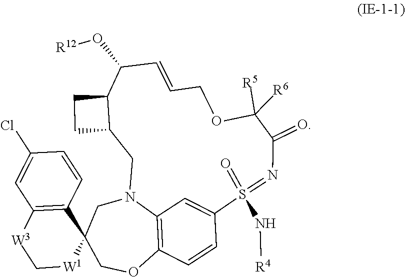

Aspect 48. The compound according to any one of aspects 32-46, wherein the compound of Formula (IE-1) is a compound of Formula (IE-1-2):

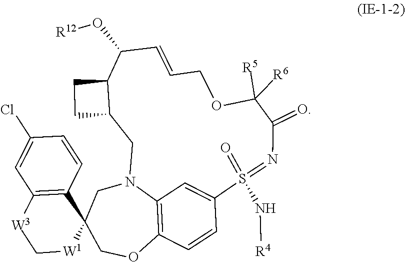

Aspect 49. The compound according to aspect 31, wherein the compound of Formula (IE) has the structure of Formula (IE-2):

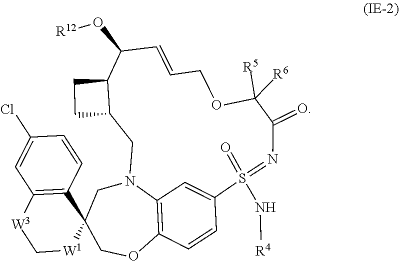

Aspect 50. The compound according to aspect 49, wherein both W³ and W¹ are —CH₂—.
Aspect 51. The compound according to any one of aspect 49 or aspect 50, wherein R¹² is H, optionally substituted C₁-C₁₀alkyl, or —C(O)NR^{c}R^{d}.
Aspect 52. The compound according to aspect 51, wherein R¹² is H.
Aspect 53. The compound according to aspect 51, wherein R¹² is optionally substituted —C₁-C₁₀alkyl.
Aspect 54. The compound according to aspect 53, wherein R¹² is —CH₃.
Aspect 55. The compound according to aspect 51, wherein R¹² is C₁-C₁₀alkyl substituted with —NR^{c1}R^{d1}.
Aspect 56. The compound according to aspect 55, wherein R¹² is —CH₂CH₂NR^{c1}R^{d1}.
Aspect 57. The compound according to one of aspects 55 or 56, wherein R^{c1} and R^{d1} are independently C₁-C₁₀ alkyl.
Aspect 58. The compound according to any one of aspects 55-57, wherein R¹² is —CH₂CH₂—N(CH₃)₂.
Aspect 59. The compound according to aspect 51, wherein R¹² is C₁-C₁₀alkyl substituted with heterocycloalkyl.

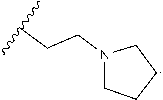 [SMILES: *CCN1CCCC1]

Aspect 60. The compound according to aspect 59, wherein R¹² is
Aspect 61. The compound according to aspect 51, wherein R¹² is C(O)NR^{c}R^{d}.
Aspect 62. The compound according to aspect 61, wherein R¹² is —C(O)NR^{c}R^{d} wherein R^{c} and R^{d} are each independently —C₁-C₁₀ alkyl.
Aspect 63. The compound according to aspect 62, wherein R¹² is —C(O)N(CH₃)₂.
Aspect 64. The compound according to any one of aspects 49-63, wherein the compound of Formula (IE-2) is a compound of Formula (IE-2-1).

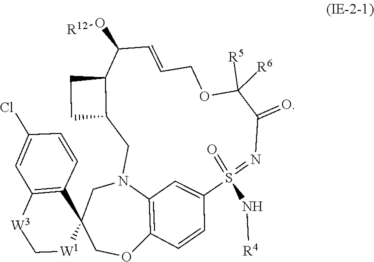

Aspect 65. The compound according to any one of aspects 49-63, wherein the compound of Formula (IE-2) is a compound of Formula (IE-2-2).

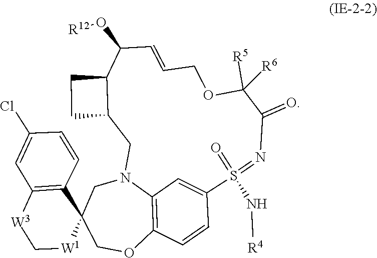

Aspect 66. The compound according to any one of aspects 1-16, 18-20, and 22-65, wherein R⁵ is optionally substituted —C₁-C₆alkyl; and R⁶ is optionally substituted —C₁-C₆alkyl.
Aspect 67. The compound according to aspect 66, wherein R⁵ is —CH₃; and R⁶ is CH₃.
Aspect 68. The compound according to any one of the preceding aspects, wherein R⁴ is —C(O)R^{4B}, or —C(O)NR^{4C}R^{4D}.
Aspect 69. A pharmaceutical composition comprising a compound according to any one of aspects 1 to 68 and a pharmaceutically acceptable excipient.
Aspect 70. A method of inhibiting an MCL-1 enzyme comprising contacting the MCL-1 enzyme with an effective amount of a compound of any one of aspects 1 to 68.
Aspect 71. A method of treating a disease or disorder associated with aberrant MCL-1 activity in a subject comprising administering to the subject, a compound of any one of aspects 1 to 68.
Aspect 72. The method of aspect 71, wherein the disease or disorder associated with aberrant MCL-1 activity is colon cancer, breast cancer, small-cell lung cancer, non-small-cell lung cancer, bladder cancer, ovarian cancer, prostate cancer, chronic lymphoid leukemia, lymphoma, myeloma, acute myeloid leukemia, or pancreatic cancer.

1. A compound of Formula I: 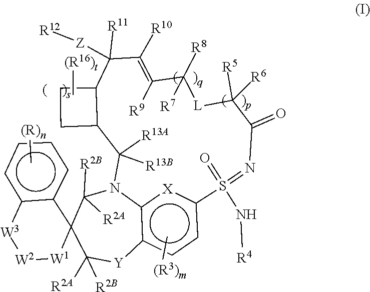
or a pharmaceutically acceptable salt or solvate thereof;
wherein
L is absent, —NR¹⁴—, —O—, —S—, —S(O)—, or —S(O)₂—, arylene, —O-arylene, cycloalkylene, —O— cycloalkylene, cycloalkenylene, spirocycloalkylene, heteroarylene, heterocycloalkylene, —O-heterocycloalkylene, heterocycloalkenylene, or spiroheterocycloalkylene wherein said arylene, cycloalkylene, cycloalkenylene, spirocycloalkylene, heteroarylene, heterocycloalkylene, heterocycloalkenylene, or spiroheterocycloalkylene is optionally substituted; [symbol]
is single bond or double bond;
X is CH or N;
Y is —O—, —S—, —S(O)—, or —S(O)₂—;
Z is —NR¹⁵, —O—, or —S—;
the moiety —W¹—W²—W³ is —CR^{1A}R^{1B}—CR^{1C}R^{1D}—CR^{1A}R^{1B}—, —O—CR^{1C}R^{1D}—CR^{1A}R^{1B}—, —CR^{1A}R^{1B}—CR^{1C}R^{1D}—O—, —NR^{1C}—CR^{1C}R^{1D}—CR^{1A}R^{1B}—, —CR^{1A}R^{1B}—CR^{1C}R^{1D}—NR^{1C}—, —S—CR^{1C}R^{1D}—CR^{1A}R^{1B}, or —CR^{1A}R^{1B}—CR^{1C}R^{1D}—S—;
each n is independently 0-3;
each m is independently 0-2;
each p is independently 0-4;
each q is independently 0-4;
each s is independently 0-3;
each t is independently 0-6;
each R is independently -D, -halo, —CN, —NO₂, —C₁-C₆alkyl, —C₂-C₆alkenyl, —C₁-C₆alkoxy, -cycloalkyl, —OR^{a}, —SR^{a}, —C(O)R^{b}, —C(O)OR^{b}, —NR^{c}R^{d}, —NR^{a}R^{c}, C(O)NR^{c}R^{d}, —S(O)R^{b}, —S(O)₂R^{b}, wherein said —C₁-C₆alkyl, —C₂-C₆alkenyl, —C₁-C₆alkoxy, or -cycloalkyl is optionally substituted;
each R^{1A}, or R^{1B} is independently H, D, halo, optionally substituted C₁-C₆alkyl, or R^{1A} and R^{1B} that are attached to the same carbon atom may, together with the carbon atom to which they are both attached, form an optionally substituted cycloalkyl ring;
each R^{1C} and R^{1D} is independently H, D, fluoro, optionally substituted C₁-C₆alkyl, or R^{1C} and R^{1D} may, together with the carbon atom to which they are both attached, form an optionally substituted cycloalkyl ring;
or R^{1B} and R^{1C} that are attached to the adjacent carbon atoms may, together with the carbon atoms to which they are attached, form an optionally substituted cycloalkyl ring;
each R^{2A} and R^{2B} is independently H, D, fluoro, optionally substituted C₁-C₆alkyl, or R^{2A} and R^{2B} may, together with the carbon atom to which they are both attached, form an optionally substituted cycloalkyl ring;
each R³ is independently -D, -halo, —CN, —NO₂, —C₁-C₆alkyl, —C₂-C₆alkenyl, —C₂-C₆alkynyl, —OR^{a}, —SR^{a}, —NR^{c}R^{d}, —NR^{a}R^{c}, —C(O)R^{b}, —OC(O)R^{b}, —C(O)OR^{b}, —C(O)NR^{c}R^{d}, —S(O)₂R^{b}; -aryl, -heteroaryl, -cycloalkyl, or -heterocycloalkyl, wherein said —C₁-C₆alkyl, —C₂-C₆alkenyl, —C₂-C₆alkynyl, -cycloalkyl, -heterocycloalkyl, -aryl, or -heteroaryl is optionally substituted;
R⁴ is H, —C(O)OR^{4A}, —C(O)R^{4B}, —C(O)NR^{4C}R^{4D}, —S(O)R^{4B}, —S(O)₂R^{4B}, —S(O)NR^{4C}R^{4D}, or —S(O)₂NR^{4C}R^{4D};
each R^{4A} is independently —C₁-C₁₀alkyl, —C₃-C₁₀ alkenyl, —C₃-C₁₀ alkynyl, aryl, cycloalkyl, C₁-C₆heteroalkyl, heteroaryl, heterocycloalkyl, or heterocycloalkenyl wherein said C₁-C₁₀ alkyl, C₃-C₁₀ alkenyl, C₃-C₁₀ alkynyl, aryl, cycloalkyl, heteroaryl, heterocycloalkyl, or heterocycloalkenyl is optionally substituted;
each R^{4B} is independently H, D, —C₁-C₆ alkyl, —C₂-C₆ alkenyl, —C₂-C₆ alkynyl, aryl, cycloalkyl, C₁-C₆heteroalkyl, heteroaryl, heterocycloalkyl, or heterocycloalkenyl wherein said —C₁-C₆ alkyl, —C₂-C₆ alkenyl, —C₂-C₆ alkynyl, aryl, cycloalkyl, heteroaryl, heterocycloalkyl, or heterocycloalkenyl is optionally substituted;
each R^{4C} or R^{4D} is independently H, D, —C₁-C₁₀ alkyl, —C₂-C₆ alkenyl, —C₂-C₆ alkynyl, —OC₁-C₆alkyl, —O-cycloalkyl, aryl, heteroaryl, cycloalkyl, C₁-C₆heteroalkyl, heterocycloalkyl, or heterocycloalkenyl, wherein said C₁-C₁₀ alkyl, C₂-C₆ alkenyl, C₂-C₆ alkynyl, —OC₁-C₆alkyl, —O-cycloalkyl, aryl, heteroaryl, cycloalkyl, heterocycloalkyl, or heterocycloalkenyl are each optionally substituted;
or R^{4C} and R^{4D}, together with the N atom to which they are both attached, form an optionally substituted monocyclic or multicyclic heterocycloalkyl, or optionally substituted monocyclic or multicyclic heterocycloalkenyl group;
each R⁵, R⁷ or R¹¹ is independently H, D, halo, —OH, —CN, —NO₂, —C₁-C₆alkyl, —C₂-C₆alkenyl, —C₂-C₆alkynyl, aryl, heteroaryl, cycloalkyl, heterocycloalkyl, heterocycloalkenyl, —OR^{a}, —SR^{a}, —NR^{c}R^{d}, —NR^{a}R^{c}, —C(O)R^{b}, —OC(O)R^{b}, —C(O)OR^{b}, —C(O)NR^{c}R^{d}, —S(O)R^{b}, or —S(O)₂R^{b}, wherein said C₁-C₆alkyl, —C₂-C₆alkenyl, —C₂-C₆alkynyl, aryl, heteroaryl, cycloalkyl, heterocycloalkenyl, or heterocycloalkyl is optionally substituted;
each R⁶ or R⁸ is independently H, D, halo, —C₁-C₆alkyl, —C₂-C₆alkenyl, —C₂-C₆alkynyl, —C(O)R^{b}, —C(O)OR^{a}, —C(O)NR^{c}R^{d}, aryl, heteroaryl, cycloalkyl, heterocycloalkyl, or heterocycloalkenyl, wherein said C₁-C₆alkyl, —C₂-C₆alkenyl, —C₂-C₆alkynyl, aryl, heteroaryl, cycloalkyl, heterocycloalkyl, or heterocycloalkenyl is optionally substituted;
or R⁵ and R⁶ together with the C atom to which they are attached form a cycloalkyl, heterocycloalkyl, or heterocycloalkenyl ring, each optionally substituted;
or an R⁵ and an R⁶ attached to adjacent carbon atoms, together with the C atoms to which they are attached, form a cycloalkyl, heterocycloalkyl, or heterocycloalkenyl ring, each optionally substituted;
or R⁷ and R⁸ together with the C atom to which they are both attached form a cycloalkyl, heterocycloalkyl, or heterocycloalkenyl ring, each optionally substituted, each optionally substituted;
or an R⁷ and an R⁸ attached to adjacent carbon atoms, together with the C atoms to which they are attached, form a cycloalkyl, heterocycloalkyl, or heterocycloalkenyl ring, each optionally substituted;
each R⁹ or R¹⁰ is independently H, D, -Me, CN, —CH₂CN, —CH₂F, —CHF₂, —CF₃ or —F;
each R¹² is H, D, —C(O)R^{b}, —C(O)OR^{c}, —C(O)NR^{c}R^{d}, P(OR)₂, P(O)R^{c}R^{b}, P(O)OR^{c}OR^{b}, S(O)R^{b}, S(O)NR^{c}R^{d}, S(O)₂R^{b}, S(O)₂NR^{c}R^{d}, B(OR^{c})(OR^{b}), SiR^{b}₃, C₁-C₁₀alkyl, C₂-C₁₀ alkenyl, C₂-C₁₀ alkynyl, —(CH₂CH₂O)ₒR^{a} wherein o=1 to 10, aryl, cycloalkyl, heteroaryl, or heterocycloalkyl, wherein said C₁-C₁₀ alkyl, C₂-C₁₀alkenyl, C₂-C₁₀ alkynyl, aryl, cycloalkyl, heteroaryl, or heterocycloalkyl is optionally substituted;
or R¹² and R¹¹ form an optionally substituted monocyclic or multicyclic heterocycloalkyl, or optionally substituted monocyclic or multicyclic heterocycloalkenyl group;
each R^{13A} or R^{13B} is independently H, D, optionally substituted C₁-C₆alkyl;
or R^{13A} and R^{13B} that are attached to the same carbon atom may, together with the carbon atom to which they are both attached, form an optionally substituted cycloalkyl ring;
each R¹⁴ or R¹⁵ is independently H, D, —C₁-C₆alkyl, —C₂-C₆alkenyl, —C₂-C₆alkynyl, —OC₁-C₆alkyl, —C(O)R^{b}, —C(O)OR^{b}, —C(O)NR^{c}R^{d}, —S(O)R^{b} or —S(O)₂R^{b}, aryl, heteroaryl, cycloalkyl, heterocycloalkyl, or heterocycloalkenyl group, wherein said C₁-C₆alkyl, —C₂-C₆alkenyl, —C₂-C₆alkynyl, —OC₁-C₆alkyl, aryl, heteroaryl, cycloalkyl, heterocycloalkyl, or heterocycloalkenyl ring, is optionally substituted;
or R¹⁴ together with an R⁴, R⁵, R⁷ or an R⁸ form an optionally substituted monocyclic or multicyclic heterocycloalkyl, or optionally substituted monocyclic or multicyclic heterocycloalkenyl group;
each R¹⁶ is independently H, D, —OH, -Me, —CH₂F, —CHF₂, —CF₃ or —F;
each R^{a} is independently H, D, —C(O)R^{b}, —C(O)OR^{c}, —C(O)NR^{c}R^{d}, —P(OR^{c})₂, —P(O)R^{c}R^{b}, —P(O)OR^{c}OR^{b}, —S(O)R^{b}, —S(O)NR^{c}R^{d}, —S(O)₂R^{b}, —S(O)₂NR^{c}R^{d}, —B(OR^{c})(OR^{b}), SiR^{b}₃, —C₁-C₁₀alkyl, —C₂-C₁₀ alkenyl, —C₂-C₁₀ alkynyl, aryl, cycloalkyl, heteroaryl, heterocycloalkyl, or heterocycloalkenyl wherein said C₁-C₁₀ alkyl, C₂-C₁₀ alkenyl, C₂-C₁₀ alkynyl, aryl, cycloalkyl, heteroaryl, heterocycloalkyl, or
heterocycloalkenyl is optionally substituted;
each R^{b}, is independently H, D, —C₁-C₆ alkyl, —C₂-C₆ alkenyl, —C₂-C₆ alkynyl, aryl, cycloalkyl, heteroaryl, heterocycloalkyl, or heterocycloalkenyl wherein said —C₁-C₆ alkyl, —C₂-C₆ alkenyl, —C₂-C₆ alkynyl, aryl, cycloalkyl, heteroaryl, heterocycloalkyl, or heterocycloalkenyl is optionally substituted;
each R^{c} or R^{d} is independently H, D, —C₁-C₁₀ alkyl, —C₂-C₆ alkenyl, —C₂-C₆ alkynyl, —OC₁-C₆alkyl, —O-cycloalkyl, aryl, heteroaryl, cycloalkyl, heterocycloalkyl, or heterocycloalkenyl, wherein said C₁-C₁₀ alkyl, C₂-C₆ alkenyl, C₂-C₆ alkynyl, —OC₁-C₆alkyl, —O-cycloalkyl, aryl, heteroaryl, cycloalkyl, heterocycloalkyl, or heterocycloalkenyl are each optionally substituted;
or R^{c} and R^{d}, together with the N atom to which they are both attached, form an optionally substituted monocyclic or multicyclic heterocycloalkyl, or optionally substituted monocyclic or multicyclic heterocycloalkenyl group.

2. The compound of claim 1, wherein s=1.

3. The compound of claim 2, wherein t=0; R^{2A} is H; R^{2B} is H; R^{13A} is H; and R^{13B} is H.

4. The compound of claim 3, wherein m=0.

5. The compound according to claim 1, wherein the compound has the structure IA:

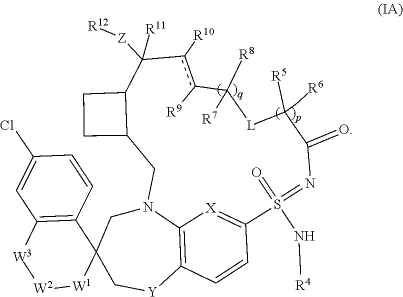

6. (canceled)

7. (canceled)

8. The compound according claim 1, wherein the compound has the structure IB:

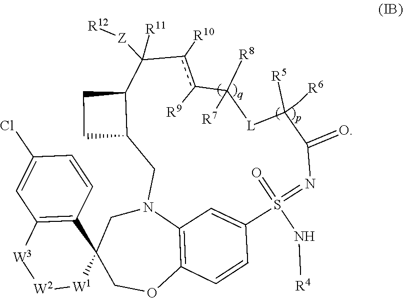

9.-27. (canceled)

28. The compound according to claim 8, wherein the compound of Formula IB has the structure IC:

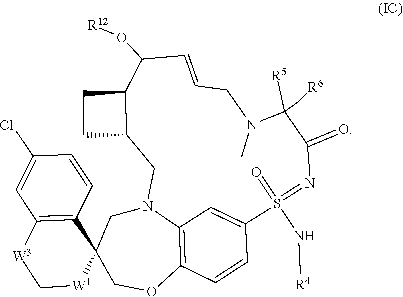

29. The compound according to claim 8, wherein the compound of Formula IB has the structure ID:

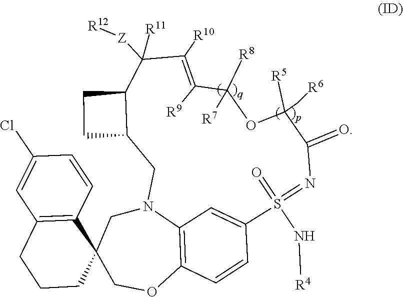

30. (canceled)

31. The compound according to claim 8, wherein the compound of Formula (IB) is a compound of Formula (IE):

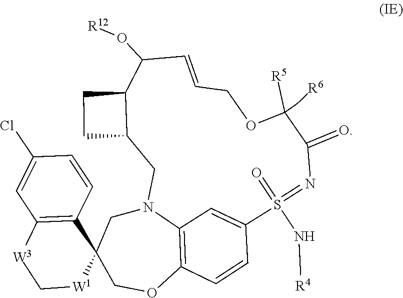

32. The compound according to claim 31, wherein the compound of Formula (IE) has the structure of Formula (IE-1):

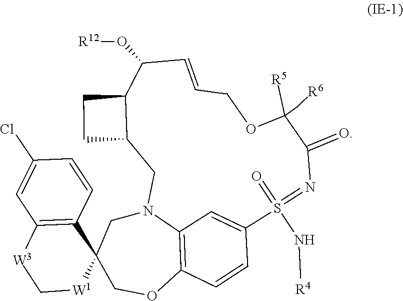

33.-46. (canceled)

47. The compound according to claim 32, wherein the compound of Formula (IE-1) is a compound of Formula (IE-1-1):

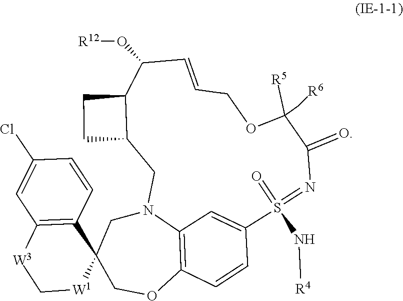

48. The compound according to claim 32, wherein the compound of Formula (IE-1) is a compound of Formula (IE-1-2):

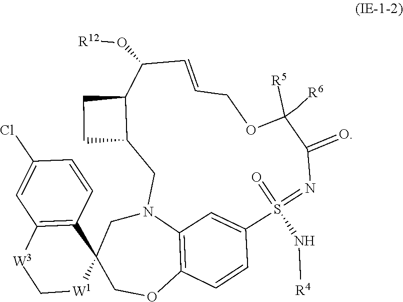

49. The compound according to claim 31, wherein the compound of Formula (IE) has the structure of Formula (IE-2):

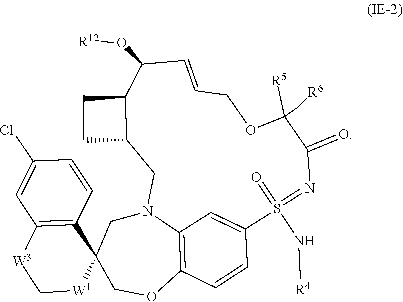

50.-63. (canceled)

64. The compound according to claim 49, wherein the compound of Formula (IE-2) is a compound of Formula (IE-2-1):

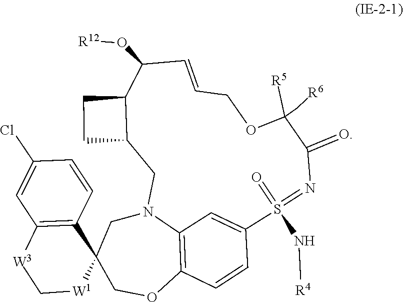

65. The compound according to claim 49, wherein the compound of Formula (IE-2) is a compound of Formula (IE-2-2):

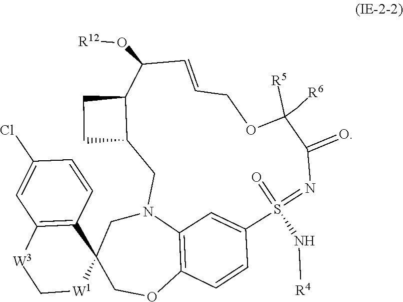

66. (canceled)

67. (canceled)

68. (canceled)

69. A pharmaceutical composition comprising a compound according to claim 1 and a pharmaceutically acceptable excipient.

70. A method of inhibiting an MCL-1 enzyme comprising contacting the MCL-1 enzyme with an effective amount of a compound of claim 1.

71. A method of treating a disease or disorder associated with aberrant MCL-1 activity in a subject comprising administering to the subject, a compound of claim 1.

72. The method of claim 71, wherein the disease or disorder associated with aberrant MCL-1 activity is colon cancer, breast cancer, small-cell lung cancer, non-small-cell lung cancer, bladder cancer, ovarian cancer, prostate cancer, chronic lymphoid leukemia, lymphoma, myeloma, acute myeloid leukemia, or pancreatic cancer.

73. The compound according to claim 1, or a pharmaceutically acceptable salt or solvate thereof, wherein the compound is
N-[(3R,6R,7S,8E,15R,22S)-7′-Chloro-7-methoxy-12,12-dimethyl-13,15-dioxospiro[11,20-dioxa-15-thia-1,14-diazatetracyclo[14.7.2.03,6.019,24]pentacosa-8,14,16,18,24-pentaene-22,4′-2,3-dihydro-1H-naphthalene]-15-yl]-2-methylpropanamide;
N-[(3R,6R,7S,8E,15S,22S)-7′-chloro-7-methoxy-12,12-dimethyl-13,15-dioxospiro[11,20-dioxa-15-thia-1,14-diazatetracyclo[14.7.2.03,6.019,24]pentacosa-8,14,16,18,24-pentaene-22,4′-2,3-dihydro-1H-naphthalene]-15-yl]-2-methylpropanamide;
N-[(3R,6R,7S,8E,15R,22S)-7′-Chloro-7-methoxy-12,12-dimethyl-13,15-dioxospiro[11,20-dioxa-15-thia-1,14-diazatetracyclo[14.7.2.03,6.019,24]pentacosa-8,14,16,18,24-pentaene-22,4′-2,3-dihydro-1H-naphthalene]-15-yl]acetamide;
N-[(3R,6R,7S,8E,15S,22S)-7′-chloro-7-methoxy-12,12-dimethyl-13,15-dioxospiro[11,20-dioxa-15-thia-1,14-diazatetracyclo[14.7.2.03,6.019,24]pentacosa-8,14,16,18,24-pentaene-22,4′-2,3-dihydro-1H-naphthalene]-15-yl]acetamide;
N-[(3R,6R,7S,8E,15R,22S)-7′-Chloro-7-methoxy-12,12-dimethyl-13,15-dioxospiro[11,20-dioxa-15-thia-1,14-diazatetracyclo[14.7.2.03,6.019,24]pentacosa-8,14,16,18,24-pentaene-22,4′-2,3-dihydro-1H-naphthalene]-15-yl]propenamide;
N-[(3R,6R,7S,8E,15S,22S)-7′-chloro-7-methoxy-12,12-dimethyl-13,15-dioxospiro[11,20-dioxa-15-thia-1,14-diazatetracyclo[14.7.2.03,6.019,24]pentacosa-8,14,16,18,24-pentaene-22,4′-2,3-dihydro-1H-naphthalene]-15-yl]propenamide;
N-[(3R,6R,7S,8E,15R,22S)-7′-Chloro-7-methoxy-12,12-dimethyl-13,15-dioxospiro[11,20-dioxa-15-thia-1,14-diazatetracyclo[14.7.2.03,6.019,24]pentacosa-8,14,16,18,24-pentaene-22,4′-2,3-dihydro-1H-naphthalene]-15-yl]-2-cyclopropylacetamide;
N-[(3R,6R,7S,8E,15S,22S)-7′-chloro-7-methoxy-12,12-dimethyl-13,15-dioxospiro[11,20-dioxa-15-thia-1,14-diazatetracyclo[14.7.2.03,6.019,24]pentacosa-8,14,16,18,24-pentaene-22,4′-2,3-dihydro-1H-naphthalene]-15-yl]-2-cyclopropylacetamide;
N-[(3R,6R,7S,8E,15R,22S)-7′-Chloro-7-methoxy-12,12-dimethyl-13,15-dioxospiro[11,20-dioxa-15-thia-1,14-diazatetracyclo[14.7.2.03,6.019,24]pentacosa-8,14,16,18,24-pentaene-22,4′-2,3-dihydro-1H-naphthalene]-15-yl]cyclobutanecarboxamide;
N-[(3R,6R,7S,8E,15S,22S)-7′-chloro-7-methoxy-12,12-dimethyl-13,15-dioxospiro[11,20-dioxa-15-thia-1,14-diazatetracyclo[14.7.2.03,6.019,24]pentacosa-8,14,16,18,24-pentaene-22,4′-2,3-dihydro-1H-naphthalene]-15-yl]cyclobutanecarboxamide;
N-[(3R,6R,7S,8E,22S)-7′-Chloro-7-methoxy-12,12-dimethyl-13,15-dioxospiro[11,20-dioxa-15-thia-1,14-diazatetracyclo[14.7.2.03,6.019,24]pentacosa-8,14,16,18,24-pentaene-22,4′-2,3-dihydro-1H-naphthalene]-15-yl]oxane-4-carboxamide;
N-[(3R,6R,7S,8E,22S)-7′-Chloro-7-methoxy-12,12-dimethyl-13,15-dioxospiro[11,20-dioxa-15-thia-1,14-diazatetracyclo[14.7.2.03,6.019,24]pentacosa-8,14,16,18,24-pentaene-22,4′-2,3-dihydro-1H-naphthalene]-15-yl]benzamide;
4-Bromo-N-[(3R,6R,7S,8E,15R,22S)-7′-chloro-7-methoxy-12,12-dimethyl-13,15-dioxospiro[11,20-dioxa-15-thia-1,14-diazatetracyclo[14.7.2.03,6.019,24]pentacosa-8,14,16,18,24-pentaene-22,4′-2,3-dihydro-1H-naphthalene]-15-yl]benzamide;
4-Bromo-N-[(3R,6R,7S,8E,15S,22S)-7′-chloro-7-methoxy-12,12-dimethyl-13,15-dioxospiro[11,20-dioxa-15-thia-1,14-diazatetracyclo[14.7.2.03,6.019,24]pentacosa-8,14,16,18,24-pentaene-22,4′-2,3-dihydro-1H-naphthalene]-15-yl]benzamide;
N-[(3R,6R,7S,8E,22S)-7′-Chloro-7-methoxy-12,12-dimethyl-13,15-dioxospiro[11,20-dioxa-15-thia-1,14-diazatetracyclo[14.7.2.03,6.019,24]pentacosa-8,14,16,18,24-pentaene-22,4′-2,3-dihydro-1H-naphthalene]-15-yl]-1-methylpyrazole-4-carboxamide;
N-[(3R,6R,7S,8E,22S)-7′-Chloro-7-methoxy-12,12-dimethyl-13,15-dioxospiro[11,20-dioxa-15-thia-1,14-diazatetracyclo[14.7.2.03,6.019,24]pentacosa-8,14,16,18,24-pentaene-22,4′-2,3-dihydro-1H-naphthalene]-15-yl]-3-methoxy-1-methylpyrazole-4-carboxamide;
N-[(3R,6R,7S,8E,22S)-7′-Chloro-7-methoxy-12,12-dimethyl-13,15-dioxospiro[11,20-dioxa-15-thia-1,14-diazatetracyclo[14.7.2.03,6.019,24]pentacosa-8,14,16,18,24-pentaene-22,4′-2,3-dihydro-1H-naphthalene]-15-yl]-1-methylpyrazole-4-carboxamide;
N-[(3R,6R,7S,8E,22S)-7′-Chloro-7-methoxy-12,12-dimethyl-13,15-dioxospiro[11,20-dioxa-15-thia-1,14-diazatetracyclo[14.7.2.03,6.019,24]pentacosa-8,14,16,18,24-pentaene-22,4′-2,3-dihydro-1H-naphthalene]-15-yl]-1,5-dimethylpyrazole-4-carboxamide;
N-[(3R,6R,7S,8E,22S)-7′-Chloro-7-methoxy-12,12-dimethyl-13,15-dioxospiro[11,20-dioxa-15-thia-1,14-diazatetracyclo[14.7.2.03,6.019,24]pentacosa-8,14,16,18,24-pentaene-22,4′-2,3-dihydro-1H-naphthalene]-15-yl]-1,5-dimethylpyrazole-3-carboxamide;
1-[(3R,6R,7S,8E,22S)-7′-Chloro-7-methoxy-12,12-dimethyl-13,15-dioxospiro[11,20-dioxa-15-thia-1,14-diazatetracyclo[14.7.2.03,6.019,24]pentacosa-8,14,16,18,24-pentaene-22,4′-2,3-dihydro-1H-naphthalene]-15-yl]-3-ethylurea;
1-[(3R,6R,7S,8E,15R,22S)-7′-Chloro-7-methoxy-12,12-dimethyl-13,15-dioxospiro[11,20-dioxa-15-thia-1,14-diazatetracyclo[14.7.2.03,6.019,24]pentacosa-8,14,16,18,24-pentaene-22,4′-2,3-dihydro-1H-naphthalene]-15-yl]-3-propan-2-ylurea;
1-[(3R,6R,7S,8E,15S,22S)-7′-Chloro-7-methoxy-12,12-dimethyl-13,15-dioxospiro[11,20-dioxa-15-thia-1,14-diazatetracyclo[14.7.2.03,6.019,24]pentacosa-8,14,16,18,24-pentaene-22,4′-2,3-dihydro-1H-naphthalene]-15-yl]-3-propan-2-ylurea;
1-[(3R,6R,7S,8E,15R,22S)-7′-Chloro-7-methoxy-12,12-dimethyl-13,15-dioxospiro[11,20-dioxa-15-thia-1,14-diazatetracyclo[14.7.2.03,6.019,24]pentacosa-8,14,16,18,24-pentaene-22,4′-2,3-dihydro-1H-naphthalene]-15-yl]-3-cyclopropylurea;
1-[(3R,6R,7S,8E,15S,22S)-7′-chloro-7-methoxy-12,12-dimethyl-13,15-dioxospiro[11,20-dioxa-15-thia-1,14-diazatetracyclo[14.7.2.03,6.019,24]pentacosa-8,14,16,18,24-pentaene-22,4′-2,3-dihydro-1H-naphthalene]-15-yl]-3-cyclopropylurea;
1-[(3R,6R,7S,8E,15R,22S)-7′-Chloro-7-methoxy-12,12-dimethyl-13,15-dioxospiro[11,20-dioxa-15-thia-1,14-diazatetracyclo[14.7.2.03,6.019,24]pentacosa-8,14,16,18,24-pentaene-22,4′-2,3-dihydro-1H-naphthalene]-15-yl]-3-cyclobutylurea;
1-[(3R,6R,7S,8E,15S,22S)-7′-chloro-7-methoxy-12,12-dimethyl-13,15-dioxospiro[11,20-dioxa-15-thia-1,14-diazatetracyclo[14.7.2.03,6.019,24]pentacosa-8,14,16,18,24-pentaene-22,4′-2,3-dihydro-1H-naphthalene]-15-yl]-3-cyclobutylurea;
N-[(3R,6R,7S,8E,15R,22S)-7′-Chloro-7-methoxy-12,12-dimethyl-13,15-dioxospiro[11,20-dioxa-15-thia-1,14-diazatetracyclo[14.7.2.03,6.019,24]pentacosa-8,14,16,18,24-pentaene-22,4′-2,3-dihydro-1H-naphthalene]-15-yl]azetidine-1-carboxamide;
N-[(3R,6R,7S,8E,15S,22S)-7′-chloro-7-methoxy-12,12-dimethyl-13,15-dioxospiro[11,20-dioxa-15-thia-1,14-diazatetracyclo[14.7.2.03,6.019,24]pentacosa-8,14,16,18,24-pentaene-22,4′-2,3-dihydro-1H-naphthalene]-15-yl]azetidine-1-carboxamide;
N-[(3R,6R,7S,8E,15R,22S)-7′-Chloro-7-methoxy-12,12-dimethyl-13,15-dioxospiro[11,20-dioxa-15-thia-1,14-diazatetracyclo[14.7.2.03,6.019,24]pentacosa-8,14,16,18,24-pentaene-22,4′-2,3-dihydro-1H-naphthalene]-15-yl]-3-hydroxyazetidine-1-carboxamide;
N-[(3R,6R,7S,8E,15S,22S)-7′-chloro-7-methoxy-12,12-dimethyl-13,15-dioxospiro[11,20-dioxa-15-thia-1,14-diazatetracyclo[14.7.2.03,6.019,24]pentacosa-8,14,16,18,24-pentaene-22,4′-2,3-dihydro-1H-naphthalene]-15-yl]-3-hydroxyazetidine-1-carboxamide;
N-[(3R,6R,7S,8E,15R,22S)-7′-Chloro-7-methoxy-12,12-dimethyl-13,15-dioxospiro[11,20-dioxa-15-thia-1,14-diazatetracyclo[14.7.2.03,6.019,24]pentacosa-8,14,16,18,24-pentaene-22,4′-2,3-dihydro-1H-naphthalene]-15-yl]-3-methoxyazetidine-1-carboxamide;
N-[(3R,6R,7S,8E,15S,22S)-7′-chloro-7-methoxy-12,12-dimethyl-13,15-dioxospiro[11,20-dioxa-15-thia-1,14-diazatetracyclo[14.7.2.03,6.019,24]pentacosa-8,14,16,18,24-pentaene-22,4′-2,3-dihydro-1H-naphthalene]-15-yl]-3-methoxyazetidine-1-carboxamide;
N-[(3R,6R,7S,8E,22S)-7′-Chloro-7-methoxy-12,12-dimethyl-13,15-dioxospiro[11,20-dioxa-15-thia-1,14-diazatetracyclo[14.7.2.03,6.019,24]pentacosa-8,14,16,18,24-pentaene-22,4′-2,3-dihydro-1H-naphthalene]-15-yl]-3-(dimethylamino)azetidine-1-carboxamide;
3-[(3R,6R,7S,8E,15R,22S)-7′-Chloro-7-methoxy-12,12-dimethyl-13,15-dioxospiro[11,20-dioxa-15 λ6-thia-1,14-diazatetracyclo[14.7.2.03,6.019,24]pentacosa-8,14,16,18,24-pentaene-22,4′-2,3-dihydro-1H-naphthalene]-15-yl]-1,1-dimethylurea;
3-[(3R,6R,7S,8E,15S,22S)-7′-chloro-7-methoxy-12,12-dimethyl-13,15-dioxospiro[11,20-dioxa-15 λ6-thia-1,14-diazatetracyclo[14.7.2.03,6.019,24]pentacosa-8,14,16,18,24-pentaene-22,4′-2,3-dihydro-1H-naphthalene]-15-yl]-1,1-dimethylurea;
1-[(3R,6R,7S,8E,15R,22S)-7′-Chloro-7-methoxy-12,12-dimethyl-13,15-dioxospiro[11,20-dioxa-15-thia-1,14-diazatetracyclo[14.7.2.03,6.019,24]pentacosa-8,14,16,18,24-pentaene-22,4′-2,3-dihydro-1H-naphthalene]-15-yl]-3-(4-(trans)-methoxycyclohexyl)urea;
1-[(3R,6R,7S,8E,15S,22S)-7′-chloro-7-methoxy-12,12-dimethyl-13,15-dioxospiro[11,20-dioxa-15-thia-1,14-diazatetracyclo[14.7.2.03,6.019,24]pentacosa-8,14,16,18,24-pentaene-22,4′-2,3-dihydro-1H-naphthalene]-15-yl]-3-(4-(trans)-methoxycyclohexyl)urea;
1-[(3R,6R,7S,8E,22S)-7′-Chloro-7-methoxy-12,12-dimethyl-13,15-dioxospiro[11,20-dioxa-15-thia-1,14-diazatetracyclo[14.7.2.03,6.019,24]pentacosa-8,14,16,18,24-pentaene-22,4′-2,3-dihydro-1H-naphthalene]-15-yl]-3-(oxan-4-yl)urea;
1-[(3R,6R,7S,8E,15R,22S)-7′-Chloro-7-methoxy-12,12-dimethyl-13,15-dioxospiro[11,20-dioxa-15-thia-1,14-diazatetracyclo[14.7.2.03,6.019,24]pentacosa-8,14,16,18,24-pentaene-22,4′-2,3-dihydro-1H-naphthalene]-15-yl]-3-(1-methylpyrazol-4-yl)urea;
1-[(3R,6R,7S,8E,15S,22S)-7′-chloro-7-methoxy-12,12-dimethyl-13,15-dioxospiro[11,20-dioxa-15-thia-1,14-diazatetracyclo[14.7.2.03,6.019,24]pentacosa-8,14,16,18,24-pentaene-22,4′-2,3-dihydro-1H-naphthalene]-15-yl]-3-(1-methylpyrazol-4-yl)urea;
N-[(3R,6R,7R,8E,15R,22S)-7′-Chloro-7-methoxy-12,12-dimethyl-13,15-dioxospiro[11,20-dioxa-15-thia-1,14-diazatetracyclo[14.7.2.03,6.019,24]pentacosa-8,14,16,18,24-pentaene-22,4′-2,3-dihydro-1H-naphthalene]-15-yl]acetamide;
N-[(3R,6R,7R,8E,15S,22S)-7′-chloro-7-methoxy-12,12-dimethyl-13,15-dioxospiro[11,20-dioxa-15-thia-1,14-diazatetracyclo[14.7.2.03,6.019,24]pentacosa-8,14,16,18,24-pentaene-22,4′-2,3-dihydro-1H-naphthalene]-15-yl]acetamide;
N-[(3R,6R,7R,8E,15R,22S)-7′-Chloro-7-methoxy-12,12-dimethyl-13,15-dioxospiro[11,20-dioxa-15-thia-1,14-diazatetracyclo[14.7.2.03,6.019,24]pentacosa-8,14,16,18,24-pentaene-22,4′-2,3-dihydro-1H-naphthalene]-15-yl]propenamide;
N-[(3R,6R,7R,8E,15S,22S)-7′-chloro-7-methoxy-12,12-dimethyl-13,15-dioxospiro[11,20-dioxa-15-thia-1,14-diazatetracyclo[14.7.2.03,6.019,24]pentacosa-8,14,16,18,24-pentaene-22,4′-2,3-dihydro-1H-naphthalene]-15-yl]propenamide;
N-[(3R,6R,7R,8E,15R,22S)-7′-Chloro-7-methoxy-12,12-dimethyl-13,15-dioxospiro[11,20-dioxa-15-thia-1,14-diazatetracyclo[14.7.2.03,6.019,24]pentacosa-8,14,16,18,24-pentaene-22,4′-2,3-dihydro-1H-naphthalene]-15-yl]-2-methylpropanamide;
N-[(3R,6R,7R,8E,15S,22S)-7′-chloro-7-methoxy-12,12-dimethyl-13,15-dioxospiro[11,20-dioxa-15-thia-1,14-diazatetracyclo[14.7.2.03,6.019,24]pentacosa-8,14,16,18,24-pentaene-22,4′-2,3-dihydro-1H-naphthalene]-15-yl]-2-methylpropanamide;
N-[(3R,6R,7R,8E,15R,22S)-7′-Chloro-7-methoxy-12,12-dimethyl-13,15-dioxospiro[11,20-dioxa-15-thia-1,14-diazatetracyclo[14.7.2.03,6.019,24]pentacosa-8,14,16,18,24-pentaene-22,4′-2,3-dihydro-1H-naphthalene]-15-yl]-1,5-dimethylpyrazole-4-carboxamide;
N-[(3R,6R,7R,8E,15S,22S)-7′-chloro-7-methoxy-12,12-dimethyl-13,15-dioxospiro[11,20-dioxa-15-thia-1,14-diazatetracyclo[14.7.2.03,6.019,24]pentacosa-8,14,16,18,24-pentaene-22,4′-2,3-dihydro-1H-naphthalene]-15-yl]-1,5-dimethylpyrazole-4-carboxamide;
N-[(3R,6R,7S,8E,15R,22S)-7′-Chloro-7-hydroxy-12,12-dimethyl-13,15-dioxospiro[11,20-dioxa-15-thia-1,14-diazatetracyclo[14.7.2.03,6.019,24]pentacosa-8,14,16,18,24-pentaene-22,4′-2,3-dihydro-1H-naphthalene]-15-yl]-1,3-dimethylpyrazole-4-carboxamide;
N-[(3R,6R,7S,8E,15S,22S)-7′-Chloro-7-hydroxy-12,12-dimethyl-13,15-dioxospiro[11,20-dioxa-15-thia-1,14-diazatetracyclo[14.7.2.03,6.019,24]pentacosa-8,14,16,18,24-pentaene-22,4′-2,3-dihydro-1H-naphthalene]-15-yl]-1,3-dimethylpyrazole-4-carboxamide;
[(3R,6R,7S,8E,22S)-7′-Chloro-15-[(1,3-dimethylpyrazole-4-carbonyl)amino]-12,12-dimethyl-13,15-dioxospiro[11,20-dioxa-15-thia-1,14-diazatetracyclo[14.7.2.03,6.019,24]pentacosa-8,14,16,18,24-pentaene-22,4′-2,3-dihydro-1H-naphthalene]-7-yl] N,N-dimethylcarbamate;
N-[(3R,6R,7S,8E,22S)-7′-Chloro-7-hydroxy-12,12-dimethyl-13,15-dioxospiro[11,20-dioxa-15-thia-1,14-diazatetracyclo[14.7.2.03,6.019,24]pentacosa-8,14,16,18,24-pentaene-22,4′-2,3-dihydro-1H-naphthalene]-15-yl]-1-methylpyrazole-4-carboxamide;
[(3R,6R,7S,8E,22S)-7′-Chloro-12,12-dimethyl-15-[(1-methylpyrazole-4-carbonyl)amino]-13,15-dioxospiro[11,20-dioxa-15-thia-1,14-diazatetracyclo[14.7.2.03,6.019,24]pentacosa-8,14,16,18,24-pentaene-22,4′-2,3-dihydro-1H-naphthalene]-7-yl] N,N-dimethylcarbamate;
N-[(3R,6R,7S,8E,22S)-7′-Chloro-12,12-dimethyl-13,15-dioxo-7-(2-pyrrolidin-1-ylethoxy)spiro[11,20-dioxa-15-thia-1,14-diazatetracyclo[14.7.2.03,6.019,24]pentacosa-8,14,16,18,24-pentaene-22,4′-2,3-dihydro-1H-naphthalene]-15-yl]-2-methylpropanamide; or
N-[(3R,6R,7S,8E,22S)-7′-Chloro-7-[2-(dimethylamino)ethoxy]-12,12-dimethyl-13,15-dioxospiro[11,20-dioxa-15-thia-1,14-diazatetracyclo[14.7.2.03,6.019,24]pentacosa-8,14,16,18,24-pentaene-22,4′-2,3-dihydro-1H-naphthalene]-15-yl]-2-methylpropanamide.
